SEQUENCE LISTING (255 sequences)

SEQ ID NO: 1 - DNA, 83 bp, synthetic construct
  source 1..83: mol_type = other DNA
ggggtataaaagcccccggcgcgtgattactgtcagtcttttggagggcctccctttagcgtgtgaggtgctgtaattccccc

SEQ ID NO: 2 - DNA, 83 bp, synthetic construct
  source 1..83: mol_type = other DNA
ggggtataaaaggcgggggcgcgtgattactgtcagtctgttgcaagatggcggtggtgcgtggaagcagttataaaacgaac

SEQ ID NO: 3 - DNA, 46 bp, synthetic construct
  source 1..46: mol_type = other DNA
gggttatataagggcgccgcgcgggtgattactgtcagtcttttgg

SEQ ID NO: 4 - DNA, 45 bp, synthetic construct
  source 1..45: mol_type = other DNA
tagagggtatataaaggaagctcgacttccagcttggcattccgg

SEQ ID NO: 5 - DNA, 45 bp, synthetic construct
  source 1..45: mol_type = other DNA
tagagggtatataatggaagctcgacttccagcttggcaatccgg

SEQ ID NO: 6 - DNA, 81 bp, synthetic construct
  source 1..81: mol_type = other DNA
gtacttatataagggggtgggggcgcgttcgtcctcagtcgcgatcgaacactcgagccgagcagacgtgcctacggaccg

SEQ ID NO: 7 - DNA, 42 bp, synthetic construct
  source 1..42: mol_type = other DNA
ggggtataaaagcccccggcgcgattactgacattcttttgg

SEQ ID NO: 8 - DNA, 44 bp, synthetic construct
  source 1..44: mol_type = other DNA
gggtacgcccctttttatgcgcgtgattactgcacaggaattgg

SEQ ID NO: 9 - DNA, 44 bp, synthetic construct
  source 1..44: mol_type = other DNA
tctagagggtatataatgggggccactagtctactaccagaaag

SEQ ID NO: 10 - DNA, 39 bp, synthetic construct
  source 1..39: mol_type = other DNA
gggttatataagggcgcctggaaggcgatttcagtcttg

SEQ ID NO: 11 - DNA, 83 bp, synthetic construct
  source 1..83: mol_type = other DNA
ggggtataaaagcccccggcgcgtgattactgtcagtctgttgggagaagcgggtgaaagcagggcggggctataactcggcg

SEQ ID NO: 12 - DNA, 83 bp, synthetic construct
  source 1..83: mol_type = other DNA
ggggtataaaaggcgggggcgcgtgattactgtcagtctgttgcaagatggcggcggtgcgtggaagcagttataaaacgaac

SEQ ID NO: 13 - DNA, 44 bp, synthetic construct
  source 1..44: mol_type = other DNA
gggttatataagggcgccgcgcgtgattactgacattcttttgg

SEQ ID NO: 14 - DNA, 80 bp, synthetic construct
  source 1..80: mol_type = other DNA
ggggtataaaaggcggggtggaattggcgatttcagtctggaaggggggtagggtgcgtggaagcagttataaaacgaac

SEQ ID NO: 15 - DNA, 45 bp, synthetic construct
  source 1..45: mol_type = other DNA
tagagggtatataagggaagctcgacttccagcttggcactccgg

SEQ ID NO: 16 - DNA, 80 bp, synthetic construct
  source 1..80: mol_type = other DNA
ggggtataaaaggcggggtggaattggcgatttcagtctgggacgagcgcgtggggggtgacggtggctgattatcacgg

SEQ ID NO: 17 - DNA, 80 bp, synthetic construct
  source 1..80: mol_type = other DNA
ggggtataaaaggcggggtggaattggcgatttcagtctggagggcctccctttagcgtgtgaggtgctgtaattccccc

SEQ ID NO: 18 - DNA, 83 bp, synthetic construct
  source 1..83: mol_type = other DNA
ggggtataaaaggcgggggcgcgtgattactgtcagtctgttgggacgagcgcgtggggggtgacggtggctgattatcacgg

SEQ ID NO: 19 - DNA, 83 bp, synthetic construct
  source 1..83: mol_type = other DNA
ggggtataaaaggcgggggcgcgtgattactgtcagtctgttggagggcctccctttagcgtgtgaggtgctgtaattccccc

SEQ ID NO: 20 - DNA, 83 bp, synthetic construct
  source 1..83: mol_type = other DNA
ggggtataaaagcccccggcgcgtgattactgtcagtctgttgggacgagcgcgtggggggtgacggtggctgattatcacgg

SEQ ID NO: 21 - DNA, 83 bp, synthetic construct
  source 1..83: mol_type = other DNA
ggggtataaaagcccccggcgcgtgattactgtcagtctgttggagggcctccctttagcgtgtgaggtgctgtaattccccc

SEQ ID NO: 22 - DNA, 44 bp, synthetic construct
  source 1..44: mol_type = other DNA
ggggtataaaaggctgaccgcagatgattaaatcagtcttttgg

SEQ ID NO: 23 - DNA, 44 bp, synthetic construct
  source 1..44: mol_type = other DNA
ggggtataaaagccggggaggaacagataagatcagtcttttgg

SEQ ID NO: 24 - DNA, 44 bp, synthetic construct
  source 1..44: mol_type = other DNA
ggggtataaaaggcgggggggttgtgcaatcgtcagtctgttgg

SEQ ID NO: 25 - DNA, 44 bp, synthetic construct
  source 1..44: mol_type = other DNA
ggggtataaaaggctgcttccacaagattagctcagtcttttgg

SEQ ID NO: 26 - DNA, 44 bp, synthetic construct
  source 1..44: mol_type = other DNA
ggggtataaaaggcggggaagtccaaggtccatcagtctgttgg

SEQ ID NO: 27 - DNA, 44 bp, synthetic construct
  source 1..44: mol_type = other DNA
ggggtataaaaggctgcttccacaagattagctcagtctgttgg

SEQ ID NO: 28 - DNA, 44 bp, synthetic construct
  source 1..44: mol_type = other DNA
ggggtataaaagcccccgaagtccaaggtccatcagtctgttgg

SEQ ID NO: 29 - DNA, 44 bp, synthetic construct
  source 1..44: mol_type = other DNA
ggggtataaaagcctgaccgcagatgattaaatcagtctgttgg

SEQ ID NO: 30 - DNA, 44 bp, synthetic construct
  source 1..44: mol_type = other DNA
ggggtataaaaggcggggaagtccaaggtccatcagtcttttgg

SEQ ID NO: 31 - DNA, 83 bp, synthetic construct
  source 1..83: mol_type = other DNA
ggggtataaaaggcgggggcgcgtgattactgtcagtcttttgcaagatggcggcggtgcgtggaagcagttataaaacgaac

SEQ ID NO: 32 - DNA, 83 bp, synthetic construct
  source 1..83: mol_type = other DNA
ggggtataaaagcccccggcgcgtgattactgtcattgacttgcaagatggcggcggtgcgtggaagcagttataaaacgaac

SEQ ID NO: 33 - DNA, 83 bp, synthetic construct
  source 1..83: mol_type = other DNA
ggggtataaaagcccccggcgcgtgattactgtcattgacttgcaagatggcggtggtgcgtggaagcagttataaaacgaac

SEQ ID NO: 34 - DNA, 83 bp, synthetic construct
  source 1..83: mol_type = other DNA
ggggtataaaagcccccggcgcgtgattactgtcattgacttgcaagatggcggctgcagtcccagttgcgatagaaaccccc

SEQ ID NO: 35 - DNA, 83 bp, synthetic construct
  source 1..83: mol_type = other DNA
ggggtataaaagcccccggcgcgtgattactgtcattgacttgcaagatggcggttgcagtcccagttgcgatagaaaccccc

SEQ ID NO: 36 - DNA, 80 bp, synthetic construct
  source 1..80: mol_type = other DNA
ggggtataaaaggcggggtggaattggcgatttcagtcttgaacaagatggcggcgcgtggaagcagttataaaacgaac

SEQ ID NO: 37 - DNA, 80 bp, synthetic construct
  source 1..80: mol_type = other DNA
ggggtataaaaggcggggtggaattggcgatttcagtcttgaacaagatggcggtgcgtggaagcagttataaaacgaac

SEQ ID NO: 38 - DNA, 80 bp, synthetic construct
  source 1..80: mol_type = other DNA
ggggtataaaaggcggggtggaattggcgatttcagtcttgaagccgccatcttggcgtggaagcagttataaaacgaac

SEQ ID NO: 39 - DNA, 83 bp, synthetic construct
  source 1..83: mol_type = other DNA
ggggtataaaagcccccggcgcgtgattactgtcagtcttttgcaagatggcggcggtgcgtggaagcagttataaaacgaac

SEQ ID NO: 40 - DNA, 83 bp, synthetic construct
  source 1..83: mol_type = other DNA
ggggtataaaagcccccggcgcgtgattactgtcagtcttttgcaaaatggcggcggtgcgtggaagcagttataaaacgaac

SEQ ID NO: 41 - DNA, 83 bp, synthetic construct
  source 1..83: mol_type = other DNA
ggggtataaaagcccccggcgcgtgattactgtcagtcttttgcaagatggcggtggtgcgtggaagcagttataaaacgaac

SEQ ID NO: 42 - DNA, 80 bp, synthetic construct
  source 1..80: mol_type = other DNA
ggggtataaaaggcggggtggaattggcgatttcagtctggaacaagatggcggtgcgtggaagcagttataaaacgaac

SEQ ID NO: 43 - DNA, 83 bp, synthetic construct
  source 1..83: mol_type = other DNA
ggggtataaaaggcgggggcgcgtgattactgtcagtcttttgcaaaatggcggcggtgcgtggaagcagttataaaacgaac

SEQ ID NO: 44 - DNA, 83 bp, synthetic construct
  source 1..83: mol_type = other DNA
ggggtataaaagcccccggcgcgtgattactgtcagtcttttgaaatatggcgctggtgcgtggaagcagttataaaacgaac

SEQ ID NO: 45 - DNA, 83 bp, synthetic construct
  source 1..83: mol_type = other DNA
ggggtataaaagcccccggcgcgtgattactgtcagtcttttgagcgccatatttggtgcgtggaagcagttataaaacgaac

SEQ ID NO: 46 - DNA, 80 bp, synthetic construct
  source 1..80: mol_type = other DNA
ggggtataaaaggcggggtggaattggcgatttcattgacgaaaccgccatcttggcgtggaagcagttataaaacgaac

SEQ ID NO: 47 - DNA, 83 bp, synthetic construct
  source 1..83: mol_type = other DNA
ggggtataaaagcccccggcgcgtgattactgtcagtcttttgcaagatggcggctgcagtcccagttgcgatagaaaccccc

SEQ ID NO: 48 - DNA, 83 bp, synthetic construct
  source 1..83: mol_type = other DNA
ggggtataaaagcccccggcgcgtgattactgtcagtcttttgcaaaatggcggctgcagtcccagttgcgatagaaaccccc

SEQ ID NO: 49 - DNA, 83 bp, synthetic construct
  source 1..83: mol_type = other DNA
ggggtataaaaggcgggggcgcgtgattactgtcagtcttttgaaatatggcgctggtgcgtggaagcagttataaaacgaac

SEQ ID NO: 50 - DNA, 83 bp, synthetic construct
  source 1..83: mol_type = other DNA
ggggtataaaaggcgggggcgcgtgattactgtcagtctgttgcaaaatggcggcggtgcgtggaagcagttataaaacgaac

SEQ ID NO: 51 - DNA, 83 bp, synthetic construct
  source 1..83: mol_type = other DNA
ggggtataaaaggcgggggcgcgtgattactgtcagtctgttgaaatatggcgctggtgcgtggaagcagttataaaacgaac

SEQ ID NO: 52 - DNA, 83 bp, synthetic construct
  source 1..83: mol_type = other DNA
ggggtataaaaggcgggggcgcgtgattactgtcagtctgttgaccgccatcttgggtgcgtggaagcagttataaaacgaac

SEQ ID NO: 53 - DNA, 83 bp, synthetic construct
  source 1..83: mol_type = other DNA
ggggtataaaaggcgggggcgcgtgattactgtcagtctgttgcaagatggcggctgcagtcccagttgcgatagaaaccccc

SEQ ID NO: 54 - DNA, 83 bp, synthetic construct
  source 1..83: mol_type = other DNA
ggggtataaaaggcgggggcgcgtgattactgtcagtctgttgcaaaatggcggctgcagtcccagttgcgatagaaaccccc

SEQ ID NO: 55 - DNA, 83 bp, synthetic construct
  source 1..83: mol_type = other DNA
ggggtataaaaggcgggggcgcgtgattactgtcagtctgttgcaagatggcggttgcagtcccagttgcgatagaaaccccc

SEQ ID NO: 56 - DNA, 83 bp, synthetic construct
  source 1..83: mol_type = other DNA
ggggtataaaagcccccggcgcgtgattactgtcagtctgttgcaagatggcggcggtgcgtggaagcagttataaaacgaac

SEQ ID NO: 57 - DNA, 83 bp, synthetic construct
  source 1..83: mol_type = other DNA
ggggtataaaagcccccggcgcgtgattactgtcagtctgttgcaaaatggcggcggtgcgtggaagcagttataaaacgaac

SEQ ID NO: 58 - DNA, 83 bp, synthetic construct
  source 1..83: mol_type = other DNA
ggggtataaaagcccccggcgcgtgattactgtcagtctgttgcaagatggcggtggtgcgtggaagcagttataaaacgaac

SEQ ID NO: 59 - DNA, 83 bp, synthetic construct
  source 1..83: mol_type = other DNA
ggggtataaaagcccccggcgcgtgattactgtcagtctgttgaaatatggcgctggtgcgtggaagcagttataaaacgaac

SEQ ID NO: 60 - DNA, 83 bp, synthetic construct
  source 1..83: mol_type = other DNA
ggggtataaaagcccccggcgcgtgattactgtcagtctgttggccgccatcttgggtgcgtggaagcagttataaaacgaac

SEQ ID NO: 61 - DNA, 83 bp, synthetic construct
  source 1..83: mol_type = other DNA
ggggtataaaagcccccggcgcgtgattactgtcagtctgttgcaagatggcggctgcagtcccagttgcgatagaaaccccc

SEQ ID NO: 62 - DNA, 83 bp, synthetic construct
  source 1..83: mol_type = other DNA
ggggtataaaagcccccggcgcgtgattactgtcagtctgttgcaagatggcggttgcagtcccagttgcgatagaaaccccc

SEQ ID NO: 63 - DNA, 83 bp, synthetic construct
  source 1..83: mol_type = other DNA
ggggtataaaagcccccggcgcgtgattactgtcagtctgttgaaatatggcgcttgcagtcccagttgcgatagaaaccccc

SEQ ID NO: 64 - DNA, 83 bp, synthetic construct
  source 1..83: mol_type = other DNA
ggggtataaaagcccccggcgcgtgattactgtcagtctgttgaccgccatcttgtgcagtcccagttgcgatagaaaccccc

SEQ ID NO: 65 - DNA, 83 bp, synthetic construct
  source 1..83: mol_type = other DNA
ggggtataaaaggcgggggcgcgtgattactgtcagtcttttgagcgccatatttggtgcgtggaagcagttataaaacgaac

SEQ ID NO: 66 - DNA, 83 bp, synthetic construct
  source 1..83: mol_type = other DNA
ggggtataaaaggcgggggcgcgtgattactgtcagtcttttgcaagatggcggctgcagtcccagttgcgatagaaaccccc

SEQ ID NO: 67 - DNA, 83 bp, synthetic construct
  source 1..83: mol_type = other DNA
ggggtataaaaggcgggggcgcgtgattactgtcattgacttgcaagatggcggcggtgcgtggaagcagttataaaacgaac

SEQ ID NO: 68 - DNA, 83 bp, synthetic construct
  source 1..83: mol_type = other DNA
ggggtataaaaggcgggggcgcgtgattactgtcattgacttgcaagatggcggtggtgcgtggaagcagttataaaacgaac

SEQ ID NO: 69 - DNA, 46 bp, synthetic construct
  source 1..46: mol_type = other DNA
ggggtataaaaggcgggggcgcgggtgattactgtcagtcttttgg

SEQ ID NO: 70 - DNA, 46 bp, synthetic construct
  source 1..46: mol_type = other DNA
ggggtataaaaggcgggggcgcgggtgattactgtcagtctcttgg

SEQ ID NO: 71 - DNA, 43 bp, synthetic construct
  source 1..43: mol_type = other DNA
ggggtataaaaggcggggtggaaggttggcgatttcagtcttg

SEQ ID NO: 72 - DNA, 43 bp, synthetic construct
  source 1..43: mol_type = other DNA
ggggtataaaagcccccgtggaaggttggcgatttcagtcttg

SEQ ID NO: 73 - DNA, 43 bp, synthetic construct
  source 1..43: mol_type = other DNA
gggttatataagggcgcctggaaggttggcgatttcagtcttg

SEQ ID NO: 74 - DNA, 46 bp, synthetic construct
  source 1..46: mol_type = other DNA
ggggtataaaaagcgggggcgcgggtgattactgtcagtctcttgg

SEQ ID NO: 75 - DNA, 43 bp, synthetic construct
  source 1..43: mol_type = other DNA
ggggtataaaaggcggggtggaaggttggcgatttcagtctcg

SEQ ID NO: 76 - DNA, 43 bp, synthetic construct
  source 1..43: mol_type = other DNA
gggttatataagggcgcctggaaggttggcgatttcagtctcg

SEQ ID NO: 77 - DNA, 46 bp, synthetic construct
  source 1..46: mol_type = other DNA
ggggtataaaagcccccggcgcgggtgattactgtcagtctcttgg

SEQ ID NO: 78 - DNA, 43 bp, synthetic construct
  source 1..43: mol_type = other DNA
ggggtataaaaggcggggtggaaggttggcgatttcagtctgg

SEQ ID NO: 79 - DNA, 43 bp, synthetic construct
  source 1..43: mol_type = other DNA
ggggtataaaaagcggggtggaaggttggcgatttcagtctgg

SEQ ID NO: 80 - DNA, 43 bp, synthetic construct
  source 1..43: mol_type = other DNA
gggttatataagggcgcctggaaggttggcgatttcagtctgg

SEQ ID NO: 81 - DNA, 43 bp, synthetic construct
  source 1..43: mol_type = other DNA
ggggtataaaaggcggggtggaaggttggcgatttcagtagtg

SEQ ID NO: 82 - DNA, 43 bp, synthetic construct
  source 1..43: mol_type = other DNA
ggggtataaaaagcggggtggaaggttggcgatttcagtagtg

SEQ ID NO: 83 - DNA, 43 bp, synthetic construct
  source 1..43: mol_type = other DNA
ggggtataaaagcccccgtggaaggttggcgatttcagtagtg

SEQ ID NO: 84 - DNA, 43 bp, synthetic construct
  source 1..43: mol_type = other DNA
gggttatataagggcgcctggaaggttggcgatttcagtagtg

SEQ ID NO: 85 - DNA, 43 bp, synthetic construct
  source 1..43: mol_type = other DNA
ggggtataaaaggcggggtggaaggttggcgattacattcttg

SEQ ID NO: 86 - DNA, 46 bp, synthetic construct
  source 1..46: mol_type = other DNA
gggttatataagggcgccgcgcgggtgattactgtcagtctcttgg

SEQ ID NO: 87 - DNA, 43 bp, synthetic construct
  source 1..43: mol_type = other DNA
ggggtataaaaggcggggtggaaggttggcgatttcatatccg

SEQ ID NO: 88 - DNA, 43 bp, synthetic construct
  source 1..43: mol_type = other DNA
gggttatataagggcgcctggaaggttggcgatttcatatccg

SEQ ID NO: 89 - DNA, 43 bp, synthetic construct
  source 1..43: mol_type = other DNA
gggttatataagggcgcctggaaggttggcgatttcattgacg

SEQ ID NO: 90 - DNA, 43 bp, synthetic construct
  source 1..43: mol_type = other DNA
ggggtataaaaggcggggtggaaggttggcgatttcacaaacg

SEQ ID NO: 91 - DNA, 43 bp, synthetic construct
  source 1..43: mol_type = other DNA
ggggtataaaaagcggggtggaaggttggcgatttcacaaacg

SEQ ID NO: 92 - DNA, 43 bp, synthetic construct
  source 1..43: mol_type = other DNA
gggttatataagggcgcctggaaggttggcgatttcacaaacg

SEQ ID NO: 93 - DNA, 43 bp, synthetic construct
  source 1..43: mol_type = other DNA
ggggtataaaaggcggggtggaaggttggcgattcacaggaag

SEQ ID NO: 94 - DNA, 46 bp, synthetic construct
  source 1..46: mol_type = other DNA
ggggtataaaaagcgggggcgcgggtgattactgtcagtctgttgg

SEQ ID NO: 95 - DNA, 46 bp, synthetic construct
  source 1..46: mol_type = other DNA
ggggtataaaagcccccggcgcgggtgattactgtcagtctgttgg

SEQ ID NO: 96 - DNA, 46 bp, synthetic construct
  source 1..46: mol_type = other DNA
gggttatataagggcgccgcgcgggtgattactgtcagtctgttgg

SEQ ID NO: 97 - DNA, 46 bp, synthetic construct
  source 1..46: mol_type = other DNA
ggggtataaaagcccccggcgcgggtgattactgtcagtcttttgg

SEQ ID NO: 98 - DNA, 46 bp, synthetic construct
  source 1..46: mol_type = other DNA
ggggtataaaaggcgggggcgcgggtgattactgtcagtagtttgg

SEQ ID NO: 99 - DNA, 46 bp, synthetic construct
  source 1..46: mol_type = other DNA
ggggtataaaagcccccggcgcgggtgattactgtcagtagtttgg

SEQ ID NO: 100 - DNA, 46 bp, synthetic construct
  source 1..46: mol_type = other DNA
gggttatataagggcgccgcgcgggtgattactgtcagtagtttgg

SEQ ID NO: 101 - DNA, 46 bp, synthetic construct
  source 1..46: mol_type = other DNA
ggggtataaaaggcgggggcgcgggtgattactgacattcttttgg

SEQ ID NO: 102 - DNA, 46 bp, synthetic construct
  source 1..46: mol_type = other DNA
ggggtataaaagcccccggcgcgggtgattactgacattcttttgg

SEQ ID NO: 103 - DNA, 46 bp, synthetic construct
  source 1..46: mol_type = other DNA
gggttatataagggcgccgcgcgggtgattactgacattcttttgg

SEQ ID NO: 104 - DNA, 46 bp, synthetic construct
  source 1..46: mol_type = other DNA
ggggtataaaagcccccggcgcgggtgattactgtcatatccttgg

SEQ ID NO: 105 - DNA, 46 bp, synthetic construct
  source 1..46: mol_type = other DNA
gggttatataagggcgccgcgcgggtgattactgtcatatccttgg

SEQ ID NO: 106 - DNA, 46 bp, synthetic construct
  source 1..46: mol_type = other DNA
gggttatataagggcgccgcgcgggtgattactgtcattgacttgg

SEQ ID NO: 107 - DNA, 46 bp, synthetic construct
  source 1..46: mol_type = other DNA
ggggtataaaagcccccggcgcgggtgattactgtcacaaacttgg

SEQ ID NO: 108 - DNA, 46 bp, synthetic construct
  source 1..46: mol_type = other DNA
gggttatataagggcgccgcgcgggtgattactgtcacaaacttgg

SEQ ID NO: 109 - DNA, 42 bp, synthetic construct
  source 1..42: mol_type = other DNA
ggggtataaaaggcgggggcgcgattactgtcagtcttttgg

SEQ ID NO: 110 - DNA, 39 bp, synthetic construct
  source 1..39: mol_type = other DNA
ggggtataaaaggcggggtggaaggcgatttcagtcttg

SEQ ID NO: 111 - DNA, 39 bp, synthetic construct
  source 1..39: mol_type = other DNA
ggggtataaaaggcggggtggaaggcgatttcagtctcg

SEQ ID NO: 112 - DNA, 39 bp, synthetic construct
  source 1..39: mol_type = other DNA
gggttatataagggcgcctggaaggcgatttcagtctcg

SEQ ID NO: 113 - DNA, 39 bp, synthetic construct
  source 1..39: mol_type = other DNA
ggggtataaaaggcggggtggaaggcgatttcagtagtg

SEQ ID NO: 114 - DNA, 39 bp, synthetic construct
  source 1..39: mol_type = other DNA
gggttatataagggcgcctggaaggcgatttcagtagtg

SEQ ID NO: 115 - DNA, 39 bp, synthetic construct
  source 1..39: mol_type = other DNA
ggggtataaaaagcggggtggaaggcgattacattcttg

SEQ ID NO: 116 - DNA, 42 bp, synthetic construct
  source 1..42: mol_type = other DNA
gggttatataagggcgccgcgcgattactgtcagtctcttgg

SEQ ID NO: 117 - DNA, 39 bp, synthetic construct
  source 1..39: mol_type = other DNA
gggttatataagggcgcctggaaggcgattacattcttg

SEQ ID NO: 118 - DNA, 39 bp, synthetic construct
  source 1..39: mol_type = other DNA
ggggtataaaaggcggggtggaaggcgattcacaggaag

SEQ ID NO: 119 - DNA, 39 bp, synthetic construct
  source 1..39: mol_type = other DNA
ggggtataaaaagcggggtggaaggcgattcacaggaag

SEQ ID NO: 120 - DNA, 39 bp, synthetic construct
  source 1..39: mol_type = other DNA
ggggtataaaagcccccgtggaaggcgattcacaggaag

SEQ ID NO: 121 - DNA, 42 bp, synthetic construct
  source 1..42: mol_type = other DNA
gggttatataagggcgccgcgcgattactgtcagtagtttgg

SEQ ID NO: 122 - DNA, 42 bp, synthetic construct
  source 1..42: mol_type = other DNA
ggggtataaaaggcgggggcgcgattactgacattcttttgg

SEQ ID NO: 123 - DNA, 42 bp, synthetic construct
  source 1..42: mol_type = other DNA
ggggtataaaaagcgggggcgcgattactgacattcttttgg

SEQ ID NO: 124 - DNA, 42 bp, synthetic construct
  source 1..42: mol_type = other DNA
gggttatataagggcgccgcgcgattactgacattcttttgg

SEQ ID NO: 125 - DNA, 42 bp, synthetic construct
  source 1..42: mol_type = other DNA
gggttatataagggcgccgcgcgattactgtcagtcttttgg

SEQ ID NO: 126 - DNA, 44 bp, synthetic construct
  source 1..44: mol_type = other DNA
ggggtataaaaggcgggggcgcgtgattactgtcagtcttttgg

SEQ ID NO: 127 - DNA, 44 bp, synthetic construct
  source 1..44: mol_type = other DNA
ggggtataaaaggcgggggcgcgtgattactgtcagtctcttgg

SEQ ID NO: 128 - DNA, 41 bp, synthetic construct
  source 1..41: mol_type = other DNA
gggttatataagggcgcctggaattggcgatttcagtcttg

SEQ ID NO: 129 - DNA, 41 bp, synthetic construct
  source 1..41: mol_type = other DNA
gggttatataagggcgcctggaattggcgatttcagtctcg

SEQ ID NO: 130 - DNA, 44 bp, synthetic construct
  source 1..44: mol_type = other DNA
ggggtataaaagcccccggcgcgtgattactgtcagtctcttgg

SEQ ID NO: 131 - DNA, 41 bp, synthetic construct
  source 1..41: mol_type = other DNA
gggttatataagggcgcctggaattggcgatttcagtctgg

SEQ ID NO: 132 - DNA, 41 bp, synthetic construct
  source 1..41: mol_type = other DNA
gggttatataagggcgcctggaattggcgatttcagtagtg

SEQ ID NO: 133 - DNA, 44 bp, synthetic construct
  source 1..44: mol_type = other DNA
gggttatataagggcgccgcgcgtgattactgtcagtctcttgg

SEQ ID NO: 134 - DNA, 44 bp, synthetic construct
  source 1..44: mol_type = other DNA
ggggtataaaaggcgggggcgcgtgattactgtcagtctgttgg

SEQ ID NO: 135 - DNA, 44 bp, synthetic construct
  source 1..44: mol_type = other DNA
ggggtataaaagcccccggcgcgtgattactgtcagtctgttgg

SEQ ID NO: 136 - DNA, 44 bp, synthetic construct
  source 1..44: mol_type = other DNA
gggttatataagggcgccgcgcgtgattactgtcagtctgttgg

SEQ ID NO: 137 - DNA, 44 bp, synthetic construct
  source 1..44: mol_type = other DNA
ggggtataaaagcccccggcgcgtgattactgtcagtcttttgg

SEQ ID NO: 138 - DNA, 44 bp, synthetic construct
  source 1..44: mol_type = other DNA
ggggtataaaaggcgggggcgcgtgattactgtcagtagtttgg

SEQ ID NO: 139 - DNA, 44 bp, synthetic construct
  source 1..44: mol_type = other DNA
ggggtataaaagcccccggcgcgtgattactgtcagtagtttgg

SEQ ID NO: 140 - DNA, 44 bp, synthetic construct
  source 1..44: mol_type = other DNA
gggttatataagggcgccgcgcgtgattactgtcagtagtttgg

SEQ ID NO: 141 - DNA, 44 bp, synthetic construct
  source 1..44: mol_type = other DNA
ggggtataaaagcccccggcgcgtgattactgacattcttttgg

SEQ ID NO: 142 - DNA, 44 bp, synthetic construct
  source 1..44: mol_type = other DNA
gggttatataagggcgccgcgcgtgattactgtcagtcttttgg

SEQ ID NO: 143 - DNA, 44 bp, synthetic construct
  source 1..44: mol_type = other DNA
ggggtataaaaggcgggggcgcgtgattactgtcacaaacttgg

SEQ ID NO: 144 - DNA, 44 bp, synthetic construct
  source 1..44: mol_type = other DNA
gggttatataagggcgccgcgcgtgattactgtcacaaacttgg

SEQ ID NO: 145 - DNA, 171 bp, synthetic construct
  source 1..171: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcac

SEQ ID NO: 146 - DNA, 133 bp, synthetic construct
  source 1..133: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttcc

SEQ ID NO: 147 - DNA, 145 bp, synthetic construct
  source 1..145: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggctt

SEQ ID NO: 148 - DNA, 254 bp, synthetic construct
  source 1..254: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaagcccccggcgcgtgattactgtcagtcttttggagggcctccctttagcgtgtgaggtgctgtaattccccc

SEQ ID NO: 149 - DNA, 254 bp, synthetic construct
  source 1..254: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaaggcgggggcgcgtgattactgtcagtctgttgcaagatggcggtggtgcgtggaagcagttataaaacgaac

SEQ ID NO: 150 - DNA, 217 bp, synthetic construct
  source 1..217: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacgggttatataagggcgccgcgcgggtgattactgtcagtcttttgg

SEQ ID NO: 151 - DNA, 216 bp, synthetic construct
  source 1..216: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcactagagggtatataaaggaagctcgacttccagcttggcattccgg

SEQ ID NO: 152 - DNA, 216 bp, synthetic construct
  source 1..216: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcactagagggtatataatggaagctcgacttccagcttggcaatccgg

SEQ ID NO: 153 - DNA, 252 bp, synthetic construct
  source 1..252: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacgtacttatataagggggtgggggcgcgttcgtcctcagtcgcgatcgaacactcgagccgagcagacgtgcctacggaccg

SEQ ID NO: 154 - DNA, 213 bp, synthetic construct
  source 1..213: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaagcccccggcgcgattactgacattcttttgg

SEQ ID NO: 155 - DNA, 215 bp, synthetic construct
  source 1..215: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacgggtacgcccctttttatgcgcgtgattactgcacaggaattgg

SEQ ID NO: 156 - DNA, 215 bp, synthetic construct
  source 1..215: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcactctagagggtatataatgggggccactagtctactaccagaaag

SEQ ID NO: 157 - DNA, 210 bp, synthetic construct
  source 1..210: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacgggttatataagggcgcctggaaggcgatttcagtcttg

SEQ ID NO: 158 - DNA, 254 bp, synthetic construct
  source 1..254: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaagcccccggcgcgtgattactgtcagtctgttgggagaagcgggtgaaagcagggcggggctataactcggcg

SEQ ID NO: 159 - DNA, 254 bp, synthetic construct
  source 1..254: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaaggcgggggcgcgtgattactgtcagtctgttgcaagatggcggcggtgcgtggaagcagttataaaacgaac

SEQ ID NO: 160 - DNA, 215 bp, synthetic construct
  source 1..215: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacgggttatataagggcgccgcgcgtgattactgacattcttttgg

SEQ ID NO: 161 - DNA, 251 bp, synthetic construct
  source 1..251: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaaggcggggtggaattggcgatttcagtctggaaggggggtagggtgcgtggaagcagttataaaacgaac

SEQ ID NO: 162 - DNA, 216 bp, synthetic construct
  source 1..216: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcactagagggtatataagggaagctcgacttccagcttggcactccgg

SEQ ID NO: 163 - DNA, 216 bp, synthetic construct
  source 1..216: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaagcccccggcgcgtgattactgtcagtcttttggagggcctccctttagcgtgtgaggtgctgtaattccccc

SEQ ID NO: 164 - DNA, 216 bp, synthetic construct
  source 1..216: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaaggcgggggcgcgtgattactgtcagtctgttgcaagatggcggtggtgcgtggaagcagttataaaacgaac

SEQ ID NO: 165 - DNA, 179 bp, synthetic construct
  source 1..179: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccgggttatataagggcgccgcgcgggtgattactgtcagtcttttgg

SEQ ID NO: 166 - DNA, 178 bp, synthetic construct
  source 1..178: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttcctagagggtatataaaggaagctcgacttccagcttggcattccgg

SEQ ID NO: 167 - DNA, 178 bp, synthetic construct
  source 1..178: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttcctagagggtatataatggaagctcgacttccagcttggcaatccgg

SEQ ID NO: 168 - DNA, 214 bp, synthetic construct
  source 1..214: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccgtacttatataagggggtgggggcgcgttcgtcctcagtcgcgatcgaacactcgagccgagcagacgtgcctacggaccg

SEQ ID NO: 169 - DNA, 175 bp, synthetic construct
  source 1..175: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaagcccccggcgcgattactgacattcttttgg

SEQ ID NO: 170 - DNA, 177 bp, synthetic construct
  source 1..177: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccgggtacgcccctttttatgcgcgtgattactgcacaggaattgg

SEQ ID NO: 171 - DNA, 177 bp, synthetic construct
  source 1..177: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttcctctagagggtatataatgggggccactagtctactaccagaaag

SEQ ID NO: 172 - DNA, 172 bp, synthetic construct
  source 1..172: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccgggttatataagggcgcctggaaggcgatttcagtcttg

SEQ ID NO: 173 - DNA, 216 bp, synthetic construct
  source 1..216: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaagcccccggcgcgtgattactgtcagtctgttgggagaagcgggtgaaagcagggcggggctataactcggcg

SEQ ID NO: 174 - DNA, 216 bp, synthetic construct
  source 1..216: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaaggcgggggcgcgtgattactgtcagtctgttgcaagatggcggcggtgcgtggaagcagttataaaacgaac

SEQ ID NO: 175 - DNA, 177 bp, synthetic construct
  source 1..177: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccgggttatataagggcgccgcgcgtgattactgacattcttttgg

SEQ ID NO: 176 - DNA, 213 bp, synthetic construct
  source 1..213: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaaggcggggtggaattggcgatttcagtctggaaggggggtagggtgcgtggaagcagttataaaacgaac

SEQ ID NO: 177 - DNA, 178 bp, synthetic construct
  source 1..178: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttcctagagggtatataagggaagctcgacttccagcttggcactccgg

SEQ ID NO: 178 - DNA, 228 bp, synthetic construct
  source 1..228: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttggggtataaaagcccccggcgcgtgattactgtcagtcttttggagggcctccctttagcgtgtgaggtgctgtaattccccc

SEQ ID NO: 179 - DNA, 228 bp, synthetic construct
  source 1..228: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttggggtataaaaggcgggggcgcgtgattactgtcagtctgttgcaagatggcggtggtgcgtggaagcagttataaaacgaac

SEQ ID NO: 180 - DNA, 191 bp, synthetic construct
  source 1..191: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttgggttatataagggcgccgcgcgggtgattactgtcagtcttttgg

SEQ ID NO: 181 - DNA, 190 bp, synthetic construct
  source 1..190: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggctttagagggtatataaaggaagctcgacttccagcttggcattccgg

SEQ ID NO: 182 - DNA, 190 bp, synthetic construct
  source 1..190: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggctttagagggtatataatggaagctcgacttccagcttggcaatccgg

SEQ ID NO: 183 - DNA, 226 bp, synthetic construct
  source 1..226: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttgtacttatataagggggtgggggcgcgttcgtcctcagtcgcgatcgaacactcgagccgagcagacgtgcctacggaccg

SEQ ID NO: 184 - DNA, 187 bp, synthetic construct
  source 1..187: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttggggtataaaagcccccggcgcgattactgacattcttttgg

SEQ ID NO: 185 - DNA, 189 bp, synthetic construct
  source 1..189: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttgggtacgcccctttttatgcgcgtgattactgcacaggaattgg

SEQ ID NO: 186 - DNA, 189 bp, synthetic construct
  source 1..189: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggctttctagagggtatataatgggggccactagtctactaccagaaag

SEQ ID NO: 187 - DNA, 184 bp, synthetic construct
  source 1..184: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttgggttatataagggcgcctggaaggcgatttcagtcttg

SEQ ID NO: 188 - DNA, 228 bp, synthetic construct
  source 1..228: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttggggtataaaagcccccggcgcgtgattactgtcagtctgttgggagaagcgggtgaaagcagggcggggctataactcggcg

SEQ ID NO: 189 - DNA, 228 bp, synthetic construct
  source 1..228: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttggggtataaaaggcgggggcgcgtgattactgtcagtctgttgcaagatggcggcggtgcgtggaagcagttataaaacgaac

SEQ ID NO: 190 - DNA, 189 bp, synthetic construct
  source 1..189: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttgggttatataagggcgccgcgcgtgattactgacattcttttgg

SEQ ID NO: 191 - DNA, 225 bp, synthetic construct
  source 1..225: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggcttggggtataaaaggcggggtggaattggcgatttcagtctggaaggggggtagggtgcgtggaagcagttataaaacgaac

SEQ ID NO: 192 - DNA, 190 bp, synthetic construct
  source 1..190: mol_type = other DNA
gccactgcactccgtgctgggcaactgagtgagaccccatctcaaaaaactcaaaaaagagttaaaataaatcactgtctattgtgccaaagagctgttgaagtcatcattcttagaagaatagacagatggtatttcaaggctttagagggtatataagggaagctcgacttccagcttggcactccgg

SEQ ID NO: 193 - DNA, 133 bp, synthetic construct; identical to SEQ ID NO: 146
  source 1..133: mol_type = other DNA

SEQ ID NO: 194 - DNA, 145 bp, synthetic construct
  source 1..145: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatat

SEQ ID NO: 195 - DNA, 59 bp, synthetic construct
  source 1..59: mol_type = other DNA
aaaacgacggccagtgaattgacgcgtattgggatggaacggcctccacggccactagt

SEQ ID NO: 196 - DNA, 39 bp, synthetic construct
  source 1..39: mol_type = other DNA
gacgagcgcgtggggggtgacggtggctgattatcacgg

SEQ ID NO: 197 - DNA, 39 bp, synthetic construct
  source 1..39: mol_type = other DNA
ggggagcgggatgttgtcgacggccggggataagccctc

SEQ ID NO: 198 - DNA, 39 bp, synthetic construct
  source 1..39: mol_type = other DNA
agggcctccctttagcgtgtgaggtgctgtaattccccc

SEQ ID NO: 199 - DNA, 15 bp, synthetic construct
  source 1..15: mol_type = other DNA
gtataaaaagcgggg

SEQ ID NO: 200 - DNA, 15 bp, synthetic construct
  source 1..15: mol_type = other DNA
gtataaaaggcgggg

SEQ ID NO: 201 - DNA, 15 bp, synthetic construct
  source 1..15: mol_type = other DNA
ttatataagggcgcc

SEQ ID NO: 202-204 - skipped (3 entries)

SEQ ID NO: 205 - DNA, 12 bp, synthetic construct
  source 1..12: mol_type = other DNA
caagatggcggt

SEQ ID NO: 206 - DNA, 12 bp, synthetic construct
  source 1..12: mol_type = other DNA
caagatggcggc

SEQ ID NO: 207 - DNA, 12 bp, synthetic construct
  source 1..12: mol_type = other DNA
caaaatggcggc

SEQ ID NO: 208 - DNA, 44 bp, synthetic construct
  source 1..44: mol_type = other DNA
gggtgactggagtgcttagcgcgtgattactgctggaggattgg

SEQ ID NO: 209 - DNA, 41 bp, synthetic construct
  source 1..41: mol_type = other DNA
gggttactgctggaggattggaattggcgattcttacgcgg

SEQ ID NO: 210 - DNA, 111 bp, synthetic construct
  source 1..111: mol_type = other DNA
aggaccggatcaactaggtctatataagcagagctcgtttagtgaaccgtcagatcgcctggagacgccatccacgctgttttgacctccatagaattggtaccgagctcg

SEQ ID NO: 211 - DNA, 39 bp, synthetic construct
  source 1..39: mol_type = other DNA
gagaagcgggtgaaagcagggcggggctataactcggcg

SEQ ID NO: 212 - DNA, 39 bp, synthetic construct
  source 1..39: mol_type = other DNA
aaggggggtagggtgcgtggaagcagttataaaacgaac

SEQ ID NO: 213 - DNA, 15 bp, synthetic construct
  source 1..15: mol_type = other DNA
gtataaaagcccccg

SEQ ID NO: 214 - DNA, 11 bp, synthetic construct
  source 1..11: mol_type = other DNA
ggtatataaag

SEQ ID NO: 215 - DNA, 11 bp, synthetic construct
  source 1..11: mol_type = other DNA
ggtatataagg

SEQ ID NO: 216-217 - skipped (2 entries)

SEQ ID NO: 218 - DNA, 210 bp, synthetic construct
  source 1..210: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacggggtataaaaggcggggtggaaggcgatttcagtcttg

SEQ ID NO: 219 - DNA, 282 bp, synthetic construct
  source 1..282: mol_type = other DNA
tgctatttttagcgggctttttttgacgggaggaaggaaggagggagagggacgggaagtaaagagaaaaagaggaagtgaaagctaagaaggaagtgacggctggcggggacagataagagttgtctagttgtgataatggaactgctgagtcatggattgcagagtcacaggaccggatcaactaggtctatataagcagagctcgtttagtgaaccgtcagatcgcctggagacgccatccacgctgttttgacctccatagaattggtaccgagctcg

SEQ ID NO: 220 - DNA, 172 bp, synthetic construct
  source 1..172: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccggggtataaaaggcggggtggaaggcgatttcagtcttg

SEQ ID NO: 221 - DNA, 244 bp, synthetic construct
  source 1..244: mol_type = other DNA
ggttaatgattaaccgtgtaaataattagcggatcacgtgatggtcacgtgttggttcaaggccagagtcaaggtcgggagtccaaagtccagaagtgcaaggtccggtgtttactttgggtgtttaccttccaggaccggatcaactaggtctatataagcagagctcgtttagtgaaccgtcagatcgcctggagacgccatccacgctgttttgacctccatagaattggtaccgagctcg

SEQ ID NO: 222 - DNA, 216 bp, synthetic construct; identical to SEQ ID NO: 163
  source 1..216: mol_type = other DNA

SEQ ID NO: 223 - DNA, 216 bp, synthetic construct; identical to SEQ ID NO: 164
  source 1..216: mol_type = other DNA

SEQ ID NO: 224 - DNA, 179 bp, synthetic construct; identical to SEQ ID NO: 165
  source 1..179: mol_type = other DNA

SEQ ID NO: 225 - DNA, 178 bp, synthetic construct; identical to SEQ ID NO: 166
  source 1..178: mol_type = other DNA

SEQ ID NO: 226 - DNA, 178 bp, synthetic construct; identical to SEQ ID NO: 167
  source 1..178: mol_type = other DNA

SEQ ID NO: 227 - DNA, 214 bp, synthetic construct; identical to SEQ ID NO: 168
  source 1..214: mol_type = other DNA

SEQ ID NO: 228 - DNA, 175 bp, synthetic construct; identical to SEQ ID NO: 169
  source 1..175: mol_type = other DNA

SEQ ID NO: 229 - DNA, 177 bp, synthetic construct; identical to SEQ ID NO: 170
  source 1..177: mol_type = other DNA

SEQ ID NO: 230 - DNA, 177 bp, synthetic construct; identical to SEQ ID NO: 171
  source 1..177: mol_type = other DNA

SEQ ID NO: 231 - DNA, 172 bp, synthetic construct; identical to SEQ ID NO: 172
  source 1..172: mol_type = other DNA

SEQ ID NO: 232 - DNA, 216 bp, synthetic construct; identical to SEQ ID NO: 173
  source 1..216: mol_type = other DNA

SEQ ID NO: 233 - DNA, 216 bp, synthetic construct; identical to SEQ ID NO: 174
  source 1..216: mol_type = other DNA

SEQ ID NO: 234 - DNA, 177 bp, synthetic construct; identical to SEQ ID NO: 175
  source 1..177: mol_type = other DNA

SEQ ID NO: 235 - DNA, 213 bp, synthetic construct; identical to SEQ ID NO: 176
  source 1..213: mol_type = other DNA

SEQ ID NO: 236 - DNA, 178 bp, synthetic construct; identical to SEQ ID NO: 177
  source 1..178: mol_type = other DNA

SEQ ID NO: 237 - DNA, 172 bp, synthetic construct; identical to SEQ ID NO: 220
  source 1..172: mol_type = other DNA

SEQ ID NO: 238 - DNA, 244 bp, synthetic construct; identical to SEQ ID NO: 221
  source 1..244: mol_type = other DNA

SEQ ID NO: 239 - DNA, 228 bp, synthetic construct
  source 1..228: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaagcccccggcgcgtgattactgtcagtcttttggagggcctccctttagcgtgtgaggtgctgtaattccccc

SEQ ID NO: 240 - DNA, 228 bp, synthetic construct
  source 1..228: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaaggcgggggcgcgtgattactgtcagtctgttgcaagatggcggtggtgcgtggaagcagttataaaacgaac

SEQ ID NO: 241 - DNA, 191 bp, synthetic construct
  source 1..191: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatgggttatataagggcgccgcgcgggtgattactgtcagtcttttgg

SEQ ID NO: 242 - DNA, 190 bp, synthetic construct
  source 1..190: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatattagagggtatataaaggaagctcgacttccagcttggcattccgg

SEQ ID NO: 243 - DNA, 190 bp, synthetic construct
  source 1..190: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatattagagggtatataatggaagctcgacttccagcttggcaatccgg

SEQ ID NO: 244 - DNA, 226 bp, synthetic construct
  source 1..226: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatgtacttatataagggggtgggggcgcgttcgtcctcagtcgcgatcgaacactcgagccgagcagacgtgcctacggaccg

SEQ ID NO: 245 - DNA, 187 bp, synthetic construct
  source 1..187: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaagcccccggcgcgattactgacattcttttgg

SEQ ID NO: 246 - DNA, 189 bp, synthetic construct
  source 1..189: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatgggtacgcccctttttatgcgcgtgattactgcacaggaattgg

SEQ ID NO: 247 - DNA, 189 bp, synthetic construct
  source 1..189: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatattctagagggtatataatgggggccactagtctactaccagaaag

SEQ ID NO: 248 - DNA, 184 bp, synthetic construct
  source 1..184: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatgggttatataagggcgcctggaaggcgatttcagtcttg

SEQ ID NO: 249 - DNA, 228 bp, synthetic construct
  source 1..228: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaagcccccggcgcgtgattactgtcagtctgttgggagaagcgggtgaaagcagggcggggctataactcggcg

SEQ ID NO: 250 - DNA, 228 bp, synthetic construct
  source 1..228: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaaggcgggggcgcgtgattactgtcagtctgttgcaagatggcggcggtgcgtggaagcagttataaaacgaac

SEQ ID NO: 251 - DNA, 189 bp, synthetic construct
  source 1..189: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatgggttatataagggcgccgcgcgtgattactgacattcttttgg

SEQ ID NO: 252 - DNA, 225 bp, synthetic construct
  source 1..225: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaaggcggggtggaattggcgatttcagtctggaaggggggtagggtgcgtggaagcagttataaaacgaac

SEQ ID NO: 253 - DNA, 190 bp, synthetic construct
  source 1..190: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatattagagggtatataagggaagctcgacttccagcttggcactccgg

SEQ ID NO: 254 - DNA, 184 bp, synthetic construct
  source 1..184: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatatggggtataaaaggcggggtggaaggcgatttcagtcttg

SEQ ID NO: 255 - DNA, 256 bp, synthetic construct
  source 1..256: mol_type = other DNA
agaaacacggcacagatgacggctttaaaagaaaagagtccattgactggaaaagcaaacagtatgatggtcaaaggccaagaatcagaaaacagagatttttcctcttctcttgtttcacaaagtaaataaacatgagacatataggaccggatcaactaggtctatataagcagagctcgtttagtgaaccgtcagatcgcctggagacgccatccacgctgttttgacctccatagaattggtaccgagctcg

(19) United States
(12) Patent Application Publication
(10) Pub. No.: US 2025/0222139 A1
(43) Pub. Date: Jul. 10, 2025

(54) PROMOTER SWITCHES FOR TISSUE-SPECIFIC EXPRESSION

(71) Applicant: SHAPE THERAPEUTICS INC., Seattle, WA (US)

(72) Inventors: Jacob Michael TOME, Seattle, WA (US); Richard Thomas SULLIVAN, Seattle, WA (US); Stephanie KALL, Seattle, WA (US); Sandra Blair SHANNON, Seattle, WA (US)

(21) Appl. No.: 18/853,976

(22) PCT Filed: Apr. 7, 2023
(86) PCT No.: PCT/US2023/017923

§ 371 Date: Oct. 3, 2024

Related U.S. Application Data
(60) Provisional application No. 63/329,211, filed on Apr. 8, 2022.
(60) Provisional application No. 63/423,977, filed on Nov. 9, 2022.

(51) Int. Cl. A61K 48/00; C12N 15/10; C12N 15/86

(52) U.S. Cl. CPC A61K 48/0058; C12N 15/1082; C12N 15/86; C12N 2750/14143; C12N 2830/008; C12N 2830/15

(57) ABSTRACT

Described herein are switchable core promoters that may selectively promote transcription initiation in the presence of an activated response element, such as an activated enhancer. These switchable core promoters may be paired with cell type- or cell state-specific response element to produce engineered promoters that selectively promote transcription of a payload sequence in a target cell type or target cell state. Also described herein are methods of using switchable core promoters and polynucleotides containing switchable core promoters to selectively express a payload in a target cell type or target cell state.

### CROSS-REFERENCE

The present application claims the benefit of U.S. Provisional Application No. 63/329,211, entitled “PROMOTER SWITCHES FOR TISSUE-SPECIFIC EXPRESSION,” filed on Apr. 8, 2022, and U.S. Provisional Application No. 63/423,977, entitled “PROMOTER SWITCHES FOR TISSUE-SPECIFIC EXPRESSION,” filed on Nov. 9, 2022, each of which applications are herein incorporated by reference in their entireties for all purposes.

### BACKGROUND

A wide variety of diseases and disorders are caused by mutations, deletions, or altered expression of genes. Many of these genes are tightly regulated in healthy individuals such that over-expression or under-expression of the gene may result in detrimental side effects. Additionally, some diseases and disorders are characterized by different cell genotypes of healthy and diseased cells within a subject. As a result, expression of a gene, such as a transgene, may be therapeutic in one cell type but detrimental in another cell type. While substantial progress is being made toward delivery of transgenes into individuals for treatment of genetic disorders, there remains a need for gene therapies that can regulate transgene expression in a cell-type or cell state dependent manner.

### SUMMARY

In various aspects, the present disclosure provides an engineered core promoter, wherein the engineered core promoter comprises a sequence having at least 80% sequence identity to SEQ ID NO: 4 and including nucleotide substitutions T15G and A40T relative to SEQ ID NO: 5.

In some aspects, the engineered core promoter comprises a sequence having at least 90% sequence identity to SEQ ID NO: 4 and including the nucleotide substitutions T15G and A40T relative to SEQ ID NO: 5. In some aspects, the engineered core promoter comprises a sequence of SEQ ID NO: 4.

In some aspects, the engineered core promoter further comprises a transcriptional pause site. In some aspects, the transcriptional pause site comprises a sequence having at least 90% sequence identity to any one of SEQ ID NO: 196-SEQ ID NO: 198, SEQ ID NO: 211, or SEQ ID NO: 212. In some aspects, the transcriptional pause site comprises a sequence of any one of SEQ ID NO: 196-SEQ ID NO: 198, SEQ ID NO: 211, or SEQ ID NO: 212. In some aspects, the engineered core promoter further comprises a YY1 motif. In some aspects, the YY1 motif comprises a sequence having at least 90% sequence identity to any one of SEQ ID NO: 205-SEQ ID NO: 207. In some aspects, the YY1 motif comprises a sequence having a sequence of any one of SEQ ID NO: 205-SEQ ID NO: 207. In some aspects, the engineered core promoter comprises a spacer of 30 to 34 nucleotides.

In some aspects, a transcriptional activity produced by the engineered core promoter paired with an activated response element is higher than a transcriptional activity produced by a core promoter of SEQ ID NO: 6 when paired with the activated response element, a basal transcriptional activity produced by the engineered core promoter paired with an activated response element is lower than a basal transcriptional activity produced by a core promoter of SEQ ID NO: 6 when paired with an inactive response element, or both. In some aspects, a transcriptional activity produced by the engineered core promoter paired with an activated response element is higher than a transcriptional activity produced by a core promoter of SEQ ID NO: 9 when paired with the activated response element, a basal transcriptional activity produce by the engineered core promoter paired with an activated response element is lower than a basal transcriptional activity produced by a core promoter of SEQ ID NO: 9 when paired with an inactive response element, or both.

In various aspects, the present disclosure provides an engineered promoter comprising a response element and an engineered core promoter as described herein.

In some aspects, the response element confers bone marrow-specific transcription, liver-specific transcription, neuron-specific transcription, muscle-specific transcription, or kidney-specific transcription of a payload under transcriptional control of the engineered promoter. In some aspects, the response element comprises a sequence having at least 90% sequence identity to SEQ ID NO: 145, SEQ ID NO: 146, SEQ ID NO: 193, or SEQ ID NO: 194. In some aspects, the response element comprises a sequence of SEQ ID NO: 145, SEQ ID NO: 146, SEQ ID NO: 193, or SEQ ID NO: 194.

In various aspects, the present disclosure provides a recombinant polynucleotide comprising an engineered core promoter as described herein or an engineered promoter as described herein and a payload comprising a coding sequence under transcriptional control of the engineered core promoter.

In some aspects, the payload encodes a protein. In some aspects, the protein is a neuronal protein, a kidney protein, a retinal protein, a muscle protein, or an apoptosis-inducing protein. In some aspects, the protein is associated with a genetic disorder, a neuronal disorder, a kidney disorder, an eye disorder, a muscular disorder, or a cancer. In some aspects, the protein is progranulin, MeCP2, polycystin-1, or polycystin-2.

In some aspects, the payload encodes a therapeutic polynucleotide. In some aspects, the therapeutic polynucleotide is a guide RNA or a suppressor tRNA. In some aspects, the therapeutic polynucleotide targets a gene. In some aspects, the gene is associated with a genetic disorder, a neuronal disorder, a kidney disorder, an eye disorder, a muscular disorder, or a cancer. In some aspects, the gene is GRN, MECP2, PKD2, or PKD2.

In various aspects, the present disclosure provides an engineered viral vector comprising an engineered core promoter as described herein, an engineered promoter as described herein, or a recombinant polynucleotide as described herein in a viral vector.

In some aspects, the viral vector is an adenoviral vector, an adeno-associated viral vector, or a lentivector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, and combinations thereof.

In various aspects, the present disclosure provides a pharmaceutical composition comprising an engineered core promoter as described herein, an engineered promoter as described herein, a recombinant polynucleotide as described herein, or a viral vector as described herein, and a pharmaceutically acceptable carrier.

In various aspects, the present disclosure provides a method of treating a disorder in a subject in need thereof, the method comprising: administering to the subject a composition comprising a recombinant polynucleotide as described herein, a viral vector as described herein, or a pharmaceutical composition as described herein; and expressing a therapeutic sequence encoded by a payload of the recombinant polynucleotide in a target cell of the subject, thereby treating the disorder.

In some aspects, the target cell is a cell type or cell state associated with the disorder. In some aspects, the disorder is a genetic disorder, a neuronal disorder, a kidney disorder, an eye disorder, a muscular disorder, or a cancer. In some aspects, the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease. In some aspects, the therapeutic sequence encodes a therapeutic protein. In some aspects, the therapeutic protein is a neuronal protein, a kidney protein, a retinal protein, a muscle protein, or an apoptosis-inducing protein. In some aspects, the therapeutic protein is associated with a genetic disorder, a neuronal disorder, a kidney disorder, an eye disorder, a muscular disorder, or a cancer. In some aspects, the therapeutic protein is MECP2, progranulin, polycystin-1, or polycystin-2. In some aspects, the payload encodes a therapeutic polynucleotide. In some aspects, the therapeutic polynucleotide is a guide RNA or a suppressor tRNA.

In various aspects, the present disclosure provides a method of identifying a switchable core promoter, the method comprising: introducing a core promoter library comprising a first sub-library and a second sub-library to a population of cells; wherein the first sub-library comprises a plurality of core promoters, wherein a core promoter of the plurality of core promoters is linked to a first response element, and a unique barcode sequence; wherein the second sub-library comprises the plurality of core promoters, wherein the core promoter of the plurality of promoters is linked to a second response element and, a unique barcode sequence; and identifying a switchable core promoter as the core promoter that promotes higher transcription of the unique barcode when paired with the first response element than when paired with the second response element.

In some aspects, the method further comprises activating the first response element. In some aspects, the second response element is not activated. In some aspects, the first response element is an activated response element and the second enhancer sequence is an inactive response element or an unactivated response element.

In various aspects, the present disclosure provides an engineered core promoter comprising a TATA box, an initiator element, a spacer separating the TATA box and the initiator element, and one or more features selected from: a) the TATA box comprises a sequence having at least 85% sequence identity to any one of SEQ ID NO: 199-SEQ ID NO: 201 or SEQ ID NO: 213-SEQ ID NO: 215; b) the initiator element comprises a sequence having at least 75% sequence identity to any one of SEQ ID NO: 202-SEQ ID NO: 204, SEQ ID NO: 216, or SEQ ID NO: 217; c) the spacer is a short spacer comprising no less than 25 and no more than 29 nucleotide residues from the TATA box to the initiator element; d) the spacer is a long spacer comprising no less than 31 and no more than 48 nucleotide residues from the TATA box to the initiator element; e) a YY1 motif comprising a sequence having at least 80% sequence identity to any one of SEQ ID NO: 205-SEQ ID NO: 207; f) a transcriptional pause site comprising a sequence having at least 90% sequence identity to any one of SEQ ID NO: 196-SEQ ID NO: 198, SEQ ID NO: 211, or SEQ ID NO: 212; g) at least 70% sequence identity and at least one nucleotide substitution relative to SEQ ID NO: 5, SEQ ID NO: 9, or SEQ ID NO: 210; and h) combinations thereof.

In some aspects, the engineered core promoter comprises at least 70% sequence identity and nucleotide substitutions T15G and A40C relative to SEQ ID NO: 5. In some aspects, the engineered core promoter comprises at least 70% sequence identity and nucleotide substitutions T15A and A40T relative to SEQ ID NO: 5.

In some aspects, the spacer comprises from 29 to 31 nucleotides from the TATA box to the initiator element. In some aspects, the spacer comprises 30 nucleotides from the TATA box to the initiator element. In some aspects, the short spacer comprises 28 nucleotide residues from the TATA box to the initiator element. In some aspects, the long spacer comprises 32 nucleotide residues from the TATA box to the initiator element.

In some aspects, the TATA box comprises a sequence having at least 90% sequence identity to any one of SEQ ID NO: 199-SEQ ID NO: 201 or SEQ ID NO: 213-SEQ ID NO: 215. In some aspects, the TATA box comprises a sequence of any one of SEQ ID NO: 199-SEQ ID NO: 201 or SEQ ID NO: 213-SEQ ID NO: 215.

In some aspects, the initiator element comprises a sequence having at least 85% sequence identity to any one of SEQ ID NO: 202-SEQ ID NO: 204, SEQ ID NO: 216, or SEQ ID NO: 217. In some aspects, the initiator element comprises a sequence of any one of SEQ ID NO: 202-SEQ ID NO: 204, SEQ ID NO: 216, or SEQ ID NO: 217.

In some aspects, the YY1 motif comprises a sequence having at least 90% sequence identity to any one of SEQ ID NO: 205-SEQ ID NO: 207. In some aspects, the YY1 motif comprises a sequence of any one of SEQ ID NO: 205-SEQ ID NO: 207.

In some aspects, the transcriptional pause site comprises a sequence having at least 95% sequence identity to any one of SEQ ID NO: 196-SEQ ID NO: 198, SEQ ID NO: 211, or SEQ ID NO: 212. In some aspects, the transcriptional pause site comprises a sequence of any one of SEQ ID NO: 196-SEQ ID NO: 198, SEQ ID NO: 211, or SEQ ID NO: 212.

In some aspects, the engineered core promoter comprises two or more of the features. In some aspects, the engineered core promoter comprises features a) and b). In some aspects, the engineered core promoter comprises three or more of the features. In some aspects, the engineered core promoter comprises features a) and b) and at least one more of the features. In some aspects, the engineered core promoter comprises features a), b), and c). In some aspects, the engineered core promoter comprises features a), b), and d). In some aspects, the engineered core promoter comprises features a), b), and e). In some aspects, the engineered core promoter comprises features a), b), and f).

In various aspects, the present disclosure provides an engineered core promoter comprising a sequence having at least 80%, at least 85%, at least 90%, at least 93%, at least 95%, at least 97%, at least 99%, or 100% sequence identity to any one of SEQ ID NO: 1-SEQ ID NO: 4, SEQ ID NO: 7, or SEQ ID NO: 10-SEQ ID NO: 144.

In some aspects, the engineered core promoter comprises a sequence having at least 90% sequence identity to any one of SEQ ID NO: 1-SEQ ID NO: 4, SEQ ID NO: 7, or SEQ ID NO: 10-SEQ ID NO: 144. In some aspects, the engineered core promoter comprises a sequence having at least 80%, at least 85%, at least 90%, at least 93%, at least 95%, at least 97%, at least 99%, or 100% sequence identity to any one of SEQ ID NO: 1-SEQ ID NO: 4, SEQ ID NO: 7, SEQ ID NO: 10-SEQ ID NO: 15, or SEQ ID NO: 110. In some aspects, the engineered core promoter comprises a sequence having at least 90% sequence identity to any one of SEQ ID NO: 1-SEQ ID NO: 4, SEQ ID NO: 7, SEQ ID NO: 10-SEQ ID NO: 15, or SEQ ID NO: 110.

In some aspects, the engineered core promoter comprises a sequence having at least 90% sequence identity to SEQ ID NO: 7. In some aspects, the engineered core promoter comprises a sequence of SEQ ID NO: 7. In some aspects, the engineered core promoter comprises a sequence having at least 90% sequence identity to SEQ ID NO: 1. In some aspects, the engineered core promoter comprises a sequence of SEQ ID NO: 1. In some aspects, the engineered core promoter comprises a sequence having at least 90% sequence identity to SEQ ID NO: 2. In some aspects, the engineered core promoter comprises a sequence of SEQ ID NO: 2. In some aspects, the engineered core promoter comprises a sequence having at least 90% sequence identity to SEQ ID NO: 3. In some aspects, the engineered core promoter comprises a sequence of SEQ ID NO: 3. In some aspects, the engineered core promoter comprises a sequence having at least 90% sequence identity to SEQ ID NO: 4 and including nucleotide substitutions T15G and A40T relative to SEQ ID NO: 5. In some aspects, the engineered core promoter comprises a sequence of SEQ ID NO: 4. In some aspects, the engineered core promoter comprises a sequence having at least 90% sequence identity to SEQ ID NO: 10. In some aspects, the engineered core promoter comprises a sequence of SEQ ID NO: 10. In some aspects, the engineered core promoter comprises a sequence having at least 90% sequence identity to SEQ ID NO: 11. In some aspects, the engineered core promoter comprises a sequence of SEQ ID NO: 11. In some aspects, the engineered core promoter comprises a sequence having at least 90% sequence identity to SEQ ID NO: 12. In some aspects, the engineered core promoter comprises a sequence of SEQ ID NO: 12. In some aspects, the engineered core promoter comprises a sequence having at least 90% sequence identity to SEQ ID NO: 13. In some aspects, the engineered core promoter comprises a sequence of SEQ ID NO: 13. In some aspects, the engineered core promoter comprises a sequence having at least 90% sequence identity to SEQ ID NO: 14. In some aspects, the engineered core promoter comprises a sequence of SEQ ID NO: 14. In some aspects, the engineered core promoter comprises a sequence having at least 90% sequence identity to SEQ ID NO: 15 and including nucleotide substitutions T15G and A40C relative to SEQ ID NO: 5. In some aspects, the engineered core promoter comprises a sequence of SEQ ID NO: 15. In some aspects, the engineered core promoter comprises a sequence having at least 90% sequence identity to SEQ ID NO: 110. In some aspects, the engineered core promoter comprises a sequence of SEQ ID NO: 110.

In some aspects, the engineered core promoter comprises a TATA box, an RNA polymerase binding sequence, a B recognition element, a CCAAT box, or a Pribnow box. In some aspects, the engineered core promoter is capable of recruiting a polymerase. In some aspects, the polymerase is an RNA polymerase II.

In some aspects, the engineered core promoter has a dynamic range that is higher than a dynamic range of SEQ ID NO: 6. In some aspects, the engineered core promoter has a dynamic range that is higher than a dynamic range of SEQ ID NO: 9. In some aspects, the engineered core promoter has an activity that is higher than an activity of SEQ ID NO: 6. In some aspects, the engineered core promoter has an activity that is higher than an activity of SEQ ID NO: 9. In some aspects, a transcriptional activity produced by the engineered core promoter paired with an activated response element is higher than a transcriptional activity produced by a core promoter of SEQ ID NO: 6 when paired with the activated response element, a basal transcriptional activity produced by the engineered core promoter paired with an activated response element is lower than a basal transcriptional activity produced by a core promoter of SEQ ID NO: 6 when paired with an inactive response element, or both. In some aspects, a transcriptional activity produced by the engineered core promoter paired with an activated response element is higher than a transcriptional activity produced by a core promoter of SEQ ID NO: 9 when paired with the activated response element, a basal transcriptional activity produced by the engineered core promoter paired with an activated response element is lower than a basal transcriptional activity produced by a core promoter of SEQ ID NO: 9 when paired with an inactive response element, or both.

In various aspects, the present disclosure provides an engineered response element comprising a sequence having at least 80%, at least 85%, at least 90%, at least 93%, at least 95%, at least 97%, at least 99%, or 100% sequence identity to SEQ ID NO: 145 or SEQ ID NO: 146.

In some aspects, the engineered response element comprises a sequence having at least 90% sequence identity to SEQ ID NO: 145 or SEQ ID NO: 146. In some aspects, the engineered response element comprises a sequence of SEQ ID NO: 145 or SEQ ID NO: 146. In some aspects, the engineered response element is capable of binding to a cognate ligand, a coactivator, or a corepressor. In some aspects, the engineered response element is an enhancer. In some aspects, binding of the cognate ligand or the coactivator to the engineered response element increases transcription of a payload under transcriptional control of the engineered response element paired with a core promoter in a target cell type, a target cell state, or a target tissue. In some aspects, binding of the corepressor to the engineered response element decreases transcription of a payload under transcriptional control of the engineered response element paired with a core promoter in a non-target cell type, a non-target cell state, or a non-target tissue. In some aspects, the engineered response element confers bone marrow-specific transcription of a payload under transcriptional control of the engineered response element paired with a core promoter. In some aspects, the engineered response element comprises a sequence of SEQ ID NO: 145. In some aspects, the engineered response element confers liver-specific transcription of a payload under transcriptional control of the engineered response element paired with a core promoter. In some aspects, the engineered response element comprises a sequence of SEQ ID NO: 146.

In various aspects, the present disclosure provides an engineered promoter comprising a response element and a core promoter; wherein the response element comprises a sequence having at least 80%, at least 85%, at least 90%, at least 93%, at least 95%, at least 97%, at least 99%, or 100% sequence identity to SEQ ID NO: 145, SEQ ID NO: 146, SEQ ID NO: 193, or SEQ ID NO: 194, and wherein the core promoter comprises a sequence having at least 80%, at least 85%, at least 90%, at least 93%, at least 95%, at least 97%, at least 99%, or 100% sequence identity to of any one of SEQ ID NO: 1-SEQ ID NO: 144 or SEQ ID NO: 210.

In some aspects, the core promoter comprises a sequence having at least 90% sequence identity to any one of SEQ ID NO: 1-SEQ ID NO: 144 or SEQ ID NO: 210. In some aspects, the core promoter comprises a sequence of SEQ ID NO: 1-SEQ ID NO: 144 or SEQ ID NO: 210. In some aspects, the engineered promoter comprises a sequence of any one of SEQ ID NO: 148-SEQ ID NO: 177 or SEQ ID NO: 218-SEQ ID NO: 255.

In various aspects, the present disclosure provides an engineered promoter comprising a response element and an engineered core promoter as described herein.

In some aspects, the response element is capable of binding to a cognate ligand, a coactivator, or a corepressor. In some aspects, the response element is an enhancer. In some aspects, binding of the cognate ligand or the coactivator to the response element increases transcription of a payload under transcriptional control of the engineered promoter in a target cell type, a target cell state, or a target tissue. In some aspects, binding of the corepressor to the response element decreases transcription of a payload under transcriptional control of the engineered promoter in a non-target cell type, a non-target cell state, or a non-target tissue. In some aspects, the response element confers bone marrow-specific transcription of a payload under transcriptional control of the engineered promoter. In some aspects, the response element comprises a sequence having at least 90% sequence identity to SEQ ID NO: 145 or SEQ ID NO: 193. In some aspects, the response element comprises a sequence of SEQ ID NO: 145 or SEQ ID NO: 193. In some aspects, the response element confers liver-specific transcription of a payload under transcriptional control of the engineered promoter. In some aspects, the response element comprises a sequence having at least 90% sequence identity to SEQ ID NO: 146 or SEQ ID NO: 194. In some aspects, the response element comprises a sequence of SEQ ID NO: 146 or SEQ ID NO: 194. In some aspects, the response element confers neuron-specific transcription of a payload under transcriptional control of the engineered promoter. In some aspects, the response element confers muscle-specific transcription of a payload under transcriptional control of the engineered promoter. In some aspects, the response element confers kidney-specific transcription of a payload under transcriptional control of the engineered promoter.

In various aspects, the present disclosure provides a recombinant polynucleotide comprising an engineered core promoter as described herein and a payload comprising a coding sequence under transcriptional control of the engineered core promoter.

In various aspects, the present disclosure provides a recombinant polynucleotide comprising a response element comprising a sequence having at least 80%, at least 85%, at least 90%, at least 93%, at least 95%, at least 97%, at least 99%, or 100% sequence identity to SEQ ID NO: 145, SEQ ID NO: 146, SEQ ID NO: 193, or SEQ ID NO: 194 and a payload comprising a coding sequence under transcriptional control of the response element.

In some aspects, the response element comprises a sequence having at least 90% sequence identity to SEQ ID NO: 145, SEQ ID NO: 146, SEQ ID NO: 193, or SEQ ID NO: 194. In some aspects, the response element comprises a sequence of SEQ ID NO: 145, SEQ ID NO: 146, SEQ ID NO: 193, or SEQ ID NO: 194.

In various aspects, the present disclosure provides a recombinant polynucleotide comprising a promoter and a payload, wherein the promoter comprises: a response element capable of binding to a cognate ligand, a coactivator, or a corepressor; and the engineered core promoter of any one of claims 1-60 capable of recruiting a polymerase; wherein the payload comprises a coding sequence under transcriptional control of the promoter.

In some aspects, the response element is an enhancer. In some aspects, binding of the cognate ligand or the coactivator to the response element increases transcription of the payload in a target cell type, a target cell state, or a target tissue. In some aspects, binding of the corepressor to the response element decreases transcription of the payload in a non-target cell type, a non-target cell state, or a non-target tissue. In some aspects, the response element confers neuron-specific transcription of the payload. In some aspects, the response element confers bone marrow-specific transcription of the payload. In some aspects, the response element comprises a sequence having at least 90% sequence identity to SEQ ID NO: 145 or SEQ ID NO: 193. In some aspects, the response element comprises a sequence of SEQ ID NO: 145 or SEQ ID NO: 193. In some aspects, the response element confers liver-specific transcription of the payload. In some aspects, the response element comprises a sequence having at least 90% sequence identity to SEQ ID NO: 146 or SEQ ID NO: 194. In some aspects, the response element comprises a sequence of SEQ ID NO: 146 or SEQ ID NO: 194. In some aspects, the response element confers muscle-specific transcription of the payload. In some aspects, the response element confers kidney-specific transcription of the payload.

In various aspects, the present disclosure provides a recombinant polynucleotide comprising a promoter and a payload, wherein the promoter comprises: a response element capable of binding to a cognate ligand, a coactivator, or a corepressor, wherein the response element comprises a sequence having at least 90% sequence identity to SEQ ID NO: 145, SEQ ID NO: 146, SEQ ID NO: 193, or SEQ ID NO: 194; and an engineered core promoter capable of recruiting a polymerase; wherein the payload comprises a coding sequence under transcriptional control of the promoter.

In some aspects, the payload encodes a protein. In some aspects, the protein is a neuronal protein, a kidney protein, a retinal protein, a muscle protein, or an apoptosis-inducing protein. In some aspects, the protein is associated with a genetic disorder, a neuronal disorder, a kidney disorder, an eye disorder, a muscular disorder, or a cancer. In some aspects, the protein is progranulin, MeCP2, polycystin-1, or polycystin-2.

In some aspects, the payload encodes a therapeutic polynucleotide. In some aspects, the therapeutic polynucleotide is a guide RNA or a suppressor tRNA. In some aspects, the therapeutic polynucleotide targets a gene. In some aspects, the gene is associated with a genetic disorder, a neuronal disorder, a kidney disorder, an eye disorder, a muscular disorder, or a cancer. In some aspects, the gene is GRN, MECP2, PKD2, or PKD2.

In various aspects, the present disclosure provides an engineered viral vector comprising the engineered core promoter of any one of claims 1-60 in a viral vector.

In various aspects, the present disclosure provides an engineered viral vector comprising an engineered response element as described herein in a viral vector.

In various aspects, the present disclosure provides an engineered viral vector comprising an engineered promoter as described herein in a viral vector.

In various aspects, the present disclosure provides an engineered viral vector comprising a recombinant polynucleotide as described herein in a viral vector.

In some aspects, the viral vector is an adenoviral vector, an adeno-associated viral vector, or a lentivector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, and combinations thereof.

In various aspects, the present disclosure provides a cell comprising an engineered core promoter as described herein.

In various aspects, the present disclosure provides a cell comprising an engineered response element as described herein.

In various aspects, the present disclosure provides a cell comprising an engineered promoter as described herein.

In various aspects, the present disclosure provides a cell comprising a recombinant polynucleotide as described herein.

In various aspects, the present disclosure provides a cell comprising an engineered viral vector as described herein.

In some aspects, the cell is a target cell. In some aspects, the cell is a diseased cell. In some aspects, the cell is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast.

In various aspects, the present disclosure provides a pharmaceutical composition comprising an engineered core promoter as described herein and a pharmaceutically acceptable carrier.

In various aspects, the present disclosure provides a pharmaceutical composition comprising an engineered response element as described herein and a pharmaceutically acceptable carrier.

In various aspects, the present disclosure provides a pharmaceutical composition comprising an engineered promoter as described herein and a pharmaceutically acceptable carrier.

In various aspects, the present disclosure provides a pharmaceutical composition comprising a recombinant polynucleotide as described herein and a pharmaceutically acceptable carrier.

In various aspects, the present disclosure provides a pharmaceutical composition comprising an engineered viral vector as described herein and a pharmaceutically acceptable carrier.

In various aspects, the present disclosure provides a method of expressing a payload in a target cell of a subject, the method comprising: administering to the subject a recombinant polynucleotide as described herein a viral vector as described herein, or a pharmaceutical composition as described herein to the subject; binding a cognate ligand, a coactivator, or a corepressor to the response element of the recombinant polynucleotide, wherein the cognate ligand or the coactivator is specific to the target cell, or wherein the corepressor is specific to a non-target cell; initiating transcription of the payload by recruiting a polymerase to the core promoter; and transcribing the payload, thereby expressing the payload in the target cell.

In some aspects, the method comprises expressing the payload at a higher level in the target cell than in the non-target cell. In some aspects, the method comprises initiating transcription at a higher rate in the target cell than in the non-target cell. In some aspects, the cognate ligand or the coactivator is present at a higher level in the target cell than in the non-target cell. In some aspects, the cognate ligand is a transcription factor. In some aspects, the corepressor is present at a higher level in the non-target cell than in the target cell. In some aspects, the target cell is a target cell state, and wherein the non-target cell is a non-target cell state. In some aspects, the target cell state is a diseased cell. In some aspects, the non-target cell state is a healthy cell. In some aspects, the target cell is a target cell type, and wherein the non-target cell is a non-target cell type. In some aspects, the target cell type is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. In some aspects, the non-target cell type is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, a fibroblast, or combinations thereof.

In various aspects, the present disclosure provides a method of treating a disorder in a subject in need thereof, the method comprising: administering to the subject a composition comprising a recombinant polynucleotide as described herein, a viral vector as described herein, or a pharmaceutical composition as described herein; and expressing a therapeutic sequence encoded by a payload of the recombinant polynucleotide in a target cell of the subject, thereby treating the disorder.

In some aspects, the target cell is a cell type associated with the disorder. In some aspects, the target cell is a cell state associated with the disorder. In some aspects, the disorder is a genetic disorder, a neuronal disorder, a kidney disorder, an eye disorder, a muscular disorder, or a cancer. In some aspects, the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease. In some aspects, the disorder is any one of the disorders provided in TABLE 5. In some aspects, the therapeutic sequence encodes a therapeutic protein. In some aspects, the therapeutic protein is a neuronal protein, a kidney protein, a retinal protein, a muscle protein, or an apoptosis-inducing protein. In some aspects, the therapeutic protein is associated with a genetic disorder, a neuronal disorder, a kidney disorder, an eye disorder, a muscular disorder, or a cancer. In some aspects, the therapeutic protein is MECP2, progranulin, polycystin-1, or polycystin-2. In some aspects, the therapeutic protein is encoded by a gene provided in TABLE 5. In some aspects, the payload encodes a therapeutic polynucleotide. In some aspects, the therapeutic polynucleotide is a guide RNA or a suppressor tRNA. In some aspects, the therapeutic polynucleotide targets a gene provided in TABLE 5.

In various aspects, the present disclosure provides a method of identifying a switchable core promoter, the method comprising: introducing a core promoter library comprising a first sub-library and a second sub-library to a population of cells; wherein the first sub-library comprises a plurality of core promoters, wherein a core promoter of the plurality of core promoters is linked to a first response element, and a unique barcode sequence; wherein the second sub-library comprises the plurality of core promoters, wherein the core promoter of the plurality of promoters is linked to a second response element and, a unique barcode sequence; and identifying a switchable core promoter as the core promoter that promotes higher transcription of the unique barcode when paired with the first response element than when paired with the second response element.

In some aspects, the method further comprises activating the first response element. In some aspects, the second response element is not activated. In some aspects, the first response element is an activated response element and the second enhancer sequence is an inactive response element or an unactivated response element. In some aspects, the first response element is specific for the population of cells. In some aspects, the population of cells are neurons, kidney cells, liver cells, muscle cells, or cancer cells. In some aspects, the first response element is specific for neurons, kidney cells, liver cells, muscle cells, or cancer cells. In some aspects, the second response element is specific for neurons, kidney cells, liver cells, muscle cells, or cancer cells. In some aspects, the plurality of core promoters comprises engineered core promoters, synthetic core promoters, wild type core promoters, variant core promoters, or combinations thereof.

In various aspects, the present disclosure provides a recombinant polynucleotide as described herein, a viral vector as described herein, or a pharmaceutical composition as described herein; for use in a method of treating a disorder, the method comprising administering to a subject a composition comprising the recombinant polynucleotide, viral vector or pharmaceutical composition and expressing a therapeutic sequence encoded by a payload of the recombinant polynucleotide in a target cell of the subject.

In some aspects, the disorder treated is a genetic disorder, a neuronal disorder, a kidney disorder, an eye disorder, a muscular disorder, or a cancer. In some aspects, the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease. In some aspects, the disorder is any one of the disorders provided in TABLE 5.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a scatter plot of transcriptional activity of enhancers in a cancer cell model of myeloid cells (“K562 Activity”) compared to transcriptional activity in a cancer cell model of hepatocytes (“HepG2 Activity”) determined using a massively parallel reporter assay (MPRA). An enhancer having SEQ ID NO: 194 was identified as an endogenous enhancer with hepatocyte-specific activity. An enhancer having SEQ ID NO: 193 was identified as an endogenous enhancer with myeloid-specific activity. An enhancer having SEQ ID NO: 147 was identified as a negative control enhancer with low transcriptional activation in both hepatocytes and myeloid cells.

FIG. 2 schematically illustrates engineered polynucleotides used to screen for cell type-specific transcriptional activation. Each polynucleotide construct contained, from 5′ to 3′: either a cell type-specific enhancer (“HepG2 Syn” (SEQ ID NO: 146), “K562 Syn” (SEQ ID NO: 145), “HepG2 End” (SEQ ID NO: 194), or “K562 End” (SEQ ID NO: 193)), a negative control enhancer (“Negative,” SEQ ID NO: 147), or a random sequence with no enhancer activity (“Random”); a core promoter; a barcode sequence; and a reporter open reading frame (“Reporter ORF”).

FIG. 3 shows a violin plot of transcriptional activity of 3,649 core promoters screened in hepatocytes when paired with a synthetic hepatocyte-specific enhancer (SEQ ID NO: 146), a synthetic myeloid-specific enhancer (SEQ ID NO: 145), an endogenous hepatocyte-specific enhancer (SEQ ID NO: 194), an endogenous myeloid-specific enhancer (SEQ ID NO: 193), a random sequence with no enhancer activity (“Random”), or a negative control enhancer (SEQ ID NO: 147).

FIG. 4 shows a plot comparing transcriptional activity of a library of core promoters across two replicates (“Rep 1” and “Rep 2”) of an MPRA core promoter screen.

FIG. 5A shows histogram plots of transcriptional activity, quantified by mCherry fluorescence (“mCherry”), of a ybTATA core promoter (SEQ ID NO: 9) assayed using a dual reporter flow assay in HepG2 cells. Transcriptional activity was assessed with the ybTATA inserted alone into the dual reporter construct (“ybTATA Only”), or when paired with a negative control enhancer (SEQ ID NO: 147), a synthetic myeloid-specific enhancer (SEQ ID NO: 145), an endogenous myeloid-specific enhancer (SEQ ID NO: 193), a synthetic hepatocyte-specific enhancer (SEQ ID NO: 146), or an endogenous hepatocyte-specific enhancer (SEQ ID NO: 194). Wild type cells with no plasmid (“WT”) were used as a negative control.

FIG. 5B shows a bar chart comparing average fold change in the geometric mean of the fluorescence intensity (gMFI) core promoters screened in HepG2 cells using a dual reporter flow assay. Transcriptional activity of core promoters was assessed with the ybTATA inserted alone into the dual reporter construct (“ybTATA Only”), or when paired with an endogenous hepatocyte-specific enhancer (SEQ ID NO: 194), a synthetic hepatocyte-specific enhancer (SEQ ID NO: 146), an endogenous myeloid-specific enhancer (SEQ ID NO: 193), a synthetic myeloid-specific enhancer (SEQ ID NO: 145), or a negative control enhancer (SEQ ID NO: 147).

FIG. 5C shows a bar chart comparing average fold change in the geometric mean of the fluorescence intensity (gMFI) core promoters screened in K562 cells using a dual reporter flow assay. Transcriptional activity of core promoters was assessed with the ybTATA inserted alone into the dual reporter construct (“ybTATA Only”), or when paired with an endogenous hepatocyte-specific enhancer (SEQ ID NO: 194), a synthetic hepatocyte-specific enhancer (SEQ ID NO: 146), an endogenous myeloid-specific enhancer (SEQ ID NO: 193), a synthetic myeloid-specific enhancer (SEQ ID NO: 145), or a negative control enhancer (SEQ ID NO: 147).

FIG. 5D shows histogram plots of transcriptional activity, quantified by mCherry fluorescence (“mCherry”), of a ybTATA core promoter (SEQ ID NO: 9) assayed using a dual reporter flow assay in K562 cells. Transcriptional activity was assessed with the ybTATA inserted alone into the dual reporter construct (“ybTATA Only”), or when paired with a negative control enhancer (SEQ ID NO: 147), a synthetic myeloid-specific enhancer (SEQ ID NO: 145), an endogenous myeloid-specific enhancer (SEQ ID NO: 193), a synthetic hepatocyte-specific enhancer (SEQ ID NO: 146), or an endogenous hepatocyte-specific enhancer (SEQ ID NO: 194). Wild type cells with no plasmid (“WT”) were used as a negative control.

FIG. 6 shows a scatter plot comparing fold activation of transcriptional activity in HepG2 cells of a library of core promoters screened using an MPRA screen when paired with different enhancers. Fold activation of each core promoter paired with a synthetic HepG2-specific enhancer of SEQ ID NO: 146 relative to the core promoter paired with a negative control enhancer of SEQ ID NO: 147 (“HepSyn vs Negative”) was compared to fold activation of each core promoter paired with a synthetic K562-specific enhancer of SEQ ID NO: 145 relative to the core promoter paired with no enhancer (“KSyn vs No Enhancer”). The dashed line denotes a line of y=x, and outlined points indicate ybTATA (SEQ ID NO: 9) and minP (SEQ ID NO: 5) core promoters.

FIG. 7 shows a scatter plot comparing transcriptional activation in HepG2 cells of two libraries of core promoters having different spacing sequences (either “Background 1” having a sequence of SEQ ID NO: 208 or “Background 2” having a sequence of SEQ ID NO: 209) that were screened using an MPRA. The dashed line denotes a line of y=x.

FIG. 8 shows a scatter plot comparing transcriptional activity in HepG2 cells of a sub-library of de novo designed core promoters screened using an MPRA screen when paired with different synthetic enhancers. Transcriptional activity of each core promoter paired with a synthetic HepG2-specific enhancer of SEQ ID NO: 146 (“HepG2 Synthetic”) was compared to transcriptional activity when paired with a synthetic K562-specific enhancer of SEQ ID NO: 145 (“K562 Synthetic”). The dashed line denotes a line of y=x.

FIG. 9A shows a scatter plot comparing transcriptional activity of sub-libraries of de novo designed core promoters with different length spacing from the TATA sequence to the initiator element (Inr) sequence. Transcriptional activity was measured in HepG2 cells when paired with a synthetic HepG2 enhancer (SEQ ID NO: 146) using an MPRA screen. Transcriptional activity of each core promoter with a standard TATA to Inr spacing of 30 nucleotides (“Founder Combo”) was compared to transcriptional activity of a corresponding core promoters with a shortened 28 nucleotides TATA to Inr spacing (“With Short Spacing”). A highly active core promoter, corresponding to SEQ ID NO: 141 with standard spacing or SEQ ID NO: 7 with short spacing, is circled in red. The dashed line denotes a line of y=x.

FIG. 9B shows a violin plot of the transcriptional activity of SEQ ID NO: 141, a core promoter with standard TATA to Inr spacing of 30 nucleotides, and SEQ ID NO: 7, the corresponding core promoter with shorter TATA to Inr spacing of 28 nucleotides. Each bar of the plot includes the activity values for each of the approximately 40 redundant barcodes paired with corresponding core promoter. Transcriptional activity was measured in HepG2 cells when paired with a synthetic HepG2 enhancer (SEQ ID NO: 146).

FIG. 10A shows a scatter plot comparing transcriptional activity of libraries of core promoters with or without a transcription pause site. Transcriptional activity was measured in HepG2 cells when paired with a synthetic HepG2 enhancer (SEQ ID NO: 146) using an MPRA screen. Transcriptional activity of each core promoter without a transcription pause site (“Founder Combo”) was compared to transcriptional activity of a corresponding core promoter with a transcription pause site (“With Pause Site”). Core promoters containing a pause site of SEQ ID NO: 196 are denoted with dark circles. The dashed line denotes a line of y=x.

FIG. 10B shows a scatter plot comparing fold activation of transcription of libraries of core promoters paired with a synthetic HepG2 enhancer (SEQ ID NO: 146) with or without a transcription pause site versus negative control (SEQ ID NO: 147). Fold activation was measured versus negative control (SEQ ID NO: 147) in HepG2 cells for core promoters with a transcription pause site relative to core promoters without a transcription pause site using an MPRA screen. Fold activation of each core promoter with a transcription pause site (“With Pause Site”) relative to a corresponding core promoter without a transcription pause site (“Founder Combo”) was measured in HepG2 cells using an MPRA screen. Sequences with the best pause sites are denoted with dark circles. The dashed line denotes a line of y=x.

FIG. 11A shows a scatter plot comparing transcriptional activity in HepG2 cells of a library of core promoters screened using an MPRA screen when paired with different synthetic enhancers. Transcriptional activity of each core promoter paired with a synthetic HepG2-specific enhancer of SEQ ID NO: 146 (“HepG2 Synthetic”) was compared to transcriptional activity when paired with a synthetic K562-specific enhancer of SEQ ID NO: 145 (“K562 Synthetic”). Core promoters containing a YY1 motif are denoted by darker circles. The dashed line denotes a line of y=x.

FIG. 11B shows a scatter plot comparing transcriptional activity in HepG2 cells of a library of core promoters screened using an MPRA screen when paired with different enhancers that are not activated in HepG2 cells. Transcriptional activity of each core promoter paired with an endogenous K562-specific enhancer of SEQ ID NO: 193 (“K562 Endogenous”) was compared to transcriptional activity when paired with a negative control enhancer of SEQ ID NO: 147 (“Negative Control”). Core promoters containing a YY1 motif are denoted by darker circles. The dashed line denotes a line of y=x.

FIG. 11C shows a scatter plot comparing transcriptional activity of libraries of core promoters with or without a YY1 motif. Transcriptional activity was measured in HepG2 cells when paired with a synthetic HepG2 enhancer (SEQ ID NO: 146) using an MPRA screen. Transcriptional activity of each core promoter without a YY1 motif (“Founder Combo”) was compared to transcriptional activity of a corresponding core promoter having a transcription pause site (“With YY1”). The dashed line denotes a line of y=x.

FIG. 11D shows a scatter plot comparing transcriptional activity of libraries of core promoters with or without a YY1 motif. Transcriptional activity was measured in HepG2cells when paired with a synthetic K562 enhancer (SEQ ID NO: 145) using an MPRA screen. Transcriptional activity of each core promoter without a YY1 motif (“Founder Combo”) was compared to transcriptional activity of a corresponding core promoter having a transcription pause site (“With YY1”). The dashed line denotes a line of y=x.

FIG. 12A shows a scatter plot comparing transcriptional activity of point mutants of a ybTATA core promoter (SEQ ID NO: 9) in HepG2 cells when paired with a synthetic HepG2-specific enhancer of SEQ ID NO: 146. Activity of each possible point mutation at each position in the sequence is shown as a shaded circle, with the darkest shade representing T, the next darkest shade representing A, the lightest shade representing G, and the next lightest shade representing C. Activity of the ybTATA sequence (SEQ ID NO: 9), provided at the bottom of the plot, is shown with dashes colored based on the identity of the nucleotide present at that position in SEQ ID NO: 9.

FIG. 12B shows a scatter plot comparing transcriptional activity of point mutants of a minP core promoter (SEQ ID NO: 5) in HepG2 cells when paired with a synthetic HepG2-specific enhancer of SEQ ID NO: 146. Activity of each possible point mutation at each position in the sequence is shown as a shaded circle, with the darkest shade representing T, the next darkest shade representing A, the lightest shade representing G, and the next lightest shade representing C. Activity of the minP sequence (SEQ ID NO: 5), provided at the bottom of the plot, is shown with shaded dashes based on the identity of the nucleotide present at that position in SEQ ID NO: 5.

FIG. 13A shows a sequence logo plot illustrating the relative enrichment of each possible nucleotide in the TATA sequence and neighboring residues.

FIG. 13B shows a sequence logo plot illustrating the relative enrichment of each possible nucleotide in the Inr sequence and neighboring residues.

FIG. 13C shows a sequence logo plot illustrating the relative enrichment of each possible nucleotide in a YY1 transcription factor binding motif sequence and neighboring residues.

FIG. 13D shows a sequence logo plot illustrating the relative enrichment of each possible nucleotide in an HNF4a transcription factor binding motif sequence and neighboring residues.

FIG. 14A shows a heatmap representing activity of double point mutants of minP (SEQ ID NO: 5) in HepG2 cells when paired with a synthetic HepG2-specific enhancer of SEQ ID NO: 146. Lighter shading indicates higher transcriptional activity. The core promoter with a sequence of SEQ ID NO: 15, containing T15G and A40C substitutions relative to SEQ ID NO:

5), is denoted with a white box.

FIG. 14B shows a heatmap representing activity of double point mutants of minP (SEQ ID NO: 5) in K562 cells when paired with a synthetic K562-specific enhancer of SEQ ID NO: 145. Lighter shading indicates higher transcriptional activity. The core promoter with a sequence of SEQ ID NO: 15, containing T15G and A40C substitutions relative to SEQ ID NO: 5), is denoted with a white box.

FIG. 15A shows a scatter plot comparing transcriptional activity in HepG2 cells of a library of core promoters screened using an MPRA screen when paired with different synthetic enhancers. Transcriptional activity of each core promoter paired with a synthetic K562-specific enhancer of SEQ ID NO: 145 (“K562 Synthetic”) was compared to transcriptional activity when paired with a synthetic HepG2-specific enhancer of SEQ ID NO: 146 (“HepG2 Synthetic”). SEQ ID NO: 5 (minP) and SEQ ID NO: 9 (ybTATA) are denoted with circles. The dashed line denotes a line of y=x.

FIG. 15B shows a scatter plot comparing fold activation of transcriptional activity in HepG2 cells of a library of core promoters screened using an MPRA screen when paired with different enhancers. Fold activation of each core promoter paired with a synthetic HepG2-specific enhancer of SEQ ID NO: 146 relative to the core promoter paired with a negative control enhancer of SEQ ID NO: 147 (“HepG2 Synthetic vs Negative”) was compared to fold activation of each core promoter paired with a synthetic K562-specific enhancer of SEQ ID NO: 145 relative to the core promoter paired with no enhancer (“K562 Synthetic vs None”). SEQ ID NO: 5 (minP) and SEQ ID NO: 9 (ybTATA) are denoted with circles. The dashed line denotes a line of y=x.

FIG. 16A shows a violin plot of transcriptional activity of select core promoters in HepG2 cells when paired with a synthetic HepG2-specific enhancer of SEQ ID NO: 146. Each bar of the plot includes the activity values for each of the approximately 40 redundant barcodes paired with corresponding core promoter. Activity of core promoters of SEQ ID NO: 7, SEQ ID NO: 6, SEQ ID NO: 5 (minP), and SEQ ID NO: 9 (ybTATA) was compared.

FIG. 16B shows a violin plot of transcriptional activity of select core promoters in HepG2 cells when paired with a negative control enhancer of SEQ ID NO: 147. Each bar of the plot includes the activity values for each of the approximately 40 redundant barcodes paired with corresponding core promoter. Activity of core promoters of SEQ ID NO: 7, SEQ ID NO: 6, SEQ ID NO: 5 (minP), and SEQ ID NO: 9 (ybTATA) was compared.

FIG. 17 shows a scatter plot comparing fold activation of transcriptional activity in HepG2 cells of a library of core promoters screened using an MPRA screen when paired with different enhancers. Fold activation of each core promoter paired with a synthetic HepG2-specific enhancer of SEQ ID NO: 146 relative to the core promoter paired with a negative control enhancer of SEQ ID NO: 147 (“HepSyn vs Negative”) was compared to fold activation of each core promoter paired with a synthetic K562-specific enhancer of SEQ ID NO: 145 relative to the core promoter paired with no enhancer (“KSyn vs No Enhancer”). The dashed line denotes a line of y=x, and black points indicate core promoters of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 18 shows a scatter plot comparing transcriptional activity in HepG2 cells of a library of core promoters screened using an MPRA screen when paired with different enhancers. Transcriptional activity of each core promoter was compared when paired with a synthetic HepG2-specific enhancer of SEQ ID NO: 146 (“HepG2 Synthetic”) or a synthetic K562-specific enhancer of SEQ ID NO: 145 (“K562 Synthetic”). The dashed line denotes a line of y=x, and black points indicate core promoters of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 19A shows schematics of core promoters of SEQ ID NO: 1, SEQ ID NO: 2, SEQ ID NO: 3, and SEQ ID NO: 7 indicating regions corresponding to the TATA box and the initiator element (Inr). If present, regions corresponding to the transcriptional pause site and YY1 motif are also indicated.

FIG. 19B shows schematics of core promoters of SEQ ID NO: 8, SEQ ID NO: 10, SEQ ID NO: 11, and SEQ ID NO: 12 indicating regions corresponding to the TATA box and the initiator element (Inr). If present, regions corresponding to the transcriptional pause site and YY1 motif are also indicated.

FIG. 19C shows schematics of core promoters of SEQ ID NO: 13, SEQ ID NO: 14, SEQ ID NO: 15, SEQ ID NO: 4, and SEQ ID NO: 110, indicating regions corresponding to the TATA box and the initiator element (Inr). If present, regions corresponding to the transcriptional pause site and YY1 motif are also indicated. In SEQ ID NO: 15 and SEQ ID NO: 4, which represent variants of the minP core promoter, point mutations relative to the minP sequence (SEQ ID NO: 5) are denoted with boxes.

FIG. 20A shows a comparison of promoter activity determined by an MPRA screen (“Screen Activity”) and by dual reporter qPCR assay (“qPCR Activity”) for core promoters paired with a K562-specific enhancer in HepG2 cells. Activity was compared for core promoters of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 20B shows a comparison of promoter activity determined by a an MPRA screen (“Screen Activity”) and by dual reporter qPCR assay (“qPCR Activity”) for core promoters paired with a HepG2-specific enhancer in HepG2 cells. Activity was compared for core promoters of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 20C shows a comparison of promoter activity determined by an MPRA screen (“Screen Activity”) and by a dual reporter qPCR assay (“qPCR Activity”) for core promoters paired with a K562-specific enhancer in K562 cells. Activity was compared for core promoters of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 21A shows a comparison of the transcriptional activity of a minP variant of SEQ ID NO: 4 (“minP Activity”) and a ybTATA promoter of SEQ ID NO: 9 (“yb_TATA Activity”) in H4 cells when paired with either an H4-specific enhancer (“H4-specific enhancer 1” or “H4-specific enhancer 2”) or a liver-specific enhancer.

FIG. 21B shows a comparison of the transcriptional activity of a minP variant of SEQ ID NO: 4 (“minP Activity”) and a ybTATA promoter of SEQ ID NO: 9 (“yb_TATA Activity”) in HepG2 liver cells when paired with either an H4-specific enhancer (“H4-specific enhancer 1” or “H4-specific enhancer 2”) or a liver-specific enhancer.

FIG. 21C shows a comparison of the cell-type specificity of a minP variant of SEQ ID NO: 4 (“minP Specificity”) and a ybTATA promoter of SEQ ID NO: 9 (“yb_TATA Specificity”).

FIG. 22A shows a violin plot of transcriptional activity of core promoters screened in myeloid cells when paired with a synthetic hepatocyte-specific enhancer (SEQ ID NO: 146), a synthetic myeloid-specific enhancer (SEQ ID NO: 145), an endogenous hepatocyte-specific enhancer (SEQ ID NO: 194), an endogenous myeloid-specific enhancer (SEQ ID NO: 193), a random sequence with no enhancer activity (“Random”), or a negative control enhancer (SEQ ID NO: 147).

FIG. 22B shows a violin plot of transcriptional activity of core promoters screened in hepatocytes when paired with a synthetic hepatocyte-specific enhancer (SEQ ID NO: 146), a synthetic myeloid-specific enhancer (SEQ ID NO: 145), an endogenous hepatocyte-specific enhancer (SEQ ID NO: 194), an endogenous myeloid-specific enhancer (SEQ ID NO: 193), a random sequence with no enhancer activity (“Random”), or a negative control enhancer (SEQ ID NO: 147).

FIG. 23 shows a scatter plot comparing transcriptional activity in HepG2 and K562 cells of a library of core promoter sequences screened using an MPRA screen when paired with cell type-specific enhancers: the transcriptional activity in HepG2 cells of each core promoter when paired with a synthetic HepG2-specific enhancer of SEQ ID NO: 146 (“HepG2 Synthetic”) was compared to the transcriptional activity in K562 cells of each core promoter when paired with a synthetic K562-specific enhancer of SEQ ID NO: 145 (“K562 Synthetic”). The dashed line denotes a line of y=x, and black points indicate core promoters of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

FIG. 24. shows a hexbin plot comparing transcriptional activity in HepG2 and K562 cells of a library of 21,849 enhancer: core promoters screened using an MPRA screen when paired with different enhancers, including a synthetic HepG2-specific enhancer of SEQ ID NO: 146 and a synthetic K562-specific enhancer of SEQ ID NO: 145. The transcriptional activity of each core promoter paired with the different enhancers was compared between HepG2 and K562 cell lines. The cluster of points corresponding to core promoters paired with the synthetic HepG2-specific enhancer (SEQ ID NO: 146) is indicated with a broken ellipse, and the cluster of points corresponding to core promoters paired with the synthetic K562-specific enhancer (SEQ ID NO: 145) is indicated with a solid ellipse.

### DETAILED DESCRIPTION

While gene therapy has the potential to treat a wide variety of diseases associated with genetic mutations or altered gene expression, there may be risks associated with such therapies due, in part, to imprecise targeting of cells or tissues with the therapeutic payload. Limiting payload expression to a target cell type, cell state, or tissue type, such as a diseased cell or tissue, may reduce the side effects of gene therapy caused by imprecise targeting. Described herein are polynucleotide sequences that encode for cell type- or cell state-specific expression of a payload. The polynucleotide sequences may comprise a payload, such as a transgene or therapeutic polynucleotide, under transcriptional control of a promoter. The promoter may facilitate cell type- or cell state-specific transcription initiation, leading to cell type- or cell state-specific expression of the payload. In some embodiments, the promoter may comprise a response element that is capable of binding to cognate ligands, coactivators, or corepressors to modulate payload transcription and a core promoter that is capable of recruiting transcriptional machinery to initiate transcription of the payload when in combination with the response element bound to cognate ligands, coactivators, or corepressors. As described herein, the core promoter may be a switchable core promoter that promotes high levels of transcription in the presence of an activated response element, such as an enhancer bound to a cognate ligand or coactivator, and low or no transcription in the presence of an unactivated response element, such as an enhancer not bound to the cognate ligand or coactivator or an enhancer bound to a corepressor. The cognate ligand may be a transcription factor or sequence-specific DNA binding factor.

The polynucleotides of the present disclosure (e.g., promoters, core promoters, response elements, payloads, or combinations thereof) may comprise a recombinant polynucleotide sequence. The recombinant polynucleotide sequence may be engineered to encode for tissue type-, cell type, or cell state-specific transcription of a payload. In some embodiments, the level of transcription of the payload may depend on a cell type (e.g., neuron, renal cell, hepatocyte, podocyte, retinal cell, epithelial cell, muscle cell, erythrocyte, platelet, bone marrow cell, endothelial cell, epidermal cell, lymphocyte, glial cell, interstitial cell, adipocyte, or fibroblast), cell state (e.g., diseased cell or healthy cell; activated or unactivated engineered cells, such as an activated or unactivated CAR T cell), tissue type (e.g., diseased tissue, healthy tissue, nervous tissue (e.g., central nervous system, peripheral nervous system), kidney tissue, eye tissue, muscle tissue, blood, skin, fat, bone, cancerous tissue, thymus tissue, gastrointestinal tissue (e.g., stomach, intestine), spleen tissue, placenta tissue, pancreatic tissue, lung tissue, liver tissue, cardiac tissue, or brain tissue (e.g., cerebrum, cerebellum, adrenal)), or a combination thereof. In some embodiments, a recombinant polynucleotide of the present disclosure may comprise a core promoter (e.g., an engineered core promoter) as described herein, a response element (e.g., an engineered response element) as described herein, a payload (e.g., transgene or therapeutic polynucleotide) as described herein, or combinations thereof.

The promoter may be selected for enhanced transcription in a cell type, cell state, or tissue type of interest, reduced transcription in other cell types, cell states, or tissue types, or combinations thereof. In some embodiments, the promoter may be selected or engineered to tune the level of payload transcription (e.g., to a therapeutic level, such as about the same level as a wild type version of a transgene in the target cell type or cell state). In some embodiments, the promoter may be selected or engineered to tune the cell state-dependence of payload transcription. Tuning a transcription level may comprise adjusting transcription to a desired level. The transcription level of the payload may control the expression level of the protein encoded by the payload. For example, a high level of transcription of a transgene may lead to a high level of expression of the protein encoded by the transgene. A core promoter of the present disclosure (e.g., an engineered core promoter) may be engineered or selected for both strength (e.g., strong transcriptional activation) and specificity (e.g., specific activation when paired with a response element in a transcriptionally active state compared to when paired with a response element in a transcriptionally inactive state).

Also described herein are methods of delivering a polynucleotide sequence of the present disclosure to a subject. In some embodiments, the polynucleotide may be part of a viral vector capable of delivering the polynucleotide to a cell of the subject. The viral vector may comprise a viral inverted terminal repeat sequence that includes a viral origin of replication, enabling viral replication of the polynucleotide sequence. The viral vector may further comprise a viral capsid encapsulating the polynucleotide and facilitating delivery of the polynucleotide into the cell. A method of delivering a polynucleotide composition to a subject may comprise administering a viral vector comprising the polynucleotide to the subject. Upon delivery of the polynucleotide to the subject, a payload encoded by the polynucleotide may be transcribed in a cell of the subject in a cell type-, cell state-, or tissue type-dependent manner.

Further described herein are methods of treating a disease or condition by delivering a polynucleotide composition of the present disclosure to a subject and expressing a therapeutic protein or therapeutic polynucleotide encoded by the polynucleotide in the subject in a cell type-, cell state-, or tissue type-dependent manner. The therapeutic protein may be a wild type version of a protein mutated or under-expressed in the disease or condition. The therapeutic polynucleotide may be a polynucleotide (e.g., a gRNA or a tRNA) that targets a gene associated with the disease or condition. The polynucleotide composition may be delivered to the subject as part of a viral vector. The subject may have a disease or condition, for example a disease or condition caused by mutation or altered expression of a protein. In some embodiments, the polynucleotide composition may comprise a transgene encoding a wild type copy of the protein having the mutation or altered expression. The transgene may be selectively transcribed in a target cell type, cell state, or tissue type of the subject upon delivery of the polynucleotide composition to the subject. In some embodiments, a protein encoded by the transgene is expressed in the subject at a level dependent on the level of transcription of the transgene. Transcription of the transgene, expression of the protein encoded by the transgene, or both, in a cell type-, cell state-, or tissue type-dependent manner may treat the disease or condition in the subject.

Further described herein are methods of identifying a switchable core promoter as disclosed herein. The switchable core promoter may be an engineered core promoter as disclosed herein. A method of identifying a switchable core promoter may comprise: introducing a core promoter library comprising a first sub-library and a second sub-library to a population of cells; wherein the first sub-library comprises a plurality of core promoters, wherein a core promoter of the plurality of core promoters is linked to a first response element, and a unique barcode sequence; wherein the second sub-library comprises the plurality of core promoters, wherein the core promoter of the plurality of promoters is linked to a second response element and, a unique barcode sequence; and identifying a switchable core promoter as the core promoter that promotes higher transcription of the unique barcode when paired with the first response element than when paired with the second response element. The method may further comprise activating the first response element. The second response element may not be activated. The first response element may be an activated response element and the second enhancer sequence may be an inactive response element or an unactivated response element. The first response element may be specific for the population of cells. The population of cells may be neurons, kidney cells, liver cells, muscle cells, or cancer cells. The first response element may be specific for neurons, kidney cells, liver cells, muscle cells, or cancer cells. The second response element may be specific for neurons, kidney cells, liver cells, muscle cells, or cancer cells. The plurality of core promoters may comprise engineered core promoters, synthetic core promoters, wild type core promoters, variant core promoters, or combinations thereof.

As used herein, “switchable” may refer to the ability of a core promoter to be combined with various response elements (e.g., cell type-specific enhancers, tissue type-specific enhancers, or cell state-specific enhancers) to promote high levels of transcription in the presence of a response element in a transcriptionally active state (e.g., an activated response element) and low or no transcription in the presence of a response element in a transcriptionally inactive or repressed state (e.g., an unactivated response element or an inactive response element).

As used herein, “core promoter” or “core promoter sequence” may refer to a polynucleotide encoding a sequence that recruits transcriptional machinery (e.g., an RNA polymerase) to initiate transcription of a downstream polynucleotide coding for a sequence (e.g., a payload sequence), for example by binding general transcription factors (GTFs) that recruit an RNA polymerase. As used herein, “engineered core promoter” or “engineered core promoter sequence” may refer to a non-naturally occurring core promoter or core promoter sequence. In some embodiments, the “engineered core promoter” or “engineered core promoter sequence” may refer to a core promoter that has been mutated, altered, or engineered to have a moderate affinity for molecules (e.g., general transcription factors (GTFs)) that recruit transcriptional machinery (e.g., an RNA polymerase). More specifically, the moderate affinity is such that the concentration of these molecules (e.g., GTFs) in the nucleus results in little or no recruitment of transcriptional machinery to the engineered core promoter when in the absence of an activated response element or in the presence of an inactive response element. However, when in the presence of an activated response element, the activated response element causes an increase in the concentration of these molecules (e.g., GTFs) at the core promoter and the resulting increased localized concentration of these molecules (e.g., GTFs) at the core promoter overcomes the moderate affinity, allowing for higher recruitment of transcriptional machinery (e.g., an RNA polymerase) to initiate higher transcription of a downstream polynucleotide coding for a sequence (e.g., a payload sequence) compared the recruitment of transcription machinery and transcription level in the absence of the activated response element.

As used herein, “response element” may refer to a polynucleotide encoding a sequence that binds to a cognate ligand (e.g., a transcription factor), coactivator, corepressor, or combinations thereof in a sequence-dependent manner. An “active response element” may refer to a polynucleotide encoding a sequence that is capable of binding to a cognate ligand (e.g., a transcription factor), coactivator, corepressor, or combinations thereof in a sequence-dependent manner to facilitate binding of transcriptional machinery to a nearby core promoter, e.g., an engineered core promoter. An “inactive response element” may refer to a polynucleotide encoding a sequence that is not capable of binding to a cognate ligand (e.g., a transcription factor), coactivator, or combinations thereof in a sequence-dependent manner to facilitate binding of transcriptional machinery to a nearby core promoter, e.g., an engineered core promoter. An “activated response element” may be a response element bound to a cognate ligand (e.g., a transcription factor) or coactivator, or having decreased binding to a corepressor (as compared to when unactivated), or combinations thereof, that facilitates high binding of transcriptional machinery to a nearby core promoter, e.g., an engineered core promoter. An “unactivated response element” may be an active response element that is not bound to a cognate ligand (e.g., a transcription factor) or coactivator, or having increased binding to a corepressor (as compared to when activated), or combinations thereof that has low or does not facilitate binding of transcriptional machinery to a nearby core promoter, e.g., an engineered core promoter. For example, an activated response element may facilitate increased binding of transcriptional machinery to the nearby core promoter than an unactivated response element to the same core promoter.

As used herein, “enhancer” or “enhancer region” may refer to a response element that binds to a cognate ligand or coactivator, or has low binding to a corepressor to increase binding of transcriptional machinery to a nearby core promoter and, therefore, increase transcription of a nearby polynucleotide encoding a sequence of a payload, such as a transgene or therapeutic polynucleotide, wherein this binding is cell type-, cell state-, or tissue type-dependent. An “active enhancer” may refer to a polynucleotide encoding a sequence that is capable of cell type-, cell state-, or tissue type-dependent binding to a cognate ligand (e.g., a transcription factor), coactivator, or having decreased binding to a corepressor (as compared to when unactivated), or combinations thereof in a sequence-dependent manner that facilitates binding of transcriptional machinery to a nearby core promoter, e.g., an engineered core promoter, when in that cell type, cell state, or tissue type, respectively. An “inactive enhancer” may refer to a polynucleotide encoding a sequence that is not capable of cell type-, cell state-, or tissue type-dependent binding to a cognate ligand (e.g., a transcription factor), coactivator, or combinations thereof, in a sequence-dependent manner to facilitate binding of transcriptional machinery to a nearby core promoter, e.g., an engineered core promoter, when in that cell type, cell state, or tissue type, respectively. An “activated enhancer” may be an enhancer in its specific cell type, cell state, or tissue type so that it is bound to a cognate ligand (e.g., a transcription factor) or coactivator, or having decreased binding to a corepressor (as compared to when unactivated), or combinations thereof, that facilitates binding of transcriptional machinery to a nearby core promoter, e.g., an engineered core promoter in that specific cell type, cell state, or tissue type. An “unactivated enhancer” may be an active enhancer that is not in its specific cell type, cell state, or tissue type so that it is not bound to a cognate ligand (e.g., a transcription factor) or coactivator, or has increased binding to a corepressor (as compared to when activated), or combinations thereof, that has low or does not facilitate binding of transcriptional machinery to a nearby core promoter, e.g., an engineered core promoter.

### Promoters

A polynucleotide (e.g., an RNA or a DNA polynucleotide) may comprise a promoter to regulate or enhance transcription of a payload, such as a transgene or therapeutic polynucleotide. The promoter may be an engineered promoter. In some embodiments, the promoter may comprise a response element (e.g., an enhancer region) and a core promoter that functions as a site for preinitiation complex formation, or combinations thereof. The core promoter may be an engineered core promoter. In some embodiments, the sequence of the response element may comprise a protein binding sequence that binds one or more cognate ligands, coactivators, or corepressors in a sequence-dependent manner. For example, a response element may bind a transcription factor (TF) that enhances transcription of a nearby polynucleotide encoding a sequence of a payload, such as a transgene or therapeutic polynucleotide. In some embodiments, the core promoter may comprise an engineered core promoter to promote high levels of transcription in the presence of an active response element (e.g., an active enhancer) when activated and low or no transcription in the presence of an inactive response element (e.g., an inactive enhancer). In some embodiments, the core promoter may comprise an engineered core promoter to promote high levels of transcription in the presence of an activated response element (e.g., an activated enhancer) and low or no transcription in the presence of an unactivated response element (e.g., an unactivated enhancer). In some embodiments, the core promoter may comprise a synthetic promoter engineered to promote high levels of transcription in the presence of an activated response element (e.g., an activated enhancer) and low or no transcription in the presence of an inactive response element (e.g., an inactive enhancer).

The elements within the promoter (e.g., the response element or the core promoter) may be selected or engineered to alter transcription rates of a downstream polynucleotide encoding for a payload. In some embodiments, the promoter may be selected for ubiquitous transcription. For example, the promoter may be selected to promote high levels of transcription in any cell type, tissue type, or cell state. In some embodiments, the promoter may be selected for cell type-specific transcription. In some embodiments, the promoter may be selected for tissue type-specific transcription. In some embodiments, the promoter may be selected for cell state-specific transcription. For example, the promoter may be selected to promote high levels of transcription in a target cell or tissue type (e.g., excitatory neurons or kidney tissue) or a target cell state (e.g., a diseased cell state) and low levels or no transcription in non-target cell types or states (e.g., non-neuronal cells, non-kidney tissue, or non-diseased cells). In some embodiments, the target tissue may be nervous tissue, kidney tissue, eye tissue, muscle tissue, blood, skin, fat, bone, cancerous tissue, thymus tissue, gastrointestinal tissue (e.g., stomach, intestine), spleen tissue, placenta tissue, pancreatic tissue, lung tissue, liver tissue, cardiac tissue, or brain tissue (e.g., cerebrum, cerebellum, adrenal). In some embodiments, the target cell type may be neuron, renal cell, hepatocyte, podocyte, retinal cell, epithelial cell, muscle cell, erythrocyte, platelet, bone marrow cell, endothelial cell, epidermal cell, lymphocyte, glial cell, interstitial cell, adipocyte, or fibroblast. In some embodiments, the target cell state may be a diseased cell, an actively dividing cell, a quiescent cell, or a mutated cell. In some embodiments, the target cell may be a specific state of a cell. For example, for a CAR T cell, the target state of a cell may be activated and a non-target state may be inactive (e.g., naïve).

In some embodiments, a promoter may promote cell type-specific transcription if it promotes transcription of polynucleotides encoding for a payload (i.e., a payload sequence) in a target cell type at a level that is at least about 0.1-fold, at least about 0.25-fold, at least about 0.5-fold, at least about 0.75-fold, at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of the payload sequence in a non-target cell type. In some embodiments, a promoter may promote cell state-specific transcription if it promotes transcription of a payload sequence in a target cell state at a level that is at least about 0.1-fold, at least about 0.25-fold, at least about 0.5-fold, at least about 0.75-fold, at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of the payload sequence in a non-target cell state.

In some embodiments, a promoter may promote a desired level of transcription if it promotes transcription of polynucleotides encoding for a payload (i.e., a payload sequence) in a target cell type at a level that is at least about-0.75 fold, at least about-0.5 fold, at least about-0.25-fold, at least about-0.1-fold, at least about 0-fold, at least about 0.1-fold, at least about 0.25-fold, at least about 0.5-fold, at least about 0.75-fold, at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of an endogenous version of the payload sequence in a target cell type. In some embodiments, a promoter may promote a desired level of transcription of a payload sequence if it promotes transcription of the payload sequence in a target cell state at a level that is at least about-0.75 fold, at least about-0.5 fold, at least about-0.25-fold, at least about-0.1-fold, at least about 0-fold, at least about 0.1-fold, at least about 0.25-fold, at least about 0.5-fold, at least about 0.75-fold, at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of an endogenous version of the payload sequence in a target cell state.

### Response Elements

The promoter of a polynucleotide may comprise a response element, such as an enhancer region. The response element may bind to a sequence-specific protein, such as a cognate ligand (e.g., a transcription factor), a coactivator, and/or a corepressor to modulate transcription. For example, the response element may recruit a cognate ligand (e.g., a transcription factor), a coactivator, and/or a corepressor that enhances, represses, or alters transcription of a downstream polynucleotide encoding a sequence of a payload, such as a transgene or therapeutic polynucleotide. In some embodiments, the response element may function to enhance the rate of transcription upon binding of a cognate ligand or coactivator and/or upon decreased binding of a corepressor. For example, a response element may comprise a transcription factor binding site that binds transcription factors that enhance transcription of nearby polynucleotides comprising coding sequences. In some embodiments, a response element may be an activated response element or inactivated response element, in which an activated response element is a response element bound to a transcription factor that enhances transcription and that may enhance transcription initiation by a core promoter relative to a response element without a transcription factor bound and an inactivated response element is a response element that is not bound to a transcription factor and therefore it does not enhance transcription or enhance transcription initiation by a core promoter. In some embodiments, the response element may function to alter the rate of transcription upon binding of a cognate ligand a coactivator, and/or a corepressor. An enhancer region may be a response element that functions to enhance the rate of transcription upon binding of a cognate ligand or coactivator or decreased binding of a corepressor, wherein this binding of a cognate ligand or coactivator or decreased binding of a corepressor and subsequent enhancement of the rate of transcription is cell type-, cell state-, or tissue type-dependent. For example, an enhancer region may comprise a transcription factor binding site that binds one or more transcription factors that enhance transcription of nearby polynucleotides comprising coding sequences, wherein the presence of the one or more transcription factors is cell type-, cell state-, or tissue type-dependent. In some embodiments, transcriptional enhancement by the enhancer region may be regulated by one or more transcription factors, such as cell type-, cell state-, or tissue type-specific transcription factors, and therefore be cell type-, cell state-, or tissue type-dependent. For example, the enhancer region may be unactivated when in an unbound state and may be activated upon binding of one or more transcription factors, wherein this binding is dependent on the presence of the one or more transcription factors and wherein the presence of the one or more transcription factors is cell type-, cell state-, or tissue type-dependent. Alternatively or in addition, the enhancer region may be unactivated when in a bound state to one or more corepressors and may be activated upon decreased or no binding of the one or more corepressors, wherein this binding is dependent on cell type-, cell state-, or tissue type-dependent. The activated enhancer may enhance recruitment of a polymerase to a core promoter relative to the unactivated enhancer. The activated enhancer may enhance recruitment of a polymerase to a core promoter relative to the inactive enhancer.

A response element (e.g., an enhancer region) may be paired with a core promoter, such as an engineered core promoter described herein, to promote transcription of a polynucleotide encoding a payload. The sequence of the response element may be positioned in a polynucleotide sequence such that it affects recruitment of a polymerase to the core promoter. For example, the response element (e.g., an enhancer region) may be positioned 5′ of the core promoter, or the response element may be positioned 3′ of the polynucleotide encoding the payload.

A response element (e.g., an enhancer region) may comprise a sequence that binds to cognate ligands or coactivators expressed in a target cell type (e.g., neurons, renal cells, hepatocytes, podocytes, retinal cells, epithelial cells, muscle cells, erythrocytes, platelets, bone marrow cells, endothelial cells, epidermal cells, lymphocytes, glial cells, interstitial cells, adipocytes, or fibroblasts) or cell state (e.g., diseased cells or healthy cells) at higher levels than a non-target cell type or non-target cell state. Alternatively or in addition, a response element (e.g., an enhancer region) may comprise a sequence that binds to corepressors expressed in a target cell type or cell state at lower levels than a non-target cell type or non-target cell state. For example, an enhancer region of a promoter for expressing a payload in a kidney cell may comprise a transcription factor binding sequence that binds to a kidney-specific transcription factor. The kidney-specific enhancer region may enhance transcription initiation when bound to the kidney-specific transcription factor in the kidney cells, increasing transcription of the payload in kidney cells relative to non-kidney cells. In another example, an enhancer region of a promoter for expressing a payload in a central nervous system (CNS) cell may comprise a transcription factor binding sequence that binds to a CNS-specific transcription factor. The CNS-specific enhancer region may enhance transcription initiation when bound to the CNS-specific transcription factor in CNS cells, increasing transcription of the payload in CNS cells relative to non-CNS cells. As a further example, an enhancer region for expressing a payload in a liver cell may comprise a transcription factor binding sequence that binds to a liver-specific transcription factor. The liver-specific enhancer region may enhance transcription initiation when bound to the liver-specific transcription factor in liver cells, increasing transcription of the payload in liver cells relative to non-liver cells. Examples of response elements are provided in TABLE 1.

TABLE 1
Exemplary Response Element Sequences

| SEQ ID NO | Sequence |
|---|---|
| SEQ ID | TGCTATTTTTAGCGGGCTTTTTTTGACGGG |
| NO: 145 | AGGAAGGAAGGAGGGAGAGGGACGGGAAGT |
| | AAAGAGAAAAAGAGGAAGTGAAAGCTAAGA |
| | AGGAAGTGACGGCTGGCGGGGACAGATAAG |
| | AGTTGTCTAGTTGTGATAATGGAACTGCTG |
| | AGTCATGGATTGCAGAGTCAC |
| SEQ ID | GGTTAATGATTAACCGTGTAAATAATTAGC |
| NO: 146 | GGATCACGTGATGGTCACGTGTTGGTTCAA |
| | GGCCAGAGTCAAGGTCGGGAGTCCAAAGTC |
| | CAGAAGTGCAAGGTCCGGTGTTTACTTTGG |
| | GTGTTTACCTTCC |
| SEQ ID | GGTTAATGATTAACCgTGTAAATAATTAGC |
| NO: 193 | GgATCACGTGATgGTCACGTGTTgGTTCAA |
| | GGCCAgAGTCAAGGTCGgGAGTCCAAAGTC |
| | CAgAAGTGCAAGGTCCGgTGTTTACTTTGG |
| | gTGTTTACCTTCC |
| SEQ ID | AGAAACACGGCACAGATGACGGCTTTAAAA |
| NO: 194 | GAAAAGAGTCCATTGACTGGAAAAGCAAAC |
| | AGTATGATGGTCAAAGGCCAAGAATCAGAA |
| | AACAGAGATTTTTCCTCTTCTCTTGTTTCA |
| | CAAAGTAAATAAACATGAGACATAT |

A response element may comprise a sequence having at least 80%, at least 85%, at least 90%, at least 93%, at least 95%, at least 97%, at least 99%, or 100% sequence identity to SEQ ID NO: 145, SEQ ID NO: 146, SEQ ID NO: 193, or SEQ ID NO: 194. The response element may comprise a sequence having at least 90% sequence identity to SEQ ID NO: 145, SEQ ID NO: 146, SEQ ID NO: 193, or SEQ ID NO: 194. The response element may comprise a sequence of SEQ ID NO: 145, SEQ ID NO: 146, SEQ ID NO: 193, or SEQ ID NO: 194. In some embodiments, a response element may be an engineered response element. For example, an engineered response element may comprise a sequence of SEQ ID NO: 145 or SEQ ID NO: 146. In some embodiments, the response element may be an enhancer region. In some embodiments, the response element may be a liver-specific enhancer region, wherein the enhancer region is activated in liver cells. For example, a liver-specific enhancer region may comprise a sequence of SEQ ID NO: 146 or SEQ ID NO: 197. In some embodiments, a liver-specific enhancer/promoter may comprise a sequence of SEQ ID NO: 163-SEQ ID NO: 177. In some embodiments, the response element may be a bone marrow-specific enhancer, wherein the enhancer region is activated in bone marrow cells. For example, a bone marrow-specific enhancer region may comprise a sequence of SEQ ID NO: 145 or SEQ ID NO: 193. In some embodiments, a bone marrow-specific enhancer region/promoter may comprise a sequence of SEQ ID NO: 148-SEQ ID NO: 162.

The response elements (e.g., a response element of SEQ ID NO: 145, SEQ ID NO: 146, SEQ ID NO: 193, or SEQ ID NO: 194) may be appended to core promoter or promoter for expression of a polynucleotide comprising a payload sequence (e.g., a transgene encoding a therapeutic protein or a sequence encoding a therapeutic polynucleotide) to promote cell type-, tissue type-, or cell state-specific transcription of a polynucleotide encoding for a payload (i.e., a payload sequence). In some embodiments, response elements may be combined or used in combination with other response elements to tune transcriptional levels of the payload sequence. In some embodiments, the 1, 2, 3, 4, 5, 6, 7, 8, 9, 10, or more response elements may be combined to tune transcriptional levels of the payload sequence. The combined 1, 2, 3, 4, 5, 6, 7, 8, 9, 10, or more response elements may all be the same response element, different response elements, or any combination thereof. For example, a promoter may comprise a combination of sequences for transcription enhancement and sequences for transcription repression (e.g., via increased binding to a corepressor) to tune payload expression in the cell type or cell state of interest. The response element (e.g., a response element of SEQ ID NO: 145, SEQ ID NO: 146, SEQ ID NO: 193, or SEQ ID NO: 194) may be paired with a core promoter (e.g., a core promoter of SEQ ID NO: 1-SEQ ID NO: 4, SEQ ID NO: 7, or SEQ ID NO: 10-SEQ ID NO: 144) to form an engineered promoter. The engineered promoter may facilitate cell-specific payload expression under transcriptional control of the engineered promoter.

### Core Promoters

The promoter of a polynucleotide may comprise a core promoter that facilitates recruitment of transcription machinery and initiation of transcription. The core promoter may be an engineered core promoter. In some embodiments, the core promoter may be engineered for switchable activity when paired with a cell-specific response element (e.g., a cell-specific enhancer). A switchable core promoter may exhibit low or no activity (low or no transcriptional activation) when paired with a cell-specific response element (e.g., a cell-specific enhancer) that is in a cell that is not its specific cell type (e.g., a cell type that is not the specific cell type of the cell-specific response element), and may exhibit high activity (high transcriptional activation) when paired with that same cell-specific response element (e.g., that same cell-specific enhancer), but in a cell that is its specific cell type (e.g., a cell type that is the specific cell type of that same cell-specific response element), and then that same switchable core promoter may exhibit low or no activity (low or no transcriptional activation) when paired with a different cell-specific response element (e.g., a different cell-specific enhancer) that is in a cell that is not its specific cell type (e.g., a cell type that is not the specific cell type of the different cell-specific response element), and may exhibit high activity (high transcriptional activation) when paired with that same different cell-specific response element (e.g., the same different cell-specific enhancer), but in a cell that is its specific cell type (e.g., a cell type that is the specific cell type of the same different cell-specific response element). For example, a switchable core promoter (e.g., any of SEQ ID NO: 1-SEQ ID NO: 4, SEQ ID NO: 7, or SEQ ID NO: 10-SEQ ID NO: 144) paired with a neuron-specific enhancer may exhibit high transcriptional activity in neurons and low transcriptional activity in non-neuronal cells (e.g., kidney cells, liver cells, bone marrow cells, etc.). In another example, that switchable core promoter (e.g., any of SEQ ID NO: 1-SEQ ID NO: 4, SEQ ID NO: 7, or SEQ ID NO: 10-SEQ ID NO: 144) paired with a hepatocyte-specific enhancer may exhibit high transcriptional activity in hepatocytes and low transcriptional activity in non-hepatocytes (e.g., kidney cells, neurons, bone marrow cells, etc.). In some embodiments, the core promoter may be engineered for switchable activity when paired with a tissue-specific response element (e.g., a tissue-specific enhancer). A switchable core promoter may exhibit low or no activity (low or no transcriptional activation) when paired with a tissue-specific response element (e.g., a tissue-specific enhancer) that is in a cell that is not its specific tissue type (e.g., a cell that is not the specific tissue type of the tissue-specific response element), and may exhibit high activity (high transcriptional activation) when paired with that same tissue-specific response element (e.g., that same tissue-specific enhancer), but in a cell that is its specific tissue type (e.g., a cell that is the specific tissue type of that same tissue-specific response element), and then that same switchable core promoter may exhibit low or no activity (low or no transcriptional activation) when paired with a different tissue-specific response element (e.g., a different tissue-specific enhancer) that is in a cell that is not its specific tissue type (e.g., a cell that is not the specific tissue type of the different tissue-specific response element), and may exhibit high activity (high transcriptional activation) when paired with that same different tissue-specific response element (e.g., that same different tissue-specific enhancer), but in a cell that is its specific tissue type (e.g., a cell that is the specific tissue type of that same different tissue-specific response element). For example, a switchable core promoter (e.g., any of SEQ ID NO: 1-SEQ ID NO: 4, SEQ ID NO: 7, or SEQ ID NO: 10-SEQ ID NO: 144) paired with a CNS-specific enhancer may exhibit high transcriptional activity in cells of the CNS and low transcriptional activity in non-CNS cells (e.g., cells of the kidney, liver, bone marrow, etc.). In another example, that switchable core promoter (e.g., any of SEQ ID NO: 1-SEQ ID NO: 4, SEQ ID NO: 7, or SEQ ID NO: 10-SEQ ID NO: 144) paired with a liver-specific enhancer may exhibit high transcriptional activity in liver cells and low transcriptional activity in non-liver cells (e.g., cells of the kidney, CNS, bone marrow, etc.). A switchable core promoter may exhibit low or no activity (low or no transcriptional activation) when paired with a cell state-specific response element (e.g., a cell state-specific enhancer) that is in a cell in a specific cell state that is not its specific cell state (e.g., a cell not in a specific cell state of the cell state-specific response element), and may exhibit high activity (high transcriptional activation) when paired with that same cell state-specific response element (e.g., that same cell state-specific enhancer), but in a cell that is in its specific cell state (e.g., a cell that is in the specific cell state of that same cell state-specific response element), and then that same switchable core promoter may exhibit low or no activity (low or no transcriptional activation) when paired with a different cell state-specific response element (e.g., a different cell state-specific enhancer) that is in a cell in a specific cell state that is not its different specific cell state (e.g., a cell not in a specific cell state of the different cell state-specific response element), and may exhibit high activity (high transcriptional activation) when paired with that same different cell state-specific response element (e.g., that same different cell state-specific enhancer), but in a cell that is in its specific different cell state (e.g., a cell that is in the specific cell state of that same different cell state-specific response element). For example, a switchable core promoter (e.g., any of SEQ ID NO: 1-SEQ ID NO: 4, SEQ ID NO: 7, or SEQ ID NO: 10-SEQ ID NO: 144) paired with a disease-specific enhancer may exhibit high transcriptional activity in cells exhibiting that disease and low transcriptional activity in cells not exhibiting that disease. In another example, that switchable core promoter (e.g., any of SEQ ID NO: 1-SEQ ID NO: 4, SEQ ID NO: 7, or SEQ ID NO: 10-SEQ ID NO: 144) paired with a Rett-specific enhancer may exhibit high transcriptional activity in neurons expressing mutant MeCP2 associated with Rett Syndrome and low transcriptional activity in neurons expressing functional MeCP2 associated with a normal neuronal phenotype. A switchable core promoter may exhibit high activity when paired with a enhancer that is cell type-, cell state-, or tissue type-dependent and in that specific cell type, cell state, or tissue type. A switchable core promoter may exhibit a wide dynamic range of activity when paired with a cell type-, cell state-, or tissue type-specific enhancer when in that cell type, cell state, or tissue type as compared to when not in that cell type, cell state, or tissue type, respectively.

In some embodiments, the core promoter may be positioned downstream (i.e., 3′) of a response element. In some embodiments, the core promoter may be positioned upstream (i.e., 5′) of a polynucleotide encoding a payload sequence (e.g., a transgene or sequence encoding a therapeutic polynucleotide). The core promoter may recruit polymerase or proteins that bind to polymerases to initiate transcription of a sequence downstream of the core promoter. In some embodiments, the core promoter may recruit an RNA polymerase (e.g., RNA polymerase II) or a TATA binding protein (TBP) that recruits an RNA polymerase when in combination with a response element bound to cognate ligands, coactivators, or corepressors. For example, the core promoter may bind to general transcription factors (GTFs) which recruit RNA polymerase II (Pol II) to initiate transcription. The ability of the core promoter to recruit transcription machinery (e.g., an RNA polymerase) or the affinity of the core promoter for the transcription machinery may affect transcription levels. In some embodiments, the core promoter may be altered to tune transcription levels by altering recruitment of and/or affinity for transcription machinery. In some embodiments, an engineered core promoter is a mutated or altered core promoter that has a decreased ability to recruit transcription machinery (e.g., an RNA polymerase) and/or decreased affinity for the transcription machinery as compared to a corresponding unmutated or unaltered core promoter. In some embodiments, an engineered core promoter is a de novo synthetic core promoter that has a decreased ability to recruit transcription machinery (e.g., an RNA polymerase) and/or decreased affinity for the transcription machinery as compared to other core promoters, such as a core promoter of SEQ ID NO: 5 or SEQ ID NO: 9. In some embodiments, the ability of this engineered core promoter to initiate transcriptional activity is dependent on being paired with a response element, wherein the initiation of transcriptional activity occurs when the response element binds to cognate ligands and/or coactivators, and is not initiated or initiated at a low basal level when the response element is not bound to cognate ligands and/or coactivators, or optionally, is bound to corepressors.

In some embodiments, the core promoter is a minimal synthetic core promoter, such as a minP core promoter (SEQ ID NO: 5) or a variant of a minP core promoter (e.g., SEQ ID NO: 4 or SEQ ID NO: 15). Core promoters may be selected or engineered for one or more desired transcriptional properties, such as transcription level, cell type specificity, cell state specificity, tissue specificity, or cell genotype specificity. In some embodiments, a core promoter may be engineered to selectively promote transcription initiation when paired with a cell type-, cell state-, or tissue type-specific response element (e.g., a cell type-, cell state-, or tissue type-specific enhancer). Engineering a core promoter may comprise screening variants of a core promoter for transcription level, cell type specificity, cell state specificity, tissue specificity, or cell genotype specificity.

The core promoters described herein may comprise engineered sequences that serve as a promoter switch that can be selectively activated in a target cell type, target tissue type, or target cell state. A switchable core promoter may readily promote transcription initiation when paired with a cell type-, tissue type-, or cell state-specific response element in the target cell type, target tissue type, or target cell state, respectively, while exhibiting low background transcription initiation in non-target cell types, non-target tissue types, or non-target cell states, respectively. The switchable core promoter may be used in combination with a variety of response elements or other response elements to promote cell type-, tissue type-, or cell state-specific transcription in a variety of cell types, tissue types, or cell states.

A switchable core promoter may promote transcription initiation in a target cell type, tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in a non-target cell type, non-target tissue type, or non-target cell state, when paired with a response element specific for the target cell type, target tissue type, or target cell state. In some embodiments, a switchable core promoter may promote transcription initiation in a target cell type, tissue type, or target cell state when paired with a response element specific for the target cell type, target tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in the target cell type, target tissue type, or target cell state, when paired with a response element not specific for the target cell type, target tissue type, or target cell state.

In some embodiments, a switchable core promoter may promote transcription initiation when paired with an active response element at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation compared to the switchable core promoter when paired with an inactive response element. In some embodiments, a switchable core promoter may promote transcription initiation in a target cell type, target tissue type, or target cell state when paired with a response element specific for the target cell type, target tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in a target cell type, tissue type, or target cell state compared to the switchable core promoter when paired with an inactive response element. A switchable core promoter may promote transcription initiation when paired with a response element bound to a cognate ligand or coactivator (e.g., an enhancer bound to a cognate ligand or coactivator) (an activated response element) at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with that response element not bound to a cognate ligand or coactivator (e.g., an enhancer not bound to a cognate ligand or coactivator) (an unactivated response element). The activated response element may be an enhancer specific for a target cell type, target tissue type, or target cell state in that target cell type, target tissue type, or target cell state, respectively. In some embodiments, the unactivated response element may be an enhancer that is not specific for the cell type, tissue type, or cell state that it is in.

A switchable core promoter may promote transcription initiation when paired with a response element bound to a cognate ligand or coactivator (e.g., an enhancer bound to a cognate ligand or coactivator) and/or having decreased binding to a corepressor at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with that response element not bound to a cognate ligand or coactivator (e.g., an enhancer not bound to a cognate ligand or coactivator) and/or having increased binding to a corepressor.

A switchable core promoter may promote transcription initiation when paired with a response element capable of being bound to a cognate ligand or coactivator (e.g., an enhancer bound to a cognate ligand or coactivator) (an active response element) that is bound to a cognate ligand or coactivator (e.g., an enhancer bound to a cognate ligand or coactivator) (an activated response element) at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with a response element not capable of being bound to a cognate ligand or coactivator (e.g., an enhancer not capable of being bound to a cognate ligand or coactivator) (an inactive response element), and therefore which may not be bound to a cognate ligand or coactivator (e.g., an enhancer not bound to a cognate ligand or coactivator) (an unactivated response element). The active response element may be specific for a target cell type, target tissue type, or target cell state, and when in that target cell type, target tissue type, or target cell state, respectively, may be an activated response element. The unactivated response element may be specific for a target cell type, target tissue type, or target cell state, but that may not be in that specific target cell type, target tissue type, or target cell state, respectively. The inactive response element may be a response element that may not be capable of activation regardless of the cell type, tissue type, or cell state it is in, and therefore, may always be an unactivated response element in a cell.

A switchable core promoter may promote transcription initiation in a target cell type, tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in a non-target cell type, non-target tissue type, or non-target cell state, when paired with an enhancer specific for the target cell type, target tissue type, or target cell state. In some embodiments, a switchable core promoter may promote transcription initiation in a target cell type, tissue type, or target cell state when paired with an enhancer specific for the target cell type, target tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in the target cell type, target tissue type, or target cell state, when paired with an enhancer not specific for the target cell type, target tissue type, or target cell state.

In some embodiments, a switchable core promoter may promote transcription initiation when paired with an active enhancer at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation compared to the switchable core promoter when paired with an inactive enhancer. In some embodiments, a switchable core promoter may promote transcription initiation in a target cell type, tissue type, or target cell state when paired with an enhancer specific for the target cell type, target tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in a target cell type, tissue type, or target cell state compared to the switchable core promoter when paired with an inactive enhancer.

A switchable core promoter may promote transcription initiation when paired with an enhancer bound to a cognate ligand or coactivator (an activated enhancer) at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with that enhancer not bound to a cognate ligand or coactivator (an unactivated enhancer). The activated enhancer may be specific for a target cell type, target tissue type, or target cell state in that target cell type, target tissue type, or target cell state, respectively. In some embodiments, the unactivated enhancer may be not specific for the cell type, tissue type, or cell state that it is in.

A switchable core promoter may promote transcription initiation when paired with an enhancer bound to a cognate ligand or coactivator and/or having decreased binding to a corepressor at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with that enhancer not bound to a cognate ligand or coactivator and/or having increased binding to a corepressor.

A switchable core promoter may promote transcription initiation when paired with an enhancer capable of being bound to a cognate ligand or coactivator (an active enhancer) that is bound to a cognate ligand or coactivator (an activated enhancer) at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with an enhancer not capable of being bound to a cognate ligand or coactivator (an inactive enhancer), and therefore which is not bound to a cognate ligand or coactivator (an unactivated enhancer). The active enhancer may be specific for a target cell type, target tissue type, or target cell state, and when in that target cell type, target tissue type, or target cell state, respectively, may be an activated enhancer. The unactivated enhancer may be specific for a target cell type, target tissue type, or target cell state, but that may not be in that specific target cell type, target tissue type, or target cell state, respectively. The inactive enhancer may not be capable of activation regardless of the cell type, tissue type, or cell state it is in, and therefore, may always an unactivated enhancer in a cell.

The engineered core promoters of the presence disclosure provide high dynamic range, also referred to as fold activation, relative to endogenous core promoters. In some embodiments, dynamic range may describe the transcriptional activity of a core promoter when paired with an activated response element relative to the transcriptional activity of the core promoter when paired with an unactivated response element. A core promoter with a high dynamic range may promote transcription initiation in a cell at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in the cell having an endogenous core promoter, paired with the same response element. In some embodiments, a core promoter with a high dynamic range may promote transcription initiation when paired with an activated response element at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with an inactive response element.

The engineered core promoters of the presence disclosure provide high dynamic range, also referred to as fold activation, for improved cell type-, tissue type-, or cell state-specificity relative to endogenous core promoters. In some embodiments, dynamic range may describe the transcriptional activity of a core promoter when paired with an activated response element (e.g., an enhancer bound to a cognate ligand or coactivator) relative to the transcriptional activity of the core promoter when paired with an inactive response element. A core promoter with a high dynamic range may promote transcription initiation in a target cell type, target tissue type, or target cell state at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation in a non-target cell type, non-target tissue type, or non-target cell state. In some embodiments, a core promoter with a high dynamic range may promote transcription initiation when paired with an activated response element at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with an inactive response element. In some embodiments, a core promoter with a high dynamic range may promote transcription initiation when paired with an activated enhancer at a rate that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold that of the rate of transcription initiation when paired with an unactivated enhancer.

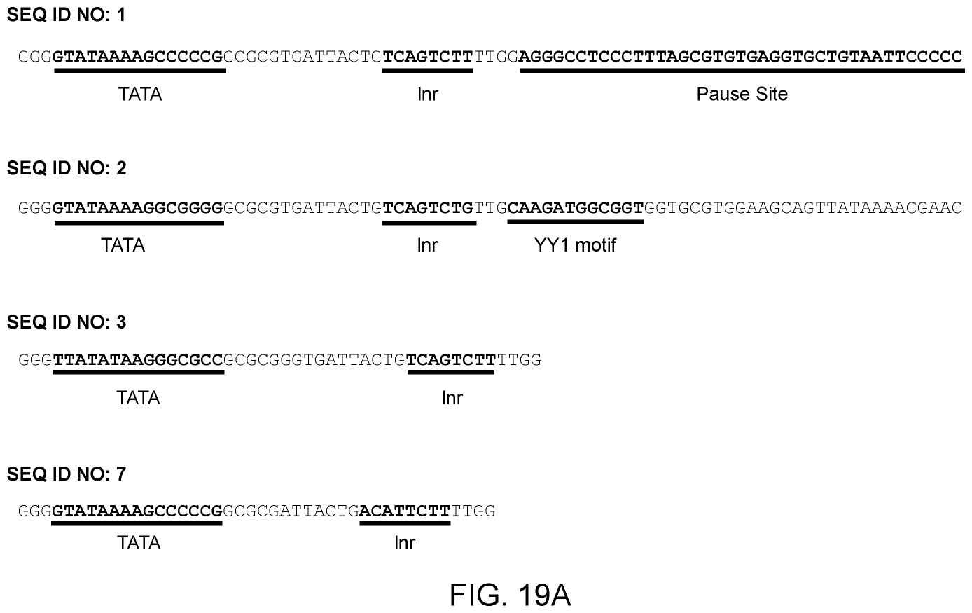

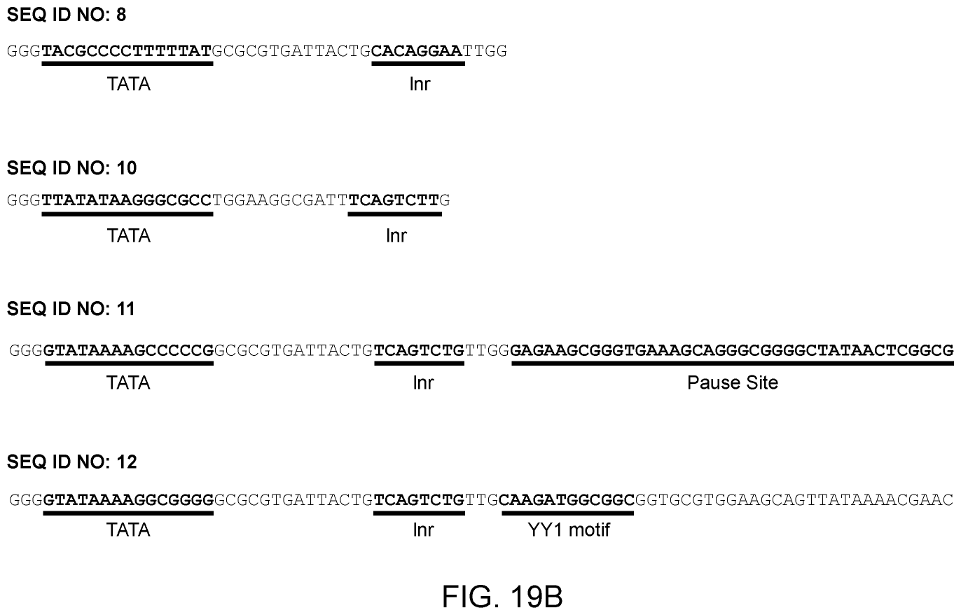

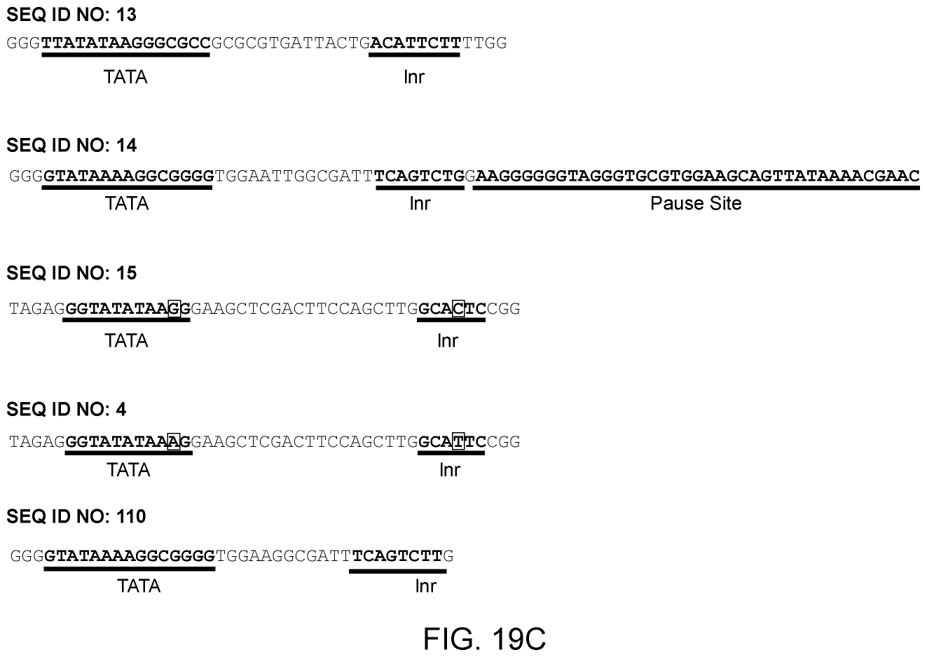

A core promoter may comprise one or more sequence elements. In some embodiments, a core promoter may comprise, from 5′ to 3′, a polynucleotide encoding a TATA box sequence, a spacer sequence, and an initiator element sequence. In some embodiments, a core promoter may comprise, from 5′ to 3′, a TATA box, a spacer, and an initiator element. In some embodiments, a core promoter may further comprise a transcriptional pause site, a YY1 transcription factor binding motif, or a combination thereof. In some embodiments, a core promoter may comprise a transcription factor binding motif. For example, the spacer sequence may comprise a transcription factor binding sequence. In some embodiments, sequence elements within a core promoter may be configured as shown in FIG. 19A, FIG. 19B, or FIG. 19C. Examples of sequence elements that may be included in a core promoter of the present disclosure are provided in TABLE 2.

TABLE 2
Exemplary Core Promoter Sequence Elements

| Element Type | SEQ ID NO | Sequence |
|---|---|---|
| Pause Site | SEQ ID NO: 196 | GACGAGCGCGTGGGGGGTGACGGTGGCTGATT |
| | | ATCACGG |
| Pause Site | SEQ ID NO: 197 | GGGGAGCGGGATGTTGTCGACGGCCGGGGATA |
| | | AGCCCTC |
| Pause Site | SEQ ID NO: 198 | AGGGCCTCCCTTTAGCGTGTGAGGTGCTGTAAT |
| | | TCCCCC |
| Pause Site | SEQ ID NO: 211 | GAGAAGCGGGTGAAAGCAGGGCGGGGCTATA |
| | | ACTCGGCG |
| Pause Site | SEQ ID NO: 212 | AAGGGGGGTAGGGTGCGTGGAAGCAGTTATAA |
| | | AACGAAC |
| TATA Box | SEQ ID NO: 199 | GTATAAAAAGCGGGG |
| TATA Box | SEQ ID NO: 200 | GTATAAAAGGCGGGG |
| TATA Box | SEQ ID NO: 201 | TTATATAAGGGCGCC |
| TATA Box | SEQ ID NO: 213 | GTATAAAAGCCCCCG |
| TATA Box | SEQ ID NO: 214 | GGTATATAAAG |
| TATA Box | SEQ ID NO: 215 | GGTATATAAGG |
| Initiator | SEQ ID NO: 202 | TCAGTCTT |
| Element (Inr) | | |
| Initiator | SEQ ID NO: 203 | TCAGTCTG |
| Element (Inr) | | |
| Initiator | SEQ ID NO: 204 | ACATTCTT |
| Element (Inr) | | |
| Initiator | SEQ ID NO: 216 | GCATTC |
| Element (Inr) | | |
| Initiator | SEQ ID NO: 217 | GCACTC |
| Element (Inr) | | |
| YY1 Motif | SEQ ID NO: 205 | CAAGATGGCGGT |
| YY1 Motif | SEQ ID NO: 206 | CAAGATGGCGGC |
| Element Type | SEQ ID NO | Sequence |
| YY1 Motif | SEQ ID NO: 207 | CAAAATGGCGGC |
| Background | SEQ ID NO: 208 | GGGTGACTGGAGTGCTTAGCGCGTGATTACTG |
| | | CTGGAGGATTGG |
| Background | SEQ ID NO: 209 | GGGTTACTGCTGGAGGATTGGAATTGGCGATT |
| | | CTTACGCGG |

A core promoter may comprise one or more sequence elements, such as a TATA box (e.g., TATAAA), an initiator element, an RNA polymerase binding sequence, a B recognition element (BRE, e.g., G/C,G/C,G/A,CGCC), a CCAAT box or CAT box (e.g., GGCCAATCT), or a Pribnow box (e.g., TATAAT). In some embodiments, a core promoter may comprise a TATA box (e.g., an engineered TATA box of SEQ ID NO: 199-SEQ ID NO: 201 or SEQ ID NO: 213-SEQ ID NO: 215), for example as shown in SEQ ID NO: 4 and SEQ ID NO: 15. In some embodiments, a core promoter may comprise a TATA box sequence having at least about 70%, at least about 72%, at least about 75%, at least about 78%, at least about 80%, at least about 82%, at least about 85%, at least about 87%, at least about 90%, at least about 92%, at least about 95%, at least about 97%, or about 100% sequence identity to SEQ ID NO: 199-SEQ ID NO: 201 or SEQ ID NO: 213-SEQ ID NO: 215.

In some embodiments, a core promoter may comprise an initiator element (e.g., an engineered initiator element of SEQ ID NO: 202-SEQ ID NO: 204, SEQ ID NO: 216, or SEQ ID NO: 217). In some embodiments, a core promoter may comprise an initiator element having at least about 70%, at least about 72%, at least about 75%, at least about 78%, at least about 80%, at least about 82%, at least about 85%, at least about 87%, at least about 90%, at least about 92%, at least about 95%, at least about 97%, or about 100% sequence identity to SEQ ID NO: 202-SEQ ID NO: 204, SEQ ID NO: 216, or SEQ ID NO: 217.

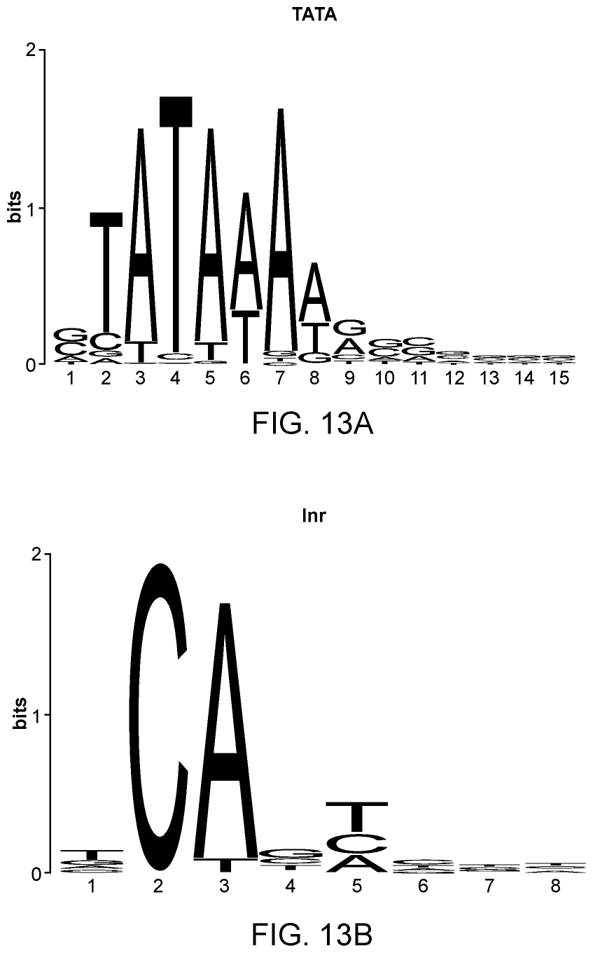

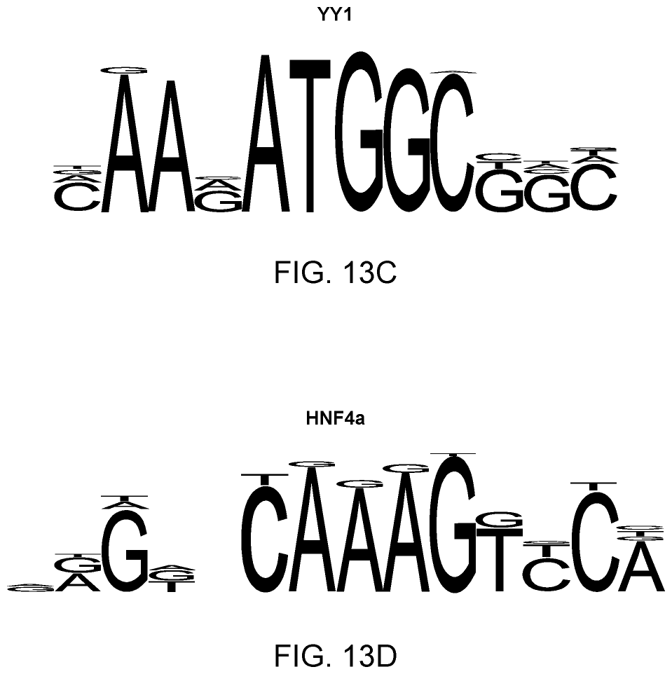

In some embodiments, a core promoter may comprise a long spacer (e.g., an engineered long spacer between a TATA box and an initiator element, for example as shown in SEQ ID NO: 3 and SEQ ID NO: 69-SEQ ID NO: 108. A long spacer may have a length of no less than 31 nucleotide residues from the TATA box to the initiator element, wherein the number of nucleotide residues from the TATA box to the initiator element are counted from the first T in the TATA box to the A in the CAG in the initiator element (or the corresponding CAG in the parent sequence if the initiator element is mutated), inclusive. In some embodiments, a long spacer may have a length of from 31 to 48 nucleotide residues from the TATA box to the initiator element. In some embodiments, a long spacer may have a length of from 31 to 44 nucleotide residues from the TATA box to the initiator element. In some embodiments, a long spacer may have a length of from 31 to 42 nucleotide residues from the TATA box to the initiator element. In some embodiments, a long spacer may have a length of from 31 to 37 nucleotide residues or from 31 to 35 nucleotide residues from the TATA box to the initiator element. For example, a long spacer may have a length of 32 nucleotide residues from the TATA box to the initiator element. In some embodiments, a core promoter may comprise an engineered a short spacer (e.g., an engineered short spacer) between a TATA box and an initiator element, wherein the number of nucleotide residues from the TATA box to the initiator element are counted from the first T in the TATA box to the A in the CAG in the initiator element (or the corresponding CAG in the parent sequence if the initiator element is mutated), inclusive, for example as shown in SEQ ID NO: 7, SEQ ID NO: 10, and SEQ ID NO: 109-SEQ ID NO: 125. A short spacer may have a length of no more than 28 nucleotide residues from the TATA box to the initiator element. In some embodiments, a short spacer may have a length of from 22 to 29 nucleotide residues or from 24 to 29 nucleotide residues from the TATA box to the initiator element. For example, a short spacer may have a length of 28 nucleotide residues from the TATA box to the initiator element. In some embodiments, a core promoter may comprise an engineered spacer between a TATA box and an initiator element, for example as shown in SEQ ID NO: 13, and SEQ ID NO: 126-SEQ ID NO: 144. A spacer may have a length of about 30 nucleotide residues from the TATA box to the initiator element. Spacing between TATA and Inr elements of select core promoters are illustrated in FIG. 19A, FIG. 19B, and FIG. 19C. In some embodiments, a spacer sequence of a core promoter may comprise a response element, such as a transcription factor binding site (e.g., a HNF4a binding site provided in FIG. 13D, a GATA1 binding site, a YY1 binding site, or a CEBPA binding site). For example, a core promoter may contain a spacer sequence that binds HNF4a, as shown in SEQ ID NO: 22-SEQ ID NO: 30. Alternatively or in addition, a transcription factor binding site may be included after the Inr element. In some embodiments, a core promoter may be engineered into a background sequence, such as a sequence of SEQ ID NO: 208 or SEQ ID NO: 209.

In some embodiments, a core promoter may comprise a transcription pause site (e.g., SEQ ID NO: 196-SEQ ID NO: 198, SEQ ID NO: 211, or SEQ ID NO: 212), for example as shown in SEQ ID NO: 1, SEQ ID NO: 11, SEQ ID NO: 14, and SEQ ID NO: 16-SEQ ID NO: 21. In some embodiments, a core promoter may comprise a transcription pause site having at least about 70%, at least about 72%, at least about 75%, at least about 78%, at least about 80%, at least about 82%, at least about 85%, at least about 87%, at least about 90%, at least about 92%, at least about 95%, at least about 97%, or about 100% sequence identity to SEQ ID NO: 196-SEQ ID NO: 198, SEQ ID NO: 211, or SEQ ID NO: 212.

In some embodiments, a core promoter may comprise a YY1 motif (e.g., SEQ ID NO: 205-SEQ ID NO: 207), for example as shown in SEQ ID NO: 2, SEQ ID NO: 12, and SEQ ID NO: 31-SEQ ID NO: 68. In some embodiments, a core promoter may comprise a YY1 motif having at least about 70%, at least about 72%, at least about 75%, at least about 78%, at least about 80%, at least about 82%, at least about 85%, at least about 87%, at least about 90%, at least about 92%, at least about 95%, at least about 97%, or about 100% sequence identity to SEQ ID NO: 205-SEQ ID NO: 207. In some embodiments, a core promoter may comprise a YY1 motif (e.g., SEQ ID NO: 205-SEQ ID NO: 207), for example as shown in SEQ ID NO: 2, SEQ ID NO: 12, and SEQ ID NO: 31-SEQ ID NO: 68. In some embodiments, a core promoter may comprise a YY1 motif having at least about 70%, at least about 72%, at least about 75%, at least about 78%, at least about 80%, at least about 82%, at least about 85%, at least about 87%, at least about 90%, at least about 92%, at least about 95%, at least about 97%, or about 100% sequence identity to SEQ ID NO: 205-SEQ ID NO: 207 and is capable of binding to YY1. In some embodiments, an engineered core promoter sequence may comprise one or more mutations relative to a founder core promoter sequence. In some embodiments, an engineered core promoter sequence may comprise one or more point mutations (e.g., T15A, T15G, A40T, or A40C) relative to SEQ ID NO: 5 (TAGAGGGTATATAATGGAAGCTCGACTTCCAGCTTGGCAATCCGG), for example as shown in SEQ ID NO: 4 and SEQ ID NO: 15. In some embodiments, an engineered core promoter sequence may comprise one or more point mutations relative to SEQ ID NO: 9 (TCTAGAGGGTATATAATGGGGGCCACTAGTCTACTACCAGAAAG). In some embodiments, an engineered core promoter sequence may comprise one or more point mutations relative to SEQ ID NO: 210

(AGGACCGGATCAACTAGGTCTATATAAGCAGAGCTCGT
TTAGTGAACCGTCAGATCGCCTGGAGACGCCATCCACG
CTGTTTTGACCTCCATAGAATTGGTACCGAGCTCG).

In an embodiment, a core promoter may comprise a TATA box sequence having at least about 70%, at least about 80%, at least about 90%, at least about 95%, at least about 97%, or about 100% sequence identity to SEQ ID NO: 213, a spacer, an initiator element having at least about 70%, at least about 80%, at least about 90%, at least about 95%, at least about 97%, or about 100% sequence identity to SEQ ID NO: 202, and a pause site having at least about 70%, at least about 80%, at least about 90%, at least about 95%, at least about 97%, or about 100% sequence identity to SEQ ID NO: 198, optionally the spacer may be of 29 to 33 nucleotides.

In an embodiment, a core promoter may comprise a TATA box sequence having the sequence of SEQ ID NO: 213, a spacer of 31 nucleotides, an initiator element having the sequence of SEQ ID NO: 202, and a pause site of SEQ ID NO: 198.

In an embodiment, a core promoter may comprise a TATA box sequence having at least about 70%, at least about 80%, at least about 90%, at least about 95%, at least about 97%, or about 100% sequence identity to SEQ ID NO: 200, a spacer, an initiator element having at least about 70%, at least about 80%, at least about 90%, at least about 95%, at least about 97%, or about 100% sequence identity to SEQ ID NO: 203, and a YY1 motif having at least about 70%, at least about 80%, at least about 90%, at least about 95%, at least about 97%, or about 100% sequence identity to SEQ ID NO: 205, optionally the spacer may be of 29 to 33 nucleotides.

In an embodiment, a core promoter may comprise a TATA box sequence having the sequence of SEQ ID NO: 200, a spacer of 31 nucleotides, an initiator element having the sequence of SEQ ID NO: 203, and a YY1 motif of SEQ ID NO: 205.

In an embodiment, a core promoter may comprise a TATA box sequence having at least about 70%, at least about 80%, at least about 90%, at least about 95%, at least about 97%, or about 100% sequence identity to SEQ ID NO: 201, a spacer, and an initiator element having at least about 70%, at least about 80%, at least about 90%, at least about 95%, at least about 97%, or about 100% sequence identity to SEQ ID NO: 202, optionally the spacer may be of 31 to 35 nucleotides.

In an embodiment, a core promoter may comprise a TATA box sequence having the sequence of SEQ ID NO: 201, a spacer of 33 nucleotides, and an initiator element having the sequence of SEQ ID NO: 202.

In an embodiment, a core promoter may comprise a TATA box sequence having at least about 70%, at least about 80%, at least about 90%, at least about 95%, at least about 97%, or about 100% sequence identity to SEQ ID NO: 214, a spacer, and an initiator element having at least about 70%, at least about 80%, at least about 90%, at least about 95%, at least about 97%, or about 100% sequence identity to SEQ ID NO: 216, optionally the spacer may be of 30 to 34 nucleotides.

In an embodiment, a core promoter may comprise a TATA box sequence having the sequence of SEQ ID NO: 214, a spacer of 32 nucleotides, and an initiator element having the sequence of SEQ ID NO: 216.

In an embodiment, a core promoter may comprise a TATA box sequence having at least about 70%, at least about 80%, at least about 90%, at least about 95%, at least about 97%, or about 100% sequence identity to SEQ ID NO: 213, a spacer, and an initiator element having at least about 70%, at least about 80%, at least about 90%, at least about 95%, at least about 97%, or about 100% sequence identity to SEQ ID NO: 204, optionally the spacer may be of 27 to 31 nucleotides.

In an embodiment, a core promoter may comprise a TATA box sequence having the sequence of SEQ ID NO: 213, a spacer of 29 nucleotides, and an initiator element having the sequence of SEQ ID NO: 204.

In an embodiment, a core promoter may comprise a TATA box sequence having at least about 70%, at least about 80%, at least about 90%, at least about 95%, at least about 97%, or about 100% sequence identity to SEQ ID NO: 201, a spacer, and an initiator element having at least about 70%, at least about 80%, at least about 90%, at least about 95%, at least about 97%, or about 100% sequence identity to SEQ ID NO: 202, optionally the spacer may be of 27 to 31 nucleotides.

In an embodiment, a core promoter may comprise a TATA box sequence having the sequence of SEQ ID NO: 201, a spacer of 29 nucleotides, and an initiator element having the sequence of SEQ ID NO: 202.

In an embodiment, a core promoter may comprise a TATA box sequence having at least about 70%, at least about 80%, at least about 90%, at least about 95%, at least about 97%, or about 100% sequence identity to SEQ ID NO: 213, a spacer, an initiator element having at least about 70%, at least about 80%, at least about 90%, at least about 95%, at least about 97%, or about 100% sequence identity to SEQ ID NO: 203, and a pause site having at least about 70%, at least about 80%, at least about 90%, at least about 95%, at least about 97%, or about 100% sequence identity to SEQ ID NO: 211, optionally the spacer may be of 29 to 33 nucleotides.

In an embodiment, a core promoter may comprise a TATA box sequence having the sequence of SEQ ID NO: 213, a spacer of 31 nucleotides, an initiator element having the sequence of SEQ ID NO: 203, and a pause site having the sequence of SEQ ID NO: 211.

In an embodiment, a core promoter may comprise a TATA box sequence having at least about 70%, at least about 80%, at least about 90%, at least about 95%, at least about 97%, or about 100% sequence identity to SEQ ID NO: 200, a spacer, an initiator element having at least about 70%, at least about 80%, at least about 90%, at least about 95%, at least about 97%, or about 100% sequence identity to SEQ ID NO: 203, and a YY1 motif having at least about 70%, at least about 80%, at least about 90%, at least about 95%, at least about 97%, or about 100% sequence identity to SEQ ID NO: 206, optionally the spacer may be of 29 to 33 nucleotides.

In an embodiment, a core promoter may comprise a TATA box sequence having the sequence of SEQ ID NO: 200, a spacer of 31 nucleotides, an initiator element having the sequence of SEQ ID NO: 203, and a YY1 motif having the sequence of SEQ ID NO: 206.

In an embodiment, a core promoter may comprise a TATA box sequence having at least about 70%, at least about 80%, at least about 90%, at least about 95%, at least about 97%, or about 100% sequence identity to SEQ ID NO: 201, a spacer, and an initiator element having at least about 70%, at least about 80%, at least about 90%, at least about 95%, at least about 97%, or about 100% sequence identity to SEQ ID NO: 204, optionally the spacer may be of 27 to 31 nucleotides.

In an embodiment, a core promoter may comprise a TATA box sequence having the sequence of SEQ ID NO: 201, a spacer of 29 nucleotides, and an initiator element having the sequence of SEQ ID NO: 204.

In an embodiment, a core promoter may comprise a TATA box sequence having at least about 70%, at least about 80%, at least about 90%, at least about 95%, at least about 97%, or about 100% sequence identity to SEQ ID NO: 200, a spacer, an initiator element having at least about 70%, at least about 80%, at least about 90%, at least about 95%, at least about 97%, or about 100% sequence identity to SEQ ID NO: 203, and a pause site having at least about 70%, at least about 80%, at least about 90%, at least about 95%, at least about 97%, or about 100% sequence identity to SEQ ID NO: 212, optionally the spacer may be of 29 to 33 nucleotides.

In an embodiment, a core promoter may comprise a TATA box sequence having the sequence of SEQ ID NO: 200, a spacer of 31 nucleotides, an initiator element having the sequence of SEQ ID NO: 203, and a pause site having the sequence of SEQ ID NO: 212.

In an embodiment, a core promoter may comprise a TATA box sequence having at least about 70%, at least about 80%, at least about 90%, at least about 95%, at least about 97%, or about 100% sequence identity to SEQ ID NO: 215, a spacer, and an initiator element having at least about 70%, at least about 80%, at least about 90%, at least about 95%, at least about 97%, or about 100% sequence identity to SEQ ID NO: 217, optionally the spacer may be of 30 to 34 nucleotides.

In an embodiment, a core promoter may comprise a TATA box sequence having the sequence of SEQ ID NO: 215, a spacer of 32 nucleotides, and an initiator element having the sequence of SEQ ID NO: 217.

In an embodiment, a core promoter may comprise TATA box sequence having at least about 70%, at least about 80%, at least about 90%, at least about 95%, at least about 97%, or about 100% sequence identity to SEQ ID NO: 200, a spacer, and an initiator element having at least about 70%, at least about 80%, at least about 90%, at least about 95%, at least about 97%, or about 100% sequence identity to SEQ ID NO: 202, optionally the spacer may be of 27 to 31 nucleotides.

In an embodiment, a core promoter may comprise TATA box sequence having the sequence of SEQ ID NO: 200, a spacer of 29 nucleotides, and an initiator element having the sequence of SEQ ID NO: 202.

In some embodiments, a switchable core promoter sequence may comprise a sequence of SEQ ID NO: 1-SEQ ID NO: 4, SEQ ID NO: 7, or SEQ ID NO: 10-SEQ ID NO: 144. In some embodiments, a switchable core promoter sequence may comprise a sequence having at least about 70%, at least about 72%, at least about 75%, at least about 78%, at least about 80%, at least about 82%, at least about 85%, at least about 87%, at least about 90%, at least about 92%, at least about 93%, at least about 95%, at least about 97%, at least about 98%, at least about 99%, or about 100% sequence identity to SEQ ID NO: 1-SEQ ID NO: 4, SEQ ID NO: 7, or SEQ ID NO: 10-SEQ ID NO: 144. Examples of switchable core promoter sequences are provided in TABLE 3.

TABLE 3
Exemplary Switchable Core Promoter Sequences

| SEQ ID NO: | Sequence |
|---|---|
| SEQ ID NO: 1 | GGGGTATAAAAGCCCCCGGCGCGTGATTACTGTCAGTCTTTTGG |
| | AGGGCCTCCCTTTAGCGTGTGAGGTGCTGTAATTCCCCC |
| SEQ ID NO: 2 | GGGGTATAAAAGGCGGGGGCGCGTGATTACTGTCAGTCTGTTGC |
| | AAGATGGCGGTGGTGCGTGGAAGCAGTTATAAAACGAAC |
| SEQ ID NO: 3 | GGGTTATATAAGGGCGCCGCGCGGGTGATTACTGTCAGTCTTTT |
| | GG |
| SEQ ID NO: 4 | TAGAGGGTATATAAAGGAAGCTCGACTTCCAGCTTGGCATTCCG |
| | G |
| SEQ ID NO: 7 | GGGGTATAAAAGCCCCCGGCGCGATTACTGACATTCTTTTGG |
| SEQ ID NO: 10 | GGGTTATATAAGGGCGCCTGGAAGGCGATTTCAGTCTTG |
| SEQ ID NO: 11 | GGGGTATAAAAGCCCCCGGCGCGTGATTACTGTCAGTCTGTTGG |
| | GAGAAGCGGGTGAAAGCAGGGCGGGGCTATAACTCGGCG |
| SEQ ID NO: 12 | GGGGTATAAAAGGCGGGGGCGCGTGATTACTGTCAGTCTGTTGC |
| | AAGATGGCGGCGGTGCGTGGAAGCAGTTATAAAACGAAC |
| SEQ ID NO: 13 | GGGTTATATAAGGGCGCCGCGCGTGATTACTGACATTCTTTTGG |
| SEQ ID NO: 14 | GGGGTATAAAAGGCGGGGTGGAATTGGCGATTTCAGTCTGGAA |
| | GGGGGGTAGGGTGCGTGGAAGCAGTTATAAAACGAAC |
| SEQ ID NO: 15 | TAGAGGGTATATAAGGGAAGCTCGACTTCCAGCTTGGCACTCCG |
| | G |
| SEQ ID NO: 16 | GGGGTATAAAAGGCGGGGTGGAATTGGCGATTTCAGTCTGGGA |
| | CGAGCGCGTGGGGGGTGACGGTGGCTGATTATCACGG |
| SEQ ID NO: 17 | GGGGTATAAAAGGCGGGGTGGAATTGGCGATTTCAGTCTGGAG |
| | GGCCTCCCTTTAGCGTGTGAGGTGCTGTAATTCCCCC |
| SEQ ID NO: 18 | GGGGTATAAAAGGCGGGGGCGCGTGATTACTGTCAGTCTGTTG |
| | GGACGAGCGCGTGGGGGGTGACGGTGGCTGATTATCACGG |
| SEQ ID NO: 19 | GGGGTATAAAAGGCGGGGGCGCGTGATTACTGTCAGTCTGTTG |
| | GAGGGCCTCCCTTTAGCGTGTGAGGTGCTGTAATTCCCCC |
| SEQ ID NO: 20 | GGGGTATAAAAGCCCCCGGCGCGTGATTACTGTCAGTCTGTTGG |
| | GACGAGCGCGTGGGGGGTGACGGTGGCTGATTATCACGG |
| SEQ ID NO: 21 | GGGGTATAAAAGCCCCCGGCGCGTGATTACTGTCAGTCTGTTGG |
| | AGGGCCTCCCTTTAGCGTGTGAGGTGCTGTAATTCCCCC |
| SEQ ID NO: 22 | GGGGTATAAAAGGCTGACCGCAGATGATTAAATCAGTCTTTTGG |
| SEQ ID NO: 23 | GGGGTATAAAAGCCGGGGAGGAACAGATAAGATCAGTCTTTTG |
| | G |
| SEQ ID NO: 24 | GGGGTATAAAAGGCGGGGGGGTTGTGCAATCGTCAGTCTGTTG |
| | G |
| SEQ ID NO: 25 | GGGGTATAAAAGGCTGCTTCCACAAGATTAGCTCAGTCTTTTGG |
| SEQ ID NO: 26 | GGGGTATAAAAGGCGGGGAAGTCCAAGGTCCATCAGTCTGTTG |
| | G |
| SEQ ID NO: 27 | GGGGTATAAAAGGCTGCTTCCACAAGATTAGCTCAGTCTGTTGG |
| SEQ ID NO: 28 | GGGGTATAAAAGCCCCCGAAGTCCAAGGTCCATCAGTCTGTTGG |
| SEQ ID NO: 29 | GGGGTATAAAAGCCTGACCGCAGATGATTAAATCAGTCTGTTGG |
| SEQ ID NO: 30 | GGGGTATAAAAGGCGGGGAAGTCCAAGGTCCATCAGTCTTTTG |
| | G |
| SEQ ID NO: 31 | GGGGTATAAAAGGCGGGGGCGCGTGATTACTGTCAGTCTTTTGC |
| | AAGATGGCGGCGGTGCGTGGAAGCAGTTATAAAACGAAC |
| SEQ ID NO: 32 | GGGGTATAAAAGCCCCCGGCGCGTGATTACTGTCATTGACTTGC |
| | AAGATGGCGGCGGTGCGTGGAAGCAGTTATAAAACGAAC |
| SEQ ID NO: 33 | GGGGTATAAAAGCCCCCGGCGCGTGATTACTGTCATTGACTTGC |
| | AAGATGGCGGTGGTGCGTGGAAGCAGTTATAAAACGAAC |
| SEQ ID NO: 34 | GGGGTATAAAAGCCCCCGGCGCGTGATTACTGTCATTGACTTGC |
| | AAGATGGCGGCTGCAGTCCCAGTTGCGATAGAAACCCCC |
| SEQ ID NO: 35 | GGGGTATAAAAGCCCCCGGCGCGTGATTACTGTCATTGACTTGC |
| | AAGATGGCGGTTGCAGTCCCAGTTGCGATAGAAACCCCC |
| SEQ ID NO: 36 | GGGGTATAAAAGGCGGGGTGGAATTGGCGATTTCAGTCTTGAA |
| | CAAGATGGCGGCGCGTGGAAGCAGTTATAAAACGAAC |
| SEQ ID NO: 37 | GGGGTATAAAAGGCGGGGTGGAATTGGCGATTTCAGTCTTGAA |
| | CAAGATGGCGGTGCGTGGAAGCAGTTATAAAACGAAC |
| SEQ ID NO: 38 | GGGGTATAAAAGGCGGGGTGGAATTGGCGATTTCAGTCTTGAA |
| | GCCGCCATCTTGGCGTGGAAGCAGTTATAAAACGAAC |
| SEQ ID NO: 39 | GGGGTATAAAAGCCCCCGGCGCGTGATTACTGTCAGTCTTTTGC |
| | AAGATGGCGGCGGTGCGTGGAAGCAGTTATAAAACGAAC |
| SEQ ID NO: 40 | GGGGTATAAAAGCCCCCGGCGCGTGATTACTGTCAGTCTTTTGC |
| | AAAATGGCGGCGGTGCGTGGAAGCAGTTATAAAACGAAC |
| SEQ ID NO: 41 | GGGGTATAAAAGCCCCCGGCGCGTGATTACTGTCAGTCTTTTGC |
| | AAGATGGCGGTGGTGCGTGGAAGCAGTTATAAAACGAAC |
| SEQ ID NO: 42 | GGGGTATAAAAGGCGGGGTGGAATTGGCGATTTCAGTCTGGAA |
| | CAAGATGGCGGTGCGTGGAAGCAGTTATAAAACGAAC |
| SEQ ID NO: 43 | GGGGTATAAAAGGCGGGGGCGCGTGATTACTGTCAGTCTTTTGC |
| | AAAATGGCGGCGGTGCGTGGAAGCAGTTATAAAACGAAC |
| SEQ ID NO: 44 | GGGGTATAAAAGCCCCCGGCGCGTGATTACTGTCAGTCTTTTGA |
| | AATATGGCGCTGGTGCGTGGAAGCAGTTATAAAACGAAC |
| SEQ ID NO: 45 | GGGGTATAAAAGCCCCCGGCGCGTGATTACTGTCAGTCTTTTGA |
| | GCGCCATATTTGGTGCGTGGAAGCAGTTATAAAACGAAC |
| SEQ ID NO: 46 | GGGGTATAAAAGGCGGGGTGGAATTGGCGATTTCATTGACGAA |
| | ACCGCCATCTTGGCGTGGAAGCAGTTATAAAACGAAC |
| SEQ ID NO: 47 | GGGGTATAAAAGCCCCCGGCGCGTGATTACTGTCAGTCTTTTGC |
| | AAGATGGCGGCTGCAGTCCCAGTTGCGATAGAAACCCCC |
| SEQ ID NO: 48 | GGGGTATAAAAGCCCCCGGCGCGTGATTACTGTCAGTCTTTTGC |
| | AAAATGGCGGCTGCAGTCCCAGTTGCGATAGAAACCCCC |
| SEQ ID NO: 49 | GGGGTATAAAAGGCGGGGGCGCGTGATTACTGTCAGTCTTTTGA |
| | AATATGGCGCTGGTGCGTGGAAGCAGTTATAAAACGAAC |
| SEQ ID NO: 50 | GGGGTATAAAAGGCGGGGGCGCGTGATTACTGTCAGTCTGTTGC |
| | AAAATGGCGGCGGTGCGTGGAAGCAGTTATAAAACGAAC |
| SEQ ID NO: 51 | GGGGTATAAAAGGCGGGGGCGCGTGATTACTGTCAGTCTGTTG |
| | AAATATGGCGCTGGTGCGTGGAAGCAGTTATAAAACGAAC |
| SEQ ID NO: 52 | GGGGTATAAAAGGCGGGGGCGCGTGATTACTGTCAGTCTGTTG |
| | ACCGCCATCTTGGGTGCGTGGAAGCAGTTATAAAACGAAC |
| SEQ ID NO: 53 | GGGGTATAAAAGGCGGGGGCGCGTGATTACTGTCAGTCTGTTGC |
| | AAGATGGCGGCTGCAGTCCCAGTTGCGATAGAAACCCCC |
| SEQ ID NO: 54 | GGGGTATAAAAGGCGGGGGCGCGTGATTACTGTCAGTCTGTTGC |
| | AAAATGGCGGCTGCAGTCCCAGTTGCGATAGAAACCCCC |
| SEQ ID NO: 55 | GGGGTATAAAAGGCGGGGGCGCGTGATTACTGTCAGTCTGTTGC |
| | AAGATGGCGGTTGCAGTCCCAGTTGCGATAGAAACCCCC |
| SEQ ID NO: 56 | GGGGTATAAAAGCCCCCGGCGCGTGATTACTGTCAGTCTGTTGC |
| | AAGATGGCGGCGGTGCGTGGAAGCAGTTATAAAACGAAC |
| SEQ ID NO: 57 | GGGGTATAAAAGCCCCCGGCGCGTGATTACTGTCAGTCTGTTGC |
| | AAAATGGCGGCGGTGCGTGGAAGCAGTTATAAAACGAAC |
| SEQ ID NO: 58 | GGGGTATAAAAGCCCCCGGCGCGTGATTACTGTCAGTCTGTTGC |
| | AAGATGGCGGTGGTGCGTGGAAGCAGTTATAAAACGAAC |
| SEQ ID NO: 59 | GGGGTATAAAAGCCCCCGGCGCGTGATTACTGTCAGTCTGTTGA |
| | AATATGGCGCTGGTGCGTGGAAGCAGTTATAAAACGAAC |
| SEQ ID NO: 60 | GGGGTATAAAAGCCCCCGGCGCGTGATTACTGTCAGTCTGTTGG |
| | CCGCCATCTTGGGTGCGTGGAAGCAGTTATAAAACGAAC |
| SEQ ID NO: 61 | GGGGTATAAAAGCCCCCGGCGCGTGATTACTGTCAGTCTGTTGC |
| | AAGATGGCGGCTGCAGTCCCAGTTGCGATAGAAACCCCC |
| SEQ ID NO: 62 | GGGGTATAAAAGCCCCCGGCGCGTGATTACTGTCAGTCTGTTGC |
| | AAGATGGCGGTTGCAGTCCCAGTTGCGATAGAAACCCCC |
| SEQ ID NO: 63 | GGGGTATAAAAGCCCCCGGCGCGTGATTACTGTCAGTCTGTTGA |
| | AATATGGCGCTTGCAGTCCCAGTTGCGATAGAAACCCCC |
| SEQ ID NO: 64 | GGGGTATAAAAGCCCCCGGCGCGTGATTACTGTCAGTCTGTTGA |
| | CCGCCATCTTGTGCAGTCCCAGTTGCGATAGAAACCCCC |
| SEQ ID NO: 65 | GGGGTATAAAAGGCGGGGGCGCGTGATTACTGTCAGTCTTTTGA |
| | GCGCCATATTTGGTGCGTGGAAGCAGTTATAAAACGAAC |
| SEQ ID NO: 66 | GGGGTATAAAAGGCGGGGGCGCGTGATTACTGTCAGTCTTTTGC |
| | AAGATGGCGGCTGCAGTCCCAGTTGCGATAGAAACCCCC |
| SEQ ID NO: 67 | GGGGTATAAAAGGCGGGGGCGCGTGATTACTGTCATTGACTTGC |
| | AAGATGGCGGCGGTGCGTGGAAGCAGTTATAAAACGAAC |
| SEQ ID NO: 68 | GGGGTATAAAAGGCGGGGGCGCGTGATTACTGTCATTGACTTGC |
| | AAGATGGCGGTGGTGCGTGGAAGCAGTTATAAAACGAAC |
| SEQ ID NO: 69 | GGGGTATAAAAGGCGGGGGCGCGGGTGATTACTGTCAGTCTTTT |
| | GG |
| SEQ ID NO: 70 | GGGGTATAAAAGGCGGGGGCGCGGGTGATTACTGTCAGTCTCTT |
| | GG |
| SEQ ID NO: 71 | GGGGTATAAAAGGCGGGGTGGAAGGTTGGCGATTTCAGTCTTG |
| SEQ ID NO: 72 | GGGGTATAAAAGCCCCCGTGGAAGGTTGGCGATTTCAGTCTTG |
| SEQ ID NO: 73 | GGGTTATATAAGGGCGCCTGGAAGGTTGGCGATTTCAGTCTTG |
| SEQ ID NO: 74 | GGGGTATAAAAAGCGGGGGCGCGGGTGATTACTGTCAGTCTCTT |
| | GG |
| SEQ ID NO: 75 | GGGGTATAAAAGGCGGGGTGGAAGGTTGGCGATTTCAGTCTCG |
| SEQ ID NO: 76 | GGGTTATATAAGGGCGCCTGGAAGGTTGGCGATTTCAGTCTCG |
| SEQ ID NO: 77 | GGGGTATAAAAGCCCCCGGCGCGGGTGATTACTGTCAGTCTCTT |
| | GG |
| SEQ ID NO: 78 | GGGGTATAAAAGGCGGGGTGGAAGGTTGGCGATTTCAGTCTGG |
| SEQ ID NO: 79 | GGGGTATAAAAAGCGGGGTGGAAGGTTGGCGATTTCAGTCTGG |
| SEQ ID NO: 80 | GGGTTATATAAGGGCGCCTGGAAGGTTGGCGATTTCAGTCTGG |
| SEQ ID NO: 81 | GGGGTATAAAAGGCGGGGTGGAAGGTTGGCGATTTCAGTAGTG |
| SEQ ID NO: 82 | GGGGTATAAAAAGCGGGGTGGAAGGTTGGCGATTTCAGTAGTG |
| SEQ ID NO: 83 | GGGGTATAAAAGCCCCCGTGGAAGGTTGGCGATTTCAGTAGTG |
| SEQ ID NO: 84 | GGGTTATATAAGGGCGCCTGGAAGGTTGGCGATTTCAGTAGTG |
| SEQ ID NO: 85 | GGGGTATAAAAGGCGGGGTGGAAGGTTGGCGATTACATTCTTG |
| SEQ ID NO: 86 | GGGTTATATAAGGGCGCCGCGCGGGTGATTACTGTCAGTCTCTT |
| | GG |
| SEQ ID NO: 87 | GGGGTATAAAAGGCGGGGTGGAAGGTTGGCGATTTCATATCCG |
| SEQ ID NO: 88 | GGGTTATATAAGGGCGCCTGGAAGGTTGGCGATTTCATATCCG |
| SEQ ID NO: 89 | GGGTTATATAAGGGCGCCTGGAAGGTTGGCGATTTCATTGACG |
| SEQ ID NO: 90 | GGGGTATAAAAGGCGGGGTGGAAGGTTGGCGATTTCACAAACG |
| SEQ ID NO: 91 | GGGGTATAAAAAGCGGGGTGGAAGGTTGGCGATTTCACAAACG |
| SEQ ID NO: 92 | GGGTTATATAAGGGCGCCTGGAAGGTTGGCGATTTCACAAACG |
| SEQ ID NO: 93 | GGGGTATAAAAGGCGGGGTGGAAGGTTGGCGATTCACAGGAAG |
| SEQ ID NO: 94 | GGGGTATAAAAAGCGGGGGCGCGGGTGATTACTGTCAGTCTGT |
| | TGG |
| SEQ ID NO: 95 | GGGGTATAAAAGCCCCCGGCGCGGGTGATTACTGTCAGTCTGTT |
| | GG |
| SEQ ID NO: 96 | GGGTTATATAAGGGCGCCGCGCGGGTGATTACTGTCAGTCTGTT |
| | GG |
| SEQ ID NO: 97 | GGGGTATAAAAGCCCCCGGCGCGGGTGATTACTGTCAGTCTTTT |
| | GG |
| SEQ ID NO: 98 | GGGGTATAAAAGGCGGGGGCGCGGGTGATTACTGTCAGTAGTT |
| | TGG |
| SEQ ID NO: 99 | GGGGTATAAAAGCCCCCGGCGCGGGTGATTACTGTCAGTAGTTT |
| | GG |
| SEQ ID NO: 100 | GGGTTATATAAGGGCGCCGCGCGGGTGATTACTGTCAGTAGTTT |
| | GG |
| SEQ ID NO: 101 | GGGGTATAAAAGGCGGGGGCGCGGGTGATTACTGACATTCTTTT |
| | GG |
| SEQ ID NO: 102 | GGGGTATAAAAGCCCCCGGCGCGGGTGATTACTGACATTCTTTT |
| | GG |
| SEQ ID NO: 103 | GGGTTATATAAGGGCGCCGCGCGGGTGATTACTGACATTCTTTT |
| | GG |
| SEQ ID NO: 104 | GGGGTATAAAAGCCCCCGGCGCGGGTGATTACTGTCATATCCTT |
| | GG |
| SEQ ID NO: 105 | GGGTTATATAAGGGCGCCGCGCGGGTGATTACTGTCATATCCTT |
| | GG |
| SEQ ID NO: 106 | GGGTTATATAAGGGCGCCGCGCGGGTGATTACTGTCATTGACTT |
| | GG |
| SEQ ID NO: 107 | GGGGTATAAAAGCCCCCGGCGCGGGTGATTACTGTCACAAACTT |
| | GG |
| SEQ ID NO: 108 | GGGTTATATAAGGGCGCCGCGCGGGTGATTACTGTCACAAACTT |
| | GG |
| SEQ ID NO: 109 | GGGGTATAAAAGGCGGGGGCGCGATTACTGTCAGTCTTTTGG |
| SEQ ID NO: 110 | GGGGTATAAAAGGCGGGGTGGAAGGCGATTTCAGTCTTG |
| SEQ ID NO: 111 | GGGGTATAAAAGGCGGGGTGGAAGGCGATTTCAGTCTCG |
| SEQ ID NO: 112 | GGGTTATATAAGGGCGCCTGGAAGGCGATTTCAGTCTCG |
| SEQ ID NO: 113 | GGGGTATAAAAGGCGGGGTGGAAGGCGATTTCAGTAGTG |
| SEQ ID NO: 114 | GGGTTATATAAGGGCGCCTGGAAGGCGATTTCAGTAGTG |
| SEQ ID NO: 115 | GGGGTATAAAAAGCGGGGTGGAAGGCGATTACATTCTTG |
| SEQ ID NO: 116 | GGGTTATATAAGGGCGCCGCGCGATTACTGTCAGTCTCTTGG |
| SEQ ID NO: 117 | GGGTTATATAAGGGCGCCTGGAAGGCGATTACATTCTTG |
| SEQ ID NO: 118 | GGGGTATAAAAGGCGGGGTGGAAGGCGATTCACAGGAAG |
| SEQ ID NO: 119 | GGGGTATAAAAAGCGGGGTGGAAGGCGATTCACAGGAAG |
| SEQ ID NO: 120 | GGGGTATAAAAGCCCCCGTGGAAGGCGATTCACAGGAAG |
| SEQ ID NO: 121 | GGGTTATATAAGGGCGCCGCGCGATTACTGTCAGTAGTTTGG |
| SEQ ID NO: 122 | GGGGTATAAAAGGCGGGGGCGCGATTACTGACATTCTTTTGG |
| SEQ ID NO: 123 | GGGGTATAAAAAGCGGGGGCGCGATTACTGACATTCTTTTGG |
| SEQ ID NO: 124 | GGGTTATATAAGGGCGCCGCGCGATTACTGACATTCTTTTGG |
| SEQ ID NO: 125 | GGGTTATATAAGGGCGCCGCGCGATTACTGTCAGTCTTTTGG |
| SEQ ID NO: 126 | GGGGTATAAAAGGCGGGGGCGCGTGATTACTGTCAGTCTTTTGG |
| SEQ ID NO: 127 | GGGGTATAAAAGGCGGGGGCGCGTGATTACTGTCAGTCTCTTGG |
| SEQ ID NO: 128 | GGGTTATATAAGGGCGCCTGGAATTGGCGATTTCAGTCTTG |
| SEQ ID NO: 129 | GGGTTATATAAGGGCGCCTGGAATTGGCGATTTCAGTCTCG |
| SEQ ID NO: 130 | GGGGTATAAAAGCCCCCGGCGCGTGATTACTGTCAGTCTCTTGG |
| SEQ ID NO: 131 | GGGTTATATAAGGGCGCCTGGAATTGGCGATTTCAGTCTGG |
| SEQ ID NO: 132 | GGGTTATATAAGGGCGCCTGGAATTGGCGATTTCAGTAGTG |
| SEQ ID NO: 133 | GGGTTATATAAGGGCGCCGCGCGTGATTACTGTCAGTCTCTTGG |
| SEQ ID NO: 134 | GGGGTATAAAAGGCGGGGGCGCGTGATTACTGTCAGTCTGTTG |
| | G |
| SEQ ID NO: 135 | GGGGTATAAAAGCCCCCGGCGCGTGATTACTGTCAGTCTGTTGG |
| SEQ ID NO: 136 | GGGTTATATAAGGGCGCCGCGCGTGATTACTGTCAGTCTGTTGG |
| SEQ ID NO: 137 | GGGGTATAAAAGCCCCCGGCGCGTGATTACTGTCAGTCTTTTGG |
| SEQ ID NO: 138 | GGGGTATAAAAGGCGGGGGCGCGTGATTACTGTCAGTAGTTTG |
| | G |
| SEQ ID NO: 139 | GGGGTATAAAAGCCCCCGGCGCGTGATTACTGTCAGTAGTTTGG |
| SEQ ID NO: 140 | GGGTTATATAAGGGCGCCGCGCGTGATTACTGTCAGTAGTTTGG |
| SEQ ID NO: 141 | GGGGTATAAAAGCCCCCGGCGCGTGATTACTGACATTCTTTTGG |
| SEQ ID NO: 142 | GGGTTATATAAGGGCGCCGCGCGTGATTACTGTCAGTCTTTTGG |
| SEQ ID NO: 143 | GGGGTATAAAAGGCGGGGGCGCGTGATTACTGTCACAAACTTG |
| | G |
| SEQ ID NO: 144 | GGGTTATATAAGGGCGCCGCGCGTGATTACTGTCACAAACTTGG |

In some embodiments, a core promoter of the present disclosure may function as a switchable core promoter in multiple cell types or cell states. Cell type- or cell state-specificity may be conferred by a response element (e.g., an enhancer sequence that binds to cell type-specific transcription factors) paired with the switchable core promoter. For example, a switchable core promoter of SEQ ID NO: 1-SEQ ID NO: 4, SEQ ID NO: 7, or SEQ ID NO: 10-SEQ ID NO: 144 may be paired with a CNS-specific enhancer to promote CNS-specific transcription initiation and may be paired with a kidney-specific enhancer to promote kidney-specific transcription initiation. The core promoters described herein may have low background transcriptional activation (e.g., low levels of transcriptional activation in the absence of an enhancer, in the presence of an inactive enhancer, or in the presence of an unactivated enhancer) and high activity (e.g., high levels of transcriptional activation) when paired with a response element in the presence of cell type-, tissue type-, or cell state-specific cognate ligands or coactivators (e.g., when paired with an activated enhancer). For example, a core promoter may have low transcriptional activation in the presence of an inactive enhancer. The core promoter may have high transcriptional initiation when paired with a cell type-, tissue type-, or cell state-specific response element in a cell type, tissue type, or cell state of interest (e.g., in the presence of, or at high levels of, transcription factors that bind to the transcription factor binding sequence of an enhancer region sequence). The core promoter may have low transcriptional activation when paired with a cell type-, tissue type-, or cell state-specific response element not in a cell type, tissue type, or cell state of interest (e.g., in the absence of, or at low levels of, transcription factors that bind to the transcription factor binding sequence of an enhancer region sequence). In some embodiments, a core promoter may be engineered to have low background transcriptional activation and high transcriptional activation when paired with a cell type-, tissue type, or cell state-specific response element in a cell type, tissue type, or cell state of interest. The sequence of the core promoter may be varied or engineered to tune the transcription level, tissue specificity, or cell type- or cell state-specificity. In some embodiments, a core promoter may comprise an engineered version of an endogenous core sequence. For example, a switchable core promoter of SEQ ID NO: 1-SEQ ID NO: 4, SEQ ID NO: 7, or SEQ ID NO: 10-SEQ ID NO: 144 may be paired with a response element of SEQ ID NO: 145, SEQ ID NO: 146, SEQ ID NO: 193, or SEQ ID NO: 194.

### Enhancer/Promoters

A promoter (e.g., an enhancer/promoter) may comprise a switchable core promoter (e.g., SEQ ID NO: 1-SEQ ID NO: 4, SEQ ID NO: 7, or SEQ ID NO: 10-SEQ ID NO: 144) paired with a response element (e.g., an enhancer comprising a transcription factor binding site or a coactivator binding site). In some embodiments, the response element of the promoter may confer cell type-, tissue type, or cell state-specificity. For example, an enhancer of SEQ ID NO: 146 or SEQ ID NO: 194 may confer liver specificity. In some embodiments, an enhancer of SEQ ID NO: 145 or SEQ ID NO: 193 may confer bone marrow specificity. In some embodiments, the switchable core promoter of the promoter may readily promote transcription initiation in the presence of an activated response element (e.g., an activated enhancer) and exhibit low background transcription initiation in the presence of an inactive response element (e.g., an inactive enhancer). In some embodiments, the switchable core promoter of the promoter may readily promote transcription initiation in the presence of an activated response element (e.g., an activated enhancer) and exhibit low background transcription initiation in the presence of the unactivated response element (e.g., the unactivated enhancer). A switchable core promoter of SEQ ID NO: 1-SEQ ID NO: 4, SEQ ID NO: 7, or SEQ ID NO: 10-SEQ ID NO: 144 may be paired with a response element of SEQ ID NO: 145 or SEQ ID NO: 146.

In some embodiments, a liver-specific enhancer/promoter may comprise a sequence of SEQ ID NO: 163-SEQ ID NO: 166, SEQ ID NO: 169, or SEQ ID NO: 172-SEQ ID NO: 177. In some embodiments, a bone marrow-specific enhancer/promoter may comprise a sequence of SEQ ID NO: 148-SEQ ID NO: 151, SEQ ID NO: 154, or SEQ ID NO: 157-SEQ ID NO: 162. Examples of enhancer/promoter sequences are provided in TABLE 4.

TABLE 4
Exemplary Enhancer/Promoter Sequences

| SEQ ID NO: | Sequence |
|---|---|
| SEQ ID NO: | TGCTATTTTTAGCGGGCTTTTTTTGACGGGAGGAAGGAAGGAGGGA |
| 148 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGCCCCCGGCGCGTGATTACTGTCAGTCTTTTGGAGGGCCTCCCTTT |
| | AGCGTGTGAGGTGCTGTAATTCCCCC |
| SEQ ID NO: | TGCTATTTTTAGCGGGCTTTTTTTGACGGGAGGAAGGAAGGAGGGA |
| 149 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGGCGGGGGCGCGTGATTACTGTCAGTCTGTTGCAAGATGGCGGTG |
| | GTGCGTGGAAGCAGTTATAAAACGAAC |
| SEQ ID NO: | TGCTATTTTTAGCGGGCTTTTTTTGACGGGAGGAAGGAAGGAGGGA |
| 150 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGTTATATA |
| | AGGGCGCCGCGCGGGTGATTACTGTCAGTCTTTTGG |
| SEQ ID NO: | TGCTATTTTTAGCGGGCTTTTTTTGACGGGAGGAAGGAAGGAGGGA |
| 151 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACTAGAGGGTAT |
| | ATAAAGGAAGCTCGACTTCCAGCTTGGCATTCCGG |
| SEQ ID NO: | TGCTATTTTTAGCGGGCTTTTTTTGACGGGAGGAAGGAAGGAGGGA |
| 154 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGCCCCCGGCGCGATTACTGACATTCTTTTGG |
| SEQ ID NO: | TGCTATTTTTAGCGGGCTTTTTTTGACGGGAGGAAGGAAGGAGGGA |
| 157 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGTTATATA |
| | AGGGCGCCTGGAAGGCGATTTCAGTCTTG |
| SEQ ID NO: | TGCTATTTTTAGCGGGCTTTTTTTGACGGGAGGAAGGAAGGAGGGA |
| 158 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGCCCCCGGCGCGTGATTACTGTCAGTCTGTTGGGAGAAGCGGGTG |
| | AAAGCAGGGCGGGGCTATAACTCGGCG |
| SEQ ID NO: | TGCTATTTTTAGCGGGCTTTTTTTGACGGGAGGAAGGAAGGAGGGA |
| 159 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGGCGGGGGCGCGTGATTACTGTCAGTCTGTTGCAAGATGGCGGCG |
| | GTGCGTGGAAGCAGTTATAAAACGAAC |
| SEQ ID NO: | TGCTATTTTTAGCGGGCTTTTTTTGACGGGAGGAAGGAAGGAGGGA |
| 160 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGTTATATA |
| | AGGGCGCCGCGCGTGATTACTGACATTCTTTTGG |
| SEQ ID NO: | TGCTATTTTTAGCGGGCTTTTTTTGACGGGAGGAAGGAAGGAGGGA |
| 161 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGGCGGGGTGGAATTGGCGATTTCAGTCTGGAAGGGGGGTAGGGT |
| | GCGTGGAAGCAGTTATAAAACGAAC |
| SEQ ID NO: | TGCTATTTTTAGCGGGCTTTTTTTGACGGGAGGAAGGAAGGAGGGA |
| 162 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACTAGAGGGTAT |
| | ATAAGGGAAGCTCGACTTCCAGCTTGGCACTCCGG |
| SEQ ID NO: | GGTTAATGATTAACCGTGTAAATAATTAGCGGATCACGTGATGGTC |
| 163 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGCCCCCGGCGCGTGATTACTGTCAGTCTTTTGGAGGGCCT |
| | CCCTTTAGCGTGTGAGGTGCTGTAATTCCCCC |
| SEQ ID NO: | GGTTAATGATTAACCGTGTAAATAATTAGCGGATCACGTGATGGTC |
| 164 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGGCGGGGGCGCGTGATTACTGTCAGTCTGTTGCAAGATGG |
| | CGGTGGTGCGTGGAAGCAGTTATAAAACGAAC |
| SEQ ID NO: | GGTTAATGATTAACCGTGTAAATAATTAGCGGATCACGTGATGGTC |
| 165 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGTTA |
| | TATAAGGGCGCCGCGCGGGTGATTACTGTCAGTCTTTTGG |
| SEQ ID NO: | GGTTAATGATTAACCGTGTAAATAATTAGCGGATCACGTGATGGTC |
| 166 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCTAGAG |
| | GGTATATAAAGGAAGCTCGACTTCCAGCTTGGCATTCCGG |
| SEQ ID NO: | GGTTAATGATTAACCGTGTAAATAATTAGCGGATCACGTGATGGTC |
| 169 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGCCCCCGGCGCGATTACTGACATTCTTTTGG |
| SEQ ID NO: | GGTTAATGATTAACCGTGTAAATAATTAGCGGATCACGTGATGGTC |
| 172 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGTTA |
| | TATAAGGGCGCCTGGAAGGCGATTTCAGTCTTG |
| SEQ ID NO: | GGTTAATGATTAACCGTGTAAATAATTAGCGGATCACGTGATGGTC |
| 173 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGCCCCCGGCGCGTGATTACTGTCAGTCTGTTGGGAGAAGC |
| | GGGTGAAAGCAGGGCGGGGCTATAACTCGGCG |
| SEQ ID NO: | GGTTAATGATTAACCGTGTAAATAATTAGCGGATCACGTGATGGTC |
| 174 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGGCGGGGGCGCGTGATTACTGTCAGTCTGTTGCAAGATGG |
| | CGGCGGTGCGTGGAAGCAGTTATAAAACGAAC |
| SEQ ID NO: | GGTTAATGATTAACCGTGTAAATAATTAGCGGATCACGTGATGGTC |
| 175 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGTTA |
| | TATAAGGGCGCCGCGCGTGATTACTGACATTCTTTTGG |
| SEQ ID NO: | GGTTAATGATTAACCGTGTAAATAATTAGCGGATCACGTGATGGTC |
| 176 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGGCGGGGTGGAATTGGCGATTTCAGTCTGGAAGGGGGGT |
| | AGGGTGCGTGGAAGCAGTTATAAAACGAAC |
| SEQ ID NO: | GGTTAATGATTAACCGTGTAAATAATTAGCGGATCACGTGATGGTC |
| 177 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCTAGAG |
| | GGTATATAAGGGAAGCTCGACTTCCAGCTTGGCACTCCGG |
| SEQ ID NO: | TGCTATTTTTAGCGGGCTTTTTTTGACGGGAGGAAGGAAGGAGGGA |
| 218 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACGGGGTATAAA |
| | AGGCGGGGTGGAAGGCGATTTCAGTCTTG |
| SEQ ID NO: | TGCTATTTTTAGCGGGCTTTTTTTGACGGGAGGAAGGAAGGAGGGA |
| 219 | GAGGGACGGGAAGTAAAGAGAAAAAGAGGAAGTGAAAGCTAAGA |
| | AGGAAGTGACGGCTGGCGGGGACAGATAAGAGTTGTCTAGTTGTG |
| | ATAATGGAACTGCTGAGTCATGGATTGCAGAGTCACAGGACCGGAT |
| | CAACTAGGTCTATATAAGCAGAGCTCGTTTAGTGAACCGTCAGATC |
| | GCCTGGAGACGCCATCCACGCTGTTTTGACCTCCATAGAATTGGTA |
| | CCGAGCTCG |
| SEQ ID NO: | GGTTAATGATTAACCGTGTAAATAATTAGCGGATCACGTGATGGTC |
| 220 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCGGGGT |
| | ATAAAAGGCGGGGTGGAAGGCGATTTCAGTCTTG |
| SEQ ID NO: | GGTTAATGATTAACCGTGTAAATAATTAGCGGATCACGTGATGGTC |
| 221 | ACGTGTTGGTTCAAGGCCAGAGTCAAGGTCGGGAGTCCAAAGTCCA |
| | GAAGTGCAAGGTCCGGTGTTTACTTTGGGTGTTTACCTTCCAGGAC |
| | CGGATCAACTAGGTCTATATAAGCAGAGCTCGTTTAGTGAACCGTC |
| | AGATCGCCTGGAGACGCCATCCACGCTGTTTTGACCTCCATAGAAT |
| | TGGTACCGAGCTCG |
| SEQ ID NO: | GGTTAATGATTAACCgTGTAAATAATTAGCGgATCACGTGATgGTCA |
| 222 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGCCCCCGGCGCGTGATTACTGTCAGTCTTTTGGAGGGCCTCCC |
| | TTTAGCGTGTGAGGTGCTGTAATTCCCCC |
| SEQ ID NO: | GGTTAATGATTAACCgTGTAAATAATTAGCGgATCACGTGATgGTCA |
| 223 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGGCGGGGGCGCGTGATTACTGTCAGTCTGTTGCAAGATGGCGG |
| | TGGTGCGTGGAAGCAGTTATAAAACGAAC |
| SEQ ID NO: | GGTTAATGATTAACCgTGTAAATAATTAGCGgATCACGTGATgGTCA |
| 224 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGGCGGGGGCGCGTGATTACTGTCAGTCTGTTGCAAGATGGCGG |
| | TGGTGCGTGGAAGCAGTTATAAAACGAAC |
| SEQ ID NO: | GGTTAATGATTAACCgTGTAAATAATTAGCGgATCACGTGATgGTCA |
| 225 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCTAGAGGGT |
| | ATATAAAGGAAGCTCGACTTCCAGCTTGGCATTCCGG |
| SEQ ID NO: | GGTTAATGATTAACCgTGTAAATAATTAGCGgATCACGTGATgGTCA |
| 226 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCTAGAGGGT |
| | ATATAATGGAAGCTCGACTTCCAGCTTGGCAATCCGG |
| SEQ ID NO: | GGTTAATGATTAACCgTGTAAATAATTAGCGgATCACGTGATgGTCA |
| 227 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGTACTTATA |
| | TAAGGGGGTGGGGGCGCGTTCGTCCTCAGTCGCGATCGAACACTCG |
| | AGCCGAGCAGACGTGCCTACGGACCG |
| SEQ ID NO: | GGTTAATGATTAACCgTGTAAATAATTAGCGgATCACGTGATgGTCA |
| 228 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGCCCCCGGCGCGATTACTGACATTCTTTTGG |
| SEQ ID NO: | GGTTAATGATTAACCgTGTAAATAATTAGCGgATCACGTGATgGTCA |
| 229 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGTACGC |
| | CCCTTTTTATGCGCGTGATTACTGCACAGGAATTGG |
| SEQ ID NO: | GGTTAATGATTAACCgTGTAAATAATTAGCGgATCACGTGATgGTCA |
| 230 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCTCTAGAGG |
| | GTATATAATGGGGGCCACTAGTCTACTACCAGAAAG |
| SEQ ID NO: | GGTTAATGATTAACCgTGTAAATAATTAGCGgATCACGTGATgGTCA |
| 231 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGTTATAT |
| | AAGGGCGCCTGGAAGGCGATTTCAGTCTTG |
| SEQ ID NO: | GGTTAATGATTAACCTGTAAATAATTAGCGgATCACGTGATgGTCA |
| 232 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGCCCCCGGCGCGTGATTACTGTCAGTCTGTTGGGAGAAGCGGG |
| | TGAAAGCAGGGCGGGGCTATAACTCGGCG |
| SEQ ID NO: | GGTTAATGATTAACCgTGTAAATAATTAGCGgATCACGTGATgGTCA |
| 233 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGGCGGGGGCGCGTGATTACTGTCAGTCTGTTGCAAGATGGCGG |
| | CGGTGCGTGGAAGCAGTTATAAAACGAAC |
| SEQ ID NO: | GGTTAATGATTAACCgTGTAAATAATTAGCGgATCACGTGATgGTCA |
| 234 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGTTATAT |
| | AAGGGCGCCGCGCGTGATTACTGACATTCTTTTGG |
| SEQ ID NO: | GGTTAATGATTAACCgTGTAAATAATTAGCGgATCACGTGATgGTCA |
| 235 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGGCGGGGTGGAATTGGCGATTTCAGTCTGGAAGGGGGGTAGG |
| | GTGCGTGGAAGCAGTTATAAAACGAAC |
| SEQ ID NO: | GGTTAATGATTAACCgTGTAAATAATTAGCGgATCACGTGATgGTCA |
| 236 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCTAGAGGGT |
| | ATATAAGGGAAGCTCGACTTCCAGCTTGGCACTCCGG |
| SEQ ID NO: | GGTTAATGATTAACCgTGTAAATAATTAGCGgATCACGTGATgGTCA |
| 237 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCGGGGTATA |
| | AAAGGCGGGGTGGAAGGCGATTTCAGTCTTG |
| SEQ ID NO: | GGTTAATGATTAACCgTGTAAATAATTAGCGgATCACGTGATgGTCA |
| 238 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCAGGACCGG |
| | ATCAACTAGGTCTATATAAGCAGAGCTCGTTTAGTGAACCGTCAGA |
| | TCGCCTGGAGACGCCATCCACGCTGTTTTGACCTCCATAGAATTGG |
| | TACCGAGCTCG |
| SEQ ID NO: | GGTTAATGATTAACCgTGTAAATAATTAGCGgATCACGTGATgGTCA |
| 239 | CGTGTTgGTTCAAGGCCAgAGTCAAGGTCGgGAGTCCAAAGTCCAgA |
| | AGTGCAAGGTCCGgTGTTTACTTTGGgTGTTTACCTTCCAGGACCGG |
| | ATCAACTAGGTCTATATAAGCAGAGCTCGTTTAGTGAACCGTCAGA |
| | TCGCCTGGAGACGCCATCCACGCTGTTTTGACCTCCATAGAATTGG |
| | TACCGAGCTCG |
| SEQ ID NO: | AGAAACACGGCACAGATGACGGCTTTAAAAGAAAAGAGTCCATTG |
| 240 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGGTATAAAAGGCGGGGGCGCGTGATTACTGTCAGT |
| | CTGTTGCAAGATGGCGGTGGTGCGTGGAAGCAGTTATAAAACGAA |
| | C |
| SEQ ID NO: | AGAAACACGGCACAGATGACGGCTTTAAAAGAAAAGAGTCCATTG |
| 241 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGTTATATAAGGGCGCCGCGCGGGTGATTACTGTCAG |
| | TCTTTTGG |
| SEQ ID NO: | AGAAACACGGCACAGATGACGGCTTTAAAAGAAAAGAGTCCATTG |
| 242 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATTAGAGGGTATATAAAGGAAGCTCGACTTCCAGCTTGGC |
| | ATTCCGG |
| SEQ ID NO: | AGAAACACGGCACAGATGACGGCTTTAAAAGAAAAGAGTCCATTG |
| 243 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATTAGAGGGTATATAATGGAAGCTCGACTTCCAGCTTGGC |
| | AATCCGG |
| SEQ ID NO: | AGAAACACGGCACAGATGACGGCTTTAAAAGAAAAGAGTCCATTG |
| 244 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGTACTTATATAAGGGGGTGGGGGCGCGTTCGTCCTCAG |
| | TCGCGATCGAACACTCGAGCCGAGCAGACGTGCCTACGGACCG |
| SEQ ID NO: | AGAAACACGGCACAGATGACGGCTTTAAAAGAAAAGAGTCCATTG |
| 245 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGGTATAAAAGCCCCCGGCGCGATTACTGACATTCTT |
| | TTGG |
| SEQ ID NO: | AGAAACACGGCACAGATGACGGCTTTAAAAGAAAAGAGTCCATTG |
| 246 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGTACGCCCCTTTTTATGCGCGTGATTACTGCACAGG |
| | AATTGG |
| SEQ ID NO: | AGAAACACGGCACAGATGACGGCTTTAAAAGAAAAGAGTCCATTG |
| 247 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATTCTAGAGGGTATATAATGGGGGCCACTAGTCTACTACC |
| | AGAAAG |
| SEQ ID NO: | AGAAACACGGCACAGATGACGGCTTTAAAAGAAAAGAGTCCATTG |
| 248 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGTTATATAAGGGCGCCTGGAAGGCGATTTCAGTCTT |
| | G |
| SEQ ID NO: | AGAAACACGGCACAGATGACGGCTTTAAAAGAAAAGAGTCCATTG |
| 249 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGGTATAAAAGCCCCCGGCGCGTGATTACTGTCAGTC |
| | TGTTGGGAGAAGCGGGTGAAAGCAGGGCGGGGCTATAACTCGGCG |
| SEQ ID NO: | AGAAACACGGCACAGATGACGGCTTTAAAAGAAAAGAGTCCATTG |
| 250 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGGTATAAAAGGCGGGGGCGCGTGATTACTGTCAGT |
| | CTGTTGCAAGATGGCGGCGGTGCGTGGAAGCAGTTATAAAACGAA |
| | C |
| SEQ ID NO: | AGAAACACGGCACAGATGACGGCTTTAAAAGAAAAGAGTCCATTG |
| 251 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGTTATATAAGGGCGCCGCGCGTGATTACTGACATTC |
| | TTTTGG |
| SEQ ID NO: | AGAAACACGGCACAGATGACGGCTTTAAAAGAAAAGAGTCCATTG |
| 252 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGGTATAAAAGGCGGGGTGGAATTGGCGATTTCAGT |
| | CTGGAAGGGGGGTAGGGTGCGTGGAAGCAGTTATAAAACGAAC |
| SEQ ID NO: | AGAAACACGGCACAGATGACGGCTTTAAAAGAAAAGAGTCCATTG |
| 253 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATTAGAGGGTATATAAGGGAAGCTCGACTTCCAGCTTGGC |
| | ACTCCGG |
| SEQ ID NO: | AGAAACACGGCACAGATGACGGCTTTAAAAGAAAAGAGTCCATTG |
| 254 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATGGGGTATAAAAGGCGGGGTGGAAGGCGATTTCAGTCT |
| | TG |
| SEQ ID NO: | AGAAACACGGCACAGATGACGGCTTTAAAAGAAAAGAGTCCATTG |
| 255 | ACTGGAAAAGCAAACAGTATGATGGTCAAAGGCCAAGAATCAGAA |
| | AACAGAGATTTTTCCTCTTCTCTTGTTTCACAAAGTAAATAAACATG |
| | AGACATATAGGACCGGATCAACTAGGTCTATATAAGCAGAGCTCGT |
| | TTAGTGAACCGTCAGATCGCCTGGAGACGCCATCCACGCTGTTTTG |
| | ACCTCCATAGAATTGGTACCGAGCTCG |

### Payloads

A payload of the present disclosure may comprise a coding sequence under transcriptional control of a promoter (e.g., a promoter comprising a response element and a core promoter). In some embodiments, the payload may encode a transgene or therapeutic polynucleotide for delivery to a cell (e.g., a cell of a human or non-human subject). In some embodiments, the payload may comprise a coding sequence encoding a protein (e.g., a protein without a mutation associated with a disease or condition). In some embodiments, the payload may comprise a coding sequence encoding a therapeutic polynucleotide (e.g., a gRNA targeting a gene associated with the disease or condition). Upon delivery of the payload to a cell, the coding sequence may be transcribed in the cell, thereby expressing the encoded protein or therapeutic polynucleotide. In some embodiments, expression of a protein or therapeutic polynucleotide encoded by the coding sequence may treat, prevent, or alleviate symptoms of a disease or disorder. In some embodiments, the payload may comprise a transgene that encodes a wild type copy of a protein that is mutated or dysregulated in the disease or condition.

In some embodiments, cell state specific transcription of a payload sequence (e.g., a transgene) is desired. For example, a transgene lacking a mutation may be specifically transcribed in neurons having a gene comprising the mutation or having a phenotype associated with the mutation. In another example, a transgene lacking a mutation may be specifically transcribed in retinal tissue having gene comprising the mutation or having a phenotype associated with the mutation. In another example, a transgene lacking a genetic variation may be specifically transcribed in cells having the genetic variation or having a phenotype associated with the genetic variation. In some embodiments, cell specific transcription of a payload sequence (e.g., a transgene) is desired. For example, a transgene may be specifically transcribed in neurons of a subject. In another example, a transgene may be specifically transcribed in hepatocytes of a subject. In some embodiments, tissue specific transcription of a payload sequence (e.g., a transgene) is desired. For example, a transgene may be specifically transcribed in cells of the CNS. In another example, a transgene may be specifically transcribed in liver cells. Examples of genes that may be encoded in the payload sequence (e.g., the transgene) are provided in TABLE 5. In some embodiments, the genes may be delivered as transgenes to a cell of a subject to treat a disease or condition in the subject. Alternatively or in addition, a payload may encode a therapeutic polynucleotide (e.g., a gRNA or a tRNA) that targets a gene associated with a disease or condition. For example, a payload may encode a gRNA for gene editing a targeting a gene in a target cell type, tissue type, or cell state. Examples of genes that may be targeted by a therapeutic polynucleotide encoded by a payload are provided in TABLE 5.

TABLE 5
Exemplary Transgene Payloads or Gene Targets and Indications

| Transgene or | |
|---|---|
| Gene Target | Indication |
| MECP2 | Rett syndrome; MECP2 duplication syndrome |
| GRN | Frontotemporal dementia; neuronal ceroid |
| | lipofuscinosis; Alzheimer's disease; amyotrophic |
| | lateral sclerosis (ALS); limbic predominant age-related |
| | TDP-43 encephalopathy (LATE) |
| PRPH2 | Retinitis Pigmentosa 7; macular degeneration |
| RHO | Retinitis Pigmentosa 4 |
| UBE3A | Angelman Syndrome |
| DYRK1A | DYRK1A haploinsufficiency |
| MEF2C | MEF2C haploinsufficiency syndrome |
| NSD1 | Sotos syndrome; Reverse Sotos syndrome |
| ATRX | Alpha-thalassemia X-linked intellectual disability |
| | syndrome |
| RPS6KA3 | Xp22.12 duplication; Coffin-Lowry syndrome |
| TCF4 | Pitt Hopkins syndrome |
| ZEB2 | Mowat-Wilson Syndrome |
| FOXG1 | FOXG1 syndrome |
| CDKL5 | CDKL5 deficiency disorder; West Syndrome |
| Partial piece of | 2q23.1 microdeletion syndrome |
| Chromosome 2 | |
| SLC6A1 | Doose Syndrome; SLC6A1 epileptic encephalopathy |
| DMD | Duchenne's muscular dystrophy; Becker muscular |
| | dystrophy |
| SERPINA1 | Alpha-1 antitrypsin deficiency (AATD) |
| ABCA4 | Macular Degeneration/Stargardt disease |
| CFTR | Cystic Fibrosis |
| HEXA | Tay-Sachs |
| RAB7A | Charcot-Marie-Tooth neuropathy |
| ATP7B | Wilson's disease |
| HFE | Hereditary Hemochromatosis |
| LIPA | Wolman disease; cholesteryl ester storage disease |
| SCNN1A | Psueodhypoaldosteronism type 1 |
| PKD1 | Polycystic Kidney, Autosomal Dominant |
| PKD2 | Polycystic Kidney, Autosomal Dominant |
| PKHD1 | Autosomal Recessive Polycystic Kidney Disease |
| ACE | Kidney Failure, Chronic |
| ALB | Kidney Failure, Chronic |
| VHL | Malignant neoplasm of kidney |
| EPO | Kidney Failure, Chronic |
| PKD2 | Polycystic kidney disease, type 2 |
| FH | Malignant neoplasm of kidney |
| ACE | Kidney Failure |
| TNF | Kidney Failure, Chronic |
| SPP1 | Kidney Calculi |
| IL6 | Kidney Failure, Chronic |
| MYH9 | Kidney Failure, Chronic |
| PKD1 | Autosomal Recessive Polycystic Kidney Disease |
| TSC2 | Kidney Neoplasm |
| ADIPOQ | Kidney Failure, Chronic |
| IL2 | Malignant neoplasm of kidney |
| CCL2 | Kidney Failure, Chronic |
| TGFB1 | Kidney Failure, Chronic |
| VHL | Kidney Neoplasm |
| UMOD | Medullary Cystic Kidney Disease Type 2 |
| BCOR | Clear cell sarcoma of kidney |
| FLCN | Kidney Neoplasm |
| FLCN | Malignant neoplasm of kidney |
| PKD1 | Polycystic kidney disease, type 2 |
| TP53 | Malignant neoplasm of kidney |
| CRP | Kidney Failure, Chronic |
| PTEN | Malignant neoplasm of kidney |
| IFT88 | Autosomal Recessive Polycystic Kidney Disease |
| CLDN14 | Kidney Calculi |
| FH | Kidney Neoplasm |
| VHL | Collecting Duct Carcinoma of the Kidney |
| AGT | Kidney Failure |
| MET | Malignant neoplasm of kidney |
| MYH9 | Kidney Failure |
| YWHAE | Clear cell sarcoma of kidney |
| PKD1 | Polycystic Kidney - body part |
| HAMP | Kidney Failure, Chronic |
| EPO | Kidney Failure |
| MUC1 | Medullary cystic kidney disease 1 |
| BAP1 | Malignant neoplasm of kidney |
| APOE | Kidney Failure |
| CYBA | Kidney Failure, Chronic |
| GSTT1 | Malignant neoplasm of kidney |
| IFNG | Kidney Failure |
| IGF1 | Kidney Failure, Chronic |
| IL2 | Kidney Neoplasm |
| ABCB1 | Malignant neoplasm of kidney |
| SDHB | Malignant neoplasm of kidney |
| TP53 | Collecting Duct Carcinoma of the Kidney |
| TSC2 | Malignant neoplasm of kidney |
| BRAF | Kidney Neoplasm |
| CDKN1B | Malignant neoplasm of kidney |
| GLA | Kidney Failure; Chronic kidney |
| | disease/disorder with a monogenetic |
| | origin |
| KRT7 | Collecting Duct Carcinoma of the Kidney |
| PPARG | Polycystic Kidney, Autosomal Dominant |
| RET | Congenital absence of kidneys syndrome |
| TRPC6 | Kidney Failure, Chronic kidney |
| | disease/disorder with a monogenetic |
| | origin |
| NDRG1 | Malignant neoplasm of kidney |
| GANAB | Polycystic Kidney, Autosomal Dominant |
| NOX4 | Kidney Failure, Chronic |
| ADIPOR1 | Kidney Failure, Chronic |
| GREB1L | Congenital absence of kidneys syndrome |
| ANKS6 | Cystic kidney |
| NUTM2B | Clear cell sarcoma of kidney |
| CAT | Kidney Failure, Chronic |
| CYBA | Kidney Failure |
| CYBB | Kidney Failure, Chronic |
| EGFR | Autosomal Recessive Polycystic Kidney Disease |
| HMOX1 | Kidney Failure, Chronic |
| LRP2 | Kidney Failure, Chronic |
| SERPINE1 | Kidney Failure, Chronic |
| PAX2 | Kidney Failure, Chronic; Chronic kidney |
| | disease/disorder with a monogenetic |
| | origin |
| ABCB1 | Kidney Neoplasm |
| PPARA | Kidney Failure, Chronic |
| PPARG | Kidney Failure |
| PTGS2 | Kidney Failure, Chronic |
| RELA | Kidney Failure, Chronic |
| RET | Unilateral agenesis of kidney |
| TLR4 | Kidney Failure |
| UMOD | Glomerulocystic Kidney Disease with Hyperuricemia |
| | and Isosthenuria |
| BAP1 | Kidney Neoplasm |
| RETN | Kidney Failure, Chronic |
| GREB1L | Unilateral agenesis of kidney |
| FRAS1 | Unilateral agenesis of kidney |
| CRB2 | Cystic Kidney Disease with Ventriculomegaly |
| APRT | Kidney Failure, Chronic |
| AXL | Malignant neoplasm of kidney |
| CCND1 | Malignant neoplasm of kidney |
| BRAF | Malignant neoplasm of kidney |
| CBR1 | Kidney Failure, Chronic |
| CPT1A | Kidney Failure, Chronic |
| CYP1A1 | Malignant neoplasm of kidney |
| CYP2B6 | Kidney Failure, Chronic |
| EDN1 | Kidney Failure |
| ERBB2 | Collecting Duct Carcinoma of the Kidney |
| HMGCR | Kidney Failure, Chronic |
| MME | Kidney Failure |
| NFKB1 | Kidney Failure, Chronic |
| NGF | Kidney Failure, Chronic |
| MAPK1 | Malignant neoplasm of kidney |
| MAPK3 | Malignant neoplasm of kidney |
| PTGS2 | Kidney Neoplasm |
| PTGS2 | Malignant neoplasm of kidney |
| HLTF | Kidney Neoplasm |
| SOD1 | Kidney Calculi |
| SOD2 | Malignant neoplasm of kidney |
| SREBF2 | Kidney Failure, Chronic |
| HNF1B | Unilateral Multicystic Dysplastic Kidney |
| TERT | Clear cell sarcoma of kidney |
| TNFSF10 | Malignant neoplasm of kidney |
| NDRG1 | Kidney Neoplasm |
| MBTPS2 | Brain Anomalies, Retardation, Ectodermal Dysplasia, |
| | Skeletal Malformations, Hirschsprung Disease, |
| | Ear-Eye Anomalies, Cleft Palate-Cryptorchidism, |
| | And Kidney Dysplasia-Hypoplasia |
| WNT4 | Sex Reversal, Female, With Dysgenesis Of |
| | Kidneys, Adrenals, And Lungs |
| BCOR | Kidney Neoplasm |
| INF2 | Kidney Failure; Chronic kidney disease/disorder with a |
| | monogenetic origin |
| ALG9 | Polycystic Kidney Disease, Potter Type I, with |
| | Microbrachycephaly, Hypertelorism, and Brachymelia |
| BICC1 | Polycystic Kidney, Autosomal Dominant |
| TMEM67 | Cystic kidney |
| IRX2 | Clear cell sarcoma of kidney |
| FREM1 | Congenital absence of kidneys syndrome |
| ANKS6 | Polycystic Kidney, Autosomal Dominant |
| FREM2 | Unilateral agenesis of kidney |
| CD46 | Chronic kidney disease/disorder with a monogenetic |
| | origin |
| COL4A3 | Chronic kidney disease/disorder with a monogenetic |
| | origin |
| COL4A4 | Chronic kidney disease/disorder with a monogenetic |
| | origin |
| COL4A5 | Chronic kidney disease/disorder with a monogenetic |
| | origin |
| TTC21B | Chronic kidney disease/disorder with a monogenetic |
| | origin |
| NPHP4 | Chronic kidney disease/disorder with a monogenetic |
| | origin |
| CD2AP | Chronic kidney disease/disorder with a monogenetic |
| | origin |
| CFI | Chronic kidney disease/disorder with a monogenetic |
| | origin |
| LAMB2 | Chronic kidney disease/disorder with a monogenetic |
| | origin |
| LMX1B | Chronic kidney disease/disorder with a monogenetic |
| | origin |
| MYH9 | Chronic kidney disease/disorder with a monogenetic |
| | origin |
| CNGA3 | Achromatopsia |
| CNGB3 | Achromatopsia |
| ABCD1 | Adrenomyeloneuropathy |
| Tafazzin | Barth Syndrome |
| CLN1 | Batten Disease |
| CLN2 | Batten Disease |
| CLN3 | Batten Disease |
| CLN4 | Batten Disease |
| CLN5 | Batten Disease |
| CLN6 | Batten Disease |
| ASPA | Canavan Disease |
| CYP21A2 | Congenital Adrenal Hyperplasia |
| PMM2 | Congenital Disorder of Glycosylation 1a |
| LAMP2 | Danon Disease |
| GLA | Fabry Disease |
| GBA | Gaucher Disease |
| GLB1 | GM1 Gangliosidosis |
| G6PC | Glycogen SD 1a |
| F9 | Hemophilia |
| Serping1 | Hereditary Angioedema |
| GALC | Krabbe Disease |
| GUCY2D | Leber's Disease |
| ND1 | Leber's Disease |
| ND6 | Leber's Disease |
| ND4 | Leber's Disease |
| RPE65 | Leber's Disease |
| AIPL1 | Leber's Disease |
| MTM1 | Myotubular Myopathy |
| I | Mucopolysaccharidosis |
| II | Mucopolysaccharidosis |
| IIA | Mucopolysaccharidosis |
| IIC | Mucopolysaccharidosis |
| IIID | Mucopolysaccharidosis |
| IVA | Mucopolysaccharidosis |
| VI | Mucopolysaccharidosis |
| VII | Mucopolysaccharidosis |
| IXA | Mucopolysaccharidosis |
| OTC | Ornithine Transcarbamylase Deficiency |
| GAA | Pompe Disease |
| HEXB | Sandhoff Disease |
| SMN1 | Spinal Muscular Atrophy |
| USH2D-WHRN | Usher Syndrome |
| USH3A-CLN1 | Usher Syndrome |
| SOD1 | ALS |
| HBB | Beta-thalassemia or Sickle Cell disease |
| BEST1 | Bestrophinopathy |
| CHM | Chorioderemia |
| FXN | Friedreich's Ataxia |
| SLC37A4 | GSD1b |
| IGF1 | Osteoporosis |
| RPGR | Retinitis Pigmentosa |
| USH1C | Usher 1C, 1F |
| CIB2 | Usher 1C, 1F |
| SERPINA1 | Alpha-1 Antitrypsin Deficiency |
| AC6 | Heart Failure |
| Serca2 | Heart Failure |
| VEGF-B | Heart Failure |
| PP1 | Heart Failure |
| GAD | Parkinson's Disease |

Examples of genes that may be encoded by a payload sequence (e.g., the transgene) and delivered to a cell of a subject to treat, prevent, or alleviate symptoms of a disease or condition include MECP2, GRN, PRPH2, RHO, UBE3A, DYRKIA, MEF2C, NSD1, ATRX, RPS6KA3, TCF4, ZEB2, FOXG1, CDKL5, a partial piece of chromosome 2, SLC6A1, DMD, SERPINA1, ABCA4, CFTR, HEXA, RAB7A, ATP7B, HFE, LIPA, SCNNIA, PKD1, PKD2, PKHD1, ACE, ALB, VHL, EPO, FH, ACE, TNF, SPP1, IL6, MYH9, TSC2, ADIPOQ, IL2, CCL2, TGFB1, UMOD, BCOR, FLCN, FLCN, TP53, CRP, PTEN, IFT88, CLDN14, AGT, MET, MYH9, YWHAE, HAMP, EPO, MUC1, BAP1, APOE, CYBA, GSTT1, IFNG, IGF1, IL2, ABCB1, SDHB, TSC2, BRAF, CDKNIB, GLA, KRT7, PPARG, RET, TRPC6, NDRG1, GANAB, NOX4, ADIPOR1, GREBIL, ANKS6, NUTM2B, CAT, CYBA, CYBB, EGFR, HMOX1, LRP2, SERPINE1, PAX2, ABCB1, PPARA, PPARG, PTGS2, RELA, RET, TLR4, UMOD, BAP1, RETN, GREBIL, FRAS1, CRB2, APRT, AXL, CCND1, CBR1, CPTIA, CYP1A1, CYP2B6, EDN1, ERBB2, HMGCR, MME, NFKB1, NGF, MAPK1, MAPK3, PTGS2, PTGS2, HLTF, SOD1, SOD2, SREBF2, HNF1B, TERT, TNFSF10, NDRG1, MBTPS2, WNT4, BCOR, INF2, ALG9, BICC1, TMEM67, IRX2, FREM1, ANKS6, FREM2, CD46, COL4A3, COL4A4, COL4A5, TTC21B, NPHP4, CD2AP, CFI, LAMB2, LMX1B, or MYH9. Alternately or in addition, the payload sequence may encode a therapeutic polynucleotide that targets a gene, such as MECP2, GRN, PRPH2, RHO, UBE3A, DYRKIA, MEF2C, NSD1, ATRX, RPS6KA3, TCF4, ZEB2, FOXG1, CDKL5, a partial piece of chromosome 2, SLC6A1, DMD, SERPINA1, ABCA4, CFTR, HEXA, RAB7A, ATP7B, HFE, LIPA, SCNNIA, PKD1, PKD2, PKHD1, ACE, ALB, VHL, EPO, FH, ACE, TNF, SPP1, IL6, MYH9, TSC2, ADIPOQ, IL2, CCL2, TGFB1, UMOD, BCOR, FLCN, FLCN, TP53, CRP, PTEN, IFT88, CLDN14, AGT, MET, MYH9, YWHAE, HAMP, EPO, MUC1, BAP1, APOE, CYBA, GSTT1, IFNG, IGF1, IL2, ABCB1, SDHB, TSC2, BRAF, CDKNIB, GLA, KRT7, PPARG, RET, TRPC6, NDRG1, GANAB, NOX4, ADIPOR1, GREBIL, ANKS6, NUTM2B, CAT, CYBA, CYBB, EGFR, HMOX1, LRP2, SERPINE1, PAX2, ABCB1, PPARA, PPARG, PTGS2, RELA, RET, TLR4, UMOD, BAP1, RETN, GREBIL, FRAS1, CRB2, APRT, AXL, CCND1, CBR1, CPTIA, CYP1A1, CYP2B6, EDN1, ERBB2, HMGCR, MME, NFKB1, NGF, MAPK1, MAPK3, PTGS2, PTGS2, HLTF, SOD1, SOD2, SREBF2, HNF1B, TERT, TNFSF10, NDRG1, MBTPS2, WNT4, BCOR, INF2, ALG9, BICC1, TMEM67, IRX2, FREM1, ANKS6, FREM2, CD46, COL4A3, COL4A4, COL4A5, TTC21B, NPHP4, CD2AP, CFI, LAMB2, LMX1B, or MYH9, to treat, prevent, or alleviate symptoms of a disease or condition associated with the gene.

In some embodiments, the genes that may be encoded by a payload sequence, or targeted by a therapeutic polynucleotide encoded by the payload sequence, and delivered to a cell of a subject to treat, prevent, or alleviate symptoms of a disease or condition may be associated with a disease or disorder. For example, the genes and associated conditions may include PKD1 or PDK2 associated with Autosomal Dominant Polycystic Kidney Disease; MECP2 associated with Rett Syndrome; CNGA3 or CNGB3 associated with Achromatopsia; ABCDI associated with Adrenomyeloneuropathy; UBE3A associated with Angelman Syndrome; Tafazzin associated with Barth Syndrome; CLN1, CLN2, CLN3, CLN4, CLN5, or CLN6 associated with Batten Disease; ASPA associated with Canavan Disease; PKD1 or PDK2 associated with Autosomal Dominant Polycistic Kidney Disease; CYP21A2 associated with Congenital Adrenal Hyperplasia; PMM2 associated with Congenital Disorder of Glycosylation 1a; LAMP2 associated with Danon Disease; GLA associated with Fabry Disease; GBA associated with Gaucher Disease; GLB1 associated with GM1 Gangliosidosis; G6PC associated with Glycogen SD 1a; F9 associated with Hemophilia; Serping1 associated with Hereditary Angioedema; GALC associated with Krabbe Disease; GUCY2D, ND1, ND6, ND4, RPE65, or AIPL1 associated with Leber's Disease; MIMI associated with Myotubular Myopathy; I, II, IIA, IIC, IIID, IVA, VI, VII, and IXA associated with Mucopolysaccharidosis; OTC associated with Ornithine Transcarbamylase Deficiency; GAA associated with Pompe Disease; HEXB associated with Sandhoff Disease; SMNI associated with Spinal Muscular Atrophy; HEXA associated with Tay-Sachs; USH2D-WHRN and USH3A-CLN1 associated with Usher Syndrome; SOD1 associated with ALS; HBB associated with Beta-thalassemia or Sickle Cell disease; BESTI associated with Bestrophinopathy; CHM associated with Chorioderemia; FXN associated with Friedreich's Ataxia; SLC37A4 associated with GSD1b; IGF1 associated with Osteoporosis; RPGR or RHO associated with Retinitis Pigmentosa; USHIC or CIB2 associated with Usher 1C, 1F; SERPINA1 associated with Alpha-1 Antitrypsin Deficiency; MECP2 associated with Rett Syndrome; AC6, Serca2, VEGF-B, or PPI associated with Heart Failure; GAD associated with Parkinson's Disease; MBTPS2 associated with Brain Anomalies, Retardation, Ectodermal Dysplasia, Skeletal Malformations, Hirschsprung Disease, Ear-Eye Anomalies, Cleft Palate-Cryptorchidism, And Kidney Dysplasia-Hypoplasia; CD46, COL4A3, COL4A4, COL4A5, TTC21B, NPHP4, CD2AP, CFI, LAMB2, LMX1B, or MYH9 associated with Chronic kidney disease/disorder with a monogenetic origin; TERT or IRX2 associated with Clear cell sarcoma of kidney; ERBB2 associated with Collecting Duct Carcinoma of the Kidney; FREM1 associated with Congenital absence of kidneys syndrome; TMEM67 associated with Cystic kidney; SOD1 associated with Kidney Calculi; EDN1 or MME associated with Kidney Failure; CPTIA, CYP2B6, HMGCR, NFKB1, NGF, or SREBF2 associated with Kidney Failure, Chronic; INF2 associated with Kidney Failure or Chronic kidney disease/disorder with a monogenetic origin; PTGS2, HLTF, NDRG1, or BCOR associated with Kidney Neoplasm; CYP1A1, MAPK1, MAPK3, PTGS2, SOD2, or TNFSF10 associated with Malignant neoplasm of kidney; ALG9 associated with Polycystic Kidney Disease, Potter Type I, with Microbrachycephaly, Hypertelorism, and Brachymelia; BICC1 or ANKS6 associated with Polycystic Kidney, Autosomal Dominant; WNT4 associated with Sex Reversal, Female, With Dysgenesis Of Kidneys, Adrenals, And Lungs; FREM2 associated with Unilateral agenesis of kidney; or HNF1B associated with Unilateral Multicystic Dysplastic Kidney.

In some embodiments, a gene (e.g., a transgene) or a therapeutic polynucleotide encoded by a payload may be transcribed in target cell type, cell state, or tissue type at a level that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of the payload in a non-target cell type, cell state, or tissue type.

In some embodiments, a gene (e.g., a transgene) or a therapeutic polynucleotide encoded by a payload may be transcribed in target cell type, cell state, or tissue type at a level that is at least about-0.75-fold, at least about-0.5-fold, at least about-0.25-fold, at least about-0.1-fold, at least about 0-fold, at least about 0.1-fold, at least about 0.25-fold, at least about 0.5-fold, at least about 0.75-fold, at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of a wild type version of the payload in a target cell type, cell state, or tissue type.

In some embodiments, a gene (e.g., a transgene) or a therapeutic polynucleotide encoded by a payload may be transcribed using any core promoter as disclosed herein (e.g., any one of SEQ ID NO: 1-SEQ ID NO: 4, SEQ ID NO: 7, or SEQ ID NO: 10-SEQ ID NO: 144) paired with a response element for a target cell type, cell state, or tissue type, in target cell type, cell state, or tissue type at a level that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of the payload in a non-target cell type, cell state, or tissue type.

In some embodiments, a gene (e.g., a transgene) or a therapeutic polynucleotide encoded by a payload may be transcribed using any core promoter as disclosed herein (e.g., any one of SEQ ID NO: 1-SEQ ID NO: 4, SEQ ID NO: 7, or SEQ ID NO: 10-SEQ ID NO: 144) paired with a response element for a target cell type, cell state, or tissue type, in target cell type, cell state, or tissue type at a level that is at least about-0.75-fold, at least about-0.5-fold, at least about-0.25-fold, at least about-0.1-fold, at least about 0-fold, at least about 0.1-fold, at least about 0.25-fold, at least about 0.5-fold, at least about 0.75-fold, at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of a wild type version of the payload in a target cell type, cell state, or tissue type.

### Recombinant Vectors

In some embodiments, a polynucleotide of the present disclosure is introduced into a subject via a recombinant vector. In some embodiments, an engineered core promoter of the present disclosure (e.g., any one of SEQ ID NO: 1-SEQ ID NO: 4, SEQ ID NO: 7, or SEQ ID NO: 10-SEQ ID NO: 144) is introduced into a subject via a recombinant vector. In some embodiments, an engineered core promoter of the present disclosure paired with a response element is introduced into a subject via a recombinant vector. In some embodiments, an engineered core promoter of the present disclosure paired with a response element that is upstream of a polynucleotide encoding a payload is introduced into a subject via a recombinant vector. In some embodiments the vector is a plasmid, a viral vector, an expression cassette, or a transformed cell.

In some embodiments, the viral vector is an adenoviral vector, an adeno-associated viral vector, or a lentiviral vector. Adeno-associated virus (AAV) vectors include vectors derived from any AAV serotype, including, but not limited to AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP. V1, AAV.PHP.B, AAV.PhB.C1, AAV.PhB.C2, AAV.PhB.C3, AAV.PhB.C6, AAV.cy5, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68.

In some embodiments, a polynucleotide is introduced into a subject by non-viral vector systems. In some embodiments, an engineered core promoter of the present disclosure (e.g., any one of SEQ ID NO: 1-SEQ ID NO: 4, SEQ ID NO: 7, or SEQ ID NO: 10-SEQ ID NO: 144) is introduced into a subject by non-viral vector systems. In some embodiments, an engineered core promoter of the present disclosure paired with a response element is introduced into a subject by non-viral vector systems. In some embodiments, an engineered core promoter of the present disclosure paired with a response element that is upstream of a polynucleotide encoding a payload is introduced into a subject by non-viral vector systems. In some embodiments, cationic lipids, polymers, hydrodynamic injection and/or ultrasound may be used in delivering a polynucleotide to a subject in the absence of virus. In some embodiments, cationic lipids, polymers, hydrodynamic injection and/or ultrasound may be used in delivering an engineered core promoter of the present disclosure (e.g., any one of SEQ ID NO: 1-SEQ ID NO: 4, SEQ ID NO: 7, or SEQ ID NO: 10-SEQ ID NO: 144) to a subject in the absence of virus. In some embodiments, cationic lipids, polymers, hydrodynamic injection and/or ultrasound may be used in delivering an engineered core promoter of the present disclosure paired with a response element to a subject in the absence of virus. In some embodiments, cationic lipids, polymers, hydrodynamic injection and/or ultrasound may be used in delivering an engineered core promoter of the present disclosure paired with a response element that is upstream of a polynucleotide encoding a payload to a subject in the absence of virus.

In some examples, the vector may be a eukaryotic vector, a prokaryotic vector (e.g., a bacterial vector) a viral vector, or any combination thereof. In some examples, the vector may be a viral vector. In some embodiments, the viral vector may be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector may be a recombinant vector, a hybrid vector, a chimeric vector, a self-complementary vector, a single-stranded vector, or any combination thereof.

In some embodiments, the viral vector may be an adeno-associated virus (AAV). In some embodiments, the AAV may be any AAV known in the art. In some embodiments, the viral vector may be of a specific serotype. In some embodiments, the viral vector may be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV-DJ serotype, AAV-DJ/8 serotype, AAV-DJ/9 serotype, AAV1/2 serotype, AAV.rh8 serotype, AAV.rh10 serotype, AAV.rh20 serotype, AAV.rh39 serotype, AAV.Rh43 serotype, AAV.Rh74 serotype, AAV.v66 serotype, AAV.Oligo001 serotype, AAV.SCH9 serotype, AAV.r3.45 serotype, AAV.RHM4-1 serotype, AAV.hu37 serotype, AAV.Anc80 serotype, AAV.Anc80L65 serotype, AAV.7m8 serotype, AAV.PhP.eB serotype, AAV.PhP.VI serotype, AAV.PHP.B serotype, AAV.PhB.C1 serotype, AAV.PhB.C2 serotype, AAV.PhB.C3 serotype, AAV.PhB.C6 serotype, AAV.cy5 serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, AAV.HSC17 serotype, or AAVhu68 serotype, a derivative of any of these serotypes, or any combination thereof.

In some embodiments, the AAV vector may be a recombinant vector, a hybrid AAV vector, a chimeric AAV vector, a self-complementary AAV (scAAV) vector, a single-stranded AAV, or any combination thereof.

In some embodiments, the AAV vector may be a recombinant AAV (rAAV) vector. Methods of producing recombinant AAV vectors may be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and payload sequences, etc. In some examples, the viral vectors described herein may be engineered through synthetic or other suitable means by references to published sequences, such as those that may be available in the literature. For example, the genomic and protein sequences of various serotypes of AAV, as well as the sequences of the native terminal repeats (TRs), Rep proteins, and capsid subunits may be known in the art and may be found in the literature or in public databases such as GenBank or Protein Data Bank (PDB).

In some examples, methods of producing delivery vectors herein comprising packaging a polynucleotide of the present disclosure in an AAV vector. In some examples, methods of producing delivery vectors herein comprising packaging an engineered core promoter of the present disclosure (e.g., any one of SEQ ID NO: 1-SEQ ID NO: 4, SEQ ID NO: 7, or SEQ ID NO: 10-SEQ ID NO: 144) in an AAV vector. In some examples, methods of producing delivery vectors herein comprising packaging an engineered core promoter of the present disclosure paired with a response element in an AAV vector. In some examples, methods of producing delivery vectors herein comprising packaging an engineered core promoter of the present disclosure paired with a response element that is upstream of a polynucleotide encoding a payload in an AAV vector. In some examples, methods of producing the delivery vectors described herein comprise, (a) introducing into a cell: (i) a polynucleotide disclosed herein (e.g., an engineered core promoter of the present disclosure; an engineered core promoter of the present disclosure paired with a response element; or an engineered core promoter of the present disclosure paired with a response element that is upstream of a polynucleotide encoding a payload); and (ii) a viral genome comprising a Replication (Rep) gene and Capsid (Cap) gene that encodes a wild type AAV capsid protein or modified version thereof; (b) expressing in the cell the wild type AAV capsid protein or modified version thereof; (c) assembling an AAV particle; and (d) packaging the polynucleotide disclosed herein in the AAV particle, thereby generating an AAV delivery vector. In some examples, any polynucleotide disclosed herein may be packaged in the AAV vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector may be produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector may be a chimeric AAV vector. In some examples, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the AAV vector comprises a self-complementary AAV genome. Self-complementary AAV genomes may be generally known in the art and contain both DNA strands which can anneal together to form double-stranded DNA.

In some examples, the delivery vector may be a retroviral vector. In some examples, the retroviral vector may be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector may be transfected such that the majority of sequences coding for the structural genes of the virus (e.g., gag, pol, and env) may be deleted and replaced by the gene(s) of interest (e.g., the payload).

In some examples, the delivery vehicle may be a non-viral vector. Examples of non-viral vectors may include plasmids, lipid nanoparticles, lipoplexes, polymersomes, polyplexes, dendrimers, nanoparticles, and cell-penetrating peptides. The non-viral vector may comprise a polynucleotide, such as a plasmid, encoding for a promoter (e.g., comprising a cell type- or cell state-specific response element and a switchable core promoter) and a payload sequence. The non-viral vector may comprise an engineered core promoter of the present disclosure (e.g., any one of SEQ ID NO: 1-SEQ ID NO: 4, SEQ ID NO: 7, or SEQ ID NO: 10-SEQ ID NO: 144). The non-viral vector may comprise an engineered core promoter of the present disclosure paired with a response element. The non-viral vector may comprise an engineered core promoter of the present disclosure paired with a response element that is upstream of a polynucleotide encoding a payload. In some examples, the delivery vehicle may be a plasmid. In some examples, the plasmid may be a minicircle plasmid. In some embodiments, a vector may comprise naked DNA (e.g., a naked DNA plasmid). In some embodiments, the non-viral vector comprises DNA. In some embodiments, the non-viral vector comprises RNA. In some examples, the non-viral vector comprises circular double-stranded DNA. In some examples, the non-viral vector may comprise a linear polynucleotide. In some examples, the non-viral vector comprises a polynucleotide encoding one or more genes of interest and one or more regulatory elements. In some examples, the non-viral vector comprises a bacterial backbone containing an origin of replication and an antibiotic resistance gene or other selectable marker for plasmid amplification in bacteria. In some examples, the non-viral vector contains one or more genes that provide a selective marker to induce a target cell to retain a polynucleotide (e.g., a plasmid) of the non-viral vector. In some examples, the non-viral vector may be formulated for delivery through injection by a needle carrying syringe. In some examples, the non-viral vector may be formulated for delivery via electroporation. In some examples, a polynucleotide of the non-viral vector may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence.

### Methods of Regulating Payload Translation

In some embodiments, the present disclosure provides a method of inserting a polynucleotide comprising the promoter (e.g., comprising an engineered core promoter, such as any one of SEQ ID NO: 1-SEQ ID NO: 4, SEQ ID NO: 7, or SEQ ID NO: 10-SEQ ID NO: 144, paired with a response element) as described herein and the payload into a recombinant polynucleotide cassette. The recombinant polynucleotide cassette may further modulate expression of the payload (e.g., by modulating translation). In some embodiments, the recombinant polynucleotide cassette modulates stability of the payload RNA. In some embodiments, the recombinant polynucleotide cassette comprises a 5′UTR effector region. In some embodiments, the recombinant polynucleotide cassette comprises a 3′UTR effector region. In some embodiments, the payload is codon optimized in the recombinant polynucleotide cassette. In some embodiments, an intron is inserted into the 5′UTR effector region or the sequence of the payload. In some embodiments, the intron is a natural intron or a synthetic intron.

In some embodiments, the recombinant polynucleotide cassette comprises the promoter as described herein (e.g., comprising an engineered core promoter, such as any one of SEQ ID NO: 1-SEQ ID NO: 4, SEQ ID NO: 7, or SEQ ID NO: 10-SEQ ID NO: 144, paired with a response element), the payload sequence, and one or more of: a 5′UTR effector region; a 3′UTR effector region; a codon optimized sequence of the payload; and an intron in the sequence of the payload. In some embodiments, translation of the payload increases from the recombinant polynucleotide cassette comprising one or more of the 5′UTR effector region, the 3′UTR effector region, a codon optimized sequence of the payload, and the intron in the sequence of the payload compared to from a recombinant polynucleotide cassette lacking the one or more of the 5′UTR effector region, the 3′UTR effector region, a codon optimized sequence of the payload, and the intron in the sequence of the payload. In some embodiments, translation of the payload decreases from the recombinant polynucleotide cassette comprising one or more of the 5′UTR effector region, the 3′UTR effector region, a codon optimized sequence of the payload, and the intron in the sequence of the payload compared to from a recombinant polynucleotide cassette lacking the one or more of the 5′UTR effector region, the 3′UTR effector region, a codon optimized sequence of the payload, and the intron in the sequence of the payload.

In some embodiments, the 5′ UTR effector region comprises one or more of: a structural element; a sequence motif; a nucleotide base content comprising a G/C content of at least 1%, 5%, 10%, 20%, 30%, 40%, 50%, 60%, 70%, 80%, 90%, 95%, 97%, 99%, or 100%; and a 5′ UTR intron. In some embodiments, the structural element is a site that is in a conformation with the 5′cap so that the 5′cap is inaccessible or has low accessibility to the translation machinery (e.g., also referred to as “a cap-burying site”), resulting in decreased or no translation compared to a 5′UTR lacking this structural element. In some embodiments, the structural element is an Internal Ribosome Entry Site (IRES). In some embodiments, the structural element is an RNA pseudoknot. In some embodiments, the structural element is an Iron Responsive Element (IRE). In some embodiments, the structural element is a non-coding translation modulatory structure. In some embodiments, the structural element is a hairpin. In some embodiments, the structural element is a sequence (e.g., a cap-burying site sequence, an IRES, an RNA pseudoknot, an IRE, or a non-coding translation modulatory structure), that changes conformation when a sequence element contacts a target RNA with which it has at least partial complementarity, such that the rate of translation of the payload downstream of the structural element is increased or decreased compared to translation of the payload prior to the sequence element contacting the target RNA. In some embodiments, the 5′UTR effector region further comprises the sequence element, wherein a nucleic acid sequence of the sequence element is at least partially complementary to a sequence of a target RNA and wherein conformation of the structural element changes when the sequence element contacts the target RNA with which it has at least partial complementarity. In some embodiments, the 5′ UTR intron is a natural intron, synthetic intron, or a fragment thereof.

In some embodiments, the 3′ UTR comprises one or more of: a site that recruits polyA tail machinery; an miRNA binding site; or a sequence motif. In some embodiments, the poly (A) tail recruitment machinery comprises an enzyme. In some embodiments, the length of the poly (A) tail modulates protein expression from the polynucleotide. In some embodiments, the 3′UTR effector region comprises one, two, three, four, five, six, seven, eight, nine, ten, or more miRNA binding sites. In some embodiments, the miRNA binding sites are for the same miRNA. In some embodiments, the miRNA binding sites are for different miRNA. In some embodiments, the sequence motif is an AC-rich motif. In some embodiments, the sequence motif is an AU-rich element (ARE).

In some embodiments, the codon optimized sequence of the therapeutic polynucleotide is a least 1%, 5%, 10%, 20%, 30%, 40%, 50%, 60%, 70%, 80%, 90%, 95%, 97%, 99%, or 100% codon optimized. In some embodiments, the intron in the sequence of the therapeutic polynucleotide is a natural intron, synthetic intron, or a fragment thereof. In some embodiments, the recombinant polynucleotide cassette is encoded by a DNA vector.

In some embodiments, the combination of the promoter sequence and payload sequence as described herein in the recombinant polynucleotide cassette results in the payload being expressed at a therapeutic level for reducing or alleviating at least one symptom of the disease or disorder. The therapeutic level can be-0.25-fold,-0.5-fold, 0-fold, 0.25-fold, 0.5-fold, 0.75-fold, 1-fold, 1.5-fold, 2-fold, or 4-fold greater than the biological level of the payload.

### Methods of Treatment and Pharmaceutical Compositions

Methods for treatment of diseases or disorders characterized by aberrant gene expression are also encompassed by the present disclosure. Said methods include administering a therapeutically effective amount of a payload sequence as part of a recombinant polynucleotide cassette. The recombinant polynucleotide cassette of the disclosure can be formulated in pharmaceutical compositions. These compositions can comprise, in addition to one or more of the recombinant polynucleotide cassettes (e.g., comprising an engineered core promoter, such as any one of SEQ ID NO: 1-SEQ ID NO: 4, SEQ ID NO: 7, or SEQ ID NO: 10-SEQ ID NO: 144), a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In some embodiments, the polynucleotide of the present disclosure or recombinant polynucleotide cassette of the present disclosure (e.g., a polynucleotide or recombinant polynucleotide cassette comprising an engineered core promoter, such as any one of SEQ ID NO: 1-SEQ ID NO: 4, SEQ ID NO: 7, or SEQ ID NO: 10-SEQ ID NO: 144) may be administered to cells via a lipid nanoparticle. In some embodiments, the lipid nanoparticle may be administered at the appropriate concentration according to standard methods appropriate for the target cells.

In some embodiments, the polynucleotide of the present disclosure or recombinant polynucleotide cassette of the present disclosure (e.g., a polynucleotide or recombinant polynucleotide cassette comprising an engineered core promoter, such as any one of SEQ ID NO: 1-SEQ ID NO: 4, SEQ ID NO: 7, or SEQ ID NO: 10-SEQ ID NO: 144) may be administered to cells via a viral vector. In some embodiments, the viral vector may be administered at the appropriate multiplicity of infection according to standard transduction methods appropriate for the target cells. Titers of the virus vector or capsid to administer can vary depending on the target cell type or cell state and number and can be determined by those of skill in the art. In some embodiments, at least about 10² infections units are administered. In some embodiments, at least about 10³, 10⁴, 10⁵, 10⁶, 10⁷, 10⁸, 10⁹, 10¹⁰, 10¹¹, 10¹², or 10¹³ infectious units are administered.

In some embodiments, the polynucleotide or recombinant polynucleotide cassette (e.g., a polynucleotide or recombinant polynucleotide cassette comprising an engineered core promoter, such as any one of SEQ ID NO: 1-SEQ ID NO: 4, SEQ ID NO: 7, or SEQ ID NO: 10-SEQ ID NO: 144) is introduced to cells of any type or state, including, but not limited to neural cells, cells of the eye (including retinal cells, retinal pigment epithelium, and corneal cells), lung cells, epithelial cells, skeletal muscle cells, dendritic cells, hepatic cells, pancreatic cells, bone cells, hematopoietic stem cells, spleen cells, keratinocytes, fibroblasts, endothelial cells, prostate cells, and heart cells.

In some embodiments, the polynucleotide or the disclosure or the recombinant polynucleotide cassette of the disclosure (e.g., a polynucleotide or recombinant polynucleotide cassette comprising an engineered core promoter, such as any one of SEQ ID NO: 1-SEQ ID NO: 4, SEQ ID NO: 7, or SEQ ID NO: 10-SEQ ID NO: 144) may be introduced to cells in vitro via a viral vector for administration of modified cells to a subject. In some embodiments, a viral vector encoding the polynucleotide of the disclosure or the recombinant polynucleotide cassette of the disclosure is introduced to cells that have been removed from a subject. In some embodiments, the modified cells are placed back in the subject following introduction of the viral vector.

In some embodiments, a dose of modified cells is administered to a subject according to the age and species of the subject, disease or disorder to be treated, as well as the cell type or state and mode of administration. In some embodiments, at least about 10²-10⁸ cells are administered per dose. In some embodiments, cells transduced with viral vector are administered to a subject in an effective amount.

In some embodiments, the dose of viral vector administered to a subject will vary according to the age of the subject, the disease or disorder to be treated, and mode of administration. In some embodiments, the dose for achieving a therapeutic effect is a virus titer of at least about 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ or more transducing units.

Administration of the pharmaceutically useful polynucleotide of the present disclosure or the polynucleotide cassette of the present disclosure is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Diseases and Disorders

In some embodiments, the recombinant polynucleotide of the disclosure or the recombinant polynucleotide cassette of the disclosure (e.g., a polynucleotide, recombinant polynucleotide, or recombinant polynucleotide cassette comprising an engineered core promoter, such as any one of SEQ ID NO: 1-SEQ ID NO: 4, SEQ ID NO: 7, or SEQ ID NO: 10-SEQ ID NO: 144) is used for treating a disease or disorder associated with abnormal expression of a gene or protein. In some embodiments the disease or disorder is Rett syndrome, MECP2 duplication syndrome, frontotemporal dementia, neuronal ceroid lipofuscinosis, amyotrophic lateral sclerosis (ALS), limbic predominant age-related TDP-43 encephalopathy (LATE), autosomal recessive polycystic kidney disease, kidney failure, chronic, malignant neoplasm of kidney, polycystic kidney disease, type 2, kidney failure, kidney neoplasm, medullary cystic kidney disease type 2, clear cell sarcoma of kidney, kidney calculi, collecting duct carcinoma of the kidney, polycystic kidney, medullary cystic kidney disease 1, congenital absence of kidneys syndrome, cystic kidney, chronic kidney disease/disorder with a monogenetic origin, unilateral agenesis of kidney, glomerulocystic kidney disease with hyperuricemia and isosthenuria, cystic kidney disease with ventriculomegaly, unilateral multicystic dysplastic kidney, brain anomalies, retardation, ectodermal dysplasia, skeletal malformations, Hirschsprung disease, ear-eye anomalies, cleft palate-cryptorchidism, and kidney dysplasia-hypoplasia, sex reversal, female, with dysgenesis of kidneys, adrenals, and lungs, polycystic kidney disease, potter type I, with microbrachycephaly, hypertelorism, brachymelia, retinitis pigmentosa 7, macular degeneration, retinitis pigmentosa 4, Angelman syndrome, DYRKIA haploinsufficiency, MEF2C haploinsufficiency syndrome, Sotos syndrome, reverse Sotos syndrome, alpha-thalassemia X-linked intellectual disability syndrome, Xp22.12 duplication, Coffin-Lowry syndrome, Pitt Hopkins syndrome, Mowat-Wilson syndrome, FOXG1 syndrome, CDKL5 deficiency disorder, West syndrome, 2q23.1 microdeletion syndrome, Doose syndrome, SLC6A1 epileptic encephalopathy, Duchenne's muscular dystrophy, Becker muscular dystrophy, alpha-1 antitrypsin deficiency (AATD), macular degeneration/Stargardt disease, cystic fibrosis, Tay-Sachs, Charcot-Marie-Tooth neuropathy, Wilson's disease, hereditary hemochromatosis, Wolman disease, cholesteryl ester storage disease, psueodhypoaldosteronism type 1, autosomal dominant polycystic kidney disease, achromatopsia, adrenomyeloneuropathy, Barth syndrome, Batten disease, Canavan disease, congenital adrenal hyperplasia, congenital disorder of glycosylation 1a, Danon disease, Fabry disease, Gaucher disease, GM1 gangliosidosis, glycogen SD 1a, hemophilia, hereditary angioedema, Krabbe disease, Leber's disease, myotubular myopathy, mucopolysaccharidosis, ornithine transcarbamylase deficiency, Pompe disease, Sandhoff disease, spinal muscular atrophy, Usher syndrome, beta-thalassemia, bestrophinopathy, chorioderemia, Friedreich's ataxia, GSD1b, osteoporosis, sickle cell disease, heart failure, or Parkinson's disease. In some embodiments, the disease or disorder is Rett syndrome. In some embodiments, the disease or disorder is frontotemporal dementia. In some embodiments, the disease or disorder is a retinal disorder. In some embodiments, the retinal disorder comprises Retinitis Pigmentosa 7, Retinitis Pigmentosa 4, or macular degeneration.

In some embodiments, a polynucleotide of the present disclosure or the recombinant polynucleotide cassette of the present disclosure (e.g., a polynucleotide or recombinant polynucleotide cassette comprising an engineered core promoter, such as any one of SEQ ID NO: 1-SEQ ID NO: 4, SEQ ID NO: 7, or SEQ ID NO: 10-SEQ ID NO: 144) may be administered using a viral vector (e.g., an AAV vector) or a non-viral vector to a subject in need thereof. For example, the subject in need thereof may have or be at risk for frontotemporal dementia. Upon administration of said polynucleotide or said recombinant polynucleotide cassette, a payload sequence (e.g., encoding progranulin, polycystin-1, or polycystin-2) may be expressed in a cell type, cell state, or tissue type of interest (e.g., a diseased cell, a neuronal cell, CNS tissue, renal cell, or renal tissue). The payload sequence may be selectively transcribed in the cell type, cell state, or tissue type of interest, thereby preventing unwanted adverse effects in the subject due to expression in non-target tissues. The subject can be a human or a non-human animal. Thus, the polynucleotides disclosed herein or the recombinant polynucleotide cassettes disclosed herein can serve as a therapeutically effective vector replacement therapy that senses endogenous nucleic acids to regulate expression of a payload sequence and prevent or minimize adverse side effects from overexpression of a payload sequence.

As used herein, the term “therapeutic polynucleotide” may to a polynucleotide that is introduced into a cell and is capable of being expressed in the cell or to a polynucleotide that may, in itself, have a therapeutic activity, such as a gRNA or a tRNA.

As used herein, the term “polynucleotide” refers to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), IRNA (ribosomal RNA), snoRNA (small nucleolar RNA), and cRNA (complementary RNA). The term DNA is inclusive of cDNA, genomic DNA, and DNA-RNA hybrids.

The term “ameliorating” refers to any therapeutically beneficial result in the treatment of a disease state, e.g., Rett syndrome, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows:

[(Number of Identical Positions)/(Total Number of Positions in the Test Sequence)]×100%

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023 are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows:

Percent Sequence Identity=(“Percent Identity” output value)×(“Query Coverage” output value)

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (length=10 nucleotides) to reference sequence 2: AAAAAAAAAA (length=10 nucleotides). The percent identity between test sequence 1 and reference sequence 2 would be [(5)/(10)]×100%=50%. Test sequence 1 has 50% sequence identity to reference sequence 2. In another example, percent identity is calculated to compare test sequence 3: CCCCCGGGGGGGGGGCCCCC (length=20 nucleotides) to reference sequence 4: GGGGGGGGGG (length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (length=10 nucleotides) to reference sequence 6: CCCCCGGGGGGGGGGCCCCC (length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (length=10 amino acids) to reference sequence 8: YYYYYYYYYY (length=10 amino acids). The percent identity between test sequence 7 and reference sequence 8 would be [(5)/(10)]×100%=50%. Test sequence 7 has 50% sequence identity to reference sequence 8. In another example, percent identity is calculated to compare test sequence 9: LLLLLFFFFFYYYYYLLLLL (length=20 amino acids) to reference sequence 10: FFFFFYYYYY (length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (length=10 amino acids) to reference sequence 12: LLLLLFFFFFYYYYYLLLLL (length=20 amino acids). The percent identity between test sequence 11 and reference sequence 12 would be [(10)/(10)]×100%=100%. Test sequence 11 has 100% sequence identity to reference sequence 12.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21th Ed., Mack Publ. Co., Easton, Pa. (2005), incorporated herein by reference in its entirety.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

### NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. An engineered core promoter sequence comprising a TATA box, an initiator element, a spacer separating the TATA box and the initiator element, and one or more features selected from: a) the TATA box comprises a sequence having at least 85% sequence identity to any one of SEQ ID NO: 199-SEQ ID NO: 201; b) the initiator element comprises a sequence having at least 75% sequence identity to any one of SEQ ID NO: 202-SEQ ID NO: 204; c) the spacer is a short spacer comprising no less than 25 and no more than 29 nucleotide residues between the TATA box and the initiator element; d) the spacer is a long spacer comprising no less than 31 and no more than 48 nucleotide residues between the TATA box and the initiator element; e) a YY1 motif comprising a sequence having at least 80% sequence identity to any one of SEQ ID NO: 205-SEQ ID NO: 207; f) a transcriptional pause site comprising a sequence having at least 90% sequence identity to any one of SEQ ID NO: 196-SEQ ID NO: 198; g) at least 70% sequence identity and at least one nucleotide substitution relative to SEQ ID NO: 5, SEQ ID NO: 9, or SEQ ID NO: 210; and h) combinations thereof. 2. The engineered core promoter sequence of embodiment 1, comprising at least 70% sequence identity and nucleotide substitutions T15G and A40C relative to SEQ ID NO: 5. 3. The engineered core promoter sequence of embodiment 1, comprising at least 70% sequence identity and nucleotide substitutions T15A and A40T relative to SEQ ID NO: 5. 4. The engineered core promoter sequence of any one of embodiments 1-3, wherein the spacer comprises from 29 to 31 nucleotides between the TATA box and the initiator element. 5. The engineered core promoter sequence of any one of embodiments 1-4, wherein the spacer comprises 30 nucleotides between the TATA box and the initiator element. 6. The engineered core promoter sequence of any one of embodiments 1-3, wherein the short spacer comprises 28 nucleotide residues between the TATA box and the initiator element. 7. The engineered core promoter sequence of any one of embodiments 1-3, wherein the long spacer comprises 32 nucleotide residues between the TATA box and the initiator element. 8. The engineered core promoter sequence of any one of embodiments 1-7, wherein the TATA box comprises a sequence having at least 90% sequence identity to any one of SEQ ID NO: 199-SEQ ID NO: 201. 9. The engineered core promoter sequence of any one of embodiments 1-8, wherein the TATA box comprises a sequence of any one of SEQ ID NO: 199-SEQ ID NO: 201. 10. The engineered core promoter sequence of any one of embodiments 1-9, wherein the initiator element comprises a sequence having at least 85% sequence identity to any one of SEQ ID NO: 202-SEQ ID NO: 204. 11. The engineered core promoter sequence of any one of embodiments 1-10, wherein the initiator element comprises a sequence of any one of SEQ ID NO: 202-SEQ ID NO: 204. 12. The engineered core promoter sequence of any one of embodiments 1-11, wherein the YY1 motif comprises a sequence having at least 90% sequence identity to any one of SEQ ID NO: 205-SEQ ID NO: 207. 13. The engineered core promoter sequence of any one of embodiments 1-12, wherein the YY1 motif comprises a sequence of any one of SEQ ID NO: 205-SEQ ID NO: 207. 14. The engineered core promoter sequence of any one of embodiments 1-11, wherein the transcriptional pause site comprises a sequence having at least 95% sequence identity to any one of SEQ ID NO: 196-SEQ ID NO: 198. 15. The engineered core promoter sequence of any one of embodiments 1-11, wherein the transcriptional pause site comprises a sequence of any one of SEQ ID NO: 196-SEQ ID NO: 198. 16. The engineered core promoter of any one of embodiments 1-15, wherein the engineered core promoter comprises two or more of the features. 17. The engineered core promoter sequence of any one of embodiments 1-16, wherein the engineered core promoter comprises features a) and b). 18. The engineered core promoter of any one of embodiments 1-17, wherein the engineered core promoter comprises three or more of the features. 19. The engineered core promoter sequence of any one of embodiments 1-18, wherein the engineered core promoter comprises features a) and b) and at least one more of the features. 20. The engineered core promoter sequence of any one of embodiments 1-19, wherein the engineered core promoter comprises features a), b), and c). 21. The engineered core promoter sequence of any one of embodiments 1-19, wherein the engineered core promoter comprises features a), b), and d). 22. The engineered core promoter sequence of any one of embodiments 1-19, wherein the engineered core promoter comprises features a), b), and e). 23. The engineered core promoter sequence of any one of embodiments 1-19, wherein the engineered core promoter comprises features a), b), and f). 24. An engineered core promoter sequence comprising a sequence having at least 80%, 85%, 90%, 93%, 95%, 97%, 99%, or 100% sequence identity to any one of SEQ ID NO: 1-SEQ ID NO: 4, SEQ ID NO: 7, or SEQ ID NO: 10-SEQ ID NO: 144. 25. The engineered core promoter sequence of embodiment 23, wherein the engineered core promoter sequence comprises a sequence having at least 80%, 85%, 90%, 93%, 95%, 97%, 99%, or 100% sequence identity to any one of SEQ ID NO: 1-SEQ ID NO: 4, SEQ ID NO: 7, SEQ ID NO: 10-SEQ ID NO: 15, or SEQ ID NO: 110. 26. The engineered core promoter sequence of embodiment 23 or embodiment 24, wherein the engineered core promoter sequence comprises a sequence of SEQ ID NO: 7. 27. The engineered core promoter sequence of embodiment 23 or embodiment 24, wherein the engineered core promoter sequence comprises a sequence of SEQ ID NO: 1. 28. The engineered core promoter sequence of embodiment 23 or embodiment 24, wherein the engineered core promoter sequence comprises a sequence of SEQ ID NO: 2. 29. The engineered core promoter sequence of embodiment 23 or embodiment 24, wherein the engineered core promoter sequence comprises a sequence of SEQ ID NO: 3. 30. The engineered core promoter sequence of embodiment 23 or embodiment 24, wherein the engineered core promoter sequence comprises a sequence of SEQ ID NO: 4. 31. The engineered core promoter sequence of embodiment 23 or embodiment 24, wherein the engineered core promoter sequence comprises a sequence of SEQ ID NO: 10. 32. The engineered core promoter sequence of embodiment 23 or embodiment 24, wherein the engineered core promoter sequence comprises a sequence of SEQ ID NO: 11. 33. The engineered core promoter sequence of embodiment 23 or embodiment 24, wherein the engineered core promoter sequence comprises a sequence of SEQ ID NO: 12. 34. The engineered core promoter sequence of embodiment 23 or embodiment 24, wherein the engineered core promoter sequence comprises a sequence of SEQ ID NO: 13. 35. The engineered core promoter sequence of embodiment 23 or embodiment 24, wherein the engineered core promoter sequence comprises a sequence of SEQ ID NO: 14. 36. The engineered core promoter sequence of embodiment 23 or embodiment 24, wherein the engineered core promoter sequence comprises a sequence of SEQ ID NO: 15. 37. The engineered core promoter sequence of embodiment 23 or embodiment 24, wherein the engineered core promoter sequence comprises a sequence of SEQ ID NO: 110. 38. The engineered core promoter sequence of any one of embodiments 1-37, wherein the engineered core promoter sequence comprises a TATA box, an RNA polymerase binding sequence, a B recognition element, a CCAAT box, or a Pribnow box. 39. The engineered core promoter sequence of any one of embodiments 1-38, wherein the engineered core promoter sequence is capable of recruiting a polymerase. 40. The engineered core promoter sequence of embodiment 39, wherein the polymerase is an RNA polymerase II. 41. The engineered core promoter sequence of any one of embodiments 1-40, wherein the engineered core promoter has a dynamic range that is higher than a dynamic range of SEQ ID NO: 6. 42. The engineered core promoter sequence of any one of embodiments 1-41, wherein the engineered core promoter has a dynamic range that is higher than a dynamic range of SEQ ID NO: 9. 43. The engineered core promoter sequence of any one of embodiments 1-42, wherein the engineered core promoter has an activity that is higher than an activity of SEQ ID NO: 6. 44. The engineered core promoter sequence of any one of embodiments 1-43, wherein the engineered core promoter has an activity that is higher than an activity of SEQ ID NO: 9. 45. The engineered core promoter sequence of any one of embodiments 1-44, wherein the engineered core promoter has an activity that is higher than an activity of SEQ ID NO: 6 when paired with an active response element, a basal activity that is lower than a basal activity of SEQ ID NO: 6 when paired with an inactive response element, or both. 46. The engineered core promoter sequence of any one of embodiments 1-45, wherein the engineered core promoter has an activity that is higher than an activity of SEQ ID NO: 9 when paired with an active response element, a basal activity that is lower than a basal activity of SEQ ID NO: 9 when paired with an inactive response element, or both. 47. A recombinant polynucleotide comprising a promoter and a payload, wherein the promoter comprises: a response element capable of binding to a cognate ligand, a coactivator, or a corepressor; and the engineered core promoter sequence of any one of embodiments 1-46 capable of recruiting a polymerase; wherein the payload comprises a coding sequence. 48. The recombinant polynucleotide of embodiment 47, wherein the response element is an enhancer. 49. The recombinant polynucleotide of embodiment 47 or embodiment 48, wherein binding of the cognate ligand or the coactivator to the response element increases transcription of the payload in a target cell type, a target cell state, or a target tissue. 50. The recombinant polynucleotide of embodiment 47, wherein binding of the corepressor to the response element decreases transcription of the payload in a non-target cell type, a non-target cell state, or a non-target tissue. 51. The recombinant polynucleotide of any one of embodiments 47-50, wherein the response element confers neuron-specific transcription of the payload. 52. The recombinant polynucleotide of any one of embodiments 47-50, wherein the response element confers muscle-specific transcription of the payload. 53. The recombinant polynucleotide of any one of embodiments 47-50, wherein the response element confers kidney-specific transcription of the payload. 54. The recombinant polynucleotide of any one of embodiments 47-53, wherein the payload encodes a protein. 55. The recombinant polynucleotide of embodiment 54, wherein the protein is a neuronal protein, a kidney protein, a retinal protein, a muscle protein, or an apoptosis-inducing protein. 56. The recombinant polynucleotide of embodiment 54 or embodiment 55, wherein the protein is associated with a genetic disorder, a neuronal disorder, a kidney disorder, an eye disorder, a muscular disorder, or a cancer. 57. The recombinant polynucleotide of any one of embodiments 54-56, wherein the protein is progranulin, MeCP2, polycystin-1, or polycystin-2. 58. The recombinant polynucleotide of any one of embodiments 47-53, wherein the payload encodes a therapeutic polynucleotide. 59. The recombinant polynucleotide of embodiment 58, wherein the therapeutic polynucleotide is a guide RNA or a suppressor tRNA. 60. The recombinant polynucleotide of embodiment 58 or embodiment 59, wherein the therapeutic polynucleotide targets a gene. 61. The recombinant polynucleotide of embodiment 60, wherein the gene is associated with a genetic disorder, a neuronal disorder, a kidney disorder, an eye disorder, a muscular disorder, or a cancer. 62. The recombinant polynucleotide of embodiment 60 or embodiment 61, wherein the gene is GRN, MECP2, PKD2, or PKD2. 63. An engineered viral vector comprising the recombinant polynucleotide of any one of embodiments 47-62 in a viral vector. 64. The engineered viral vector of embodiment 63, wherein the viral vector is an adenoviral vector, an adeno-associated viral vector, or a lentivector. 65. The engineered viral vector of embodiment 64, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP. V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, and combinations thereof. 66. A pharmaceutical composition comprising the recombinant polynucleotide of any one of embodiments 47-62 or the viral vector of any one of embodiments 63-65 and a pharmaceutically acceptable carrier. 67. A method of expressing a payload in a target cell of a subject, the method comprising: administering to the subject the recombinant polynucleotide of any one of embodiments 47-62, the viral vector of any one of embodiments 63-65, or the pharmaceutical composition of embodiment 66 to the subject; binding a cognate ligand, a coactivator, or a corepressor to the response element of the recombinant polynucleotide, wherein the cognate ligand or the coactivator is specific to the target cell, or wherein the corepressor is specific to a non-target cell; initiating transcription of the payload by recruiting a polymerase to the core promoter sequence; and transcribing the payload, thereby expressing the payload in the target cell. 68. The method of embodiment 67, comprising expressing the payload at a higher level in the cell type of interest than in the non-target cell. 69. The method of embodiment 67 or embodiment 68, comprising initiating transcription at a higher rate in the cell type of interest than in the non-target cell. 70. The method of any one of embodiments 67-69, wherein the cognate ligand or the coactivator is present at a higher level in the target cell than in the non-target cell. 71. The method of any one of embodiments 67-70, wherein the cognate ligand is a transcription factor. 72. The method of any one of embodiments 67-69, wherein the corepressor is present at a higher level in the non-target cell than in the target cell. 73. The method of any one of embodiments 67-72, wherein the target cell is a target cell state, and wherein the non-target cell is a non-target cell state. 74. The method of embodiment 73, wherein the target cell state is a diseased cell. 75. The method of embodiment 73 or embodiment 74, wherein the non-target cell state is a healthy cell. 76. The method of any one of embodiments 67-71, wherein the target cell is a target cell type, and wherein the non-target cell is a non-target cell type. 77. The method of embodiment 76, wherein the target cell type is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. 78. The method of embodiment 76 or embodiment 77, wherein the non-target cell type is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, a fibroblast, or combinations thereof. 79. A method of treating a disorder in a subject in need thereof, the method comprising: administering to the subject a composition comprising the recombinant polynucleotide of any one of embodiments 47-62, the viral vector of any one of embodiments 63-65, or the pharmaceutical composition of embodiment 66; and expressing a therapeutic sequence encoded by a payload of the recombinant polynucleotide in a target cell of the subject, thereby treating the disorder. 80. The method of embodiment 79, wherein the target cell is cell type associated with the disorder. 81. The method of embodiment 79, wherein the target cell is cell state associated with the disorder. 82. The method of any one of embodiments 79-81, wherein the disorder is a genetic disorder, a neuronal disorder, a kidney disorder, an eye disorder, a muscular disorder, or a cancer. 83. The method of any one of embodiments 79-82, wherein the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease. 84. The method of any one of embodiments 79-83, wherein the disorder is any one of the disorders provided in TABLE 5. 85. The method of any one of embodiments 79-84, wherein the therapeutic sequence encodes a therapeutic protein. 86. The method of embodiment 85, wherein the therapeutic protein is a neuronal protein, a kidney protein, a retinal protein, a muscle protein, or an apoptosis-inducing protein. 87. The method of embodiment 85 or embodiment 86, wherein the therapeutic protein is associated with a genetic disorder, a neuronal disorder, a kidney disorder, an eye disorder, a muscular disorder, or a cancer. 88. The method of any one of embodiments 85-87, wherein the therapeutic protein is MECP2, progranulin, polycystin-1, or polycystin-2. 89. The method of any one of embodiments 85-88, wherein the therapeutic protein is encoded by a gene provided in TABLE 5. 90. The method of any one of embodiments 79-84, wherein the payload encodes a therapeutic polynucleotide. 91. The method of embodiment 90, wherein the therapeutic polynucleotide is a guide RNA or a suppressor tRNA. 92. The method of embodiment 90 or embodiment 91, wherein the therapeutic polynucleotide targets a gene provided in TABLE 5. 93. A method of identifying a switchable core promoter, the method comprising: introducing a core promoter library comprising a first sub-library and a second sub-library to a population of cells; wherein the first sub-library comprises a plurality of core promoter sequences, wherein a core promoter sequence of the plurality of core promoter sequences is linked to a first enhancer sequence, and a unique barcode sequence; wherein the second sub-library comprises the plurality of core promoter sequences, wherein the core promoter sequence of the plurality of promoter sequences is linked to a second enhancer sequence and, a unique barcode sequence; and identifying a switchable core promoter as the core promoter sequence that promotes higher transcription of the unique barcode when paired with the first enhancer sequence than when paired with the second enhancer sequence. 94. The method of embodiment 93, further comprising activating the first enhancer sequence. 95. The method of embodiment 93 or embodiment 94, wherein the second enhancer sequence is not activated. 96. The method of any one of embodiments 93-95, wherein the first enhancer sequence is an active enhancer and the second enhancer sequence is an inactive enhancer. 97. The method of any one of embodiments 93-96, wherein the first enhancer sequence is specific for the population of cells. 98. The method of any one of embodiments 93-97, wherein the population of cells are neurons, kidney cells, liver cells, muscle cells, or cancer cells. 99. The method of any one of embodiments 93-98, wherein the first enhancer sequence is specific for neurons, kidney cells, liver cells, muscle cells, or cancer cells. 100. The method of any one of embodiments 93-99, wherein the second enhancer sequence is specific for neurons, kidney cells, liver cells, muscle cells, or cancer cells. 101. The method of any one of embodiments 93-100, wherein the plurality of core promoter sequences comprises engineered core promoter sequences, synthetic core promoter sequences, wild type core promoter sequences, variant core promoter sequences, or combinations thereof.

### FURTHER NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. An engineered core promoter comprising a TATA box, an initiator element, a spacer separating the TATA box and the initiator element, and one or more features selected from: a) the TATA box comprises a sequence having at least 85% sequence identity to any one of SEQ ID NO: 199-SEQ ID NO: 201 or SEQ ID NO: 213-SEQ ID NO: 215; b) the initiator element comprises a sequence having at least 75% sequence identity to any one of SEQ ID NO: 202-SEQ ID NO: 204, SEQ ID NO: 216, or SEQ ID NO: 217; c) the spacer is a short spacer comprising no less than 25 and no more than 29 nucleotide residues from the TATA box to the initiator element; d) the spacer is a long spacer comprising no less than 31 and no more than 48 nucleotide residues from the TATA box to the initiator element; e) a YY1 motif comprising a sequence having at least 80% sequence identity to any one of SEQ ID NO: 205-SEQ ID NO: 207; f) a transcriptional pause site comprising a sequence having at least 90% sequence identity to any one of SEQ ID NO: 196-SEQ ID NO: 198, SEQ ID NO: 211, or SEQ ID NO: 212; g) at least 70% sequence identity and at least one nucleotide substitution relative to SEQ ID NO: 5, SEQ ID NO: 9, or SEQ ID NO: 210; and h) combinations thereof. 2. The engineered core promoter of embodiment 1, comprising at least 70% sequence identity and nucleotide substitutions T15G and A40C relative to SEQ ID NO: 5. 3. The engineered core promoter of embodiment 1, comprising at least 70% sequence identity and nucleotide substitutions T15A and A40T relative to SEQ ID NO: 5. 4. The engineered core promoter of any one of embodiments 1-3, wherein the spacer comprises from 29 to 31 nucleotides from the TATA box to the initiator element. 5. The engineered core promoter of any one of embodiments 1-4, wherein the spacer comprises 30 nucleotides from the TATA box to the initiator element. 6. The engineered core promoter of any one of embodiments 1-3, wherein the short spacer comprises 28 nucleotide residues from the TATA box to the initiator element. 7. The engineered core promoter of any one of embodiments 1-3, wherein the long spacer comprises 32 nucleotide residues from the TATA box to the initiator element. 8. The engineered core promoter of any one of embodiments 1-7, wherein the TATA box comprises a sequence having at least 90% sequence identity to any one of SEQ ID NO: 199-SEQ ID NO: 201 or SEQ ID NO: 213-SEQ ID NO: 215. 9. The engineered core promoter of any one of embodiments 1-8, wherein the TATA box comprises a sequence of any one of SEQ ID NO: 199-SEQ ID NO: 201 or SEQ ID NO: 213-SEQ ID NO: 215. 10. The engineered core promoter of any one of embodiments 1-9, wherein the initiator element comprises a sequence having at least 85% sequence identity to any one of SEQ ID NO: 202-SEQ ID NO: 204, SEQ ID NO: 216, or SEQ ID NO: 217. 11. The engineered core promoter of any one of embodiments 1-10, wherein the initiator element comprises a sequence of any one of SEQ ID NO: 202-SEQ ID NO: 204, SEQ ID NO: 216, or SEQ ID NO: 217. 12. The engineered core promoter of any one of embodiments 1-11, wherein the YY1 motif comprises a sequence having at least 90% sequence identity to any one of SEQ ID NO: 205-SEQ ID NO: 207. 13. The engineered core promoter of any one of embodiments 1-12, wherein the YY1 motif comprises a sequence of any one of SEQ ID NO: 205-SEQ ID NO: 207. 14. The engineered core promoter of any one of embodiments 1-11, wherein the transcriptional pause site comprises a sequence having at least 95% sequence identity to any one of SEQ ID NO: 196-SEQ ID NO: 198, SEQ ID NO: 211, or SEQ ID NO: 212. 15. The engineered core promoter of any one of embodiments 1-11, wherein the transcriptional pause site comprises a sequence of any one of SEQ ID NO: 196-SEQ ID NO: 198, SEQ ID NO: 211, or SEQ ID NO: 212. 16. The engineered core promoter of any one of embodiments 1-15, wherein the engineered core promoter comprises two or more of the features. 17. The engineered core promoter of any one of embodiments 1-16, wherein the engineered core promoter comprises features a) and b). 18. The engineered core promoter of any one of embodiments 1-17, wherein the engineered core promoter comprises three or more of the features. 19. The engineered core promoter of any one of embodiments 1-18, wherein the engineered core promoter comprises features a) and b) and at least one more of the features. 20. The engineered core promoter of any one of embodiments 1-19, wherein the engineered core promoter comprises features a), b), and c). 21. The engineered core promoter of any one of embodiments 1-19, wherein the engineered core promoter comprises features a), b), and d). 22. The engineered core promoter of any one of embodiments 1-19, wherein the engineered core promoter comprises features a), b), and e). 23. The engineered core promoter of any one of embodiments 1-19, wherein the engineered core promoter comprises features a), b), and f). 24. An engineered core promoter comprising a sequence having at least 80%, at least 85%, at least 90%, at least 93%, at least 95%, at least 97%, at least 99%, or 100% sequence identity to any one of SEQ ID NO: 1-SEQ ID NO: 4, SEQ ID NO: 7, or SEQ ID NO: 10-SEQ ID NO: 144. 25. The engineered core promoter of embodiment 24, wherein the engineered core promoter comprises a sequence having at least 90% sequence identity to any one of SEQ ID NO: 1-SEQ ID NO: 4, SEQ ID NO: 7, or SEQ ID NO: 10-SEQ ID NO: 144. 26. The engineered core promoter of embodiment 23, wherein the engineered core promoter comprises a sequence having at least 80%, at least 85%, at least 90%, at least 93%, at least 95%, at least 97%, at least 99%, or 100% sequence identity to any one of SEQ ID NO: 1-SEQ ID NO: 4, SEQ ID NO: 7, SEQ ID NO: 10-SEQ ID NO: 15, or SEQ ID NO: 110. 27. The engineered core promoter of embodiment 26, wherein the engineered core promoter comprises a sequence having at least 90% sequence identity to any one of SEQ ID NO: 1-SEQ ID NO: 4, SEQ ID NO: 7, SEQ ID NO: 10-SEQ ID NO: 15, or SEQ ID NO: 110. 28. The engineered core promoter of any one of embodiments 23-27, wherein the engineered core promoter comprises a sequence having at least 90% sequence identity to SEQ ID NO: 7. 29. The engineered core promoter of any one of embodiments 23-27, wherein the engineered core promoter comprises a sequence of SEQ ID NO: 7. 30. The engineered core promoter of any one of embodiments 23-27, wherein the engineered core promoter comprises a sequence having at least 90% sequence identity to SEQ ID NO: 1. 31. The engineered core promoter of any one of embodiments 23-27, wherein the engineered core promoter comprises a sequence of SEQ ID NO: 1. 32. The engineered core promoter of any one of embodiments 23-27, wherein the engineered core promoter comprises a sequence having at least 90% sequence identity to SEQ ID NO: 2. 33. The engineered core promoter of any one of embodiments 23-27, wherein the engineered core promoter comprises a sequence of SEQ ID NO: 2. 34. The engineered core promoter of any one of embodiments 23-27, wherein the engineered core promoter comprises a sequence having at least 90% sequence identity to SEQ ID NO: 3. 35. The engineered core promoter of any one of embodiments 23-27, wherein the engineered core promoter comprises a sequence of SEQ ID NO: 3. 36. The engineered core promoter of any one of embodiments 23-27, wherein the engineered core promoter comprises a sequence having at least 90% sequence identity to SEQ ID NO: 4 and including nucleotide substitutions T15G and A40T relative to SEQ ID NO: 5. 37. The engineered core promoter of any one of embodiments 23-27, wherein the engineered core promoter comprises a sequence of SEQ ID NO: 4. 38. The engineered core promoter of any one of embodiments 23-27, wherein the engineered core promoter comprises a sequence having at least 90% sequence identity to SEQ ID NO: 10. 39. The engineered core promoter of any one of embodiments 23-27, wherein the engineered core promoter comprises a sequence of SEQ ID NO: 10. 40. The engineered core promoter of any one of embodiments 23-27, wherein the engineered core promoter comprises a sequence having at least 90% sequence identity to SEQ ID NO: 11. 41. The engineered core promoter of any one of embodiments 23-27, wherein the engineered core promoter comprises a sequence of SEQ ID NO: 11. 42. The engineered core promoter of any one of embodiments 23-27, wherein the engineered core promoter comprises a sequence having at least 90% sequence identity to SEQ ID NO: 12. 43. The engineered core promoter of any one of embodiments 23-27, wherein the engineered core promoter comprises a sequence of SEQ ID NO: 12. 44. The engineered core promoter of any one of embodiments 23-27, wherein the engineered core promoter comprises a sequence having at least 90% sequence identity to SEQ ID NO: 13. 45. The engineered core promoter of any one of embodiments 23-27, wherein the engineered core promoter comprises a sequence of SEQ ID NO: 13. 46. The engineered core promoter of any one of embodiments 23-27, wherein the engineered core promoter comprises a sequence having at least 90% sequence identity to SEQ ID NO: 14. 47. The engineered core promoter of any one of embodiments 23-27, wherein the engineered core promoter comprises a sequence of SEQ ID NO: 14. 48. The engineered core promoter of any one of embodiments 23-27, wherein the engineered core promoter comprises a sequence having at least 90% sequence identity to SEQ ID NO: 15 and including nucleotide substitutions T15G and A40C relative to SEQ ID NO: 5. 49. The engineered core promoter of any one of embodiments 23-27, wherein the engineered core promoter comprises a sequence of SEQ ID NO: 15. 50. The engineered core promoter of any one of embodiments 23-27, wherein the engineered core promoter comprises a sequence having at least 90% sequence identity to SEQ ID NO: 110. 51. The engineered core promoter of any one of embodiments 23-27, wherein the engineered core promoter comprises a sequence of SEQ ID NO: 110. 52. The engineered core promoter of any one of embodiments 1-51, wherein the engineered core promoter comprises a TATA box, an RNA polymerase binding sequence, a B recognition element, a CCAAT box, or a Pribnow box. 53. The engineered core promoter of any one of embodiments 1-52, wherein the engineered core promoter is capable of recruiting a polymerase. 54. The engineered core promoter of embodiment 53, wherein the polymerase is an RNA polymerase II. 55. The engineered core promoter of any one of embodiments 1-54, wherein the engineered core promoter has a dynamic range that is higher than a dynamic range of SEQ ID NO: 6. 56. The engineered core promoter of any one of embodiments 1-55, wherein the engineered core promoter has a dynamic range that is higher than a dynamic range of SEQ ID NO: 9. 57. The engineered core promoter of any one of embodiments 1-56, wherein the engineered core promoter has an activity that is higher than an activity of SEQ ID NO: 6. 58. The engineered core promoter of any one of embodiments 1-57, wherein the engineered core promoter has an activity that is higher than an activity of SEQ ID NO: 9. 59. The engineered core promoter of any one of embodiments 1-58, wherein a transcriptional activity produced by the engineered core promoter paired with an activated response element is higher than a transcriptional activity produced by a core promoter of SEQ ID NO: 6 when paired with the activated response element, a basal transcriptional activity produced by the engineered core promoter paired with an activated response element is lower than a basal transcriptional activity produced by a core promoter of SEQ ID NO: 6 when paired with an inactive response element, or both. 60. The engineered core promoter of any one of embodiments 1-59, wherein a transcriptional activity produced by the engineered core promoter paired with an activated response element is higher than a transcriptional activity produced by a core promoter of SEQ ID NO: 9 when paired with the activated response element, a basal transcriptional activity produced by the engineered core promoter paired with an activated response element is lower than a basal transcriptional activity produced by a core promoter of SEQ ID NO: 9 when paired with an inactive response element, or both. 61. An engineered response element comprising a sequence having at least 80%, at least 85%, at least 90%, at least 93%, at least 95%, at least 97%, at least 99%, or 100% sequence identity to SEQ ID NO: 145 or SEQ ID NO: 146. 62. The engineered response element of embodiment 61, wherein the engineered response element comprises a sequence having at least 90% sequence identity to SEQ ID NO: 145 or SEQ ID NO: 146. 63. The engineered response element of embodiment 61 or embodiment 62, wherein the engineered response element comprises a sequence of SEQ ID NO: 145 or SEQ ID NO: 146. 64. The engineered response element of any one of embodiments 61-63, wherein the engineered response element is capable of binding to a cognate ligand, a coactivator, or a corepressor. 65. The engineered response element of embodiment 64, wherein the engineered response element is an enhancer. 66. The engineered response element of embodiment 64 or embodiment 65, wherein binding of the cognate ligand or the coactivator to the engineered response element increases transcription of a payload under transcriptional control of the engineered response element paired with a core promoter in a target cell type, a target cell state, or a target tissue. 67. The engineered response element of embodiment 64, wherein binding of the corepressor to the engineered response element decreases transcription of a payload under transcriptional control of the engineered response element paired with a core promoter in a non-target cell type, a non-target cell state, or a non-target tissue. 68. The engineered response element of any one of embodiments 61-67, wherein the engineered response element confers bone marrow-specific transcription of a payload under transcriptional control of the engineered response element paired with a core promoter. 69. The engineered response element of embodiment 68, comprising a sequence of SEQ ID NO: 145. 70. The engineered response element of any one of embodiments 61-67, wherein the engineered response element confers liver-specific transcription of a payload under transcriptional control of the engineered response element paired with a core promoter. 71. The engineered response element of embodiment 70, comprising a sequence of SEQ ID NO: 146. 72. An engineered promoter comprising a response element and a core promoter; wherein the response element comprises a sequence having at least 80%, at least 85%, at least 90%, at least 93%, at least 95%, at least 97%, at least 99%, or 100% sequence identity to SEQ ID NO: 145, SEQ ID NO: 146, SEQ ID NO: 193, or SEQ ID NO: 194, and wherein the core promoter comprises a sequence having at least 80%, at least 85%, at least 90%, at least 93%, at least 95%, at least 97%, at least 99%, or 100% sequence identity to of any one of SEQ ID NO: 1-SEQ ID NO: 144 or SEQ ID NO: 210. 73. The engineered promoter of embodiment 72, wherein the core promoter comprises a sequence having at least 90% sequence identity to any one of SEQ ID NO: 1-SEQ ID NO: 144 or SEQ ID NO: 210. 74. The engineered promoter of embodiment 72 or embodiment 73, wherein the core promoter comprises a sequence of SEQ ID NO: 1-SEQ ID NO: 144 or SEQ ID NO: 210. 75. The engineered promoter of any one of embodiments 72-74, wherein the engineered promoter comprises a sequence of any one of SEQ ID NO: 148-SEQ ID NO: 177 or SEQ ID NO: 218-SEQ ID NO: 255. 76. An engineered promoter comprising a response element and the engineered core promoter of any one of embodiments 1-60. 77. The engineered promoter of any one of embodiments 72-76, wherein the response element is capable of binding to a cognate ligand, a coactivator, or a corepressor. 78. The engineered promoter of embodiment 77, wherein the response element is an enhancer. 79. The engineered promoter of embodiment 77 or embodiment 78, wherein binding of the cognate ligand or the coactivator to the response element increases transcription of a payload under transcriptional control of the engineered promoter in a target cell type, a target cell state, or a target tissue. 80. The engineered promoter of embodiment 77, wherein binding of the corepressor to the response element decreases transcription of a payload under transcriptional control of the engineered promoter in a non-target cell type, a non-target cell state, or a non-target tissue. 81. The engineered promoter of any one of embodiments 72-80, wherein the response element confers bone marrow-specific transcription of a payload under transcriptional control of the engineered promoter. 82. The engineered promoter of embodiment 81, wherein the response element comprises a sequence having at least 90% sequence identity to SEQ ID NO: 145 or SEQ ID NO: 193. 83. The engineered promoter of embodiment 81 or embodiment 82, wherein the response element comprises a sequence of SEQ ID NO: 145 or SEQ ID NO: 193. 84. The engineered promoter of any one of embodiments 72-80, wherein the response element confers liver-specific transcription of a payload under transcriptional control of the engineered promoter. 85. The engineered promoter of embodiment 84, wherein the response element comprises a sequence having at least 90% sequence identity to SEQ ID NO: 146 or SEQ ID NO: 194. 86. The engineered promoter of embodiment 84 or embodiment 85, wherein the response element comprises a sequence of SEQ ID NO: 146 or SEQ ID NO: 194. 87. The engineered promoter of any one of embodiments 72-80, wherein the response element confers neuron-specific transcription of a payload under transcriptional control of the engineered promoter. 88. The engineered promoter of any one of embodiments 72-80, wherein the response element confers muscle-specific transcription of a payload under transcriptional control of the engineered promoter. 89. The engineered promoter of any one of embodiments 72-80, wherein the response element confers kidney-specific transcription of a payload under transcriptional control of the engineered promoter. 90. A recombinant polynucleotide comprising the engineered core promoter of any one of embodiments 1-60 and a payload comprising a coding sequence under transcriptional control of the engineered core promoter. 91. A recombinant polynucleotide comprising a response element comprising a sequence having at least 80%, at least 85%, at least 90%, at least 93%, at least 95%, at least 97%, at least 99%, or 100% sequence identity to SEQ ID NO: 145, SEQ ID NO: 146, SEQ ID NO: 193, or SEQ ID NO: 194 and a payload comprising a coding sequence under transcriptional control of the response element. 92. The recombinant polynucleotide of embodiment 91, wherein the response element comprises a sequence having at least 90% sequence identity to SEQ ID NO: 145, SEQ ID NO: 146, SEQ ID NO: 193, or SEQ ID NO: 194. 93. The recombinant polynucleotide of embodiment 91 or embodiment 92, wherein the response element comprises a sequence of SEQ ID NO: 145, SEQ ID NO: 146, SEQ ID NO: 193, or SEQ ID NO: 194. 94. A recombinant polynucleotide comprising a promoter and a payload, wherein the promoter comprises: a response element capable of binding to a cognate ligand, a coactivator, or a corepressor; and the engineered core promoter of any one of embodiments 1-60 capable of recruiting a polymerase; wherein the payload comprises a coding sequence under transcriptional control of the promoter. 95. The recombinant polynucleotide of embodiment 94, wherein the response element is an enhancer. 96. The recombinant polynucleotide of embodiment 94 or embodiment 95, wherein binding of the cognate ligand or the coactivator to the response element increases transcription of the payload in a target cell type, a target cell state, or a target tissue. 97. The recombinant polynucleotide of embodiment 94, wherein binding of the corepressor to the response element decreases transcription of the payload in a non-target cell type, a non-target cell state, or a non-target tissue. 98. The recombinant polynucleotide of any one of embodiments 94-97, wherein the response element confers neuron-specific transcription of the payload. 99. The recombinant polynucleotide of any one of embodiments 94-97, wherein the response element confers bone marrow-specific transcription of the payload. 100. The recombinant polynucleotide of embodiment 99, wherein the response element comprises a sequence having at least 90% sequence identity to SEQ ID NO: 145 or SEQ ID NO: 193. 101. The recombinant polynucleotide of embodiment 99 or embodiment 11, wherein the response element comprises a sequence of SEQ ID NO: 145 or SEQ ID NO: 193. 102. The recombinant polynucleotide of any one of embodiments 94-97, wherein the response element confers liver-specific transcription of the payload. 103. The recombinant polynucleotide of embodiment 102, wherein the response element comprises a sequence having at least 90% sequence identity to SEQ ID NO: 146 or SEQ ID NO: 194. 104. The recombinant polynucleotide of embodiment 102 or embodiment 103, wherein the response element comprises a sequence of SEQ ID NO: 146 or SEQ ID NO: 194. 105. The recombinant polynucleotide of any one of embodiments 94-97, wherein the response element confers muscle-specific transcription of the payload. 106. The recombinant polynucleotide of any one of embodiments 94-97, wherein the response element confers kidney-specific transcription of the payload. 107. A recombinant polynucleotide comprising a promoter and a payload, wherein the promoter comprises: a response element capable of binding to a cognate ligand, a coactivator, or a corepressor, wherein the response element comprises a sequence having at least 90% sequence identity to SEQ ID NO: 145, SEQ ID NO: 146, SEQ ID NO: 193, or SEQ ID NO: 194; and an engineered core promoter capable of recruiting a polymerase; wherein the payload comprises a coding sequence under transcriptional control of the promoter. 108. The recombinant polynucleotide of any one of embodiments 90-107, wherein the payload encodes a protein. 109. The recombinant polynucleotide of embodiment 108, wherein the protein is a neuronal protein, a kidney protein, a retinal protein, a muscle protein, or an apoptosis-inducing protein. 110. The recombinant polynucleotide of embodiment 108 or embodiment 109, wherein the protein is associated with a genetic disorder, a neuronal disorder, a kidney disorder, an eye disorder, a muscular disorder, or a cancer. 111. The recombinant polynucleotide of any one of embodiments 108-110, wherein the protein is progranulin, MeCP2, polycystin-1, or polycystin-2. 112. The recombinant polynucleotide of any one of embodiments 90-107, wherein the payload encodes a therapeutic polynucleotide. 113. The recombinant polynucleotide of embodiment 112, wherein the therapeutic polynucleotide is a guide RNA or a suppressor tRNA. 114. The recombinant polynucleotide of embodiment 112 or embodiment 113, wherein the therapeutic polynucleotide targets a gene. 115. The recombinant polynucleotide of embodiment 114, wherein the gene is associated with a genetic disorder, a neuronal disorder, a kidney disorder, an eye disorder, a muscular disorder, or a cancer. 116. The recombinant polynucleotide of embodiment 114 or embodiment 115, wherein the gene is GRN, MECP2, PKD2, or PKD2. 117. An engineered viral vector comprising the engineered core promoter of any one of embodiments 1-60 in a viral vector. 118. An engineered viral vector comprising the engineered response element of any one of embodiments 61-71 in a viral vector. 119. An engineered viral vector comprising the engineered promoter of any one of embodiments 72-89 in a viral vector. 120. An engineered viral vector comprising the recombinant polynucleotide of any one of embodiments 90-116 in a viral vector. 121. The engineered viral vector of any one of embodiments 117-120, wherein the viral vector is an adenoviral vector, an adeno-associated viral vector, or a lentivector. 122. The engineered viral vector of embodiment 121, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP. V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, and combinations thereof. 123. A cell comprising the engineered core promoter of any one of embodiments 1-60. 124. A cell comprising the engineered response element of any one of embodiments 61-71. 125. A cell comprising the engineered promoter of any one of embodiments 72-89. 126. A cell comprising the recombinant polynucleotide of any one of embodiments 90-116. 127. A cell comprising the engineered viral vector of any one of embodiments 117-122. 128. The cell of any one of embodiments 123-127, wherein the cell is a target cell. 129. The cell of any one of embodiments 123-128, wherein the cell is a diseased cell. 130. The cell of any one of embodiments 123-129, wherein the cell is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. 131. A pharmaceutical composition comprising the engineered core promoter of any one of embodiments 1-60 and a pharmaceutically acceptable carrier. 132. A pharmaceutical composition comprising the engineered response element of any one of embodiments 61-71 and a pharmaceutically acceptable carrier. 133. A pharmaceutical composition comprising the engineered promoter of any one of embodiments 72-89 and a pharmaceutically acceptable carrier. 134. A pharmaceutical composition comprising the recombinant polynucleotide of any one of embodiments 90-116 and a pharmaceutically acceptable carrier. 135. A pharmaceutical composition comprising the engineered viral vector of any one of embodiments 117-122 and a pharmaceutically acceptable carrier. 136. A method of expressing a payload in a target cell of a subject, the method comprising: administering to the subject the recombinant polynucleotide of any one of embodiments 90-116, the viral vector of any one of embodiments 117-122, or the pharmaceutical composition of embodiment 134 or embodiment 135 to the subject; binding a cognate ligand, a coactivator, or a corepressor to the response element of the recombinant polynucleotide, wherein the cognate ligand or the coactivator is specific to the target cell, or wherein the corepressor is specific to a non-target cell; initiating transcription of the payload by recruiting a polymerase to the core promoter; and transcribing the payload, thereby expressing the payload in the target cell. 137. The method of embodiment 136, comprising expressing the payload at a higher level in the target cell than in the non-target cell. 138. The method of embodiment 136 or embodiment 137, comprising initiating transcription at a higher rate in the target cell than in the non-target cell. 139. The method of any one of embodiments 136-138, wherein the cognate ligand or the coactivator is present at a higher level in the target cell than in the non-target cell. 140. The method of any one of embodiments 136-139, wherein the cognate ligand is a transcription factor. 141. The method of any one of embodiments 136-140, wherein the corepressor is present at a higher level in the non-target cell than in the target cell. 142. The method of any one of embodiments 136-141, wherein the target cell is a target cell state, and wherein the non-target cell is a non-target cell state. 143. The method of embodiment 142, wherein the target cell state is a diseased cell. 144. The method of embodiment 142 or embodiment 143, wherein the non-target cell state is a healthy cell. 145. The method of any one of embodiments 136-140, wherein the target cell is a target cell type, and wherein the non-target cell is a non-target cell type. 146. The method of embodiment 145, wherein the target cell type is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. 147. The method of embodiment 145 or embodiment 146, wherein the non-target cell type is a central nervous system cell, a neuron, a renal cell, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, a fibroblast, or combinations thereof. 148. A method of treating a disorder in a subject in need thereof, the method comprising: administering to the subject a composition comprising the recombinant polynucleotide of any one of embodiments 90-116, the viral vector of any one of embodiments 117-122, or the pharmaceutical composition of embodiment 134 or embodiment 135; and expressing a therapeutic sequence encoded by a payload of the recombinant polynucleotide in a target cell of the subject, thereby treating the disorder. 149. The method of embodiment 148, wherein the target cell is a cell type associated with the disorder. 150. The method of embodiment 148, wherein the target cell is a cell state associated with the disorder. 151. The method of any one of embodiments 148-150, wherein the disorder is a genetic disorder, a neuronal disorder, a kidney disorder, an eye disorder, a muscular disorder, or a cancer. 152. The method of any one of embodiments 148-151, wherein the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease. 153. The method of any one of embodiments 148-152, wherein the disorder is any one of the disorders provided in TABLE 5. 154. The method of any one of embodiments 148-153, wherein the therapeutic sequence encodes a therapeutic protein. 155. The method of embodiment 154, wherein the therapeutic protein is a neuronal protein, a kidney protein, a retinal protein, a muscle protein, or an apoptosis-inducing protein. 156. The method of embodiment 154 or embodiment 155, wherein the therapeutic protein is associated with a genetic disorder, a neuronal disorder, a kidney disorder, an eye disorder, a muscular disorder, or a cancer. 157. The method of any one of embodiments 154-156, wherein the therapeutic protein is MECP2, progranulin, polycystin-1, or polycystin-2. 158. The method of any one of embodiments 154-157, wherein the therapeutic protein is encoded by a gene provided in TABLE 5. 159. The method of any one of embodiments 148-158, wherein the payload encodes a therapeutic polynucleotide. 160. The method of embodiment 159, wherein the therapeutic polynucleotide is a guide RNA or a suppressor tRNA. 161. The method of embodiment 159 or embodiment 160, wherein the therapeutic polynucleotide targets a gene provided in TABLE 5. 162. A method of identifying a switchable core promoter, the method comprising: introducing a core promoter library comprising a first sub-library and a second sub-library to a population of cells; wherein the first sub-library comprises a plurality of core promoters, wherein a core promoter of the plurality of core promoters is linked to a first response element, and a unique barcode sequence; wherein the second sub-library comprises the plurality of core promoters, wherein the core promoter of the plurality of promoters is linked to a second response element and, a unique barcode sequence; and identifying a switchable core promoter as the core promoter that promotes higher transcription of the unique barcode when paired with the first response element than when paired with the second response element. 163. The method of embodiment 162, further comprising activating the first response element. 164. The method of embodiment 162 or embodiment 163, wherein the second response element is not activated. 165. The method of any one of embodiments 162-164, wherein the first response element is an activated response element and the second enhancer sequence is an inactive response element or an unactivated response element. 166. The method of any one of embodiments 162-165, wherein the first response element is specific for the population of cells. 167. The method of any one of embodiments 162-166, wherein the population of cells are neurons, kidney cells, liver cells, muscle cells, or cancer cells. 168. The method of any one of embodiments 162-167, wherein the first response element is specific for neurons, kidney cells, liver cells, muscle cells, or cancer cells. 169. The method of any one of embodiments 162-168, wherein the second response element is specific for neurons, kidney cells, liver cells, muscle cells, or cancer cells. 170. The method of any one of embodiments 162-169, wherein the plurality of core promoters comprises engineered core promoters, synthetic core promoters, wild type core promoters, variant core promoters, or combinations thereof. 171. The recombinant polynucleotide of any one of embodiments 90-116, the viral vector of any one of embodiments 117-122, or the pharmaceutical composition of embodiment 134 or embodiment 135; for use in a method of treating a disorder, the method comprising administering to a subject a composition comprising the recombinant polynucleotide, viral vector or pharmaceutical composition and expressing a therapeutic sequence encoded by a payload of the recombinant polynucleotide in a target cell of the subject. 172. The recombinant polynucleotide, viral vector, or pharmaceutical composition for use of embodiment 171, wherein the disorder treated is a genetic disorder, a neuronal disorder, a kidney disorder, an eye disorder, a muscular disorder, or a cancer. 173. The recombinant polynucleotide, viral vector, or pharmaceutical composition for use of any one of embodiments 171-172, wherein the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease. 174. The recombinant polynucleotide, viral vector, or pharmaceutical composition for use of any one of embodiments 171-173, wherein the disorder is any one of the disorders provided in TABLE 5.

### EXAMPLES

The invention is further illustrated by the following non-limiting examples.

### Example 1: Massively Parallel Reporter Assay Screen for Switchable Core Promoters

This example describes a massively parallel reporter assay (MPRA) screen to identify switchable core promoters. A goal of the screen was to identify core promoters that promote high levels of transcription when paired with an activated cell type-specific enhancer sequence but exhibit low basal transcriptional initiation.

A library of 3,649 core promoters was designed, containing variants of existing activatable core promoter sequences as well as fully de novo synthetic core promoter sequences. The variants of existing core promoters were designed by mutagenizing existing synthetic core promoters, including ybTATA (SEQ ID NO: 9) and minP (SEQ ID NO: 5) from pGL4. Synthetic core promoters were developed by randomly generating matches to TATA and the initiator element, Inr across a range of predicted similarity scores. The final library contained 2157 sequence variants and 1484 fully de novo synthetic sequences.

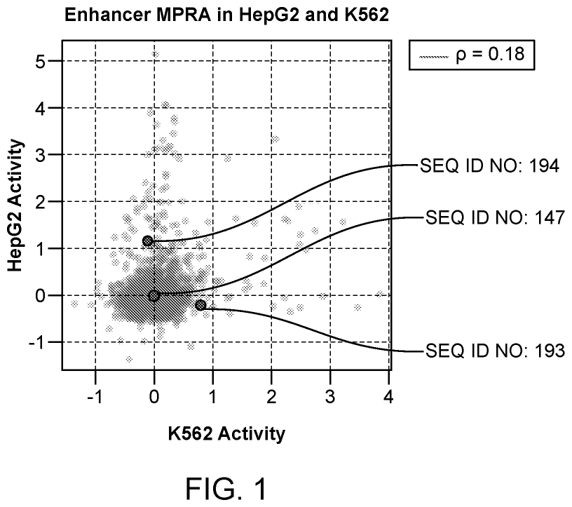

Enhancer sequences were selected to pair with core promoter library members to screen for high levels of cell state-specific transcriptional activation and low levels of basal transcriptional activation in a hepatoblast cell line (HepG2). Enhancers were selected based on cell type-specific activity in HepG2 cells and a myeloid cell line (K562). Literature MPRA results were used to identify endogenous enhancers with cell type-specific activity by comparing activation levels of different enhancers in HepG2 cells (a cancer cell model of hepatocytes) and in K562 cells (a cancer cell model of myeloid cells), as shown in FIG. 1. Enhancers with HepG2-specific activity appear in the upper left region of the plot, and enhancers with K562-specific activity appear in the lower right region of the plot. Enhancers with low activity in both cell types appear in the lower left region of the plot. Three enhancers were selected for use in the core promoter library screen: an endogenous enhancer with HepG2-specific activation (SEQ ID NO: 194), an endogenous enhancer with K562-specific activation (SEQ ID NO: 193), and a negative control enhancer (SEQ ID NO: 147) that exhibited no activation in either HepG2 or K562 cells. Additionally, synthetic enhancer sequences were designed based on sampling motif matches for transcription factors selected for specific expression in primary hepatoblasts or myeloid cells. A synthetic enhancer associated with transcription factors expressed highly in primary fetal hepatoblast cells was selected as a HepG2-specific enhancer (SEQ ID NO: 146), and a synthetic enhancer associated with transcription factors expressed highly in primary fetal liver myeloid cells was selected as a or K562-specific enhancer (SEQ ID NO: 145) for use in the core promoter screen.

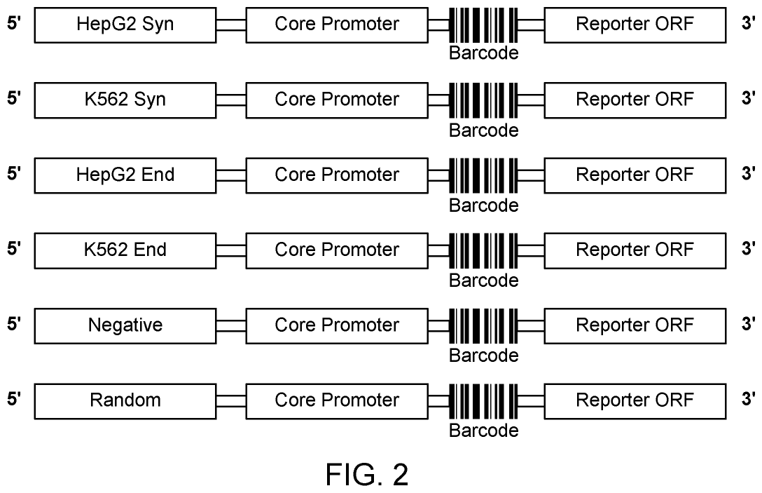

Each member of the core promoter library was cloned into six different constructs containing one of each the five different enhancer sequences identified in the enhancer screens (“HepG2 Syn” (SEQ ID NO: 146), “K562 Syn” (SEQ ID NO: 145), “HepG2 End” (SEQ ID NO: 194), or “K562 End” (SEQ ID NO: 193)), a negative control enhancer (“Negative,” SEQ ID NO: 147) or a random sequence with no enhancer activity (“Random”). The resulting constructs generated for each core promoter library member are illustrated in FIG. 2. In addition to the enhancer and core promoter sequences, each construct included a random barcode sequence appended during PCR prior to cloning for high throughput sequence identification and a reporter open reading frame (“Report ORF”) for expression readout. Random barcodes were associated with enhancer: core promoter constructs through a next generation sequencing run. The barcodes in the 5′ UTR of the reporter identify the enhancer: core promoter driving expression in reporter because the enhancer: core promoter itself is not transcribed. Library complexity was sufficient to represent each of the 21,849 enhancer-core promoter constructs with a median of 20 redundant barcodes. The library members were first cloned with different enhancers and associated with barcodes separately, then the entire pool was subcloned into a pGL4 reporter backbone.

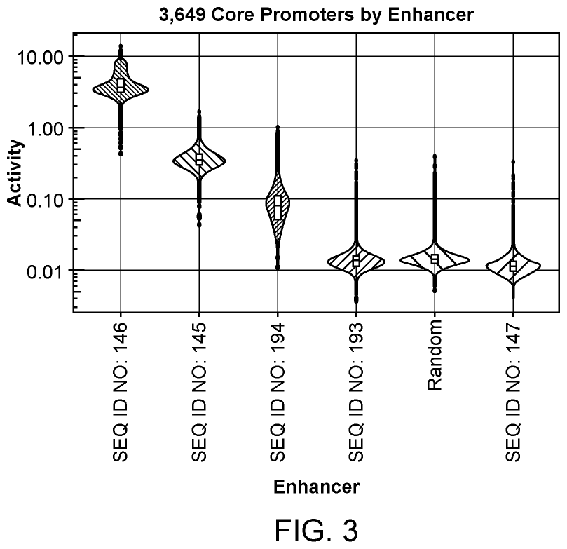

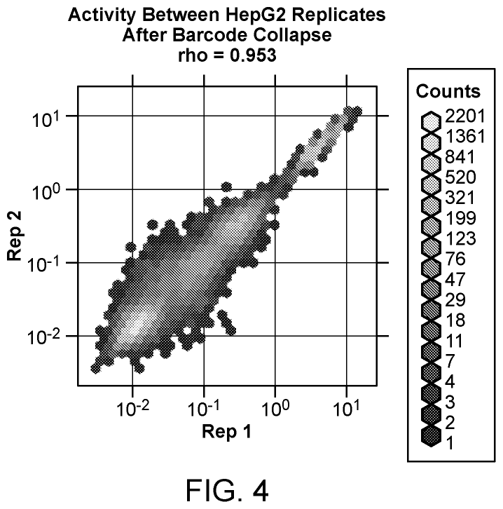

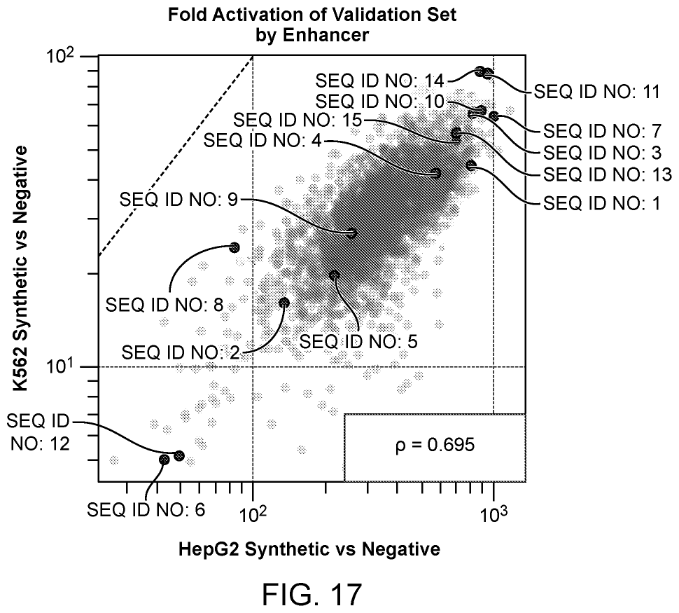

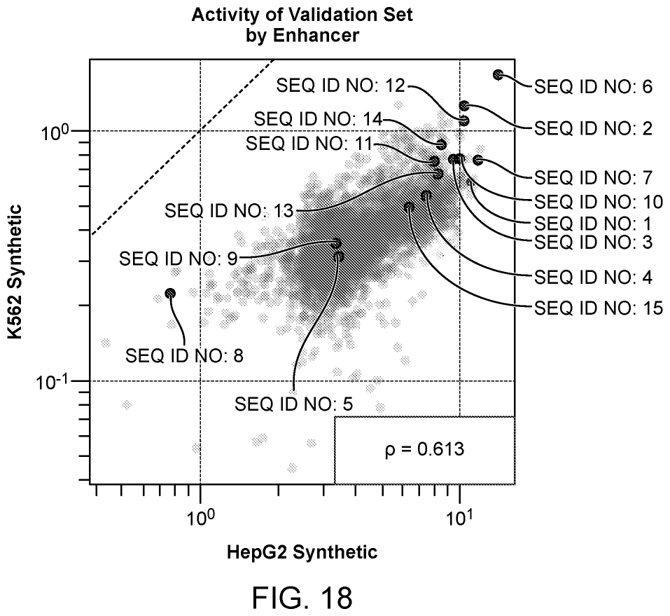

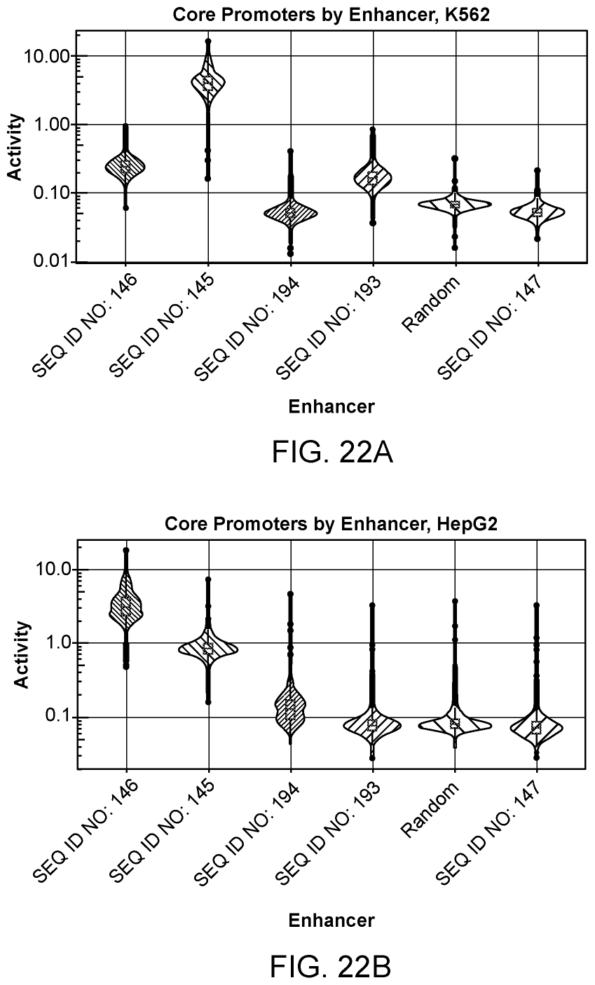

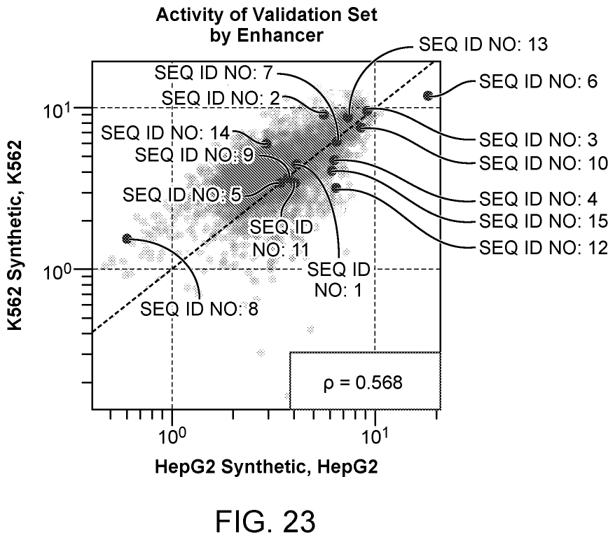

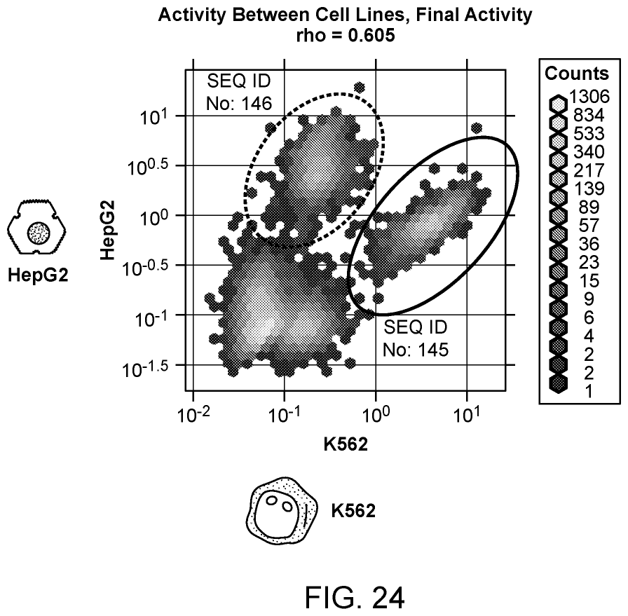

The resulting 21,849 enhancer-promoter constructs were screened in HepG2 and K562 cell lines for cell type-specific transcriptional activation. Transcriptional activity of each enhancer-promoter construct was measured using a massively parallel reporter assay, in HepG2 cells and K562 cells, as shown in FIG. 3 and FIG. 22B (HepG2), and FIG. 22A (K562). All 21,849 constructs were introduced into HepG2 and K562 cell lines via transfection. Transcriptional activity of each construct was represented by the ratio of RNA transcripts per million (tpm)/DNA tpm (“Activity”) in an amplicon next generation sequencing library prepared from RNA and DNA harvested from the transfected cells. Fold activation of each core promoter paired with a synthetic HepG2-specific enhancer of SEQ ID NO: 146 relative to the core promoter paired with a negative control enhancer of SEQ ID NO: 147 (“HepSyn vs Negative”) was compared to fold activation of each core promoter paired with a synthetic K562-specific enhancer of SEQ ID NO: 145 relative to the core promoter paired with no enhancer (“KSyn vs No Enhancer”) as shown in FIG. 17. Transcriptional activity was tested in HepG2 cells for each core promoter paired with a synthetic HepG2-specific enhancer of SEQ ID NO: 146 (“HepG2 Synthetic”) or a synthetic K562-specific enhancer of SEQ ID NO: 145 (“K562 Synthetic”), as shown in FIG. 18. The transcriptional activity in HepG2 cells of each core promoter when paired with a synthetic HepG2-specific enhancer of SEQ ID NO: 146 (“HepG2 Synthetic”) was compared to the transcriptional activity in K562 cells of each core promoter when paired with a synthetic K562-specific enhancer of SEQ ID NO: 145 (“K562 Synthetic”), as shown in FIG. 23. Switching behavior was observed when comparing the entire 21,000 member library's activity between different cell lines, as shown in FIG. 24. The transcriptional activity of each core promoter paired with either a synthetic HepG2-specific enhancer of SEQ ID NO: 146, or a synthetic K562-specific enhancer of SEQ ID NO: 145 was compared between HepG2 and K562 cell lines. As seen in FIG. 24, 21,849 designs within the MPRA were segregate depending on the pairing of the core promoter to either the synthetic HepG2-specific enhancer of SEQ ID NO: 146 specific to HepG2 cells, or the synthetic K562-specific enhancer of SEQ ID NO: 145 specific to K562 cells. The 3,000 core promoters were more active in HepG2 than in K562 when paired with the HepG2 specific enhancer (SEQ ID NO: 146). Similarly, the 3,000 core promoters showed higher activity in K562 when paired with the K562 enhancer (SEQ ID NO: 145). This was shown in FIG. 24 by the cluster of points corresponding to core promoters paired with the HepG2-specific enhancer exhibiting higher activity in HepG2 cells and lower activity in K562 cells, and the cluster of points corresponding to core promoters paired with the K562-specific enhancer exhibiting higher activity in K562 cells and lower activity in HepG2 cells. Core promoters paired with negative control enhancers, such as random enhancer sequences, appeared in clusters in the lower left portion of the plot in FIG. 24, showing that transcriptional activity was low in both cell lines for core promoters paired with inactive enhancers. Overall, the synthetic (SEQ ID NO: 146) or endogenous (SEQ ID NO: 194) HepG2-specific enhancer promoted higher levels of transcription than the corresponding synthetic (SEQ ID NO: 145) or endogenous (SEQ ID NO: 193) K562-specific enhancer, and the synthetic enhancers promoted higher levels of transcription than the endogenous enhancers. Background levels of transcription were observed for both the negative control enhancer and a random sequence with no enhancer activity (“Random”). The MPRA screen was performed in six replicates, and activity levels were well correlated between the first replicate (“Rep 1”) and the second replicate (“Rep 2”), as shown in FIG. 4.

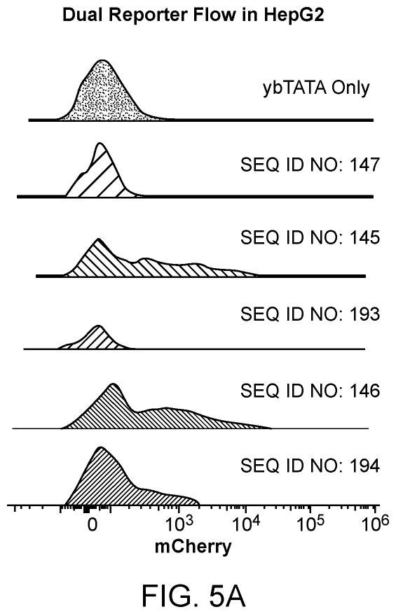

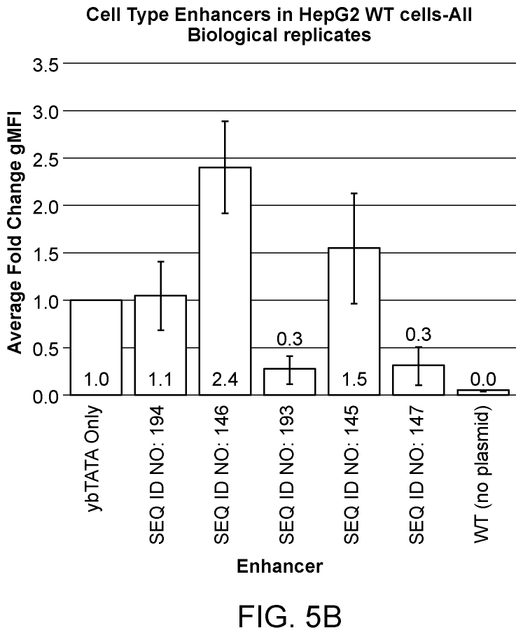

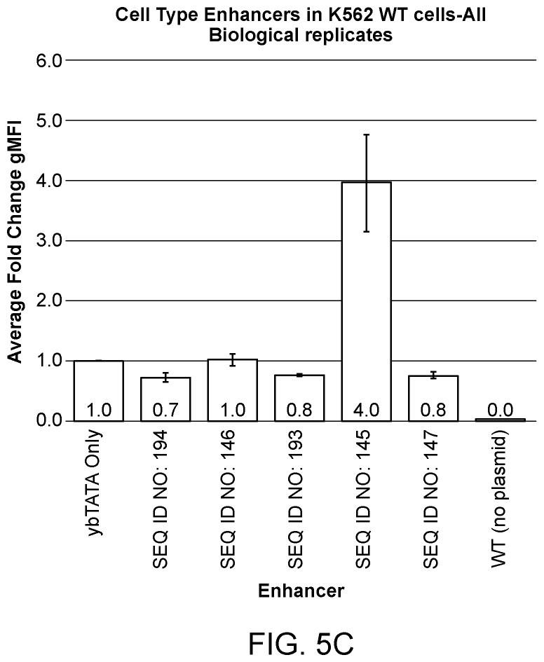

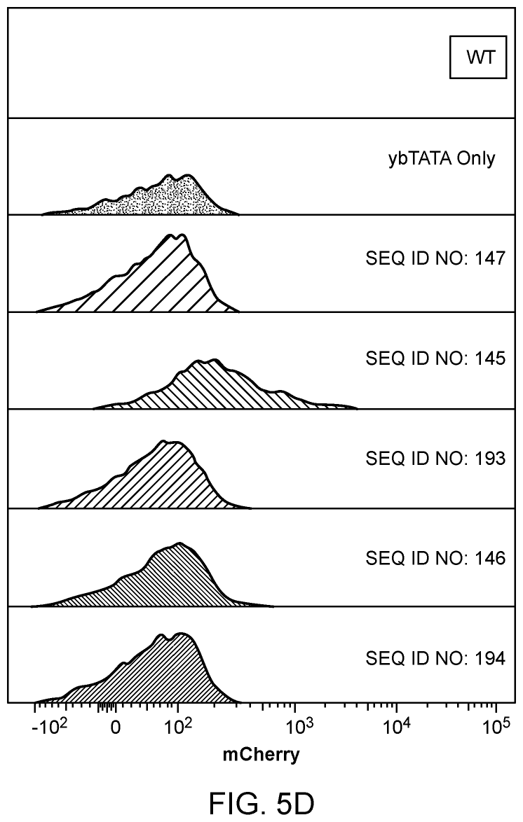

An orthogonal measurement of fluorescent marker expression from a dual reporter construct assessed by flow cytometry in HepG2 cells, as shown in FIG. 5A or in K562 cells, as shown in FIG. 5D, was also performed before the MPRA screen to validate the activity of the selected enhancers. The dual reporter construct comprised the enhancer/core promoter operably linked to a sequence coding for mCherry and a CMV promoter operably linked to a sequence coding for GFP. Transcriptional activity of core promoters was assessed with the ybTATA inserted alone into the dual reporter construct (“ybTATA Only”), or when paired with a negative control enhancer (SEQ ID NO: 147), a synthetic K562-specific enhancer (SEQ ID NO: 145), an endogenous K562-specific enhancer (SEQ ID NO: 193), a synthetic HepG2-specific enhancer (SEQ ID NO: 146), or an endogenous HepG2-specific enhancer (SEQ ID NO: 194). Wild type cells with no plasmid (“WT”) were used as a negative control. Transcriptional activity was measured as a function of fluorescence from mCherry under transcriptional control of the enhancer-promoter constructs. The average fold change in the geometric mean of the fluorescence intensity (gMFI) for each of the enhancers utilized in the dual reporter flow assay is provided in FIG. 5B in HepG2 cells and FIG. 5C in K562 cells. Measurements of enhancer activity agreed well between the MPRA screen and the dual reporter flow assay, but the MPRA was found to be more sensitive to small differences in activity levels.

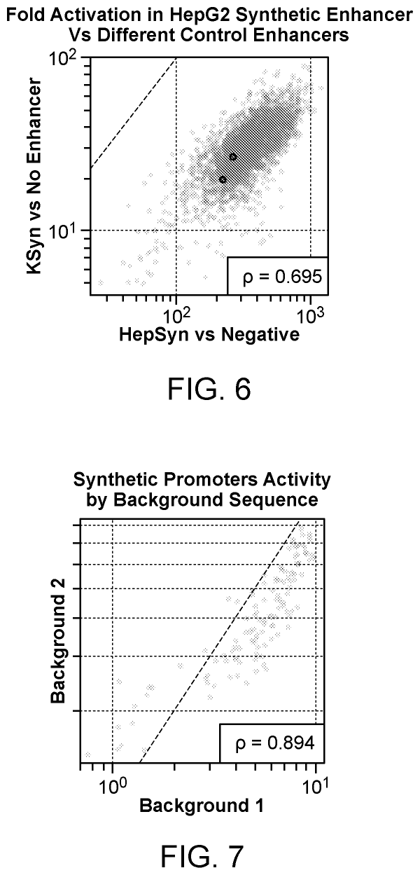

Cell-specific activation of the 3,649 core promoters in HepG2 cells was assessed by comparing transcriptional activity of each core promoter when paired with a HepG2-specific enhancer (HepEndo or HepSyn) or a K562-specific enhancer (KEndo or KSyn) relative to either a negative control enhancer or no enhancer. Fold activation in HepG2 cells with different synthetic enhancers (KSyn or HepSyn) relative to no enhancer or a negative control enhancer is shown in FIG. 6. As seen in FIG. 6, the fold-activation for each core promoter was well correlated between the synthetic K562-specific enhancer and the synthetic HepG2-specific enhancer, but activation was higher overall with the HepG2-specific enhancers.

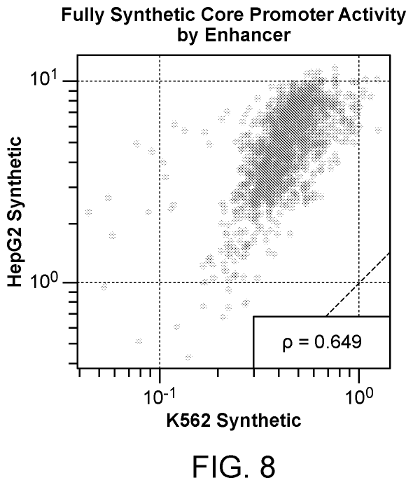

To further validate the MPRA screen, core promoter activity was compared for core promoters with different random sequences inserted into the spacing between the TATA and Inr sequences of the core promoter. FIG. 7 shows that transcriptional activity of the 1484 fully synthetic core promoters from two libraries was similar regardless of which spacer sequence (“Background 1” having a sequence of SEQ ID NO: 208 or “Background 2” having a sequence of SEQ ID NO: 209) was used. These results indicated that the sequence of the spacer between TATA and Inr did not impact core promoter activity, suggesting that any sequence with the appropriate spacing could be used between TATA and Inr. Furthermore, core promoters responded similarly to different active enhancers, as seen by the correlation in FIG. 8 between activity of constructs with the HepG2 synthetic enhancer and constructs with the K562 synthetic enhancer.

Members of the core promoter library were evaluated based on level of activation in HepG2 cells when paired with a HepG2-specific enhancer (core promoter strength) as well as relative activation levels when paired with a HepG2-specific enhancer as compared to when paired with either no enhancer, a negative control enhancer, or a different cell type-specific enhancer (core promoter dynamic range). SEQ ID NO: 1-SEQ ID NO: 3, SEQ ID NO: 7, SEQ ID NO: 10-SEQ ID NO: 14, and SEQ ID NO: 16-SEQ ID NO: 144 were identified as the top core promoter candidates in from the MPRA screen based on strength and dynamic range. Core promoters of SEQ ID NO: 1-SEQ ID NO: 3, SEQ ID NO: 7, and SEQ ID NO: 10-SEQ ID NO: 14 were selected for further evaluation. Switchable core promoter candidates retained the ability to act as a switch with a wide dynamic range across different enhancers and different cell lines. A switchable core promoter when paired with SEQ ID NO: 145, would display low activity in HepG2 cells but high activity in K562 and, when paired with SEQ ID NO: 146, would display high activity in HepG2 but low activity in K562 cells.

### Example 2: Specific Activation of Core Promoters

This example describes specific transcriptional activation by core promoters identified in the MPRA screen described in EXAMPLE 1. Specific activation of core promoters was determined by comparing transcriptional activation of each core promoter when paired with an active, cell-type specific enhancer relative to transcriptional activation when paired with an inactive enhancer (SEQ ID NO: 147; GCCACTGCACTCCGTGCTGGGCAACTGAGTGAGACCCCATCTCAAAAAACTCAAAA AAGAGTTAAAATAAATCACTGTCTATTGTGCCAAAGAGCTGTTGAAGTCATCATTCT TAGAAGAATAGACAGATGGTATTTCAAGGCTT) or a random sequence with no enhancer activity (SEQ ID NO: 195; AAAACGACGGCCAGTGAATTGACGCGTATTGGGATGGAACGGCCTCCACGGCCACT AGT). The active enhancers were selected from a synthetic K562 cell type-specific enhancer (SEQ ID NO: 145; TGCTATTTTTAGCGGGCTTTTTTTGACGGGAGGAAGGAAGGAGGGAGAGGGACGGG AAGTAAAGAGAAAAAGAGGAAGTGAAAGCTAAGAAGGAAGTGACGGCTGGCGGG GACAGATAAGAGTTGTCTAGTTGTGATAATGGAACTGCTGAGTCATGGATTGCAGA GTCAC), a synthetic HepG2 cell-type specific enhancer (SEQ ID NO: 146; GGTTAATGATTAACCGTGTAAATAATTAGCGGATCACGTGATGGTCACGTGTTGGTT CAAGGCCAGAGTCAAGGTCGGGAGTCCAAAGTCCAGAAGTGCAAGGTCCGGTGTTT ACTTTGGGTGTTTACCTTCC), an endogenous K562 cell type-specific enhancer (SEQ ID NO: 193; GGTTAATGATTAACCGTGTAAATAATTAGCGGATCACGTGATGGTCACGTGTTGGTT CAAGGCCAGAGTCAAGGTCGGGAGTCCAAAGTCCAGAAGTGCAAGGTCCGGTGTTT ACTTTGGGTGTTTACCTTCC), or an endogenous HepG2 cell-type specific enhancer (SEQ ID NO: 194; AGAAACACGGCACAGATGACGGCTTTAAAAGAAAAGAGTCCATTGACTGGAAAAG CAAACAGTATGATGGTCAAAGGCCAAGAATCAGAAAACAGAGATTTTTCCTCTTCT CTTGTTTCACAAAGTAAATAAACATGAGACATAT).

Core promoter expression constructs were generated by piecing together an enhancer (e.g., any one of SEQ ID NO: 145-SEQ ID NO: 147, SEQ ID NO: 193, SEQ ID NO: 194, or a random sequence with no enhancer activity) and a core promoter (e.g., any one of SEQ ID NO: 1-SEQ ID NO: 3, SEQ ID NO: 7, SEQ ID NO: 10-SEQ ID NO: 13, and SEQ ID NO: 16-SEQ ID NO: 144), such that the construct sequence included, from 5′ to 3′, the Enhancer followed by the Core Promoter. Transcriptional activation for each of the enhancer/promoter constructs was measured in HepG2 cells, and fold activation was determined by measuring transcriptional activation when paired with an active enhancer divided by transcriptional activation when paired with an inactive enhancer or no enhancer.

Fold activation with select core promoters (SEQ ID NO: 1-SEQ ID NO: 3, SEQ ID NO: 7, SEQ ID NO: 10-SEQ ID NO: 13, and SEQ ID NO: 16-SEQ ID NO: 144) identified from the MPRA screen are provided in TABLE 6.

TABLE 6
Fold Activation of Select Core Promoters Active

| | Fold Activation (Active Enhancer/Inactive Enhancer) | | | | |
|---|---|---|---|---|---|
| | Active Enhancer | | | | |
| | SEQ ID | SEQ ID | SEQ ID | SEQ ID | SEQ ID |
| | NO: 146 | NO: 146 | NO: 194 | NO: 145 | NO: 145 |
| | Inactive Enhancer | | | | |
| Core | SEQ ID | SEQ ID | SEQ ID | SEQ ID | SEQ ID |
| Promoter | NO: 147 | NO: 195 | NO: 147 | NO: 147 | NO: 195 |
| SEQ ID | 533.3 | 423.7 | 12.4 | 34.7 | 27.6 |
| NO: 16 | | | | | |
| SEQ ID | 799.5 | 417.1 | 21.1 | 63.4 | 33.1 |
| NO: 17 | | | | | |
| SEQ ID | 801.9 | 586.5 | 15.8 | 45.0 | 32.9 |
| NO: 1 | | | | | |
| SEQ ID | 643.0 | 524.0 | 14.3 | 42.4 | 34.5 |
| NO: 18 | | | | | |
| SEQ ID | 703.0 | 473.9 | 15.6 | 44.6 | 30.1 |
| NO: 19 | | | | | |
| SEQ ID | 936.2 | 347.0 | 30.9 | 88.7 | 32.9 |
| NO: 11 | | | | | |
| SEQ ID | 725.7 | 589.3 | 12.8 | 40.8 | 33.1 |
| NO: 20 | | | | | |
| SEQ ID | 518.3 | 417.1 | 13.3 | 30.8 | 24.8 |
| NO: 21 | | | | | |
| SEQ ID | 541.7 | 410.2 | 13.0 | 42.6 | 32.3 |
| NO: 22 | | | | | |
| SEQ ID | 696.8 | 425.1 | 17.1 | 51.8 | 31.6 |
| NO: 23 | | | | | |
| SEQ ID | 572.9 | 376.3 | 17.9 | 43.1 | 28.3 |
| NO: 24 | | | | | |
| SEQ ID | 747.2 | 356.2 | 20.6 | 53.6 | 25.6 |
| NO: 25 | | | | | |
| SEQ ID | 538.6 | 344.9 | 44.3 | 40.1 | 25.7 |
| NO: 26 | | | | | |
| SEQ ID | 776.2 | 404.3 | 21.5 | 55.2 | 28.7 |
| NO: 27 | | | | | |
| SEQ ID | 698.4 | 353.2 | 34.2 | 58.4 | 29.5 |
| NO: 28 | | | | | |
| SEQ ID | 592.6 | 319.9 | 20.9 | 44.2 | 23.9 |
| NO: 29 | | | | | |
| SEQ ID | 566.2 | 315.3 | 44.6 | 45.0 | 25.1 |
| NO: 30 | | | | | |
| SEQ ID | 67.9 | 42.5 | 5.3 | 8.0 | 5.0 |
| NO: 31 | | | | | |
| SEQ ID | 80.9 | 41.8 | 3.8 | 8.0 | 4.1 |
| NO: 32 | | | | | |
| SEQ ID | 146.5 | 68.1 | 5.7 | 14.6 | 6.8 |
| NO: 33 | | | | | |
| SEQ ID | 328.9 | 64.8 | 10.3 | 27.6 | 5.4 |
| NO: 34 | | | | | |
| SEQ ID | 206.5 | 167.5 | 6.0 | 15.0 | 12.2 |
| NO: 35 | | | | | |
| SEQ ID | 80.9 | 38.7 | 5.9 | 11.0 | 5.2 |
| NO: 36 | | | | | |
| SEQ ID | 154.6 | 73.8 | 8.5 | 14.9 | 7.1 |
| NO: 37 | | | | | |
| SEQ ID | 263.3 | 51.7 | 16.9 | 33.5 | 6.6 |
| NO: 38 | | | | | |
| SEQ ID | 60.0 | 44.8 | 4.2 | 7.4 | 5.5 |
| NO: 39 | | | | | |
| SEQ ID | 70.2 | 66.6 | 4.6 | 7.7 | 7.3 |
| NO: 40 | | | | | |
| SEQ ID | 137.8 | 59.6 | 5.9 | 12.1 | 5.2 |
| NO: 41 | | | | | |
| SEQ ID | 141.9 | 45.7 | 6.9 | 17.9 | 5.8 |
| NO: 42 | | | | | |
| SEQ ID | 67.7 | 50.8 | 4.2 | 6.7 | 5.0 |
| NO: 43 | | | | | |
| SEQ ID | 381.9 | 181.8 | 13.2 | 27.4 | 13.0 |
| NO: 44 | | | | | |
| SEQ ID | 277.4 | 192.8 | 10.0 | 24.3 | 16.9 |
| NO: 45 | | | | | |
| SEQ ID | 246.3 | 97.2 | 10.5 | 21.9 | 8.7 |
| NO: 46 | | | | | |
| SEQ ID | 116.1 | 66.8 | 5.1 | 11.5 | 6.6 |
| NO: 47 | | | | | |
| SEQ ID | 179.7 | 104.6 | 8.7 | 21.1 | 12.3 |
| NO: 48 | | | | | |
| SEQ ID | 279.6 | 374.2 | 9.7 | 27.7 | 37.1 |
| NO: 49 | | | | | |
| SEQ ID | 49.6 | 35.8 | 3.5 | 5.2 | 3.8 |
| NO: 12 | | | | | |
| SEQ ID | 76.8 | 36.6 | 5.4 | 8.4 | 4.0 |
| NO: 50 | | | | | |
| SEQ ID | 135.0 | 121.8 | 6.4 | 16.2 | 14.6 |
| NO: 2 | | | | | |
| SEQ ID | 443.2 | 212.2 | 13.2 | 27.3 | 13.1 |
| NO: 51 | | | | | |
| SEQ ID | 114.4 | 62.8 | 5.3 | 13.2 | 7.2 |
| NO: 52 | | | | | |
| SEQ ID | 104.5 | 56.6 | 4.5 | 8.1 | 4.4 |
| NO: 53 | | | | | |
| SEQ ID | 136.9 | 113.2 | 6.4 | 12.2 | 10.1 |
| NO: 54 | | | | | |
| SEQ ID | 162.2 | 74.6 | 7.0 | 15.1 | 7.0 |
| NO: 55 | | | | | |
| SEQ ID | 62.8 | 32.2 | 4.9 | 5.6 | 2.9 |
| NO: 56 | | | | | |
| SEQ ID | 52.5 | 52.4 | 4.2 | 7.1 | 7.1 |
| NO: 57 | | | | | |
| SEQ ID | 157.9 | 97.1 | 8.1 | 14.7 | 9.1 |
| NO: 58 | | | | | |
| SEQ ID | 366.9 | 296.4 | 8.6 | 24.7 | 19.9 |
| NO: 59 | | | | | |
| SEQ ID | 152.3 | 49.8 | 8.7 | 19.8 | 6.5 |
| NO: 60 | | | | | |
| SEQ ID | 110.0 | 55.2 | 6.3 | 11.5 | 5.8 |
| NO: 61 | | | | | |
| SEQ ID | 178.8 | 85.5 | 9.1 | 19.9 | 9.5 |
| NO: 62 | | | | | |
| SEQ ID | 606.3 | 422.1 | 17.0 | 47.6 | 33.2 |
| NO: 63 | | | | | |
| SEQ ID | 352.7 | 128.3 | 10.5 | 32.2 | 11.7 |
| NO: 64 | | | | | |
| SEQ ID | 509.0 | 168.0 | 19.2 | 61.6 | 20.3 |
| NO: 65 | | | | | |
| SEQ ID | 140.5 | 78.8 | 5.8 | 15.7 | 8.8 |
| NO: 66 | | | | | |
| SEQ ID | 87.6 | 67.1 | 6.5 | 10.8 | 8.3 |
| NO: 67 | | | | | |
| SEQ ID | 382.2 | 154.7 | 14.1 | 32.3 | 13.1 |
| NO: 68 | | | | | |
| SEQ ID | 583.9 | 367.6 | 11.6 | 37.4 | 23.6 |
| NO: 69 | | | | | |
| SEQ ID | 491.3 | 311.5 | 12.6 | 34.8 | 22.0 |
| NO: 70 | | | | | |
| SEQ ID | 557.1 | 268.5 | 13.7 | 48.4 | 23.3 |
| NO: 71 | | | | | |
| SEQ ID | 553.1 | 410.4 | 12.3 | 35.0 | 26.0 |
| NO: 72 | | | | | |
| SEQ ID | 572.9 | 421.5 | 21.4 | 42.1 | 31.0 |
| NO: 73 | | | | | |
| SEQ ID | 711.1 | 430.6 | 14.1 | 46.6 | 28.2 |
| NO: 74 | | | | | |
| SEQ ID | 490.4 | 180.0 | 12.3 | 35.4 | 13.0 |
| NO: 75 | | | | | |
| SEQ ID | 509.0 | 282.7 | 23.7 | 47.8 | 26.6 |
| NO: 76 | | | | | |
| SEQ ID | 599.2 | 247.6 | 16.8 | 38.8 | 16.0 |
| NO: 77 | | | | | |
| SEQ ID | 472.2 | 367.5 | 9.9 | 30.7 | 23.9 |
| NO: 78 | | | | | |
| SEQ ID | 574.4 | 377.5 | 14.3 | 34.3 | 22.5 |
| NO: 79 | | | | | |
| SEQ ID | 722.3 | 333.0 | 28.1 | 62.2 | 28.7 |
| NO: 80 | | | | | |
| SEQ ID | 421.2 | 240.4 | 11.4 | 32.9 | 18.8 |
| NO: 81 | | | | | |
| SEQ ID | 583.0 | 405.0 | 15.0 | 37.8 | 26.3 |
| NO: 82 | | | | | |
| SEQ ID | 567.4 | 393.6 | 12.7 | 36.0 | 25.0 |
| NO: 83 | | | | | |
| SEQ ID | 395.2 | 297.1 | 14.5 | 31.8 | 23.9 |
| NO: 84 | | | | | |
| SEQ ID | 579.6 | 353.1 | 15.2 | 41.5 | 25.3 |
| NO: 85 | | | | | |
| SEQ ID | 631.7 | 325.4 | 23.5 | 46.3 | 23.8 |
| NO: 86 | | | | | |
| SEQ ID | 476.9 | 332.7 | 13.0 | 38.5 | 26.9 |
| NO: 87 | | | | | |
| SEQ ID | 507.4 | 342.3 | 19.7 | 43.5 | 29.3 |
| NO: 88 | | | | | |
| SEQ ID | 588.1 | 439.0 | 17.8 | 49.3 | 36.8 |
| NO: 89 | | | | | |
| SEQ ID | 408.2 | 463.8 | 9.8 | 30.9 | 35.1 |
| NO: 90 | | | | | |
| SEQ ID | 440.1 | 474.5 | 11.4 | 32.7 | 35.3 |
| NO: 91 | | | | | |
| SEQ ID | 585.7 | 460.5 | 18.7 | 59.6 | 46.8 |
| NO: 92 | | | | | |
| SEQ ID | 523.3 | 316.1 | 19.4 | 48.9 | 29.6 |
| NO: 93 | | | | | |
| SEQ ID | 800.1 | 388.2 | 19.3 | 51.4 | 24.9 |
| NO: 94 | | | | | |
| SEQ ID | 451.6 | 372.4 | 11.1 | 34.2 | 28.2 |
| NO: 95 | | | | | |
| SEQ ID | 637.3 | 267.2 | 28.8 | 50.9 | 21.3 |
| NO: 96 | | | | | |
| SEQ ID | 694.1 | 429.9 | 16.9 | 46.1 | 28.6 |
| NO: 97 | | | | | |
| SEQ ID | 451.7 | 234.1 | 9.3 | 31.2 | 16.2 |
| NO: 98 | | | | | |
| SEQ ID | 322.9 | 269.0 | 7.4 | 20.0 | 16.7 |
| NO: 99 | | | | | |
| SEQ ID | 452.6 | 308.9 | 19.7 | 41. | 28.2 |
| NO: 100 | | | | | |
| SEQ ID | 554.6 | 416.0 | 14.4 | 32.6 | 24.5 |
| NO: 101 | | | | | |
| SEQ ID | 675.4 | 358.6 | 19.0 | 39.8 | 21.1 |
| NO: 102 | | | | | |
| SEQ ID | 580.5 | 280.4 | 27.4 | 46.6 | 22.5 |
| NO: 103 | | | | | |
| SEQ ID | 655.9 | 334.9 | 14.7 | 42.0 | 21.4 |
| NO: 104 | | | | | |
| SEQ ID | 814.5 | 312.8 | 36.8 | 65.4 | 25. |
| NO: 3 | | | | | |
| SEQ ID | 704.0 | 288.2 | 25.9 | 55.7 | 22.8 |
| NO: 105 | | | | | |
| SEQ ID | 626.8 | 381.9 | 21.0 | 48.4 | 29.5 |
| NO: 106 | | | | | |
| SEQ ID | 595.1 | 508.8 | 13.2 | 40.7 | 34.8 |
| NO: 107 | | | | | |
| SEQ ID | 624.7 | 294.9 | 23.6 | 55.1 | 26.0 |
| NO: 108 | | | | | |
| SEQ ID | 774.6 | 586.8 | 17.9 | 46.2 | 35.0 |
| NO: 109 | | | | | |
| SEQ ID | 848.1 | 561.3 | 17.2 | 48.7 | 32.2 |
| NO: 110 | | | | | |
| SEQ ID | 875.9 | 512.9 | 26.6 | 67.1 | 39.3 |
| NO: 10 | | | | | |
| SEQ ID | 505.5 | 378.4 | 11.6 | 33.8 | 25.3 |
| NO: 111 | | | | | |
| SEQ ID | 690.0 | 328.7 | 25.1 | 59.7 | 28.4 |
| NO: 112 | | | | | |
| SEQ ID | 642.4 | 393.7 | 14.9 | 46.4 | 28.4 |
| NO: 113 | | | | | |
| SEQ ID | 570.4 | 473.6 | 17.5 | 48.0 | 39.8 |
| NO: 114 | | | | | |
| SEQ ID | 773.4 | 517.6 | 17.6 | 49.2 | 32.9 |
| NO: 115 | | | | | |
| SEQ ID | 652.0 | 437.6 | 23.7 | 64.5 | 43.3 |
| NO: 116 | | | | | |
| SEQ ID | 915.7 | 462.8 | 27.1 | 70.0 | 35.4 |
| NO: 117 | | | | | |
| SEQ ID | 563.4 | 266.7 | 20.6 | 48.5 | 23.0 |
| NO: 118 | | | | | |
| SEQ ID | 506.8 | 286.2 | 19.4 | 39.3 | 22.2 |
| NO: 119 | | | | | |
| SEQ ID | 652.7 | 380.1 | 17.3 | 50.9 | 29.7 |
| NO: 120 | | | | | |
| SEQ ID | 574.4 | 334.4 | 18.3 | 53.5 | 31.1 |
| NO: 121 | | | | | |
| SEQ ID | 761.3 | 537.0 | 18.9 | 48.6 | 34.3 |
| NO: 122 | | | | | |
| SEQ ID | 805.7 | 432.5 | 19.3 | 50.6 | 27.2 |
| NO: 123 | | | | | |
| SEQ ID | 997.2 | 735.1 | 24.6 | 64.5 | 47.5 |
| NO: 7 | | | | | |
| SEQ ID | 883.1 | 359.0 | 44.5 | 62.8 | 25.5 |
| NO: 124 | | | | | |
| SEQ ID | 615.0 | 388.2 | 25.0 | 54.8 | 34.6 |
| NO: 125 | | | | | |
| SEQ ID | 774.0 | 413.3 | 18.5 | 47.3 | 25.3 |
| NO: 126 | | | | | |
| SEQ ID | 515.2 | 432.3 | 12.4 | 35.0 | 29.3 |
| NO: 127 | | | | | |
| SEQ ID | 615.0 | 367.0 | 19.5 | 49.1 | 29.3 |
| NO: 128 | | | | | |
| SEQ ID | 493.2 | 407.1 | 17.0 | 43.8 | 36.1 |
| NO: 129 | | | | | |
| SEQ ID | 614.5 | 449.4 | 14.9 | 41.3 | 30.2 |
| NO: 130 | | | | | |
| SEQ ID | 484.3 | 369.1 | 17.2 | 41.0 | 31.3 |
| NO: 131 | | | | | |
| SEQ ID | 517.2 | 248.1 | 17.7 | 40.8 | 19.6 |
| NO: 132 | | | | | |
| SEQ ID | 591.4 | 366.9 | 27.7 | 62.7 | 38.9 |
| NO: 133 | | | | | |
| SEQ ID | 623.7 | 363.7 | 14.4 | 41.7 | 24.3 |
| NO: 134 | | | | | |
| SEQ ID | 566.1 | 454.8 | 12.3 | 33.9 | 27.3 |
| NO: 135 | | | | | |
| SEQ ID | 687.1 | 345.8 | 33.7 | 70.5 | 35.5 |
| NO: 136 | | | | | |
| SEQ ID | 541.7 | 313.8 | 14.5 | 33.9 | 19.6 |
| NO: 137 | | | | | |
| SEQ ID | 567.3 | 314.7 | 14.5 | 35.9 | 19.9 |
| NO: 138 | | | | | |
| SEQ ID | 373.2 | 297.7 | 8.3 | 25.8 | 20.5 |
| NO: 139 | | | | | |
| SEQ ID | 500.5 | 278.4 | 22.9 | 48.0 | 26.7 |
| NO: 140 | | | | | |
| SEQ ID | 571.9 | 495.8 | 16.2 | 32.0 | 27.7 |
| NO: 141 | | | | | |
| SEQ ID | 699.3 | 321.7 | 38.1 | 57.3 | 26.4 |
| NO: 13 | | | | | |
| SEQ ID | 516.9 | 366.9 | 23.9 | 40.9 | 29.0 |
| NO: 142 | | | | | |
| SEQ ID | 588.8 | 344.7 | 13.7 | 46.8 | 27.4 |
| NO: 143 | | | | | |
| SEQ ID | 506.6 | 437.1 | 22.4 | 47.3 | 40.8 |
| NO: 144 | | | | | |

As shown in TABLE 6, fold activation of transcription was higher with HepG2 cell type-specific enhancers in HepG2 cells than with K562 cell-type specific enhancers in HepG2 cells, suggesting that the identified core promoters selectively initiated transcription in the presence of enhancers specific to the given cell type while exhibiting low transcriptional activation in the presence of enhancers specific for other cell types.

### Example 3: Further Analysis of Specific Activation by Core Promoters

This example describes further analysis of specific transcriptional activation by core promoters identified using the MPRA screening methods described in EXAMPLE 1.

Specific activation of select core promoters (SEQ ID NO: 1-SEQ ID NO: 15) was determined by comparing transcriptional activation of each core promoter when paired with an active, cell-type specific enhancer relative to transcriptional activation when paired with an inactive enhancer (SEQ ID NO: 147) or no enhancer (SEQ ID NO: 195). The active enhancers were selected from a synthetic K562 cell type-specific enhancer (SEQ ID NO: 145), a synthetic HepG2 cell-type specific enhancer (SEQ ID NO: 146), an endogenous K562 cell type-specific enhancer (SEQ ID NO: 193), or an endogenous HepG2 cell-type specific enhancer (SEQ ID NO: 194). The core promoter of SEQ ID NO: 8 (GGGTACGCCCCTTTTTATGCGCGTGATTACTGCACAGGAATTGG) was included as a negative control, corresponding to a core promoter that exhibited low transcriptional activation.

Core promoter expression constructs were generated by piecing together an enhancer (e.g., any one of SEQ ID NO: 145-SEQ ID NO: 147, SEQ ID NO: 193, SEQ ID NO: 194, or a random sequence with no enhancer activity) and a core promoter (e.g., any one of SEQ ID NO: 1-SEQ ID NO: 15), such that the construct sequence included, from 5′ to 3′, Enhancer followed by the Core Promoter. Select enhancer+core promoter sequences that were assayed are provided in TABLE 7.

TABLE 7
Assayed Enhancer/Promoter Sequences

| Enhancer/ | | | Core |
|---|---|---|---|
| Promoter | Enhancer/Promoter Sequence | Enhancer | Promoter |
| SEQ ID | TGCTATTTTTAGCGGGCTTTTTTTGACGGGAGGAAGGAAGGA | SEQ ID | SEQ ID |
| NO: 148 | GGGAGAGGGACGGGAAGTAAAGAGAAAAAGAGGAAGTGAA | NO: 145 | NO: 1 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGGTATAAAAGCCCCCGGCGCGTGATTACTGTCAG | | |
| | TCTTTTGGAGGGCCTCCCTTTAGCGTGTGAGGTGCTGTAATTC | | |
| | CCCC | | |
| SEQ ID | TGCTATTTTTAGCGGGCTTTTTTTGACGGGAGGAAGGAAGGA | SEQ ID | SEQ ID |
| NO: 149 | GGGAGAGGGACGGGAAGTAAAGAGAAAAAGAGGAAGTGAA | NO: 145 | NO: 2 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGGTATAAAAGGCGGGGGCGCGTGATTACTGTCAG | | |
| | TCTGTTGCAAGATGGCGGTGGTGCGTGGAAGCAGTTATAAAA | | |
| | CGAAC | | |
| SEQ ID | TGCTATTTTTAGCGGGCTTTTTTTGACGGGAGGAAGGAAGGA | SEQ ID | SEQ ID |
| NO: 150 | GGGAGAGGGACGGGAAGTAAAGAGAAAAAGAGGAAGTGAA | NO: 145 | NO: 3 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGTTATATAAGGGCGCCGCGCGGGTGATTACTGTC | | |
| | AGTCTTTTGG | | |
| SEQ ID | TGCTATTTTTAGCGGGCTTTTTTTGACGGGAGGAAGGAAGGA | SEQ ID | SEQ ID |
| NO: 151 | GGGAGAGGGACGGGAAGTAAAGAGAAAAAGAGGAAGTGAA | NO: 145 | NO: 4 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACTAGAGGGTATATAAAGGAAGCTCGACTTCCAGCTTG | | |
| | GCATTCCGG | | |
| SEQ ID | TGCTATTTTTAGCGGGCTTTTTTTGACGGGAGGAAGGAAGGA | SEQ ID | SEQ ID |
| NO: 152 | GGGAGAGGGACGGGAAGTAAAGAGAAAAAGAGGAAGTGAA | NO: 145 | NO: 5 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACTAGAGGGTATATAATGGAAGCTCGACTTCCAGCTTG | | |
| | GCAATCCGG | | |
| SEQ ID | TGCTATTTTTAGCGGGCTTTTTTTGACGGGAGGAAGGAAGGA | SEQ ID | SEQ ID |
| NO: 153 | GGGAGAGGGACGGGAAGTAAAGAGAAAAAGAGGAAGTGAA | NO: 145 | NO: 6 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGTACTTATATAAGGGGGTGGGGGCGCGTTCGTCCTC | | |
| | AGTCGCGATCGAACACTCGAGCCGAGCAGACGTGCCTACGGA | | |
| | CCG | | |
| SEQ ID | TGCTATTTTTAGCGGGCTTTTTTTGACGGGAGGAAGGAAGGA | SEQ ID | SEQ ID |
| NO: 154 | GGGAGAGGGACGGGAAGTAAAGAGAAAAAGAGGAAGTGAA | NO: 145 | NO: 7 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGGTATAAAAGCCCCCGGCGCGATTACTGACATTC | | |
| | TTTTGG | | |
| SEQ ID | TGCTATTTTTAGCGGGCTTTTTTTGACGGGAGGAAGGAAGGA | SEQ ID | SEQ ID |
| NO: 155 | GGGAGAGGGACGGGAAGTAAAGAGAAAAAGAGGAAGTGAA | NO: 145 | NO: 8 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGTACGCCCCTTTTTATGCGCGTGATTACTGCACAG | | |
| | GAATTGG | | |
| SEQ ID | TGCTATTTTTAGCGGGCTTTTTTTGACGGGAGGAAGGAAGGA | SEQ ID | SEQ ID |
| NO: 156 | GGGAGAGGGACGGGAAGTAAAGAGAAAAAGAGGAAGTGAA | NO: 145 | NO: 9 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACTCTAGAGGGTATATAATGGGGGCCACTAGTCTACTA | | |
| | CCAGAAAG | | |
| SEQ ID | TGCTATTTTTAGCGGGCTTTTTTTGACGGGAGGAAGGAAGGA | SEQ ID | SEQ ID |
| NO: 157 | GGGAGAGGGACGGGAAGTAAAGAGAAAAAGAGGAAGTGAA | NO: 145 | NO: 10 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGTTATATAAGGGCGCCTGGAAGGCGATTTCAGTC | | |
| | TTG | | |
| SEQ ID | TGCTATTTTTAGCGGGCTTTTTTTGACGGGAGGAAGGAAGGA | SEQ ID | SEQ ID |
| NO: 158 | GGGAGAGGGACGGGAAGTAAAGAGAAAAAGAGGAAGTGAA | NO: 145 | NO: 11 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGGTATAAAAGCCCCCGGCGCGTGATTACTGTCAG | | |
| | TCTGTTGGGAGAAGCGGGTGAAAGCAGGGCGGGGCTATAAC | | |
| | TCGGCG | | |
| SEQ ID | TGCTATTTTTAGCGGGCTTTTTTTGACGGGAGGAAGGAAGGA | SEQ ID | SEQ ID |
| NO: 159 | GGGAGAGGGACGGGAAGTAAAGAGAAAAAGAGGAAGTGAA | NO: 145 | NO: 12 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGGTATAAAAGGCGGGGGCGCGTGATTACTGTCAG | | |
| | TCTGTTGCAAGATGGCGGCGGTGCGTGGAAGCAGTTATAAAA | | |
| | CGAAC | | |
| SEQ ID | TGCTATTTTTAGCGGGCTTTTTTTGACGGGAGGAAGGAAGGA | SEQ ID | SEQ ID |
| NO: 160 | GGGAGAGGGACGGGAAGTAAAGAGAAAAAGAGGAAGTGAA | NO: 145 | NO: 13 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGTTATATAAGGGCGCCGCGCGTGATTACTGACAT | | |
| | TCTTTTGG | | |
| SEQ ID | TGCTATTTTTAGCGGGCTTTTTTTGACGGGAGGAAGGAAGGA | SEQ ID | SEQ ID |
| NO: 161 | GGGAGAGGGACGGGAAGTAAAGAGAAAAAGAGGAAGTGAA | NO: 145 | NO: 14 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACGGGGTATAAAAGGCGGGGTGGAATTGGCGATTTCAG | | |
| | TCTGGAAGGGGGGTAGGGTGCGTGGAAGCAGTTATAAAACG | | |
| | AAC | | |
| SEQ ID | TGCTATTTTTAGCGGGCTTTTTTTGACGGGAGGAAGGAAGGA | SEQ ID | SEQ ID |
| NO: 162 | GGGAGAGGGACGGGAAGTAAAGAGAAAAAGAGGAAGTGAA | NO: 145 | NO: 15 |
| | AGCTAAGAAGGAAGTGACGGCTGGCGGGGACAGATAAGAGT | | |
| | TGTCTAGTTGTGATAATGGAACTGCTGAGTCATGGATTGCAG | | |
| | AGTCACTAGAGGGTATATAAGGGAAGCTCGACTTCCAGCTTG | | |
| | GCACTCCGG | | |
| SEQ ID | GGTTAATGATTAACCGTGTAAATAATTAGCGGATCACGTGAT | SEQ ID | SEQ ID |
| NO: 163 | GGTCACGTGTTGGTTCAAGGCCAGAGTCAAGGTCGGGAGTCC | NO: 146 | NO: 1 |
| | AAAGTCCAGAAGTGCAAGGTCCGGTGTTTACTTTGGGTGTTT | | |
| | ACCTTCCGGGGTATAAAAGCCCCCGGCGCGTGATTACTGTCA | | |
| | GTCTTTTGGAGGGCCTCCCTTTAGCGTGTGAGGTGCTGTAATT | | |
| | CCCCC | | |
| SEQ ID | GGTTAATGATTAACCGTGTAAATAATTAGCGGATCACGTGAT | SEQ ID | SEQ ID |
| NO: 164 | GGTCACGTGTTGGTTCAAGGCCAGAGTCAAGGTCGGGAGTCC | NO: 146 | NO: 2 |
| | AAAGTCCAGAAGTGCAAGGTCCGGTGTTTACTTTGGGTGTTT | | |
| | ACCTTCCGGGGTATAAAAGGCGGGGGCGCGTGATTACTGTCA | | |
| | GTCTGTTGCAAGATGGCGGTGGTGCGTGGAAGCAGTTATAAA | | |
| | ACGAAC | | |
| SEQ ID | GGTTAATGATTAACCGTGTAAATAATTAGCGGATCACGTGAT | SEQ ID | SEQ ID |
| NO: 165 | GGTCACGTGTTGGTTCAAGGCCAGAGTCAAGGTCGGGAGTCC | NO: 146 | NO: 3 |
| | AAAGTCCAGAAGTGCAAGGTCCGGTGTTTACTTTGGGTGTTT | | |
| | ACCTTCCGGGTTATATAAGGGCGCCGCGCGGGTGATTACTGT | | |
| | CAGTCTTTTGG | | |
| SEQ ID | GGTTAATGATTAACCGTGTAAATAATTAGCGGATCACGTGAT | SEQ ID | SEQ ID |
| NO: 166 | GGTCACGTGTTGGTTCAAGGCCAGAGTCAAGGTCGGGAGTCC | NO: 146 | NO: 4 |
| | AAAGTCCAGAAGTGCAAGGTCCGGTGTTTACTTTGGGTGTTT | | |
| | ACCTTCCTAGAGGGTATATAAAGGAAGCTCGACTTCCAGCTT | | |
| | GGCATTCCGG | | |
| SEQ ID | GGTTAATGATTAACCGTGTAAATAATTAGCGGATCACGTGAT | SEQ ID | SEQ ID |
| NO: 167 | GGTCACGTGTTGGTTCAAGGCCAGAGTCAAGGTCGGGAGTCC | NO: 146 | NO: 5 |
| | AAAGTCCAGAAGTGCAAGGTCCGGTGTTTACTTTGGGTGTTT | | |
| | ACCTTCCTAGAGGGTATATAATGGAAGCTCGACTTCCAGCTT | | |
| | GGCAATCCGG | | |
| SEQ ID | GGTTAATGATTAACCGTGTAAATAATTAGCGGATCACGTGAT | SEQ ID | SEQ ID |
| NO: 168 | GGTCACGTGTTGGTTCAAGGCCAGAGTCAAGGTCGGGAGTCC | NO: 146 | NO: 6 |
| | AAAGTCCAGAAGTGCAAGGTCCGGTGTTTACTTTGGGTGTTT | | |
| | ACCTTCCGTACTTATATAAGGGGGTGGGGGCGCGTTCGTCCT | | |
| | CAGTCGCGATCGAACACTCGAGCCGAGCAGACGTGCCTACGG | | |
| | ACCG | | |
| SEQ ID | GGTTAATGATTAACCGTGTAAATAATTAGCGGATCACGTGAT | SEQ ID | SEQ ID |
| NO: 169 | GGTCACGTGTTGGTTCAAGGCCAGAGTCAAGGTCGGGAGTCC | NO: 146 | NO: 7 |
| | AAAGTCCAGAAGTGCAAGGTCCGGTGTTTACTTTGGGTGTTT | | |
| | ACCTTCCGGGGTATAAAAGCCCCCGGCGCGATTACTGACATT | | |
| | CTTTTGG | | |
| SEQ ID | GGTTAATGATTAACCGTGTAAATAATTAGCGGATCACGTGAT | SEQ ID | SEQ ID |
| NO: 170 | GGTCACGTGTTGGTTCAAGGCCAGAGTCAAGGTCGGGAGTCC | NO: 146 | NO: 8 |
| | AAAGTCCAGAAGTGCAAGGTCCGGTGTTTACTTTGGGTGTTT | | |
| | ACCTTCCGGGTACGCCCCTTTTTATGCGCGTGATTACTGCACA | | |
| | GGAATTGG | | |
| SEQ ID | GGTTAATGATTAACCGTGTAAATAATTAGCGGATCACGTGAT | SEQ ID | SEQ ID |
| NO: 171 | GGTCACGTGTTGGTTCAAGGCCAGAGTCAAGGTCGGGAGTCC | NO: 146 | NO: 9 |
| | AAAGTCCAGAAGTGCAAGGTCCGGTGTTTACTTTGGGTGTTT | | |
| | ACCTTCCTCTAGAGGGTATATAATGGGGGCCACTAGTCTACT | | |
| | ACCAGAAAG | | |
| SEQ ID | GGTTAATGATTAACCGTGTAAATAATTAGCGGATCACGTGAT | SEQ ID | SEQ ID |
| NO: 172 | GGTCACGTGTTGGTTCAAGGCCAGAGTCAAGGTCGGGAGTCC | NO: 146 | NO: 10 |
| | AAAGTCCAGAAGTGCAAGGTCCGGTGTTTACTTTGGGTGTTT | | |
| | ACCTTCCGGGTTATATAAGGGCGCCTGGAAGGCGATTTCAGT | | |
| | CTTG | | |
| SEQ ID | GGTTAATGATTAACCGTGTAAATAATTAGCGGATCACGTGAT | SEQ ID | SEQ ID |
| NO: 173 | GGTCACGTGTTGGTTCAAGGCCAGAGTCAAGGTCGGGAGTCC | NO: 146 | NO: 11 |
| | AAAGTCCAGAAGTGCAAGGTCCGGTGTTTACTTTGGGTGTTT | | |
| | ACCTTCCGGGGTATAAAAGCCCCCGGCGCGTGATTACTGTCA | | |
| | GTCTGTTGGGAGAAGCGGGTGAAAGCAGGGCGGGGCTATAA | | |
| | CTCGGCG | | |
| SEQ ID | GGTTAATGATTAACCGTGTAAATAATTAGCGGATCACGTGAT | SEQ ID | SEQ ID |
| NO: 174 | GGTCACGTGTTGGTTCAAGGCCAGAGTCAAGGTCGGGAGTCC | NO: 146 | NO: 12 |
| | AAAGTCCAGAAGTGCAAGGTCCGGTGTTTACTTTGGGTGTTT | | |
| | ACCTTCCGGGGTATAAAAGGCGGGGGCGCGTGATTACTGTCA | | |
| | GTCTGTTGCAAGATGGCGGCGGTGCGTGGAAGCAGTTATAAA | | |
| | ACGAAC | | |
| SEQ ID | GGTTAATGATTAACCGTGTAAATAATTAGCGGATCACGTGAT | SEQ ID | SEQ ID |
| NO: 175 | GGTCACGTGTTGGTTCAAGGCCAGAGTCAAGGTCGGGAGTCC | NO: 146 | NO: 13 |
| | AAAGTCCAGAAGTGCAAGGTCCGGTGTTTACTTTGGGTGTTT | | |
| | ACCTTCCGGGTTATATAAGGGCGCCGCGCGTGATTACTGACA | | |
| | TTCTTTTGG | | |
| SEQ ID | GGTTAATGATTAACCGTGTAAATAATTAGCGGATCACGTGAT | SEQ ID | SEQ ID |
| NO: 176 | GGTCACGTGTTGGTTCAAGGCCAGAGTCAAGGTCGGGAGTCC | NO: 146 | NO: 14 |
| | AAAGTCCAGAAGTGCAAGGTCCGGTGTTTACTTTGGGTGTTT | | |
| | ACCTTCCGGGGTATAAAAGGCGGGGTGGAATTGGCGATTTCA | | |
| | GTCTGGAAGGGGGGTAGGGTGCGTGGAAGCAGTTATAAAAC | | |
| | GAAC | | |
| SEQ ID | GGTTAATGATTAACCGTGTAAATAATTAGCGGATCACGTGAT | SEQ ID | SEQ ID |
| NO: 177 | GGTCACGTGTTGGTTCAAGGCCAGAGTCAAGGTCGGGAGTCC | NO: 146 | NO: 15 |
| | AAAGTCCAGAAGTGCAAGGTCCGGTGTTTACTTTGGGTGTTT | | |
| | ACCTTCCTAGAGGGTATATAAGGGAAGCTCGACTTCCAGCTT | | |
| | GGCACTCCGG | | |
| SEQ ID | GCCACTGCACTCCGTGCTGGGCAACTGAGTGAGACCCCATCT | SEQ ID | SEQ ID |
| NO: 178 | CAAAAAACTCAAAAAAGAGTTAAAATAAATCACTGTCTATTG | NO: 147 | NO: 1 |
| | TGCCAAAGAGCTGTTGAAGTCATCATTCTTAGAAGAATAGAC | | |
| | AGATGGTATTTCAAGGCTTGGGGTATAAAAGCCCCCGGCGCG | | |
| | TGATTACTGTCAGTCTTTTGGAGGGCCTCCCTTTAGCGTGTGA | | |
| | GGTGCTGTAATTCCCCC | | |
| SEQ ID | GCCACTGCACTCCGTGCTGGGCAACTGAGTGAGACCCCATCT | SEQ ID | SEQ ID |
| NO: 179 | CAAAAAACTCAAAAAAGAGTTAAAATAAATCACTGTCTATTG | NO: 147 | NO: 2 |
| | TGCCAAAGAGCTGTTGAAGTCATCATTCTTAGAAGAATAGAC | | |
| | AGATGGTATTTCAAGGCTTGGGGTATAAAAGGCGGGGGCGCG | | |
| | TGATTACTGTCAGTCTGTTGCAAGATGGCGGTGGTGCGTGGA | | |
| | AGCAGTTATAAAACGAAC | | |
| SEQ ID | GCCACTGCACTCCGTGCTGGGCAACTGAGTGAGACCCCATCT | SEQ ID | SEQ ID |
| NO: 180 | CAAAAAACTCAAAAAAGAGTTAAAATAAATCACTGTCTATTG | NO: 147 | NO: 3 |
| | TGCCAAAGAGCTGTTGAAGTCATCATTCTTAGAAGAATAGAC | | |
| | AGATGGTATTTCAAGGCTTGGGTTATATAAGGGCGCCGCGCG | | |
| | GGTGATTACTGTCAGTCTTTTGG | | |
| SEQ ID | GCCACTGCACTCCGTGCTGGGCAACTGAGTGAGACCCCATCT | SEQ ID | SEQ ID |
| NO: 181 | CAAAAAACTCAAAAAAGAGTTAAAATAAATCACTGTCTATTG | NO: 147 | NO: 4 |
| | TGCCAAAGAGCTGTTGAAGTCATCATTCTTAGAAGAATAGAC | | |
| | AGATGGTATTTCAAGGCTTTAGAGGGTATATAAAGGAAGCTC | | |
| | GACTTCCAGCTTGGCATTCCGG | | |
| SEQ ID | GCCACTGCACTCCGTGCTGGGCAACTGAGTGAGACCCCATCT | SEQ ID | SEQ ID |
| NO: 182 | CAAAAAACTCAAAAAAGAGTTAAAATAAATCACTGTCTATTG | NO: 147 | NO: 5 |
| | TGCCAAAGAGCTGTTGAAGTCATCATTCTTAGAAGAATAGAC | | |
| | AGATGGTATTTCAAGGCTTTAGAGGGTATATAATGGAAGCTC | | |
| | GACTTCCAGCTTGGCAATCCGG | | |
| SEQ ID | GCCACTGCACTCCGTGCTGGGCAACTGAGTGAGACCCCATCT | SEQ ID | SEQ ID |
| NO: 183 | CAAAAAACTCAAAAAAGAGTTAAAATAAATCACTGTCTATTG | NO: 147 | NO: 6 |
| | TGCCAAAGAGCTGTTGAAGTCATCATTCTTAGAAGAATAGAC | | |
| | AGATGGTATTTCAAGGCTTGTACTTATATAAGGGGGTGGGGG | | |
| | CGCGTTCGTCCTCAGTCGCGATCGAACACTCGAGCCGAGCAG | | |
| | ACGTGCCTACGGACCG | | |
| SEQ ID | GCCACTGCACTCCGTGCTGGGCAACTGAGTGAGACCCCATCT | SEQ ID | SEQ ID |
| NO: 184 | CAAAAAACTCAAAAAAGAGTTAAAATAAATCACTGTCTATTG | NO: 147 | NO: 7 |
| | TGCCAAAGAGCTGTTGAAGTCATCATTCTTAGAAGAATAGAC | | |
| | AGATGGTATTTCAAGGCTTGGGGTATAAAAGCCCCCGGCGCG | | |
| | ATTACTGACATTCTTTTGG | | |
| SEQ ID | GCCACTGCACTCCGTGCTGGGCAACTGAGTGAGACCCCATCT | SEQ ID | SEQ ID |
| NO: 185 | CAAAAAACTCAAAAAAGAGTTAAAATAAATCACTGTCTATTG | NO: 147 | NO: 8 |
| | TGCCAAAGAGCTGTTGAAGTCATCATTCTTAGAAGAATAGAC | | |
| | AGATGGTATTTCAAGGCTTGGGTACGCCCCTTTTTATGCGCGT | | |
| | GATTACTGCACAGGAATTGG | | |
| SEQ ID | GCCACTGCACTCCGTGCTGGGCAACTGAGTGAGACCCCATCT | SEQ ID | SEQ ID |
| NO: 186 | CAAAAAACTCAAAAAAGAGTTAAAATAAATCACTGTCTATTG | NO: 147 | NO: 9 |
| | TGCCAAAGAGCTGTTGAAGTCATCATTCTTAGAAGAATAGAC | | |
| | AGATGGTATTTCAAGGCTTTCTAGAGGGTATATAATGGGGGC | | |
| | CACTAGTCTACTACCAGAAAG | | |
| SEQ ID | GCCACTGCACTCCGTGCTGGGCAACTGAGTGAGACCCCATCT | SEQ ID | SEQ ID |
| NO: 187 | CAAAAAACTCAAAAAAGAGTTAAAATAAATCACTGTCTATTG | NO: 147 | NO: 10 |
| | TGCCAAAGAGCTGTTGAAGTCATCATTCTTAGAAGAATAGAC | | |
| | AGATGGTATTTCAAGGCTTGGGTTATATAAGGGCGCCTGGAA | | |
| | GGCGATTTCAGTCTTG | | |
| SEQ ID | GCCACTGCACTCCGTGCTGGGCAACTGAGTGAGACCCCATCT | SEQ ID | SEQ ID |
| NO: 188 | CAAAAAACTCAAAAAAGAGTTAAAATAAATCACTGTCTATTG | NO: 147 | NO: 11 |
| | TGCCAAAGAGCTGTTGAAGTCATCATTCTTAGAAGAATAGAC | | |
| | AGATGGTATTTCAAGGCTTGGGGTATAAAAGCCCCCGGCGCG | | |
| | TGATTACTGTCAGTCTGTTGGGAGAAGCGGGTGAAAGCAGGG | | |
| | CGGGGCTATAACTCGGCG | | |
| SEQ ID | GCCACTGCACTCCGTGCTGGGCAACTGAGTGAGACCCCATCT | SEQ ID | SEQ ID |
| NO: 189 | CAAAAAACTCAAAAAAGAGTTAAAATAAATCACTGTCTATTG | NO: 147 | NO: 12 |
| | TGCCAAAGAGCTGTTGAAGTCATCATTCTTAGAAGAATAGAC | | |
| | AGATGGTATTTCAAGGCTTGGGGTATAAAAGGCGGGGGCGCG | | |
| | TGATTACTGTCAGTCTGTTGCAAGATGGCGGCGGTGCGTGGA | | |
| | AGCAGTTATAAAACGAAC | | |
| SEQ ID | GCCACTGCACTCCGTGCTGGGCAACTGAGTGAGACCCCATCT | SEQ ID | SEQ ID |
| NO: 190 | CAAAAAACTCAAAAAAGAGTTAAAATAAATCACTGTCTATTG | NO: 147 | NO: 13 |
| | TGCCAAAGAGCTGTTGAAGTCATCATTCTTAGAAGAATAGAC | | |
| | AGATGGTATTTCAAGGCTTGGGTTATATAAGGGCGCCGCGCG | | |
| | TGATTACTGACATTCTTTTGG | | |
| SEQ ID | GCCACTGCACTCCGTGCTGGGCAACTGAGTGAGACCCCATCT | SEQ ID | SEQ ID |
| NO: 191 | CAAAAAACTCAAAAAAGAGTTAAAATAAATCACTGTCTATTG | NO: 147 | NO: 14 |
| | TGCCAAAGAGCTGTTGAAGTCATCATTCTTAGAAGAATAGAC | | |
| | AGATGGTATTTCAAGGCTTGGGGTATAAAAGGCGGGGTGGAA | | |
| | TTGGCGATTTCAGTCTGGAAGGGGGGTAGGGTGCGTGGAAGC | | |
| | AGTTATAAAACGAAC | | |
| SEQ ID | GCCACTGCACTCCGTGCTGGGCAACTGAGTGAGACCCCATCT | SEQ ID | SEQ ID |
| NO: 192 | CAAAAAACTCAAAAAAGAGTTAAAATAAATCACTGTCTATTG | NO: 147 | NO: 15 |
| | TGCCAAAGAGCTGTTGAAGTCATCATTCTTAGAAGAATAGAC | | |
| | AGATGGTATTTCAAGGCTTTAGAGGGTATATAAGGGAAGCTC | | |
| | GACTTCCAGCTTGGCACTCCGG | | |

Transcriptional activation for each of the enhancer/promoter constructs was measured in HepG2 cells, and fold activation was determined by measuring transcriptional activation when paired with an active enhancer divided by transcriptional activation when paired with an inactive enhancer or a random sequence with no enhancer activity (no enhancer).

Fold activation with select core promoters (SEQ ID NO: 1-SEQ ID NO: 15) is provided in TABLE 8.

TABLE 8
Fold Activation of Select Core Promoters

| | Fold Activation (Active Enhancer/Inactive Enhancer) | | | | |
|---|---|---|---|---|---|
| | Active Enhancer | | | | |
| | SEQ ID | SEQ ID | SEQ ID | SEQ ID | SEQ ID |
| | NO: 146 | NO: 146 | NO: 194 | NO: 145 | NO: 145 |
| | Inactive Enhancer | | | | |
| Core | SEQ ID | SEQ ID | SEQ ID | SEQ ID | SEQ ID |
| Promoter | NO: 147 | NO: 195 | NO: 147 | NO: 147 | NO: 195 |
| SEQ ID | 801.9 | 586.5 | 15.8 | 45.0 | 32.9 |
| NO: 1 | | | | | |
| SEQ ID | 135.0 | 121.8 | 6.4 | 16.2 | 14.6 |
| NO: 2 | | | | | |
| SEQ ID | 814.5 | 312.8 | 36.8 | 65.4 | 25.1 |
| NO: 3 | | | | | |
| SEQ ID | 576.6 | 414.6 | 13.7 | 42.1 | 30.3 |
| NO: 4 | | | | | |
| SEQ ID | 217.3 | 223.1 | 4.5 | 19.8 | 20.3 |
| NO: 5 | | | | | |
| SEQ ID | 42.9 | 36.3 | 3.1 | 5.1 | 4.3 |
| NO: 6 | | | | | |
| SEQ ID | 997.2 | 735.1 | 24.6 | 64.5 | 47.5 |
| NO: 7 | | | | | |
| SEQ ID | 84.0 | 59.9 | 3.2 | 24.3 | 17.4 |
| NO: 8 | | | | | |
| SEQ ID | 257.0 | 242.7 | 2.8 | 27.0 | 25.5 |
| NO: 9 | | | | | |
| SEQ ID | 875.9 | 512.9 | 26.6 | 67.1 | 39.3 |
| NO: 10 | | | | | |
| SEQ ID | 936.2 | 347.0 | 30.9 | 88.7 | 32.9 |
| NO: 11 | | | | | |
| SEQ ID | 49.6 | 35.8 | 3.5 | 5.2 | 3.8 |
| NO: 12 | | | | | |
| SEQ ID | 699.3 | 321.7 | 38.1 | 57.3 | 26.4 |
| NO: 13 | | | | | |
| SEQ ID | 869.1 | 614.6 | 10.6 | 89.8 | 63.5 |
| NO: 14 | | | | | |
| SEQ ID | 699.0 | 379.6 | 11.8 | 53.8 | 29.2 |
| NO: 15 | | | | | |

As shown in TABLE 8, fold activation of transcription was higher with HepG2 cell type-specific enhancers in HepG2 cells than with K562 cell-type specific enhancers in HepG2 cells, suggesting that the identified core promoters selectively initiated transcription in the presence of enhancers specific to the given cell type while exhibiting low transcriptional activation in the presence of enhancers specific for other cell types.

### Example 4: Effect of Spacing Between TATA and Inr on Transcriptional Activation

This example describes the effect of spacing between TATA and Inr in the core promoter on transcriptional activation. Transcriptional activation of core promoters with a short spacing, corresponding to a deletion of two bases in between TATA and Inr, or a longer spacing was compared, corresponding a standard 30 nucleotide spacing from TATA to Inr wherein the number of nucleotide residues from the TATA box to the initiator element are counted from the first T in the TATA box to the A in the CAG in the initiator element (or the corresponding CAG in the parent sequence if the initiator element is mutated), inclusive. Examples of core promoters with the short, 28-nucleotide spacing include SEQ ID NO: 7, SEQ ID NO: 10, and SEQ ID NO: 109-SEQ ID NO: 125. Examples of core promoters with standard, 30-nucleotide spacing include SEQ ID NO: 13 and SEQ ID NO: 126-SEQ ID NO: 144.

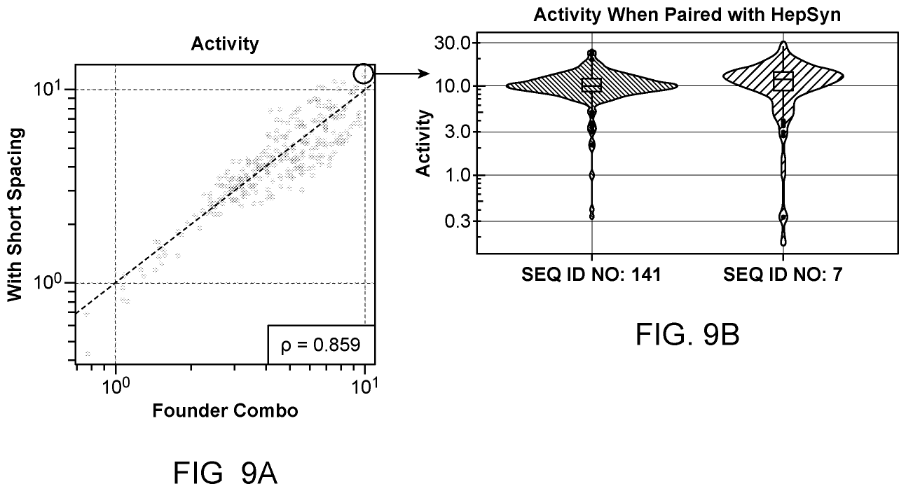

The activity of core promoters with a short spacing, having a length of 28 nucleotides, or a standard spacing, having a length of 30 nucleotides, was compared when paired with a synthetic HepG2 cell type-specific enhancer (SEQ ID NO: 146) in HepG2 cells, as shown in FIG. 9A. While there was no single spacing that increased transcriptional activation for all core promoters, the shorter spacing increased transcriptional activation of SEQ ID NO: 7, which included the 28-nucleotide spacing, relative to SEQ ID NO: 141, which included the standard 30-nucleotide spacing, when paired with the enhancer of SEQ ID NO: 146 as shown in FIG. 9B. SEQ ID NO: 7 exhibited higher transcriptional activation than SEQ ID NO: 141 across most redundant barcodes, indicating that the shorter spacing increased transcriptional activation for this core promoter.

### Example 5: Effect of Pause Site on Transcriptional Activation

This example describes the effect of a pause site in the core promoter on transcriptional activation. Transcriptional activity and fold activation of core promoters with and without a transcriptional pause site was compared. Ten different transcriptional pause sites were tested in the context of 20 different core promoters. Examples of core promoter sequences with a pause site include SEQ ID NO: 1, SEQ ID NO: 11, and SEQ ID NO: 16-SEQ ID NO: 21. Examples of pause site sequences include SEQ ID NO: 196-SEQ ID NO: 198, SEQ ID NO: 211, or SEQ ID NO: 212. Examples of core promoter sequences without the pause site include SEQ ID NO: 13 and SEQ ID NO: 126-SEQ ID NO: 144.

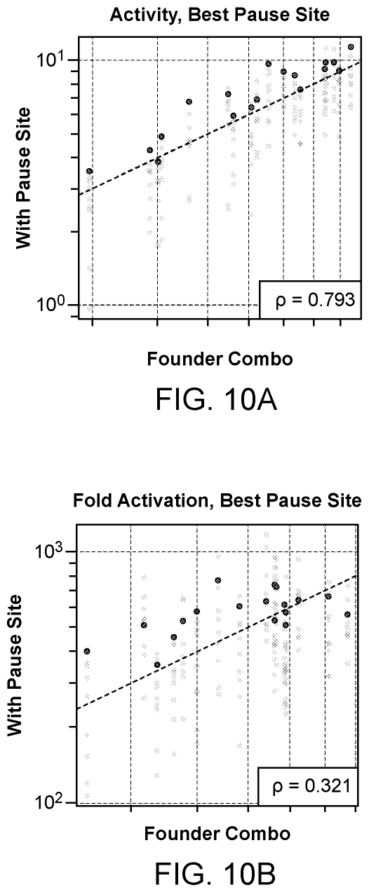

FIG. 10A and FIG. 10B show comparisons of the activity (FIG. 10A) and fold activation (FIG. 10B) for core promoters with a pause site or without a pause site (“Founder Combo”). Transcriptional activity was measured in HepG2 cells when paired with a synthetic HepG2 enhancer (SEQ ID NO: 146) using an MPRA screen. Fold activation of each core promoter with a transcription pause site (“With Pause Site”) relative to a corresponding core promoter without a transcription pause site (“Founder Combo”) was measured in HepG2 cells using an MPRA screen. Sequences with the same pause site sequence of SEQ ID NO: 196 are denoted with red circles. As seen in FIG. 10A and FIG. 10B, activity and fold activation are highly correlated between the sequences with and without a pause site. Additionally, sequences containing pause sites of SEQ ID NO: 196-SEQ ID NO: 198, SEQ ID NO: 211, or SEQ ID NO: 212 consistently showed increased activity relative to the corresponding sequence lacking a pause site. Together, these data suggest that pause sites do not increase activity or activation overall.

### Example 6: Effect of YY1 Motif on Transcriptional Activation

This example describes the effect of a YY1 transcription factor motif in the core promoter on transcription activation. Transcriptional activity of core promoters with and without a YY1 motif after transfection into HepG2 cells was compared. Examples of core promoter sequences with a YY1 motif include SEQ ID NO: 2, SEQ ID NO: 12, and SEQ ID NO: 31-SEQ ID NO: 68. Examples of a YY1 transcription factor motif include SEQ ID NO: 205-SEQ ID NO: 208.

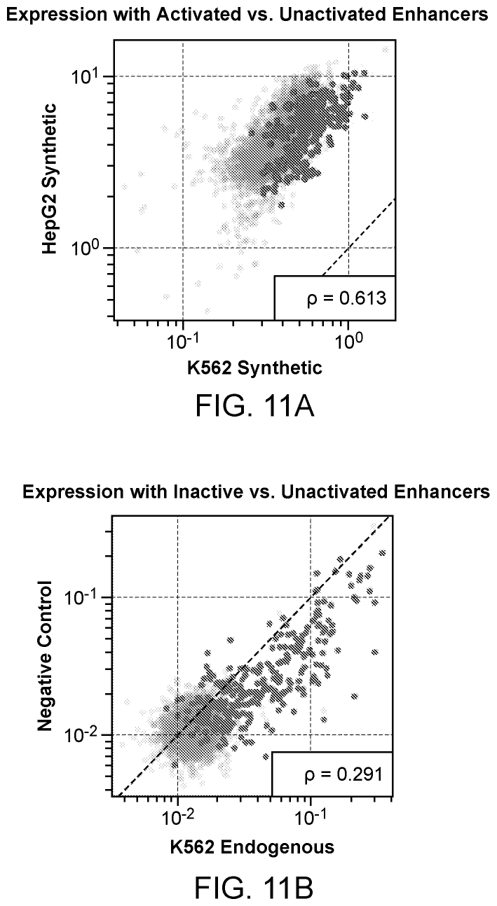

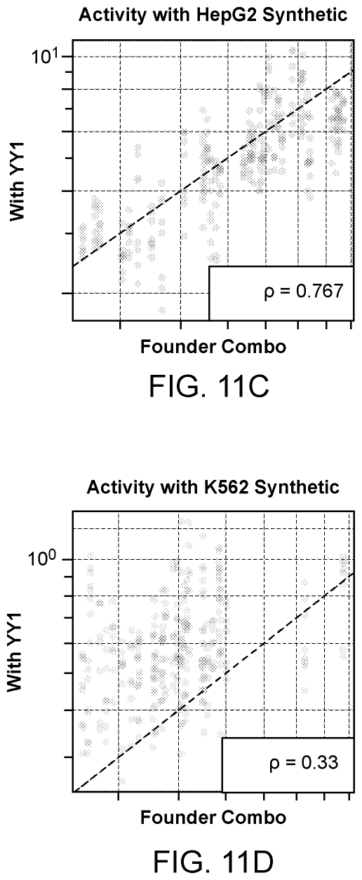

Canonically, a YY1 motif located immediately downstream of the Inr in a core promoter sequence is associated with active promoters. To test the effect of YY1 motifs on cell type-specific activation, the transcriptional activity of 288 fully synthetic promoters with a YY1 motif was compared to core promoters lacking a YY1 motif after transfection into HepG2 cells. As shown in FIG. 11A, core promoters with a YY1 motif exhibited higher transcriptional activity in HepG2 cells than sequences without a YY1 motif specifically when paired with a K562 synthetic enhancer (SEQ ID NO: 145) but not when with a HepG2 synthetic enhancer (SEQ ID NO: 146). This is demonstrated in that the sequences with a YY1 motif, denoted with red circles in FIG. 11A, tended to be clustered to the right and slightly below the majority of sequences lacking a YY1 motif. Transcriptional activity of core promoters with or without a YY1 motif when paired with either a HepG2 synthetic enhancer (SEQ ID NO: 146) or a K562 synthetic enhancer (SEQ ID NO: 145) are shown in FIG. 11C and FIG. 11D, respectively. The data showed that YY1 motifs confer enhancer specificity on a core promoter. Additionally, YY1 motifs increase overall activity, particularly when paired with the K562 synthetic enhancer (SEQ ID NO: 145), as shown in FIG. 11D.

Furthermore, as shown in FIG. 11B, core promoters with a YY1 motif exhibited higher background in HepG2 cells relative to sequences without a YY1 motif. This is demonstrated in that the sequences with a YY1 motif, denoted with red circles in FIG. 11B, showed higher basal transcriptional activity when paired with an inactive enhancer (SEQ ID NO: 146, “Negative Control”) or an endogenous enhancer for a different cell type (SEQ ID NO: 193) compared to core promoters lacking a YY1 motif. Together these data suggest that core promoters with a YY1 motif provide more enhancer-specific transcriptional activation than core promoters without a YY1 motif, with higher overall transcription activity, albeit with elevated background activity.

### Example 7: Transcriptional Activation of ybTATA and minP Variants

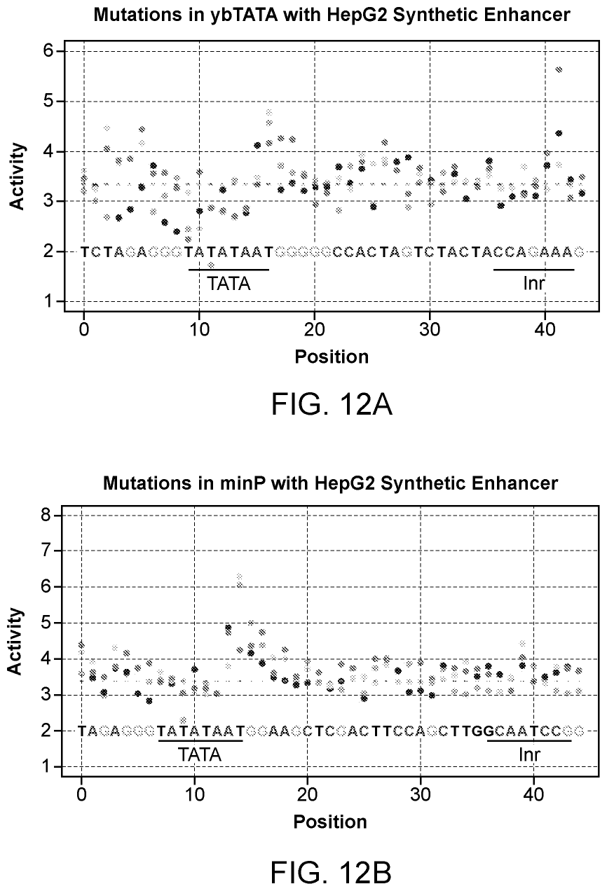

This example describes transcriptional activation of ybTATA and minP variants, including variations in the TATA sequence and the Inr sequence. Transcriptional activity of core promoters with variations in the TATA sequence after transfection into HepG2 cells was compared. Variants of a ybTATA founder sequence (SEQ ID NO: 9) and a minP founder sequence (SEQ ID NO: 5) were paired with a synthetic HepG2 enhancer (SEQ ID NO: 146) screened for transcriptional activation in HepG2. Transcriptional activity of ybTATA variants and minP variants are shown in FIG. 12A and FIG. 12B, respectively. Effect of each nucleotide substitution, A (green circle), T (red circle), C (blue circle), or G (orange circle) at each position within the core promoter sequence on transcriptional activity was compared to the corresponding founder sequence (SEQ ID NO: 9 or SEQ ID NO: 5, dashes). As seen in FIG. 12A and FIG. 12B, substitutions at positions 7 and 8 of the TATA sequence (corresponding to positions 16 and 17 of ybTATA and positions 14 and 15 of minP) increased transcriptional activation. FIG. 13A illustrates the relative transcriptional activity of TATA sequences with each possible nucleotide substitution. Substitutions in position 4 of Inr, corresponding to position 40 of minP, increased transcriptional activity. FIG. 13B illustrates the relative transcriptional activity of Inr sequences with each possible nucleotide substitution. FIG. 13C illustrates the relative transcriptional activity of YY1 motif sequences with each possible nucleotide substitution. FIG. 13D illustrates the relative transcriptional activity of HNF4α motif sequences with each possible nucleotide substitution.

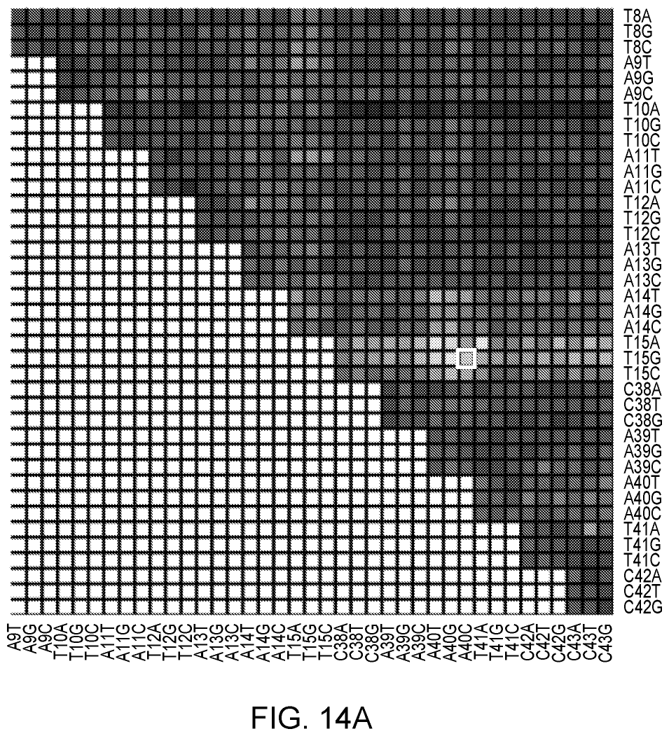

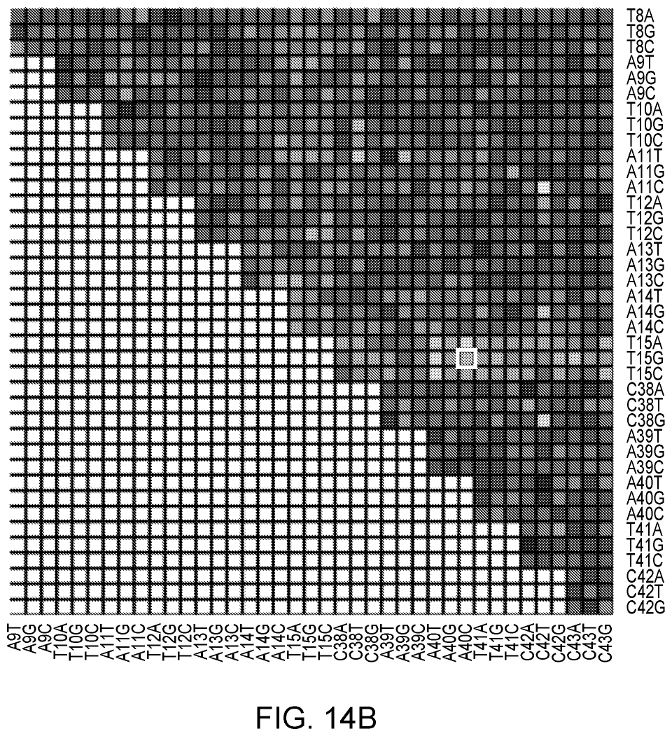

Double mutants were screened in the context of the minP founder sequence (SEQ ID NO: 5), as shown in FIG. 14A and FIG. 14B. Activity of double point mutants of minP (SEQ ID NO: 5) was measured in HepG2 cells when paired with a synthetic HepG2-specific enhancer of SEQ ID NO: 146 (FIG. 14A) or in K562 cells when paired with a synthetic K562-specific enhancer of SEQ ID NO: 145 (FIG. 14B). Lighter shading indicates higher transcriptional activity. Some double mutants with elevated transcriptional activation also showed increases in background activation. The double mutant sequence containing the substitutions T15G and A40C (SEQ ID NO: 15) showed the greatest increase in transcriptional dynamic range. The effect of double mutations on transcriptional activation was consistent with different enhancers.

### Example 8: Characterization of Candidate Switchable Core Promoters

This example describes characterization of candidate switchable core promoters identified using the assays described in EXAMPLE 1-EXAMPLE 8. Candidate switchable core promoters, corresponding to SEQ ID NO: 1-SEQ ID NO: 4, SEQ ID NO: 7, and SEQ ID NO: 10-SEQ ID NO: 15 were selected based on high transcriptional activation activity and low background transcriptional activity, corresponding to high fold activation.

SEQ ID NO: 1, SEQ ID NO: 11, and SEQ ID NO: 14 included a pause site; SEQ ID NO: 2 and SEQ ID NO: 12 included a YY1 motif, SEQ ID NO: 3 included a long spacing between TATA and Inr; SEQ ID NO: 4 included T15A and A40T substitutions relative to minP (SEQ ID NO: 5); SEQ ID NO: 6 (GTACTTATATAAGGGGGTGGGGGCGCGTTCGTCCTCAGTCGCGATCGAACACTCGA GCCGAGCAGACGTGCCTACGGACCG) showed high transcriptional activation but also high background; SEQ ID NO: 7 and SEQ ID NO: 10 included a short spacing between TATA and Inr; SEQ ID NO: 13 was a fully synthetic promoter with strong transcriptional activation; and SEQ ID NO: 15 included T15G and A40C substitutions relative to minP. SEQ ID NO: 8 showed low transcriptional activation and was included as a negative control. SEQ ID NO: 5 (minP) and SEQ ID NO: 9 (ybTATA) were included for comparison.

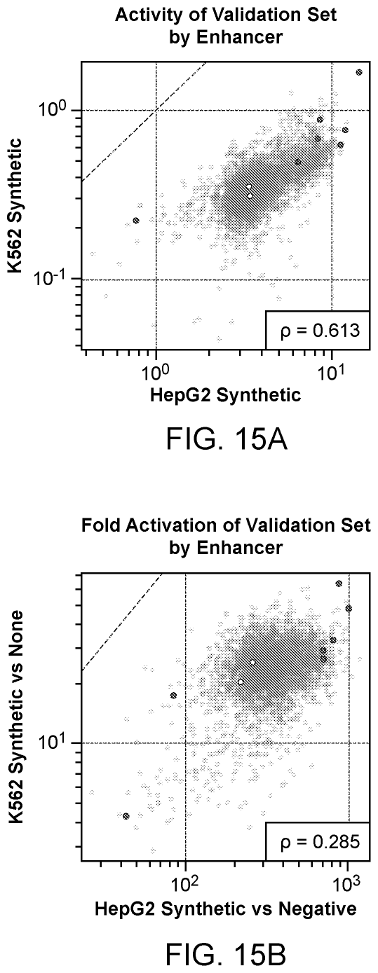

Transcriptional activation properties of SEQ ID NO: 1-SEQ ID NO: 4, SEQ ID NO: 7, and SEQ ID NO: 10-SEQ ID NO: 15 were evaluated in HepG2 cells. FIG. 15A shows transcriptional activity of screened core promoters paired with either a synthetic K562 enhancer (SEQ ID NO: 145) or a synthetic HepG2 enhancer (SEQ ID NO: 146). FIG. 15B shows fold activation of screened core promoters with a synthetic K562 enhancer (SEQ ID NO: 145) relative to no enhancer or with a synthetic HepG2 enhancer (SEQ ID NO: 146) relative to an inactive enhancer (SEQ ID NO: 147, “Negative”). Core promoters ybTATA (SEQ ID NO: 9) and minP (SEQ ID NO: 5) are shown as orange circles in FIG. 15A and FIG. 15B.

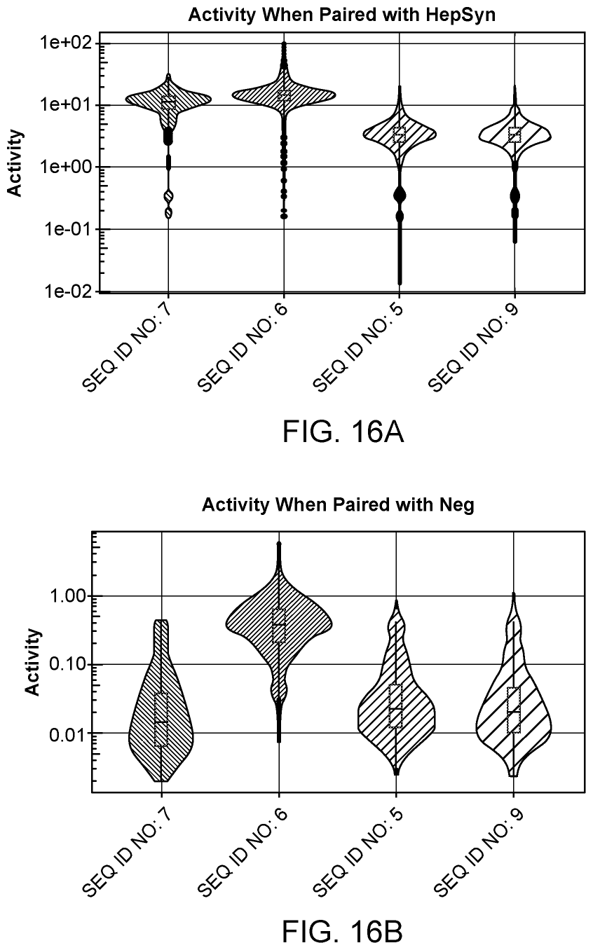

The core promoter of SEQ ID NO: 7 was identified as having high transcriptional activation and high dynamic range. As shown in FIG. 16A, SEQ ID NO: 7 showed high transcriptional activation when paired with a synthetic HepG2 enhancer (SEQ ID NO: 146) in HepG2 cells. Activation by SEQ ID NO: 7 was comparable to the highly active SEQ ID NO: 6 and higher than either of SEQ ID NO: 5 (minP) or SEQ ID NO: 9 (ybTATA). As shown in FIG. 16B, SEQ ID NO: 7 showed low background transcription when paired with an inactive enhancer (SEQ ID NO: 147). Background activation by SEQ ID NO: 7 was lower than any of SEQ ID NO: 6, SEQ ID NO: 5, or SEQ ID NO: 9. The violin plots in FIG. 16A and FIG. 16B show redundant barcodes for each enhancer/promoter construct. Together these data illustrate that the core promoter of SEQ ID NO: 7 functions as a switchable core promoter that promotes high levels of transcription when paired with a cell type-specific enhancer when in that cell type and exhibits low background transcription when paired with a different cell type specific enhancer in the cell type of the previous cell type-specific enhancer or no enhancer in the cell type of the previous cell type-specific enhancer.

### Example 9: Validation of Core Promoter Activity

This example describes the validation of several core promoters in both K562 lymphoblast and HepG2 liver cancer cell lines with either a K562-specific enhancer or a HepG2-specific enhancer (“HSyn”). Screened core promoters included SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15. The core promoters were each cloned into constructs containing either a synthetic K562-specific enhancer or a synthetic HepG2-specific enhancer, and the constructs were tested for transcriptional activity. The core promoter activity was determined using an MPRA screen described in EXAMPLE 1 and using a dual reporter quantitative PCR (qPCR) assay (the dual reporter flow assay as described in EXAMPLE 1, except using qPCR for the readout instead of MFI)

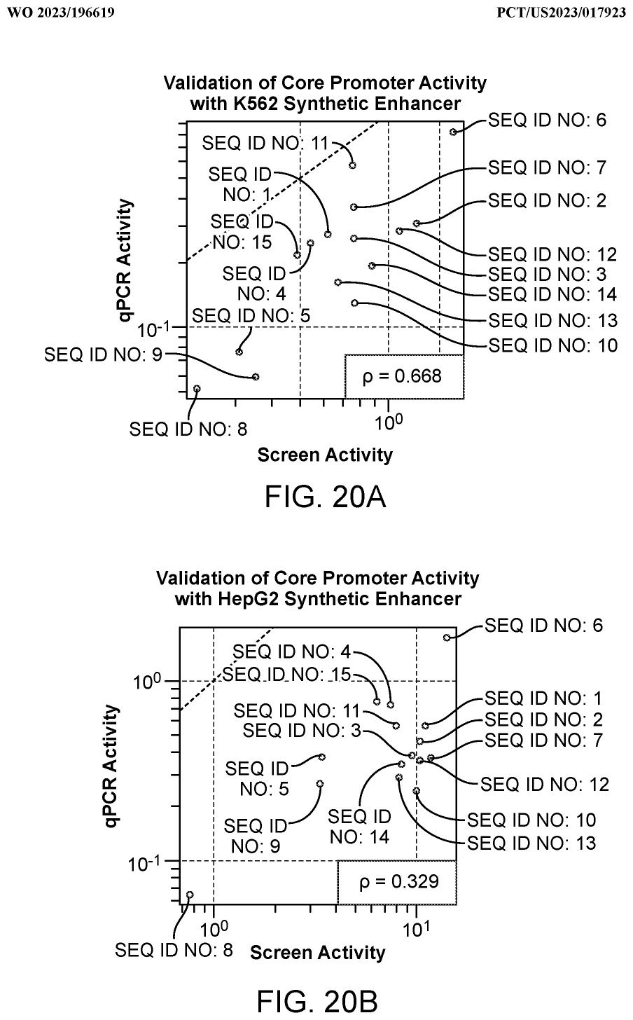

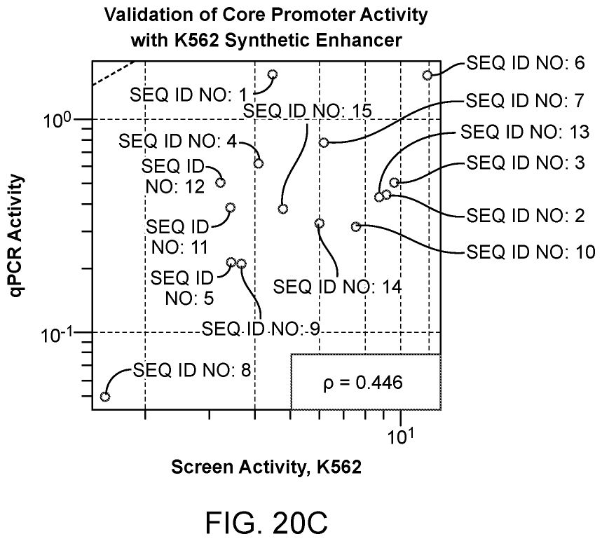

A comparison of promoter activity determined by the MPRA screen (“Screen Activity”) and by dual reporter qPCR assay (“qPCR Activity”) for promoters with a K562-specific enhancer in HepG2 cells is shown in FIG. 20A. A comparison of promoter activity determined by the MPRA screen (“Screen Activity”) and by dual reporter qPCR assay (“qPCR Activity”) for promoters with a HepG2-specific enhancer in HepG2 cells is shown in FIG. 20B. A comparison of promoter activity determined by the MPRA screen (“Screen Activity, K562”) and by dual reporter qPCR assay (“qPCR Activity”) for promoters with a K562-specific enhancer in K562 cells is shown in FIG. 20C. Variants of minP core promoters, including SEQ ID NO: 4 and SEQ ID NO: 15, performed well in both cell lines, exhibiting increased expression when paired with an enhancer of the corresponding cell type. When paired with the enhancer of the corresponding cell type, SEQ ID NO: 4 and SEQ ID NO: 15 exhibited activity that was approximately 3-fold than with a than core promoter of SEQ ID NO: 9. The core promoter of SEQ ID NO: 7 also performed well in K562 cells when paired with the K562-specific enhancer.

### Example 10: Enhanced Activity of a minP Variant Core Promoter

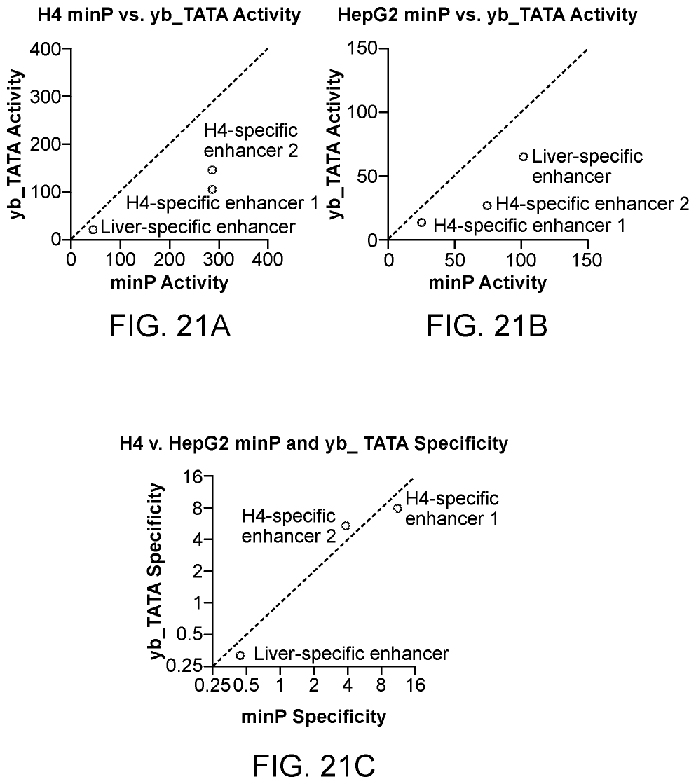

This example describes the activity of a minP variant core promoter relative to a ybTATA core promoter in both H4 neuroglioma and HepG2 liver cancer cell lines. The minP variant core promoter (SEQ ID NO: 4, “minP_T14A_A39T”) and the ybTATA core promoter (SEQ ID NO: 9) were each cloned into three constructs containing one of two different H4-specific enhancers (“H4-specific enhancer 1” and “H4-specific enhancer 2”) or a liver-specific enhancer. Activity of the core promoters in both the H4 and HepG2 cell lines was measured by a dual reporter assay to measure activity. A comparison of the transcriptional activity of the minP variant of SEQ ID NO: 4 (“minP Activity”) and the ybTATA promoter of SEQ ID NO: 9 (“yb_TATA Activity”) in H4 cells when paired with either the H4-specific enhancers or the liver-specific enhancer is shown in FIG. 21A. As seen in FIG. 21A, the minP variant core promoter of SEQ ID NO: 4 exhibited enhanced activity compared to the ybTATA core promoter when paired with the H4-specific enhancers in the H4 cell line. A comparison of the transcriptional activity of the minP variant of SEQ ID NO: 4 (“minP Activity”) and the ybTATA promoter of SEQ ID NO: 9 (“yb_TATA Activity”) in HepG2 liver cells when paired with either the H4-specific enhancers or the liver-specific enhancer is shown in FIG. 21B. As seen in FIG. 21B, the minP variant core promoter of SEQ ID NO: 4 exhibited enhanced activity compared to the ybTATA core promoter in HepG2 cells when paired with the liver-specific enhancer. FIG. 21C shows a comparison of the cell-type specificity of the minP variant of SEQ ID NO: 4 (“minP Specificity”) and the ybTATA promoter of SEQ ID NO: 9 (“yb_TATA Specificity”). As seen in FIG. 21C, the minP variant of SEQ ID NO: 4 exhibits similar specificity with H4-specific enhancers in H4 cells than ybTATA core promoter. Therefore, the minP variant of SEQ ID NO: 4 is superior to the ybTATA promoter because it has similar specificity as, and higher activity than, the ybTATA promoter.

### Example 11: Excitatory Neuron-Specific Transcription of Exogenous Progranulin in the Central Nervous System

This example describes transcription of an excitatory neuron-specific exogenous progranulin in the central nervous system (CNS). A CNS-specific progranulin DNA construct containing a progranulin coding sequence, a CNS-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a CNS-specific transcription factor. The progranulin construct is encapsidated in an adeno-associated virus and delivered to cells of a subject. Optionally, the progranulin construct is integrated into a recombinant polynucleotide cassette comprising one or more of a 5′UTR effector region, 3′UTR effector region, codon optimized progranulin sequence, or introns (natural and/or synthetic) for modulating translation of the RNA coding for progranulin before being encapsidated for AAV delivery. The exogenous progranulin encoded by the progranulin construct is transcribed at higher levels in excitatory neurons than in other non-neuronal cell types or non-CNS tissue, including hepatocytes or liver tissue. CNS-specific transcription of the progranulin transgene results in CNS-specific expression of the exogenous progranulin.

### Example 12: Kidney-Specific Transcription of Exogenous PKD2 in Renal Tissue

This example describes kidneys-specific transcription of exogenous PKD2 in renal tissue. A kidney-specific PKD2 DNA construct containing a protein coding sequence, a kidney-specific enhancer, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a kidney-specific transcription factor. The PKD2 construct is encapsidated in an adeno-associated virus and delivered to cells of a subject. Optionally, the polycystin-2 construct is integrated into a recombinant polynucleotide cassette comprising one or more of a 5′UTR effector region, 3′UTR effector region, codon optimized polycystin-2 sequence, or introns (natural and/or synthetic) for modulating translation of the RNA coding for polycystin-2 before being encapsidated for AAV delivery. The exogenous polycystin-2 protein encoded by the PKD2 construct is transcribed at higher levels in renal tissue than in other non-kidney cell types or non-kidney tissue, including liver tissue. Kidney-specific transcription of the delivered PKD2 transgene results in kidney-specific expression of exogenous polycystin-2.

### Example 13: Excitatory Neuron-Specific Transcription of Exogenous MECP2 in the Central Nervous System

This example describes transcription of an excitatory neuron-specific exogenous MECP2 in the central nervous system (CNS). A CNS-specific MECP2 DNA construct containing an MECP2 coding sequence, a CNS-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a CNS-specific transcription factor. The MECP2 construct is encapsidated in an adeno-associated virus and delivered to cells of a subject. Optionally, the MECP2 construct is integrated into a recombinant polynucleotide cassette comprising one or more of a 5′UTR effector region, 3′UTR effector region, codon optimized progranulin sequence, or introns (natural and/or synthetic) for modulating translation of the RNA coding for MECP2 before being encapsidated for AAV delivery. The exogenous MECP2 encoded by the MECP2 construct is transcribed at higher levels in excitatory neurons than in other non-neuronal cell types or non-CNS tissue, including hepatocytes or liver tissue. CNS-specific transcription of the MECP2 transgene results in CNS-specific expression of the exogenous MECP2.

### Example 14: Rett-Specific Transcription of Exogenous MECP2 in Cells

This example describes transcription of a Rett-specific exogenous MECP2 in cells. A Rett-specific MECP2 DNA construct containing a MECP2 coding sequence, a Rett-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a transcription factor that is only expressed in cells having a Rett phenotype (e.g., cells expressing a non-functional MeCP2 protein) and not in normal celles (e.g., cells expressing functional MeCP2 protein). The Rett-specific MECP2 DNA construct is encapsidated in an adeno-associated virus and delivered to cells of a subject. Optionally, the MECP2 construct is integrated into a recombinant polynucleotide cassette comprising one or more of a 5′UTR effector region, 3′UTR effector region, codon optimized progranulin sequence, or introns (natural and/or synthetic) for modulating translation of the RNA coding for MECP2 before being encapsidated for AAV delivery. The exogenous MECP2 encoded by the MECP2 construct is transcribed at higher levels in Rett cells than in normal cells. Rett-specific transcription of the MECP2 transgene results in Rett-specific expression of the exogenous MECP2.

### Example 15: Treatment of Frontotemporal Dementia Using CNS-Specific Expression of Exogenous Progranulin

This example describes treatment of frontotemporal dementia in a subject by selectively expressing exogenous progranulin in CNS cells. A CNS-specific progranulin DNA construct containing a progranulin coding sequence, a CNS-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a CNS-specific transcription factor. The progranulin construct is encapsidated in an adeno-associated virus (AAV).

The AAV containing the construct is intravenously administered to the subject. Upon administration, the exogenous progranulin encoded by the progranulin construct is transcribed at higher levels in excitatory neurons than in other non-neuronal cell types or non-CNS tissue, including hepatocytes or liver tissue. CNS-specific transcription of the progranulin transgene results in CNS-specific expression of the exogenous progranulin. CNS-specific transcription of the exogenous progranulin alleviates at least one symptom of the frontotemporal dementia or delays the onset of the frontotemporal dementia, thereby treating the frontotemporal dementia in the subject.

### Example 16: Treatment of Polycystic Kidney Disease Using Kidney-Specific Expression of Exogenous PKD2

This example describes treatment of polycystic kidney disease in a subject by selectively expressing exogenous PKD2 in kidney cells. A kidney-specific PKD2 DNA construct containing a PKD2 coding sequence, a kidney-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a kidney-specific transcription factor. The MECP2 construct is encapsidated in an adeno-associated virus (AAV).

The AAV containing the construct is intravenously administered to the subject. Upon administration, the exogenous PKD2 encoded by the MECP2 construct is transcribed at higher levels in kidney cells than in other non-kidney cell types or non-renal tissue, including CNS or liver tissue. Kidney-specific transcription of the PKD2 transgene results in kidney-specific expression of the exogenous PKD2. Kidney-specific transcription of the exogenous PKD2 alleviates at least one symptom of the polycystic kidney disease or cures the polycystic kidney disease, thereby treating the polycystic kidney disease in the subject.

### Example 17: Treatment of Rett Syndrome Using CNS-Specific Expression of Exogenous MECP2

This example describes treatment of Rett syndrome in a subject by selectively expressing exogenous MECP2 in CNS cells. A CNS-specific MECP2 DNA construct containing a MECP2 coding sequence, a CNS-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a CNS-specific transcription factor. The MECP2 construct is encapsidated in an adeno-associated virus (AAV).

The AAV containing the construct is intravenously administered to the subject. Upon administration, the exogenous MECP2 encoded by the MECP2 construct is transcribed at higher levels in excitatory neurons than in other non-neuronal cell types or non-CNS tissue, including hepatocytes or liver tissue. CNS-specific transcription of the MECP2 transgene results in CNS-specific expression of the exogenous MECP2. CNS-specific transcription of the exogenous MECP2 alleviates at least one symptom of the Rett syndrome or cures the Rett syndrome, thereby treating the Rett syndrome in the subject.

### Example 18: Treatment of Rett Syndrome Using Rett-Specific Expression of Exogenous MECP2

This example describes treatment of Rett syndrome in a subject by selectively expressing exogenous MECP2 in Rett cells. A Rett-specific MECP2 DNA construct containing a MECP2 coding sequence, a Rett-specific enhancer region, and a switchable core promoter is constructed. The switchable core promoter is SEQ ID NO: 1-SEQ ID NO: 4, SEQ ID NO: 7, SEQ ID NO: 10-SEQ ID NO: 15, or SEQ ID NO: 110. The enhancer region sequence contains a transcription factor binding site that binds a transcription factor that is only expressed in cells having a Rett phenotype (e.g., cells expressing a non-functional MeCP2 protein) and not in normal celles (e.g., cells expressing functional MeCP2 protein). The Rett-specific MECP2 DNA construct is encapsidated in an adeno-associated virus (AAV). Optionally, the MECP2 construct is integrated into a recombinant polynucleotide cassette comprising one or more of a 5′UTR effector region, 3′UTR effector region, codon optimized progranulin sequence, or introns (natural and/or synthetic) for modulating translation of the RNA coding for MECP2 before being encapsidated for AAV delivery.

The AAV containing the construct is intravenously administered to the subject. Upon administration, the exogenous MECP2 encoded by the MECP2 construct is transcribed at higher levels in Rett cells than in normal cells. Rett-specific transcription of the MECP2 transgene results in Rett-specific expression of the exogenous MECP2. Rett-specific transcription of the MECP2 transgene results in Rett-specific expression of the exogenous MECP2. Rett-specific transcription of the exogenous MECP2 alleviates at least one symptom of the Rett syndrome or cures the Rett syndrome, thereby treating the Rett syndrome in the subject.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An engineered core promoter, wherein the engineered core promoter comprises a sequence having at least 80% sequence identity to SEQ ID NO: 4 and including nucleotide substitutions T15G and A40T relative to SEQ ID NO: 5.

2. The engineered core promoter of claim 1, wherein the engineered core promoter comprises a sequence having at least 90% sequence identity to SEQ ID NO: 4 and including the nucleotide substitutions T15G and A40T relative to SEQ ID NO: 5.

3. The engineered core promoter of claim 1 or claim 2, wherein the engineered core promoter comprises a sequence of SEQ ID NO: 4.

4. The engineered core promoter of any one of claims 1-3, wherein the engineered core promoter further comprises a transcriptional pause site.

5. The engineered core promoter of claim 4, wherein the transcriptional pause site comprises a sequence having at least 90% sequence identity to any one of SEQ ID NO: 196-SEQ ID NO: 198, SEQ ID NO: 211, or SEQ ID NO: 212.

6. The engineered core promoter of claim 4 or claim 5, wherein the transcriptional pause site comprises a sequence of any one of SEQ ID NO: 196-SEQ ID NO: 198, SEQ ID NO: 211, or SEQ ID NO: 212.

7. The engineered core promoter of any one of claims 1-6, wherein the engineered core promoter further comprises a YY1 motif.

8. The engineered core promoter of claim 7, wherein the YY1 motif comprises a sequence having at least 90% sequence identity to any one of SEQ ID NO: 205-SEQ ID NO: 207.

9. The engineered core promoter of claim 7 or claim 8, wherein the YY1 motif comprises a sequence having a sequence of any one of SEQ ID NO: 205-SEQ ID NO: 207.

10. The engineered core promoter of any one of claims 1-9, wherein the engineered core promoter comprises a spacer of 30 to 34 nucleotides.

11. The engineered core promoter of any one of claims 1-10, wherein a transcriptional activity produced by the engineered core promoter paired with an activated response element is higher than a transcriptional activity produced by a core promoter of SEQ ID NO: 6 when paired with the activated response element, a basal transcriptional activity produced by the engineered core promoter paired with an activated response element is lower than a basal transcriptional activity produced by a core promoter of SEQ ID NO: 6 when paired with an inactive response element, or both.

12. The engineered core promoter of any one of claims 1-11, wherein a transcriptional activity produced by the engineered core promoter paired with an activated response element is higher than a transcriptional activity produced by a core promoter of SEQ ID NO: 9 when paired with the activated response element, a basal transcriptional activity produce by the engineered core promoter paired with an activated response element is lower than a basal transcriptional activity produced by a core promoter of SEQ ID NO: 9 when paired with an inactive response element, or both.

13. An engineered promoter comprising a response element and the engineered core promoter of any one of claims 1-12.

14. The engineered promoter of claim 13, wherein the response element confers bone marrow-specific transcription, liver-specific transcription, neuron-specific transcription, muscle-specific transcription, or kidney-specific transcription of a payload under transcriptional control of the engineered promoter.

15. The engineered promoter of claim 13 or claim 14, wherein the response element comprises a sequence having at least 90% sequence identity to SEQ ID NO: 145, SEQ ID NO: 146, SEQ ID NO: 193, or SEQ ID NO: 194.

16. The engineered promoter of any one of claims 13-15, wherein the response element comprises a sequence of SEQ ID NO: 145, SEQ ID NO: 146, SEQ ID NO: 193, or SEQ ID NO: 194.

17. A recombinant polynucleotide comprising the engineered core promoter of any one of claims 1-12 or the engineered promoter of any one of claims 13-16 and a payload comprising a coding sequence under transcriptional control of the engineered core promoter.

18. The recombinant polynucleotide of claim 17, wherein the payload encodes a protein.

19. The recombinant polynucleotide of claim 18, wherein the protein is a neuronal protein, a kidney protein, a retinal protein, a muscle protein, or an apoptosis-inducing protein.

20. The recombinant polynucleotide of claim 18 or claim 19, wherein the protein is associated with a genetic disorder, a neuronal disorder, a kidney disorder, an eye disorder, a muscular disorder, or a cancer.

21. The recombinant polynucleotide of any one of claims 18-20, wherein the protein is progranulin, MeCP2, polycystin-1, or polycystin-2.

22. The recombinant polynucleotide of claim 17, wherein the payload encodes a therapeutic polynucleotide.

23. The recombinant polynucleotide of claim 22, wherein the therapeutic polynucleotide is a guide RNA or a suppressor tRNA.

24. The recombinant polynucleotide of claim 22 or claim 23, wherein the therapeutic polynucleotide targets a gene.

25. The recombinant polynucleotide of claim 24, wherein the gene is associated with a genetic disorder, a neuronal disorder, a kidney disorder, an eye disorder, a muscular disorder, or a cancer.

26. The recombinant polynucleotide of claim 24 or claim 25, wherein the gene is GRN, MECP2, PKD2, or PKD2.

27. An engineered viral vector comprising the engineered core promoter of any one of claims 1-12, the engineered promoter of any one of claims 13-16, or the recombinant polynucleotide of any one of claims 17-26 in a viral vector.

28. The engineered viral vector of claim 27, wherein the viral vector is an adenoviral vector, an adeno-associated viral vector, or a lentivector.

29. The engineered viral vector of claim 28, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP. V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, and combinations thereof.

30. A pharmaceutical composition comprising the engineered core promoter of any one of claims 1-12, the engineered promoter of any one of claims 13-16, the recombinant polynucleotide of any one of claims 17-26, or the viral vector of any one of claims 27-29, and a pharmaceutically acceptable carrier.

31. A method of treating a disorder in a subject in need thereof, the method comprising:
administering to the subject a composition comprising the recombinant polynucleotide of any one of claims 17-26, the viral vector of any one of claims 27-29, or the pharmaceutical composition of claim 30; and
expressing a therapeutic sequence encoded by a payload of the recombinant polynucleotide in a target cell of the subject, thereby treating the disorder.

32. The method of claim 31, wherein the target cell is a cell type or cell state associated with the disorder. 33 The method of claim 31 or claim 32, wherein the disorder is a genetic disorder, a neuronal disorder, a kidney disorder, an eye disorder, a muscular disorder, or a cancer.

34. The method of any one of claims 31-33, wherein the disorder is Rett syndrome, MECP2 duplication syndrome, frontotemporal dementia, neuronal ceroid lipofuscinosis, cancer, atherosclerosis, Alzheimer's disease, amyotrophic lateral sclerosis, limbic predominant age-related TDP-43 encephalopathy, or polycystic kidney disease.

35. The method of any one of claims 31-34, wherein the therapeutic sequence encodes a therapeutic protein.

36. The method of claim 35, wherein the therapeutic protein is a neuronal protein, a kidney protein, a retinal protein, a muscle protein, or an apoptosis-inducing protein.

37. The method of claim 35 or claim 36, wherein the therapeutic protein is associated with a genetic disorder, a neuronal disorder, a kidney disorder, an eye disorder, a muscular disorder, or a cancer.

38. The method of any one of claims 35-37, wherein the therapeutic protein is MECP2, progranulin, polycystin-1, or polycystin-2.

39. The method of any one of claims 31-34, wherein the payload encodes a therapeutic polynucleotide.

40. The method of claim 39, wherein the therapeutic polynucleotide is a guide RNA or a suppressor tRNA.

41. A method of identifying a switchable core promoter, the method comprising:
introducing a core promoter library comprising a first sub-library and a second sub-library to a population of cells;
wherein the first sub-library comprises a plurality of core promoters, wherein a core promoter of the plurality of core promoters is linked to a first response element, and a unique barcode sequence;
wherein the second sub-library comprises the plurality of core promoters, wherein the core promoter of the plurality of promoters is linked to a second response element and, a unique barcode sequence; and
identifying a switchable core promoter as the core promoter that promotes higher transcription of the unique barcode when paired with the first response element than when paired with the second response element.

42. The method of claim 41, further comprising activating the first response element.

43. The method of claim 41 or claim 42, wherein the second response element is not activated.

44. The method of any one of claims 41-43, wherein the first response element is an activated response element and the second enhancer sequence is an inactive response element or an unactivated response element.
